(19) United States
(12) Patent Application Publication
(10) Pub. No.: US 2024/0398862 A1
(43) Pub. Date: Dec. 5, 2024

(54) GENE TARGETS FOR MANIPULATING T CELL BEHAVIOR

(71) Applicants: THE REGENTS OF THE UNIVERSITY OF CALIFORNIA, Oakland, CA (US); THE BOARD OF TRUSTEES OF THE LELAND STANFORD JUNIOR UNIVERSITY, Stanford, CA (US)

(72) Inventors: Jacob FREIMER, Oakland, CA (US); Alexander MARSON, Oakland, CA (US); Jonathan PRITCHARD, stanford, CA (US); Oren SHAKED, Oakland, CA (US)

(21) Appl. No.: 17/754,848

(22) PCT Filed: Oct. 15, 2020
(86) PCT No.: PCT/US2020/055764

§ 371 Date: Apr. 13, 2022

Related U.S. Application Data
(60) Provisional application No. 62/915,494, filed on Oct. 15, 2019.

(51) Int. Cl. A61K 35/17; A61K 39/00; C12N 5/0783

(52) U.S. Cl. CPC A61K 35/17; A61K 39/4611; C12N 5/0637; C12N 2510/00

(57) ABSTRACT

Provided herein are compositions and methods for modifying T cells. The disclosure is based, in part, on the use of sgRNA lentiviral infection with Cas9 protein electroporation (SLICE), to identify regulators of IL2RA, IL-2, CTLA4, arnd FOXP3 in effector T cells. IL2RA, IL-2, CTLA4, and FOXP3 are key genes in immune regulation that have been implicated in autoimmune disease and cancer. Therefore, modulating expression of these genes in T cells, for example, effector T cells or regulatory T cells, could have therapeutic applications.

### PRIOR RELATED APPLICATION

This application claims the benefit of and priority to U.S. Provisional Application No. 62/915,494, filed on Oct. 15, 2019, which is hereby incorporated by reference in its entirety.

### STATEMENT AS TO RIGHTS TO INVENTIONS MADE UNDER FEDERALLY SPONSORED RESEARCH AND DEVELOPMENT

This invention was made with government support under grant no. R01 HG008140 awarded by the National Institutes of Health. The government has certain rights in the invention.

### INCORPORATION BY REFERENCE OF SEQUENCE LISTING

This application contains a Sequence Listing in computer readable form (filename: 081906_1211947_SeqList.txt; Size: 491 KB; created Oct. 12, 2020); which is incorporated by reference in its entirety and forms part of the disclosure.

### BACKGROUND OF THE INVENTION

Decades of work in animal models and cell lines have identified regulators of T cell suppression and activation, but systematic strategies to comprehensively analyze the function of genes that regulate human T cell responses are still lacking. T cells play a role in regulating the immune response in cancer as well as other diseases, for example, autoimmune diseases. Methods of modifying T cells for the treatment of autoimmune diseases or cancer have great therapeutic potential.

### BRIEF SUMMARY OF THE INVENTION

The disclosure is based, in part, on the use of sgRNA lentiviral infection with Cas9 protein electroporation (SLICE), to identify regulators of IL2RA, IL-2, CTLA4, and FOXP3 in effector T cells. IL2RA, IL-2, CTLA4, and FOXP3 are key genes in immune regulation that have been implicated in autoimmune disease and cancer. Therefore, modulating expression of these genes in T cells, for example, effector T cells or regulatory T cells, could have therapeutic applications.

The present invention is directed to compositions and methods for modifying T cells. The inventors have identified nuclear factors that influence expression of IL2RA, IL-2, CTLA4 and FOXP3. T cells can be modified by inhibiting and/or overexpressing one or more of these nuclear factors to manipulate immune cell activity. In some examples, modified T cells are used to treat autoimmune disorders, assist in organ transplantation, to treat graft versus host disease, or inflammation. Examples of autoimmune/inflammatory diseases include but are not limited to: type 1 diabetes, rheumatoid arthritis, inflammatory bowel disease, multiple sclerosis, and multi-organ autoimmune syndromes. In other examples, modified T cells are used to treat cancer. For example, in some embodiments, T cells can be used to target hematological malignancies or solid tumors. Examples of such cancers include but are not limited to, ovarian cancer breast cancers, colon cancers, lung cancers, prostate cancers, liver cancers, bone and soft tissue cancers, head and neck cancers, melanomas and other skin cancers, brain cancers, leukemias, lymphomas.

Provided herein is a T cell comprising: (a) a genetic modification or heterologous polynucleotide that inhibits expression of a nuclear factor set forth in Table 1, Table 2, Table 3, Table 4, Table 5, Table 6, Table 7, Table 8, Table 9, Table 10, Table 11, Table 12, Table 13 or Table 14; and/or (b) a heterologous polynucleotide that encodes a nuclear factor set forth in Table 1, Table 2, Table 3, Table 4, Table 5, Table 6, Table 7, Table 8, Table 9, Table 10, Table 11, Table 12, Table 13 or Table 14.

In some embodiments, the T cell comprises (a) a genetic modification or heterologous polynucleotide that inhibits expression of CBFB, MYB, ZNF217, FOXK1, FLI1, FOS, SATB1, IL2, ATXN7L3, MTF1, RELA, IRF1, BCL11B, STAT3, MED30, MED14, MED11, IKZF3, KMT2A, IKZF1, MED12, TAF5L, PTEN, IRF4, FOXO1, FOXP1, CTLA4, ETS1, MYBL2, TP53, MBD2, ZBTB7A, DNMT1, HIVEP2, KLF2, TFDP1, SMARCB1, MAF, FOXP3, GATA3, STAT5B, STAT5A, PRDM1, TNFAIP3, RXRB, TFDP1, CXXC1, NFATC2, MAF, IRF2, ZBTB11, JAK3, YY1, IL2RA or GTF2B; and/or (b) a heterologous polynucleotide that encodes CBFB, MYB, ZNF217, FOXK1, FLI1, FOS, SATB1, IL2, ATXN7L3, MTF1, RELA, IRF1, BCL11B, STAT3, MED30, MED14, MED11, IKZF3, KMT2A, IKZF1, MED12, TAF5L, PTEN, IRF4, FOXO1, FOXP1, CTLA4, ETS1, MYBL2, TP53, MBD2, ZBTB7A, DNMT1, TFDP1, SMARCB1, MAF, FOXP3, GATA3, STAT5B, STAT5A, PRDM1, TNFAIP3, RXRB, TFDP1, CXXC1, NFATC2, MAF, IRF2, ZBTB11, JAK3, YY1, IL2RA or GTF2B.

In some embodiments, the T cell comprises a genetic modification or heterologous polynucleotide that inhibits expression of a nuclear factor set forth in Table 1; and/or a heterologous polynucleotide that encodes a nuclear factor set forth in Table 2, wherein expression of CTLA4 is increased in the T cell relative to expression of CTLA4 in a T cell not comprising the genetic modification or heterologous polynucleotide.

In some embodiments, the T cell comprises: (a) a genetic modification or a heterologous polynucleotide that inhibits expression of CBFB, MYB, ZNF217, FOXK1, FLI1, FOS, SATB1, IL2 or ATXN7L3, wherein expression of CTLA4 is increased in the T cell relative to expression of CTLA4 in a T cell not comprising the genetic modification or the heterologous polynucleotide that inhibits expression of CBFB, MYB, ZNF217, FOXK1, FLI1, FOS, SATB1, IL2 or ATXN7L3; and/or (b) a heterologous polynucleotide that encodes MTF1, RELA, IRF1, BCL11B, STAT3, MED30, MED14, MED11, IKZF3, KMT2A, IKZF1, MED12, TAF5L, PTEN, IRF4, FOXO1, FOXP1 or CTLA4, wherein expression of CTLA4 is increased in the T cell relative to expression of CTLA4 in a T cell not comprising the heterologous polynucleotide that encodes MTF1, RELA, IRF1, BCL11B, STAT3, MED30, MED14, MED11, IKZF3, KMT2A, IKZF1, MED12, TAF5L, PTEN, IRF4, FOXO1, FOXP1 or CTLA4.

In some embodiments, the T cell comprises a genetic modification or heterologous polynucleotide that inhibits expression of a nuclear factor set forth in Table 2, and/or a heterologous polynucleotide that encodes a nuclear factor set forth in Table 1, and wherein expression of CTLA4 is decreased in the T cell relative to expression of CTLA4 in a T cell not comprising the genetic modification or heterologous polynucleotide.

In some embodiments, the T cell comprises: (a) a genetic modification or heterologous polynucleotide that inhibits expression of MTF1, RELA, IRF1, BCL11B, STAT3, MED30, MED14, MED11, IKZF3, KMT2A, IKZF1, MED12, TAF5L, PTEN, IRF4, FOXO1, FOXP1 or CTLA4, wherein expression of CTLA4 is decreased in the T cell relative to expression of CTLA4 in a T cell not comprising the genetic modification or the heterologous polynucleotide that inhibits expression of MTF1, RELA, IRF1, BCL11B, STAT3, MED30, MED14, MED11, IKZF3, KMT2A, IKZF1, MED12, TAF5L, PTEN, IRF4, FOXO1, FOXP1 or CTLA4; and/or (b) a heterologous polynucleotide that encodes CBTB, MYB, ZNF217, FOXK1, FLI1, FOX, SATB1, IL2 or ATXN7L3, wherein expression of CTLA4 is decreased in the T cell relative to expression of CTLA4 in a T cell not comprising the heterologous polynucleotide that encodes CBTB, MYB, ZNF217, FOXK1, FLI1, FOX, SATB1, IL2 or ATXN7L3.

In some embodiments, the T cell comprises a genetic modification or heterologous polynucleotide that inhibits expression of a nuclear factor set forth in Table 3 and/or a heterologous polynucleotide that encodes a nuclear factor set forth in Table 4, and wherein expression of FOXP3 is increased in the T cell relative to expression of FOXP3 in a T cell not comprising the genetic modification or heterologous polynucleotide.

In some embodiments, the T cell comprises: (a) a genetic modification or heterologous polynucleotide that inhibits expression of ETS1, MYBL2, MYB, TP53, FLI1, SATB1, MBD2, ZBTB7A, DNMT1, TFDP1, SMARCB1 or MAF, wherein expression of FOXP3 is increased in the T cell relative to expression of FOXP3 in a T cell not comprising the genetic modification or heterologous polynucleotide that inhibits expression of ETS1, MYBL2, MYB, TP53, FLI1, SATB1, MBD2, ZBTB7A, DNMT1, TFDP1, SMARCB1 or MAF; and/or (b) a heterologous polynucleotide that encodes a TAF5L, FOXP3, GATA3, STAT5B, FOXP1, STAT5A, PTEN or FOXO1, wherein expression of FOXP3 is increased in the T cell relative to expression of FOXP3 in a T cell not comprising a heterologous polynucleotide that encodes a TAF5L, FOXP3, GATA3, STAT5B, FOXP1, STAT5A, PTEN or FOXO1.

In some embodiments, the T cell comprises a genetic modification or heterologous polynucleotide that inhibits expression of a nuclear factor set forth in Table 4, and/or a heterologous polynucleotide that encodes a nuclear factor set forth in Table 3, and wherein expression of FOXP3 is decreased in the T cell relative to expression of FOXP3 in a T cell not comprising the genetic modification or heterologous polynucleotide.

In some embodiments, the T cell comprises: (a) a genetic modification or heterologous polynucleotide that inhibits expression of TAF5L, FOXP3, GATA3, STAT5B, FOXP1, STAT5A, PTEN or FOXO1, wherein expression of FOXP3 is decreased in the T cell relative to expression of FOXP3 in a T cell not comprising the genetic modification or heterologous polynucleotide that inhibits expression of TAF5L, FOXP3, GATA3, STAT5B, FOXP1, STAT5A, PTEN or FOXO1; and/or (b) a heterologous polynucleotide that encodes ETS1, MYBL2, MYB, TP53, FLI1, SATB1, MBD2, ZBTB7A, DNMT1, TFDP1, SMARCB1 or MAF, wherein expression of FOXP3 is decreased in the T cell relative to expression of FOXP3 in a T cell not comprising a heterologous polynucleotide that encodes ETS1, MYBL2, MYB, TP53, FLI1, SATB1, MBD2, ZBTB7A, DNMT1, TFDP1, SMARCB1 or MAF.

In some embodiments, the T cell comprises a genetic modification or heterologous polynucleotide that inhibits expression of a nuclear factor set forth in Table 5, and/or a heterologous polynucleotide that encodes a nuclear factor set forth in Table 6, and wherein expression of IL-2 is increased in the T cell relative to expression of IL-2 in a T cell not comprising the genetic modification or heterologous polynucleotide.

In some embodiments, the T cell comprises: (a) a genetic modification or heterologous polynucleotide that inhibits expression of MED12, FOXP1, PTEN, IKZF1, TAF5L, PRDM1, TFDP1, CXXC1, IKZF3 or TP53, wherein expression of IL-2 is increased in the T cell relative to expression of IL-2 in a T cell not comprising the genetic modification or heterologous polynucleotide that inhibits expression of MED12, FOXP1, PTEN, IKZF1, TAF5L, PRDM1, TFDP1, CXXC1, IKZF3 or TP53; and/or (b) a heterologous polynucleotide that encodes NFATC2, MAF, ZBTB7A, MBD2, GATA3, MED14, IRF2, MED30, ZBTB11, RELA, JAK3, MED11, BCL11B, MTF1, ATXN7L3, YY1, ETS1, IL2, DNMT1, GTF2B or SMARCB1, wherein expression of IL-2 is increased in the T cell relative to expression of IL-2 in a T cell not comprising a heterologous polynucleotide that encodes NFATC2, MAF, ZBTB7A, MBD2, GATA3, MED14, IRF2, MED30, ZBTB11, RELA, JAK3, MED11, BCL11B, MTF1, ATXN7L3, YY1, ETS1, IL2, DNMT1, GTF2B or SMARCB1.

In some embodiments, the T cell comprises a genetic modification or heterologous polynucleotide that inhibits expression of a nuclear factor set forth in Table 6, and/or a heterologous polynucleotide that encodes a nuclear factor set forth in Table 5, and wherein expression of IL-2 is decreased in the T cell relative to expression of IL-2 in a T cell not comprising the genetic modification or heterologous polynucleotide.

In some embodiments, the T cell comprises (a) genetic modification or heterologous polynucleotide that inhibits expression of NFATC2, MAF, ZBTB7A, MBD2, GATA3, MED14, IRF2, MED30, ZBTB11, RELA, JAK3, MED11, BCL11B, MTF1, ATXN7L3, YY1, ETS1, IL2, DNMT1, GTF2B or SMARCB1, wherein expression of IL-2 is decreased in the T cell relative to expression of IL-2 in a T cell not comprising the genetic modification or heterologous polynucleotide that inhibits expression of NFATC2, MAF, ZBTB7A, MBD2, GATA3, MED14, IRF2, MED30, ZBTB11, RELA, JAK3, MED11, BCL11B, MTF1, ATXN7L3, YY1, ETS1, IL2, DNMT1, GTF2B or SMARCB1; and/or (b) a heterologous polynucleotide that encodes MED12, FOXP1, PTEN, IKZF1, TAF5L, PRDM1, TFDP1, CXXC1, IKZF3 or TP53, wherein expression of IL-2 is decreased in the T cell relative to expression of IL-2 in a T cell not comprising heterologous polynucleotide that encodes MED12, FOXP1, PTEN, IKZF1, TAF5L, PRDM1, TFDP1, CXXC1, IKZF3 or TP53.

In some embodiments, the T cell comprises: (a) a genetic modification or heterologous polynucleotide that inhibits expression of a nuclear factor set forth in Table 7, Table 9, Table 11 or Table 13; and/or (b) a heterologous polynucleotide that encodes a nuclear factor set forth in Table 8, Table 10, Table 12, or Table 14 and wherein expression of IL2RA is increased in the T cell relative to expression of IL2RA in a T cell not comprising the genetic modification or heterologous polynucleotide.

In some embodiments, the T cell comprises: (a) a genetic modification or heterologous polynucleotide that inhibits expression of MED12, CBFB, HIVEP2, KLF2, MYB, FOXK1, ZNF217, IRF2, TNFAIP3, MYC, PRDM1, TFDP1, IRF1, FOXO1, ATXN7L3 or TP53, wherein expression of IL2RA is increased in the T cell relative to expression of IL2RA in a T cell not comprising the genetic modification or heterologous polynucleotide that inhibits expression of MED12, CBFB, HIVEP2, KLF2, MYB, FOXK1, ZNF217, IRF2, TFNAIP3, MYC, PRDM1, TFDP1, IRF1, FOXO1, ATXN7L3 or TP53; and/or (b) a heterologous polynucleotide that encodes IKZF3, YY1, MBD2, IRF4, IKZF1, RXRB, RELA, ETS1, KMT2A, PTEN, JAK3, STAT5A, GATA3, FOXP1, STAT5B, or IL2RA, wherein expression of IL2RA is increased in the T cell relative to expression of IL2RA in a T cell not comprising the heterologous polynucleotide that encodes IKZF3, YY1, MBD2, IRF4, IKZF1, RXRB, RELA, ETS1, KMT2A, PTEN, JAK3, STAT5A, GATA3, FOXP1, STAT5B, or IL2RA.

In some embodiments, the T cell comprises: (a) a genetic modification or heterologous polynucleotide that inhibits expression of a nuclear factor set forth in Table 8, Table 10, Table 12 or Table 14, and/or a (b) a heterologous polynucleotide that encodes a nuclear factor set forth in Table 7, Table 9, Table 11 or Table 13 and wherein expression of IL2RA is decreased in the T cell relative to expression of IL2RA in a T cell not comprising the genetic modification or heterologous polynucleotide.

In some embodiments, the T cell comprises: (a) a genetic modification or heterologous polynucleotide that inhibits expression of IKZF3, YY1, MBD2, IRF4, IKZF1, RXRB, RELA, ETS1, KMT2A, PTEN, JAK3, STAT5A, GATA3, FOXP1, STAT5B, or IL2RA, wherein expression of IL2RA is decreased in the T cell relative to expression of IL2RA in a T cell not comprising the genetic modification or heterologous polynucleotide that inhibits expression of IKZF3, YY1, MBD2, IRF4, IKZF1, RXRB, RELA, ETS1, KMT2A. PTEN, JAK3, STAT5A, GATA3, FOXP1, STAT5B, or IL2RA; and/or (b) a heterologous polynucleotide that encodes MED12, CBFB, HIVEP2, KLF2, MYB, FOXK1, ZNF217, IRF2, TFNAIP3, MYC, PRDM1, TFDP1, IRF1, FOXO1, ATXN7L3 or TP53, wherein expression of IL2RA is decreased in the T cell relative to expression of IL2RA in a T cell not comprising heterologous polynucleotide that encodes MED12, CBFB, HIVEP2, KLF2, MYB, FOXK1, ZNF217, IRF2, TFNAIP3, MYC, PRDM1, TFDP1, IRF1, FOXO1, ATXN7L3 or TP53.

In some embodiments, the T cell is a Treg cell. In some embodiments, the T cell is a conventional T cell, for example, a CD8+, CD4+ T cell or a CD4+CD8+ cell. Also provided, are populations of cells comprising any of the genetically modified T cells provided herein.

Also provided is a method of making a modified T cell, the method comprising: inhibiting expression of one or more nuclear factors set forth in Table 1, Table 2, Table 3, Table 4, Table 5, Table 6, Table 7, Table 8, Table 9, Table 10, Table 11, Table 12, Table 13 or Table 14 and/or overexpressing one or more nuclear factors set forth in Table 1, Table 2, Table 3, Table 4, Table 5, Table 6, Table 7, Table 8, Table 9, Table 10, Table 11, Table 12, Table 13 or Table 14.

In some embodiments, the method comprises: (a) inhibiting expression of one or more nuclear factors selected from the group consisting of CBFB, MYB, ZNF217, FOXK1, FLI1, FOS, SATB1, IL2, ATXN7L3, MTF1, RELA, IRF1, BCL11B, STAT3, MED30, MED14, MED11, IKZF3, KMT2A, IKZF1, MED12, TAF5L, PTEN, IRF4, FOXO1, FOXP1, CTLA4, ETS1, MYBL2, TP53, MBD2, ZBTB7A, DNMT1, HIVEP2, KLF2, TFDP1, SMARCB1, MAF, FOXP3, GATA3, STAT5B, STAT5A, PRDM1, TNFAIP3, RXRB, TFDP1, CXXC1, NFATC2, MAF, IRF2, ZBTB11, JAK3, YY1, IL2RA and GTF2B; and/or (b) overexpressing one or more nuclear factors selected from the group consisting of CBFB, MYB, ZNF217, FOXK1, FLI1, FOS, SATB1, IL2, ATXN7L3, MTF1, RELA, IRF1, BCL11B, STAT3, MED30, MED14, MED11, IKZF3, KMT2A, IKZF1, MED12, TAF5L, PTEN, IRF4, FOXO1, FOXP1, CTLA4, ETS1, MYBL2, TP53, MBD2, ZBTB7A, DNMT1, HIVEP2, KLF2, TFDP1, SMARCB1, MAF, FOXP3, GATA3, STAT5B, STAT5A, PRDM1, TNFAIP3, RXRB, TFDP1, CXXC1, NFATC2, MAF, IRF2, ZBTB11, JAK3, YY1, IL2RA and GTF2B.

In some embodiments, inhibiting expression of one or more nuclear factors comprises reducing expression of the nuclear factor, or reducing expression of a polynucleotide encoding the nuclear factor. In some embodiments, inhibiting comprises contacting a polynucleotide encoding the nuclear factor with a targeted nuclease, a guide RNA (gRNA), an siRNA, an antisense RNA, microRNA (miRNA), or short hairpin RNA (shRNA). In some embodiments, inhibiting comprises contacting the polynucleotide encoding the nuclear factor with at least one gRNA and optionally a targeted nuclease, wherein the at least one gRNA comprises a sequence selected from one or more of Tables 1-8. In some embodiments, inhibiting comprises mutating the polynucleotide encoding the nuclear factor. In some embodiments, inhibiting comprises contacting the polynucleotide encoding the nuclear factor with a targeted nuclease. In some embodiments, inhibiting comprises performing clustered regularly interspaced short palindromic repeats (CRISPR)/Cas genome editing.

In some embodiments, the targeted nuclease introduces a double-stranded break in a target region in the polynucleotide encoding the nuclear factor. In some embodiments, the targeted nuclease is an RNA-guided nuclease. In some embodiments, the RNA-guided nuclease is a Cpf1 nuclease or a Cas9 nuclease and the method further comprises introducing into a T cell a gRNA that specifically hybridizes to a target region in the polynucleotide. In some embodiments, the Cpf1 nuclease or the Cas9 nuclease and the gRNA are introduced into the T cell as a ribonucleoprotein (RNP) complex.

In some embodiments, the genetically modified T cell is administered to a human following inhibition of one or more nuclear factors or overexpression of one or more nuclear factors. In some embodiments, the T cell is obtained from a human prior to treating the T cell to inhibit expression of one or more nuclear factors and/or overexpress one or more nuclear factors, and the treated T cell is reintroduced into a human. In any of the methods provided herein, the T cell can be, for example a Treg cell, a CD8+ cell, CD4+ cell or a CD8+CD4+ cell. In another embodiment, provided herein is a T cell made by any of the methods described herein. Populations of T cells made by any of the methods described herein are also provided.

In some embodiments, expression of one or more nuclear factors set forth in Table Table 2, Table 4, Table 5, Table 7, Table 9, Table 11 or Table 13 is inhibited in the T cell obtained from the human. In some embodiments, expression of one or more nuclear factors set forth in Table 2, Table 4, Table 5, Table 7, Table 9, Table 11 or Table 13 is inhibited in the T cell obtained from a human that has cancer.

In some embodiments, expression of one or more nuclear factors selected from the group consisting of CBFB, MYB, ZNF217, FOXK1, FLI1, FOS, SATB1, IL2, and ATXN7L3 is inhibited in the T cell obtained from a human that has cancer.

In some embodiments, expression of one or more nuclear factors selected from the group consisting of ETS1, MYBL2, MYB, TP53, FLI1, SATB1, MBD2, ZBTB7A, DNMT1, TFDP1, SMARCB1 or MAF is inhibited in the T cell obtained from a human that has cancer.

In some embodiments, expression of one or more nuclear factors selected from the group consisting of NFATC2, MAF, ZBTB7A, MBD2, GATA3, MED14, IRF2, MED30, ZBTB11, RELA, JAK3, MED11, BCL11B, MTF1, ATXN7L3, YY1, ETS1, IL2, DNMT1, GTF2B and SMARCB1 is inhibited in the T cell obtained from a human that has cancer.

In some embodiments, expression of one or more nuclear factors selected from the group consisting of IKZF3, YY1, MBD2, IRF4, IKZF1, RXRB, RELA, ETS1, KMT2A, PTEN, JAK3, STAT5A, GATA3, FOXP1, STAT5B, and IL2RA is inhibited in the T cell obtained from a human that has cancer.

In some embodiments, expression of one or more nuclear factors set forth in Table 7, Table 9 or Table 13 is inhibited to increase IL2RA expression in a conventional T cell, wherein the subject has cancer.

In some embodiments, expression of one or more nuclear factors selected from the group consisting of MED12, CBFB, HIVEP2, KLF2, MYB, FOXK1, ZNF217, IRF2, TFNAIP3, MYC, PRDM1, TFDP1, IRF1, FOXO1, ATXN7L3 and TP53 is inhibited to increase IL2RA expression in a effector T cell, and wherein the subject has cancer.

In some embodiments, expression of one or more nuclear factors set forth in Table 8, Table 10 or Table 14 is inhibited to decrease IL2RA expression in a regulatory T cell, and wherein the subject has an autoimmune disorder.

In some embodiments, expression of one or more nuclear factors selected from the group consisting of IKZF3, YY1, MBD2, IRF4, IKZF1, RXRB, RELA, ETS1, KMT2A, PTEN, JAK3, STAT5A, GATA3, FOXP1, STAT5B, and IL2RA is inhibited to decrease IL2RA expression in a regulatory T cell, and wherein the subject has an autoimmune disorder.

In some embodiments, expression of one or more nuclear factors set forth in Table 8, Table 12 or Table 14 is inhibited to decrease IL2RA expression in a regulatory T cell, and wherein the subject has cancer. In some embodiments, expression of one or more nuclear factors selected from the group consisting of IKZF3, YY1, MBD2, IRF4, IKZF1, RXRB, RELA, ETS1, KMT2A, PTEN, JAK3, STAT5A, GATA3, FOXP1, STAT5B, and IL2RA is inhibited to decrease IL2RA expression in a regulatory T cell, and wherein the subject has cancer.

In some embodiments, expression of one or more nuclear factors set forth in Table 8, Table 10 or Table 14 is inhibited to decrease IL2RA expression in a conventional T cell, and wherein the subject has an autoimmune disorder. In some embodiments, expression of one or more nuclear factors selected from the group consisting of IKZF3, YY1, MBD2, IRF4, IKZF1, RXRB, RELA, ETS1, KMT2A, PTEN, JAK3, STAT5A, GATA3, FOXP1, STAT5B, and IL2RA is inhibited to decrease IL2RA expression in a conventional T cell, and wherein the subject has an autoimmune disorder.

In some methods, expression of one or more nuclear factors selected from the group consisting of MED12, CBFB, HIVEP2, KLF2, MYB, FOXK1, ZNF217, IRF2, TFNAIP3, MYC, PRDM1, TFDP1, IRF1, FOXO1, ATXN7L3 and TP53 is inhibited to increase IL2RA expression in a conventional T cell, wherein the subject has cancer. In some examples, one or more nuclear factors selected from the group consisting of MED12, FOXP1, PTEN, IKZF1, TAF5L, PRDM1, TFDP1, CXXC1, IKZF3 and TP53 is inhibited to increase IL-2 in a conventional T cell, wherein the subject has cancer.

In another embodiment, provided herein is a method of modifying T cells in a subject in need thereof, comprising inhibiting expression of a one or more nuclear factors set forth in Table 1, Table 2, Table 3, Table 4, Table 5, Table 6, Table 7, Table 8, Table 9, Table 10, Table 11, Table 12, Table 13 or Table 14 and/; or overexpressing one or more nuclear factors set for in Table 1, Table 2, Table 3, Table 4, Table 5, Table 6, Table 7, Table 8, Table 9, Table 10, Table 11, Table 12, Table 13 or Table 14 in the human T cells of the subject.

In some embodiments, one or more nuclear factors selected from the group consisting of CBFB, MYB, ZNF217, FOXK1, FLI1, FOS, SATB1, IL2, ATXN7L3, MTF1. RELA, IRF1, BCL11B, STAT3, MED30, MED14, MED11, IKZF3, KMT2A, IKZF1, MED12, TAF5L, PTEN, IRF4, FOXO1, FOXP1, CTLA4, ETS1, MYBL2, TP53, MBD2, ZBTB7A, DNMT1, HIVEP2, KLF2, TFDP1, SMARCB1, MAF, FOXP3, GATA3, STAT5B, STAT5A, PRDM1, TNFAIP3, RXRB, TFDP1, CXXC1, NFATC2, MAF, IRF2, ZBTB11, JAK3, YY1, IL2RA and GTF2B are inhibited in the human T cells of the subject.

In some embodiments, inhibiting expression of one or more nuclear factors or overexpression of one or more nuclear factors occurs in vivo.

In some embodiments, the method comprises a) obtaining T cells from the subject; b) modifying the T cells by inhibiting expression of one or more nuclear factors set forth in Table 2, Table 4, Table 5 or Table 7; and c) administering the T cells to the subject. In some embodiments, one or more nuclear factors selected from the group consisting of MTF1, RELA, IRF1, BCL11B, STAT3, MED30, MED14, MED11, IKZF3, KMT2A, IKZF1, TAF5L, IRF4, FOXP1, CTLA4, FOXP3, GATA3, STAT5B, STAT5A, PTEN, FOXO1, MED12, FOXP1, PTEN, IKZF1, TAF5L, PRDM1, TFDP1, CXXC1, IKZF3, TP53, CBFB, HIVEP2, KLF2, MYB, FOXK1, ZNF217, IRF2, TFNAIP3, MYC, PRDM1, TFDP1, IRF1, ATXN7L3 and TP53 are inhibited. In some embodiments the subject has cancer.

In some embodiments, the method comprises a) obtaining T cells from the subject; b) modifying the T cells by overexpressing one or more nuclear factors set forth in Table 1, Table 3, Table 6 or Table 8, and c) administering the T cells to the subject. In some embodiments, one or more nuclear factors selected from the group consisting of CBFB, MYB, ZNF217, FOXK1, FLI1, FOS, SATB1, IL2, ATXN7L3, ETS1, MYBL2, MYB, TP53, FLI1, SATB1, MBD2, ZBTB7A, DNMT1, TFDP1, SMARCB1, MAF, NFATC2, MAF, ZBTB7A, MBD2, GATA3, MED14, IRF2, MED30, ZBTB11, RELA, JAK3, MED11, BCL11B, MTF1, ATXN7L3, YY1, ETS1, IL2, DNMT1, GTF2B, SMARCB1, IKZF3, YY1, MBD2, IRF4, IKZF1, RXRB, RELA, ETS1, KMT2A, PTEN, JAK3, STAT5A, GATA3, FOXP1, STAT5B and IL2RA are overexpressed. In some embodiments the subject has cancer.

In some embodiments, the method comprises a) obtaining T cells from the subject; b) modifying the T cells by inhibiting expression of one or more nuclear factors set forth in Table 1, Table 3, Table 6 or Table 8; and c) administering the T cells to the subject. In some embodiments, expression of one or more nuclear factors selected from the group consisting of CBFB, MYB, ZNF217, FOXK1, FLI1, FOS, IL2, ATXN7L3, ETS1, MYBL2, MYB, TP53, FLI1, SATB1, ZBTB7A, DNMT1, TFDP1, SMARCB1, MAF, NFATC2, MAF, ZBTB7A, MED14, IRF2, MED30, ZBTB11, MED11, BCL11B, MTF1, ATXN7L3, YY1, ETS1, 1L2, DNMT1, GTF2B, IKZF3, MBD2, IRF4, IKZF1, RXRB, RELA, ETS1, KMT2A, PTEN, JAK3, STAT5A, GATA3, FOXP1, STAT5B and IL2RA is inhibited. In some embodiments, the subject has an autoimmune disorder.

In some embodiments, the method comprises a) obtaining T cells from the subject: b) modifying the T cells by overexpressing one or more nuclear factors set forth in Table 2, Table 4, Table 5 or Table 7; and c) administering the T cells to the subject. In some embodiments, one or more nuclear factors selected from the group consisting of MTF1, RELA, IRF1, BCL11B, STAT3, MED30, MED14, MED11, IKZF3, KMT2A, IKZF1, TAF5L, IRF4, FOXP1, CTLA4, FOXP3, GATA3, STAT5B, STAT5A, PTEN, FOXO1, MED12, FOXP1, PTEN, IKZF1, TAF5L, PRDM1, TFDP1, CXXC1, IKZF3, TP53, CBFB, HIVEP2, KLF2, MYB, FOXK1, ZNF217, IRF2, TFNAIP3, MYC, PRDM1, TFDP1, IRF1, ATXN7L3 and TP53 are overexpressed. In some embodiments, the subject has an autoimmune disorder.

Also provided is a method of treating an autoimmune disorder in a subject, the method comprising administering a population of the T cells to a subject that has an autoimmune disorder, wherein the T cells comprise a genetic modification or heterologous polynucleotide that inhibits expression of a nuclear factor set forth in Table 1 and/or a heterologous polypeptide that encodes a nuclear factor set forth in Table 2; a genetic modification or heterologous polynucleotide that inhibits expression of a nuclear factor set forth in Table 3 and/or a heterologous polypeptide that encodes a nuclear factor set forth in Table 4; a genetic modification or heterologous polynucleotide that inhibits expression of a nuclear factor set forth in Table 6, and/or a heterologous polypeptide that encodes a nuclear factor set forth in Table 5; or a genetic modification or heterologous polynucleotide that inhibits expression of a nuclear factor set forth in Table 8, and/or a heterologous polypeptide that encodes a nuclear factor set forth in Table 7.

Further provided is a method of treating cancer in a subject, the method comprising administering a population of the T cells to a subject that has cancer, wherein the T cells comprise a genetic modification or heterologous polynucleotide that inhibits expression of a nuclear factor set forth in Table 2, and/or a heterologous polypeptide that encodes a nuclear factor set forth in Table 1; a genetic modification or heterologous polynucleotide that inhibits expression of a nuclear factor set forth in Table 4, and/or a heterologous polypeptide that encodes a nuclear factor set forth in Table 3; a genetic modification or heterologous polynucleotide that inhibits expression of a nuclear factor set forth in Table 5, and/or a heterologous polypeptide that encodes a nuclear factor set forth in Table 6; a genetic modification or heterologous polynucleotide that inhibits expression of a nuclear factor set forth in Table 7, and/or a heterologous polypeptide that encodes a nuclear factor set forth in Table 8.

### BRIEF DESCRIPTION OF THE DRAWINGS

The present application includes the following figures. The figures are intended to illustrate certain embodiments and/or features of the compositions and methods, and to supplement any description(s) of the compositions and methods. The figures do not limit the scope of the compositions and methods, unless the written description expressly indicates that such is the case.

FIG. 1 is a diagram showing how SLICE (sgRNA Lentiviral Infection with Cas9 Electroporation), as described in Shifrut et al. Cell 175(7): 1958-1971 (2018), can be used to identify nuclear factors that modulate targets of interest. Flow-Seq enables CRISPR loss-of-function screening in primary human T cells. A transcription factor CRISPR knockout library was introduced into CD4+ T cells along with Cas9 protein. These cells were stained for a gene of interest, sorted into high- and low-expression bins using fluorescent activated cell sorting (FACS), and the guide RNAs in each bin were sequenced. The high- and low-enriched guide RNAs were compared to identify transcription factors that regulate the protein levels of the target of interest.

FIG. 2 provides an overview of the arrayed Cas9 ribonucleoprotein (RNP) approach to individually knock out transcription factor hits from SLICE Flow-Seq screens. Synthetic guide RNAs were ordered against 56 genes and 4 non-targeting controls, complexed with Cas9 protein, and electroporated into T cells in an arrayed format. Cells were collected 3, 5, and 7 days after electroporation for multiplex phenotyping including flow cytometry validation, genotyping, RNA-Seq, and ATAC-Seq.

FIGS. 3A-3D provide the transcription factors that regulate protein levels of four key immune genes IL2RA (FIG. 3A), IL-2 (FIG. 3B), CTLA4 (FIG. 3C) and FOXP3 (FIG. 3D) discovered using SLICE Flow-Seq. Cells were stained for the target of interest, sorted into high and low expression bins using fluorescent activated cell sorting, and the guide RNAs in each bin were sequenced. Red points highlight transcription factors that are significantly differently enriched between the high and low bins. Each dot represents the signal across four independent guide RNAs targeting that transcription factor.

FIGS. 4A-4C show there is a high degree of overlap between hits from the four screens. A) The total number of significant hits in each screen and the number of hits that overlap between the different screens. B) Prioritization of 56 genes for follow-up. Genes are grouped (1-4) based on the number of screens they were significant in. The effect sizes in each Flow-Seq screen are shown. C) Genotyping of the insertion/deletion frequency following RNP editing at each transcription factor target site using two different guide RNAs.

FIGS. 5A-5D show flow cytometry validation of screen hits following RNP knockout. Cells were stained for the target of interest (IL2RA (FIG. 5A), IL-2 (FIG. 5B), CRLA4 (FIG. 5C) and FOXP3 (FIG. 5D)) and analyzed using flow cytometry. Median fluorescent intensity was normalized to four non-targeting controls per donor. Points are colored based on two independent guide RNAs. Points show the median of 3 biological donors and error bars show the range.

FIG. 6 shows identification of cell type-specific transcription factors that regulate the protein levels of IL2RA discovered using SLICE Flow-Seq in effector T cells vs. regulatory T cells. Effector and regulatory T cells were stained for IL2RA, sorted into high and low expression bins using fluorescent activated cell sorting, and the guide RNAs in each bin were sequenced. Table 9 provides transcription factors that, when inhibited, result in increased levels of IL2RA in effector T cells. Table 10 provides transcription factors that, when inhibited, result in decreased levels of IL2RA in effector T cells. Table 11 provides transcription factors that, when inhibited, result in increased levels of IL2RA in regulatory T cells. Table 12 provides transcription factors that, when inhibited, result in decreased levels of IL2RA in regulatory T cells. Table 13 provides transcription factors that, when inhibited, result in increased levels of IL2RA in effector cells and regulatory T cells. Table 14 provides transcription factors that, when inhibited, result in decreased levels of IL2RA in effector cells and regulatory T cells.

FIG. 7 shows validation of hit screen. FIG. 7A is a schematic of synthetic crRNA/Cas9 ribonucleoprotein arrayed knockout (KO) followed by in depth characterization of KOs. FIG. 7B shows representative flow cytometry density plots for top hits in the IL2RA. IL-2, and CTLA4 screens. All plots are normalized to a maximum height of 1. KO of hits that decrease target levels are shown in orange and KO of hits that increase target levels are shown in blue. FIGS. 7C-F show flow cytometry results for IL2RA, IL-2, CTLA4 and FOXP3, 5 days after arrayed RNP KO. Screen hits analyzed are displayed on the Y axis ordered by their effect size in the pooled CRISPR screen. Changes in IL2RA, IL-2, and CTLA4 median fluorescence intensity relative to non-targeting controls is shown on the X-axis. Dots represent individual data points, bars depict average, and error bars depict standard deviation across 2 guide RNAs and 3 donors per guide RNA. Bars are colored by whether the flow cytometry effect matched the pooled CRISPR screen effect and whether the KO increased or decreased the level of IL2RA. IL-2, or CTLA4.

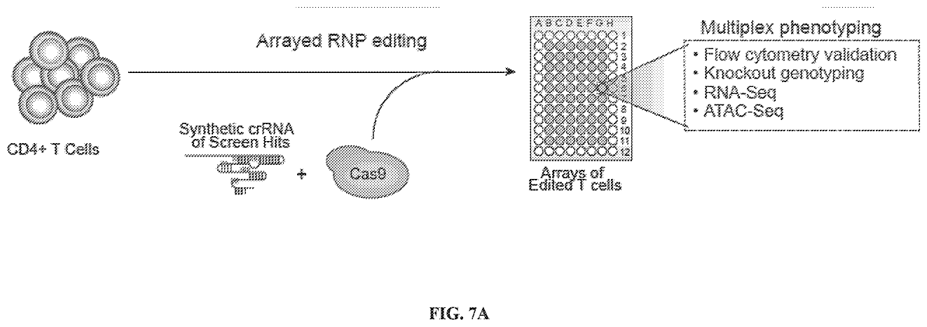

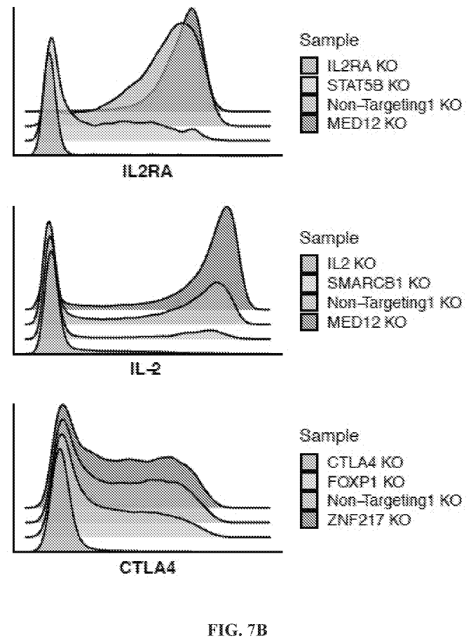

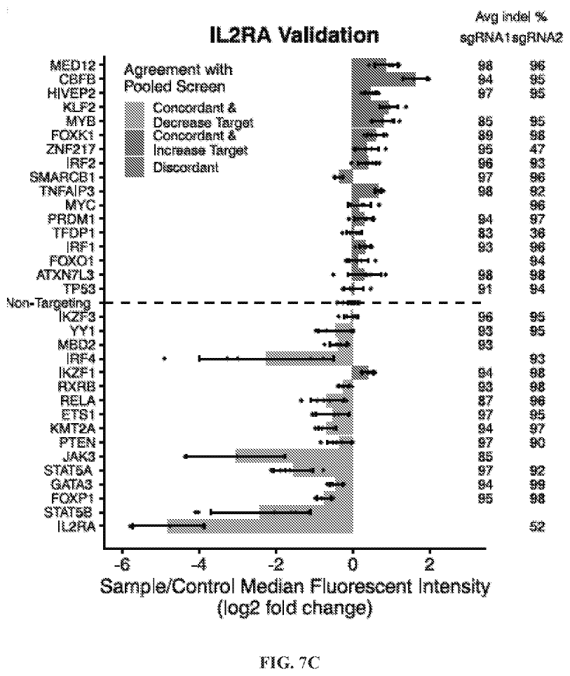

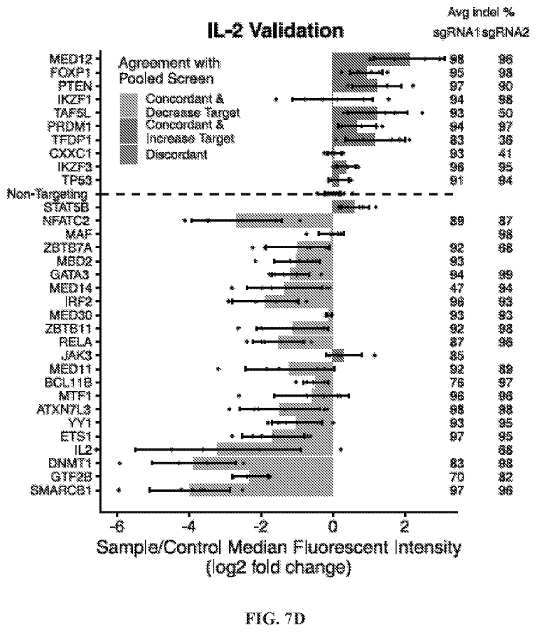

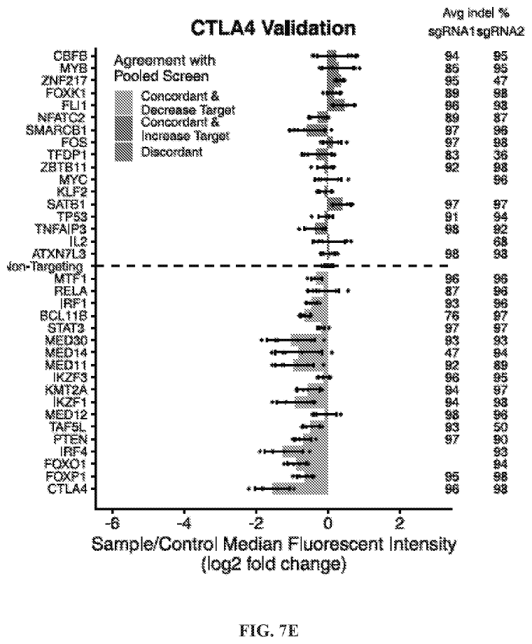

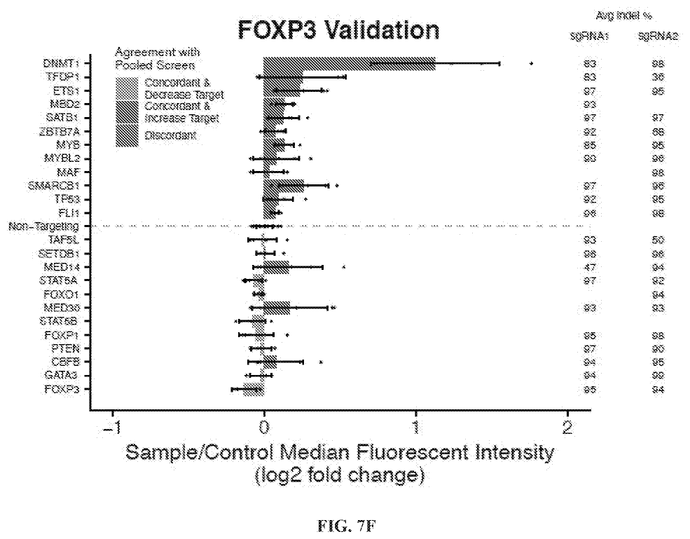

All of the hits in FIGS. 7C-F, above the Non-Targeting dashed line were concordant with pooled screens and increased expression of the target, except for SMARCB1 (for IL2RA), NFATC2 (for CTLA4), TFDP1 (for CTLA4), ZBTB11 (for CTLA4), MYC (for CTLA4), KLF2 (for CTLA4), TP53 (for CTLA4), TNFAIP3 (for CTLA4), and IKZF1 (for IL-2). All of the hits in FIGS. 7C-F, below the Non-Targeting dashed line were concordant with pooled screens and decreased expression of the target, except for IKZF1 (for IL2RA), STAT5B (for IL-2), JAK3 (for IL-2), MED14 (for FOXP3), MED30 (for FOXP3), CBFB (for FOXP3) and SETDB1 (for FOXP3). The average insertion/deletion (indel) percentage across multiple donors for guide RNA 1 (n=3) and guide RNA 2 (n=2) at the genomic target site is shown to the right of each graph.

### Definitions

As used in this specification and the appended claims, the singular forms “a,” “an,” and “the” include plural reference unless the context clearly dictates otherwise.

The term “nucleic acid” or “polynucleotide” refers to deoxyribonucleic acids (DNA) or ribonucleic acids (RNA) and polymers thereof in either single- or double-stranded form. Unless specifically limited, the term encompasses nucleic acids containing known analogues of natural nucleotides that have similar binding properties as the reference nucleic acid and are metabolized in a manner similar to naturally occurring nucleotides. Unless otherwise indicated, a particular nucleic acid sequence also implicitly encompasses conservatively modified variants thereof (e.g., degenerate codon substitutions), alleles, orthologs, SNPs, and complementary sequences as well as the sequence explicitly indicated. Specifically, degenerate codon substitutions may be achieved by generating sequences in which the third position of one or more selected (or all) codons is substituted with mixed-base and/or deoxyinosine residues (Batzer et al., Nucleic Acid Res. 19:5081 (1991); Ohtsuka et al., J. Biol. Chem. 260:2605-2608 (1985); and Rossolini et al., Mol. Cell. Probes 8:91-98 (1994)). The term nucleic acid is used interchangeably with gene, cDNA, and mRNA encoded by a gene.

The term “gene” can refer to the segment of DNA involved in producing or encoding a polypeptide chain. It may include regions preceding and following the coding region (leader and trailer) as well as intervening sequences (introns) between individual coding segments (exons).

“Polypeptide,” “peptide,” and “protein” are used interchangeably herein to refer to a polymer of amino acid residues. As used herein, the terms encompass amino acid chains of any length, including full-length proteins, wherein the amino acid residues are linked by covalent peptide bonds.

The term “inhibiting expression” refers to inhibiting or reducing the expression of a gene product, e.g., RNA or protein. As used throughout, the term “nuclear factor” refers to a protein that directly or indirectly alters expression of IL2RA, IL-2, CTLA4 or FOXP3, for example, a transcription factor. To inhibit or reduce the expression of a gene, the sequence and/or structure of the gene may be modified such that the gene would not be transcribed (for DNA) or translated (for RNA), or would not be transcribed or translated to produce a functional protein, for example, a polypeptide or protein encoded by a gene set forth in Table 1, Table 2, Table3, Table 4, Table 5, Table 6, Table 7, Table 8, Table 9, Table 10, Table 11, Table 12, Table 13 or Table 14. Various methods for inhibiting or reducing expression are described in detail further herein. Some methods may introduce nucleic acid substitutions, additions, and/or deletions into the wild-type gene. Some methods may also introduce single or double strand breaks into the gene. To inhibit or reduce the expression of a protein, one may inhibit or reduce the expression of the gene or polynucleotide encoding the protein. In other embodiments, one may target the protein directly to inhibit or reduce the protein's expression using, e.g., an antibody or a protease. “Inhibited” expression refers to a decrease by at least 10% as compared to a reference control level, for example a decrease by at least about 20%, or at least about 30%, or at least about 40%, or at least about 50%, or at least about 60%, or at least about 70%, or at least about 80%, or at least about 90% or up to and including a 100% decrease (i.e. absent level as compared to a reference sample). It is understood that one or more nuclear factors set forth in Table 1, Table 2, Table3, Table 4, Table 5, Table 6, Table 7, Table 8, Table 9, Table 10, Table 11, Table 12, Table 13 or Table 14 can be inhibited in a T cell. It is also understood that two or more nuclear factors inhibited in a T cell can be selected from one or more of Table 1, Table 2, Table3, Table 4, Table 5, Table 6, Table 7, Table 8, Table 9, Table 10, Table 11, Table 12, Table 13 or Table 14.

The term “overexpressing” or “overexpression” refers to increasing the expression of a gene or protein. “Overexpression” refers to an increase in expression, for example, in increase in the amount of mRNA or protein expressed in a T cell, of at least 10%, as compared to a reference control level, or an increase of least about 20%, or at least about 30%, or at least about 40%, or at least about 50%, or at least about 60%, or at least about 70%, or at least about 80%, or at least about 90%, or at least about 100%, or at least about 200%, or at least about 300% or at least about 400%. Various methods for overexpression are known to those of skill in the art, and include, but are not limited to, stably or transiently introducing a heterologous polynucleotide encoding a protein (i.e., a nuclear factor set forth in Table 1, Table 2, Table3, Table 4, Table 5, Table 6, Table 7, Table 8, Table 9, Table 10, Table 11, Table 12, Table 13 or Table 14) to be overexpressed into the cell or inducing overexpression of an endogenous gene encoding the protein in the cell. It is understood that one or more nuclear factors set forth in Table 1, Table 2, Table3, Table 4, Table 5. Table 6, Table 7, Table 8, Table 9, Table 10, Table 11, Table 12, Table 13 or Table 14 can be overexpressed in a T cell. It is also understood that two or more nuclear factors overexpressed in a T cell can be selected from one or more of Table 1. Table 2, Table3, Table 4, Table 5, Table 6, Table 7, Table 8, Table 9, Table 10, Table 11, Table 12, Table 13 or Table 14.

As used herein the phrase “heterologous” refers to what is not found in nature. The term “heterologous sequence” refers to a sequence not normally found in a given cell in nature. As such, a heterologous nucleotide or protein sequence may be: (a) foreign to its host cell (i.e., is exogenous to the cell); (b) naturally found in the host cell (i.e., endogenous) but present at an unnatural quantity in the cell (i.e., greater or lesser quantity than naturally found in the host cell); or (c) be naturally found in the host cell but positioned outside of its natural locus.

“Treating” refers to any indicia of success in the treatment or amelioration or prevention of the disease, condition, or disorder, including any objective or subjective parameter such as abatement; remission; diminishing of symptoms or making the disease condition more tolerable to the patient; slowing in the rate of degeneration or decline; or making the final point of degeneration less debilitating.

A “promoter” is defined as one or more a nucleic acid control sequences that direct transcription of a nucleic acid. As used herein, a promoter includes necessary nucleic acid sequences near the start site of transcription, such as, in the case of a polymerase II type promoter, a TATA element. A promoter also optionally includes distal enhancer or repressor elements, which can be located as much as several thousand base pairs from the start site of transcription.

As used herein, the term “complementary” or “complementarity” refers to specific base pairing between nucleotides or nucleic acids. Complementary nucleotides are, generally, A and T (or A and U), and G and C. The guide RNAs described herein can comprise sequences, for example, DNA targeting sequences that are perfectly complementary or substantially complementary (e.g., having 1-4 mismatches) to a genomic sequence.

As used throughout, by subject is meant an individual. For example, the subject is a mammal, such as a primate, and, more specifically, a human. Non-human primates are subjects as well. The term subject includes domesticated animals, such as cats, dogs, etc., livestock (for example, cattle, horses, pigs, sheep, goats, etc.) and laboratory animals (for example, ferret, chinchilla, mouse, rabbit, rat, gerbil, guinea pig, etc.). Thus, veterinary uses and medical uses and formulations are contemplated herein. The term does not denote a particular age or sex. Thus, adult and newborn subjects, whether male or female, are intended to be covered. As used herein, patient or subject may be used interchangeably and can refer to a subject afflicted with a disease or disorder.

As used throughout, the term “targeted nuclease” refers to nuclease that is targeted to a specific DNA sequence in the genome of a cell to produce a strand break at that specific DNA sequence. The strand break can be single-stranded or double-stranded. Targeted nucleases include, but are not limited to, a Cas nuclease, a TAL-effector nuclease and a zinc finger nuclease.

The “CRISPR/Cas” system refers to a widespread class of bacterial systems for defense against foreign nucleic acid. CRISPR/Cas systems are found in a wide range of eubacterial and archaeal organisms. CRISPR/Cas systems include type I, II, and III sub-types. Wild-type type 11 CRISPR/Cas systems utilize an RNA-mediated nuclease, for example, Cas9, in complex with guide and activating RNA to recognize and cleave foreign nucleic acid. Guide RNAs having the activity of both a guide RNA and an activating RNA are also known in the art. In some cases, such dual activity guide RNAs are referred to as a single guide RNA (sgRNA).

Cas9 homologs are found in a wide variety of eubacteria, including, but not limited to bacteria of the following taxonomic groups: Actinobacteria, Aquificae, Bacteroidetes-Chlorobi, Chlamydiae-Verrucomicrobia, Chiroflexi, Cyanobacteria, Firmicutes, Proteobacteria, Spirochaetes, and Thermotogae. An exemplary Cas9 protein is the Streptococcus pyogenes Cas9 protein. Additional Cas9 proteins and homologs thereof are described in, e.g., Chylinksi, et al., RNA Biol. 2013 May 1; 10(5): 726-737; Nat. Rev. Microbiol. 2011 June; 9(6): 467477; Hou, et al., Proc Nad/Acad Sci USA. 2013 Sep. 24:110(39):15644-9; Sampson et al., Nature. 2013 May 9; 497(7448):254-7; and Jinek, et al., Science. 2012 Aug. 17; 337(609%):816-21. Variants of any of the Cas9 nucleases provided herein can be optimized for efficient activity or enhanced stability in the host cell. Thus, engineered Cas9 nucleases are also contemplated.

As used throughout, a guide RNA (gRNA) sequence is a sequence that interacts with a site-specific or targeted nuclease and specifically binds to or hybridizes to a target nucleic acid within the genome of a cell, such that the gRNA and the targeted nuclease co-localize to the target nucleic acid in the genome of the cell. Each gRNA includes a DNA targeting sequence or protospacer sequence of about 10 to 50 nucleotides in length that specifically binds to or hybridizes to a target DNA sequence in the genome. For example, the targeting sequence may be about 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 nucleotides in length. In some embodiments, the gRNA comprises a crRNA sequence and a transactivating crRNA (tracrRNA) sequence. In some embodiments, the gRNA does not comprise a tracrRNA sequence. Table 3 shows exemplary gRNA sequences used in methods of the disclosure.

As used herein, the term “Cas9” refers to an RNA-mediated nuclease (e.g. of bacterial or archaeal origin, or derived therefrom). Exemplary RNA-mediated nucleases include the foregoing Cas9 proteins and homologs thereof. Other RNA-mediated nucleases include Cpf1 (See, e.g., Zetsche et al., Cell, Volume 163, Issue 3, p759-771, 22 Oct. 2015) and homologs thereof. Similarly, as used herein, the term “Cas9 ribonucleoprotein” complex and the like refers to a complex between the Cas9 protein and a guide RNA, the Cas9 protein and a crRNA, the Cas9 protein and a trans-activating crRNA (tracrRNA), or a combination thereof (e.g., a complex containing the Cas9 protein, a tracrRNA, and a crRNA guide RNA). It is understood that in any of the embodiments described herein, a Cas9 nuclease can be substituted with a Cpf1 nuclease or any other guided nuclease.

As used herein, the phrase “modifying” refers to inducing a structural change in the sequence of the genome at a target genomic region in a T cell. For example, the modifying can take the form of inserting a nucleotide sequence into the genome of the cell. Such modifying can be performed, for example, by inducing a double stranded break within a target genomic region, or a pair of single stranded nicks on opposite strands and flanking the target genomic region. Methods for inducing single or double stranded breaks at or within a target genomic region include the use of a Cas9 nuclease domain, or a derivative thereof, and a guide RNA, or pair of guide RNAs, directed to the target genomic region. “Modifying” can also refer to altering the expression of a nuclear factor in a T cell, for example inhibiting expression of a nuclear factor or overexpressing a nuclear factor in a T cell.

As used herein, the phrase “T cell” refers to a lymphoid cell that expresses a T cell receptor molecule. T cells include human alpha beta (αβ) T cells and human gamma delta (γδ) T cells. T cells include, but are not limited to, naïve T cells, stimulated T cells, primary T cells (e.g., uncultured), cultured T cells, immortalized T cells, helper T cells, cytotoxic T cells, memory T cells, regulatory T cells, natural killer T cells, combinations thereof, or sub-populations thereof. T cells can be CD4⁺, CD8⁺, or CD4⁺ and CD8⁺. T cells can also be CD4⁻, CD8⁻, or CD4⁻ and CD8⁻ T cells can be helper cells, for example helper cells of type T_{H}1, T_{H}2, T_{H}3, T_{H}19, T_{H}17, or T_{FH}. T cells can be cytotoxic T cells. T cells can also be regulatory T cells. Regulatory T cells (Tregs) can be FOXP3⁺ or FOXP3⁻. T cells can be alpha/beta T cells or gamma/delta T cells. In some cases, the T cell is a CD4⁺CD25^{hi}CD127^{lo} regulatory T cell. In some cases, the T cell is a regulatory T cell selected from the group consisting of type 1 regulatory (Tr1), T_{H}3, CD8+CD28−, Treg17, and Qa-1 restricted T cells, or a combination or sub-population thereof. In some cases, the T cell is a FOXP3⁺ T cell. In some cases, the T cell is a CD4⁺CD25^{lo}CD127^{hi} effector T cell. In some cases, the T cell is a CD4⁺CD25^{lo}CD127^{hi}CD45RA^{hi}CD45RO⁻ naïve T cell. A T cell can be a recombinant T cell that has been genetically manipulated.

As used herein, the phrase “primary” in the context of a primary cell is a cell that has not been transformed or immortalized. Such primary cells can be cultured, sub-cultured, or passaged a limited number of times (e.g., cultured 0, 1, 2, 3, 4, 5, 6, 7, 8, 9, 10, 11, 12, 13, 14, 15, 16, 17, 18, 19, or 20 times). In some cases, the primary cells are adapted to in vitro culture conditions. In some cases, the primary cells are isolated from an organism, system, organ, or tissue, optionally sorted, and utilized directly without culturing or sub-culturing. In some cases, the primary cells are stimulated, activated, or differentiated. For example, primary T cells can be activated by contact with (e.g., culturing in the presence of) CD3, CD28 agonists, IL-2, IFN-γ, or a combination thereof.

As used herein, the phrase “introducing” in the context of introducing a nucleic acid or a complex comprising a nucleic acid, for example, an RNP complex, refers to the translocation of the nucleic acid sequence or the RNP complex from outside a cell to inside the cell. In some cases, introducing refers to translocation of the nucleic acid or the complex from outside the cell to inside the nucleus of the cell. Various methods of such translocation are contemplated, including but not limited to, electroporation, contact with nanowires or nanotubes, receptor mediated internalization, translocation via cell penetrating peptides, liposome mediated translocation, and the like.

### DETAILED DESCRIPTION OF THE INVENTION

The following description recites various aspects and embodiments of the present compositions and methods. No particular embodiment is intended to define the scope of the compositions and methods. Rather, the embodiments merely provide non-limiting examples of various compositions and methods that are at least included within the scope of the disclosed compositions and methods. The description is to be read from the perspective of one of ordinary skill in the art; therefore, information well known to the skilled artisan is not necessarily included

### I. Methods and Compositions

As described herein, the disclosure provides compositions and methods directed to modifying T cells by inhibiting the expression of one or more nuclear factors and/or overexpressing one or more nuclear factors in a T cell. The disclosure also features compositions comprising the genetically modified T cells described herein. A population of modified T cells may provide therapeutic benefits in treating diseases with altered immune responses, for example, cancer or treating autoimmune diseases.

The inventors have discovered that by inhibiting the expression of one or more nuclear factors and/or overexpressing one or more nuclear factors, T cells may be altered to modulate T cell function.

Examples of nuclear factors whose expression may be altered to modify the stability of T cells in the methods described herein include, but are not limited to the nuclear factors set forth in Table 1, Table 2, Table3, Table 4, Table 5, Table 6, Table 7, Table 8, Table 9, Table 10, Table 11, Table 12, Table 13 or Table 14.

In some embodiments, the present invention provides a method of modifying a T cells, the method comprising: inhibiting expression of one or more nuclear factors set forth in Table 1, Table 2, Table3, Table 4, Table 5, Table 6, Table 7, Table 8, Table 9, Table 10, Table 11, Table 12, Table 13 or Table 14, and/or overexpressing one or more nuclear factors set forth in Table 1, Table 2, Table3, Table 4, Table 5, Table 6, Table 7, Table 8, Table 9, Table 10, Table 11, Table 12, Table 13 or Table 14.

In some embodiments, the T cell comprises a genetic modification or heterologous polynucleotide that inhibits expression of one or more nuclear factors selected from the group consisting of CBFB, MYB, ZNF217, FOXK1, FLI1, FOS, SATB1, IL2, ATXN7L3, MTF1, RELA, IRF1, BCL11B, STAT3, MED30, MED14, MED11, IKZF3, KMT2A, IKZF1, MED12, TAF5L, PTEN, IRF4, FOXO1, FOXP1, CTLA4, ETS1, MYBL2, TP53, MBD2, ZBTB7A, DNMT1, HIVEP2, KLF2, TFDP1, SMARCB1, MAF, FOXP3, GATA3, STAT5B, STAT5A, PRDM1, TNFAIP3, RXRB, TFDP1, CXXC1, NFATC2, MAF, IRF2, ZBTB11, JAK3, YY1, IL2RA and GTF2B.

In some embodiments one or more nuclear factors selected from the group consisting of CBFB, MYB, ZNF217, FOXK1, FLI1, FOS, SATB1, IL2, ATXN7L3, MTF1, RELA, IRF1, BCL11B, STAT3, MED30, MED14, MED11, IKZF3, KMT2A, IKZF1, MED12, TAF5L, PTEN, IRF4, FOXO1, FOXP1, CTLA4, ETS1, MYBL2, TP53, MBD2, ZBTB7A, DNMT1, TFDP1, SMARCB1, MAF, FOXP3, GATA3, STAT5B, STAT5A, PRDM1, TNFAIP3, RXRB, TFDP1, CXXC1, NFATC2, MAF, IRF2, ZBTB11, JAK3, YY1, IL2RA and GTF2B are overexpressed in the T cell. In some embodiments a one or more nuclear factors are inhibited in the T cell and one or more, different nuclear factors are overepxressed in the T cell.

TABLE 1
Nuclear factors that can be inhibited to increase CTLA4 expression or overexpressed to decrease CTLA4 expression

| | | | | | | Position | | | | | | | | |
|---|---|---|---|---|---|---|---|---|---|---|---|---|---|---|
| | | screen_ | Target | | | of Base | | | SEQ | Target | SEQ | | | Rule |
| gene_ | screen_ | di- | Gene | Target | Genomic | After Cut | | sgRNA Target | ID | Context | ID | PAM | Exon | Set 2 |
| id | target | rection | ID | Transcript | Sequence | (1-based) | Strand | Sequence | NO: | Sequence | NO: | Sequence | Number | score |
| MAX | CTLA4 | CTLA4 | 4149 | NM_ | NC_ | 65077986 | sense | AATATATCCAGTA | 1. | ACAGAATA | 145. | AGG | 4 | 0.6132 |
| | | high | | 002382.4 | 000014.9 | | | TATGCGA | | TATCCAGT | | | | |
| | | | | | | | | | | ATATGCGA | | | | |
| | | | | | | | | | | AGGAAA | | | | |
| MAX | CTLA4 | CTLA4 | 4149 | NM_ | NC_ | 65077942 | anti- | GAAGAGCATTCTG | 2. | TCCAGAAG | 146. | AGG | 4 | 0.5652 |
| | | high | | 002382.4 | 000014.9 | | sense | CCGCTTG | | AGCATTCT | | | | |
| | | | | | | | | | | GCCGCTTG | | | | |
| | | | | | | | | | | AGGTCG | | | | |
| MAX | CTLA4 | CTLA4 | 4149 | NM_ | NC_ | 65101564 | sense | TATTCCAGGAAGA | 3. | CTCTTATT | 147. | AGG | 2 | 0.668 |
| | | high | | 002382.4 | 000014.9 | | | GCAACCG | | CCAGGAA | | | | |
| | | | | | | | | | | GAGCAAC | | | | |
| | | | | | | | | | | CGAGGTTT | | | | |
| MAX | CTLA4 | CTLA4 | 4149 | NM_ | NC_ | 65076652 | sense | TTTAGTCCGTGCA | 4. | GCCTTTTA | 148. | AGG | 5 | 0.5205 |
| | | high | | 002382.4 | 000014.9 | | | CTGGAGA | | GTCCGTGC | | | | |
| | | | | | | | | | | ACTGGAG | | | | |
| | | | | | | | | | | AAGGCGA | | | | |
| ARID3B | CTLA4 | CTLA4 | 10620 | NM_ | NC_ | 74591240 | sense | AATTGATGGCAAC | 5. | CAGCAATT | 149. | AGG | 6 | 0.6479 |
| | | high | | 006465.2 | 000015.10 | | | CGCAGGG | | GATGGCA | | | | |
| | | | | | | | | | | ACCGCAG | | | | |
| | | | | | | | | | | GGAGGGC | | | | |
| | | | | | | | | | | C | | | | |
| ARID3B | CTLA4 | CTLA4 | 10620 | NM_ | NC_ | 74544090 | anti- | GCTGTGGCGGAA | 6. | CCCAGCTG | 150. | CGG | 2 | 0.6132 |
| | | high | | 006465.2 | 000015.10 | | sense | AGGAGAGT | | TGGCGGA | | | | |
| | | | | | | | | | | AAGGAGA | | | | |
| | | | | | | | | | | GTCGGCTG | | | | |
| ARID3B | CTLA4 | CTLA4 | 10620 | NM_ | NC_ | 74544185 | sense | GTGGCCCAAGTGT | 7. | AGCAGTG | 151. | GGG | 2 | 0.6876 |
| | | high | | 006465.2 | 000015.10 | | | TTGAACG | | GCCCAAGT | | | | |
| | | | | | | | | | | GTTTGAAC | | | | |
| | | | | | | | | | | GGGGCAA | | | | |
| ARID3B | CTLA4 | CTLA4 | 10620 | NM_ | NC_ | 74573200 | sense | TTCGTCTTTATGC | 8. | CCTCTTCG | 152. | GGG | 4 | 0.6704 |
| | | high | | 006465.2 | 000015.10 | | | AGAAGAG | | TCTTTATG | | | | |
| | | | | | | | | | | CAGAAGA | | | | |
| | | | | | | | | | | GGGGTGA | | | | |
| ZNF652 | CTLA4 | CTLA4 | 22834 | NM_ | NC_ | 49317162 | sense | AGAAAGTCAGCG | 9. | GTAGAGA | 153. | AGG | 2 | 0.6318 |
| | | high | | 014897.2 | 000017.11 | | | TTACACAA | | AAGTCAGC | | | | |
| | | | | | | | | | | GTTACACA | | | | |
| | | | | | | | | | | AAGGAGA | | | | |
| ZNF652 | CTLA4 | CTLA4 | 22834 | NM_ | NC_ | 49316958 | sense | CCAGGGTATTTAA | 10. | TGCCCCAG | 154. | TGG | 2 | 0.621 |
| | | high | | 014897.2 | 000017.11 | | | CACTCGC | | GGTATTTA | | | | |
| | | | | | | | | | | ACACTCGC | | | | |
| | | | | | | | | | | TGGTAC | | | | |
| ZNF652 | CTLA4 | CTLA4 | 22834 | NM_ | NC_ | 49312707 | sense | GGCTCATGTGCG | 11. | CCATGGCT | 155. | TGG | 3 | 0.674 |
| | | high | | 014897.2 | 000017.11 | | | GAAACACA | | CATGTGCG | | | | |
| | | | | | | | | | | GAAACAC | | | | |
| | | | | | | | | | | ATGGTTG | | | | |
| ZNF652 | CTLA4 | CTLA4 | 22834 | NM_ | NC_ | 49317275 | sense | TGTTCTTAAGACA | 12. | CTCCTGTT | 156. | AGG | 2 | 0.7322 |
| | | high | | 014897.2 | 000017.11 | | | AGCAGTG | | CTTAAGAC | | | | |
| | | | | | | | | | | AAGCAGT | | | | |
| | | | | | | | | | | GAGGAGG | | | | |
| KLF2 | CTLA4 | CTLA4 | 10365 | NM_ | NC_ | 16325729 | anti- | AAACCAGGGCCA | 13. | GCCGAAA | 157. | CGG | 2 | 0.5997 |
| | | high | | 016270.2 | 000019.10 | | sense | CCGAAAGG | | CCAGGGC | | | | |
| | | | | | | | | | | CACCGAAA | | | | |
| | | | | | | | | | | GGCGGCG | | | | |
| | | | | | | | | | | G | | | | |
| KLF2 | CTLA4 | CTLA4 | 10365 | NM_ | NC_ | 16325576 | anti- | CCCTCGCGCTTGA | 14. | GGCGCCCT | 158. | CGG | 2 | 0.5868 |
| | | high | | 016270.2 | 000019.10 | | sense | GGCCGCG | | CGCGCTTG | | | | |
| | | | | | | | | | | AGGCCGC | | | | |
| | | | | | | | | | | GCGGTCC | | | | |
| KLF2 | CTLA4 | CTLA4 | 10365 | NM_ | NC_ | 16325811 | sense | CTTCGGTCTCTTC | 15. | CAGCCTTC | 159. | CGG | 2 | 0.7252 |
| | | high | | 016270.2 | 000019.10 | | | GACGACG | | GGTCTCTT | | | | |
| | | | | | | | | | | CGACGAC | | | | |
| | | | | | | | | | | GCGGCCG | | | | |
| KLF2 | CTLA4 | CTLA4 | 10365 | NM_ | NC_ | 16325354 | anti- | TCGGGGTAATAG | 16. | GGGTTCG | 160. | CGG | 2 | 0.6967 |
| | | high | | 016270.2 | 000019.10 | | sense | AACGCAGG | | GGGTAAT | | | | |
| | | | | | | | | | | AGAACGC | | | | |
| | | | | | | | | | | AGGCGGC | | | | |
| | | | | | | | | | | GG | | | | |
| ZBTB11 | CTLA4 | CTLA4 | 27107 | NM_ | NC_ | 1.02E+08 | anti- | AATAAGATGTGCT | 17. | GACGAAT | 161. | AGG | 6 | 0.6273 |
| | | high | | 014415.3 | 000003.12 | | sense | CGCAAAG | | AAGATGT | | | | |
| | | | | | | | | | | GCTCGCAA | | | | |
| | | | | | | | | | | AGAGGCA | | | | |
| | | | | | | | | | | C | | | | |
| ZBTB11 | CTLA4 | CTLA4 | 27107 | NM_ | NC_ | 1.02E+08 | sense | CAGGACTTACGA | 18. | CACCCAGG | 162. | TGG | 4 | 0.605 |
| | | high | | 014415.3 | 000003.12 | | | GTACAGAA | | ACTTACGA | | | | |
| | | | | | | | | | | GTACAGA | | | | |
| | | | | | | | | | | ATGGAGG | | | | |
| ZBTB11 | CTLA4 | CTLA4 | 27107 | NM_ | NC_ | 1.02E+08 | sense | GGCATATATTCGA | 19. | AAGGGGC | 163. | AGG | 4 | 0.5992 |
| | | high | | 014415.3 | 000003.12 | | | CTACACA | | ATATATTC | | | | |
| | | | | | | | | | | GACTACAC | | | | |
| | | | | | | | | | | AAGGGAA | | | | |
| ZBTB11 | CTLA4 | CTLA4 | 27107 | NM_ | NC_ | 1.02E+08 | anti- | TATACATCCTACT | 20. | CAGCTATA | 164. | GGG | 4 | 0.6653 |
| | | high | | 014415.3 | 000003.12 | | sense | GATGACA | | CATCCTAC | | | | |
| | | | | | | | | | | TGATGACA | | | | |
| | | | | | | | | | | GGGCAG | | | | |
| TNFAIP3 | CTLA4 | CTLA4 | 7128 | NM_ | NC_ | 1.38E+08 | sense | CCACTTGTTAACA | 21. | TGTTCCAC | 165. | GGG | 6 | 0.6901 |
| | | high | | 001270507. | 000006.12 | | | GAGACCG | | TTGTTAAC | | | | |
| | | | | 1 | | | | | | AGAGACC | | | | |
| | | | | | | | | | | GGGGAAG | | | | |
| TNFAIP3 | CTLA4 | CTLA4 | 7128 | NM_ | NC_ | 1.38E+08 | sense | CTTGTGGCGCTGA | 22. | GAAGCTTG | 166. | CGG | 2 | 0.6709 |
| | | high | | 001270507. | 000006.12 | | | AAACGAA | | TGGCGCTG | | | | |
| | | | | 1 | | | | | | AAAACGA | | | | |
| | | | | | | | | | | ACGGTAA | | | | |
| TNFAIP3 | CTLA4 | CTLA4 | 7128 | NM_ | NC_ | 1.38E+08 | sense | TATGCCATGAGTG | 23. | CCTTTATG | 167. | AGG | 7 | 0.7533 |
| | | high | | 001270507. | 000006.12 | | | CTCAGAG | | CCATGAGT | | | | |
| | | | | 1 | | | | | | GCTCAGA | | | | |
| | | | | | | | | | | GAGGCGG | | | | |
| TNFAIP3 | CTLA4 | CTLA4 | 7128 | NM_ | NC_ | 1.38E+08 | anti- | TGAGAGACTCCA | 24. | GATTTGAG | 168. | CGG | 3 | 0.6393 |
| | | high | | 001270507. | 000006.12 | | sense | GTTGCCAG | | AGACTCCA | | | | |
| | | | | 1 | | | | | | GTTGCCAG | | | | |
| | | | | | | | | | | CGGAAT | | | | |
| MEF2D | CTLA4 | CTLA4 | 4209 | NM_ | NC_ | 1.56E+08 | sense | CAAGTACCGACGC | 25. | AGGACAA | 169. | AGG | 4 | 0.651 |
| | | high | | 005920.3 | 000001.11 | | | GCCAGCG | | GTACCGAC | | | | |
| | | | | | | | | | | GCGCCAG | | | | |
| | | | | | | | | | | CGAGGAG | | | | |
| | | | | | | | | | | C | | | | |
| MEF2D | CTLA4 | CTLA4 | 4209 | NM_ | NC_ | 1.56E+08 | sense | CATCATCCCTCAC | 26. | GTGACATC | 170. | CGG | 5 | 0.6308 |
| | | high | | 005920.3 | 000001.11 | | | GGACCCG | | ATCCCTCA | | | | |
| | | | | | | | | | | CGGACCC | | | | |
| | | | | | | | | | | GCGGCTC | | | | |
| MEF2D | CTLA4 | CTLA4 | 4209 | NM_ | NC_ | 1.56E+08 | anti- | GATGACTGCACTC | 27. | GAAGGAT | 171. | GGG | 6 | 0.6972 |
| | | high | | 005920.3 | 000001.11 | | sense | ACCAACA | | GACTGCAC | | | | |
| | | | | | | | | | | TCACCAAC | | | | |
| | | | | | | | | | | AGGGCTG | | | | |
| MEF2D | CTLA4 | CTLA4 | 4209 | NM_ | NC_ | 1.56E+08 | anti- | GGTGAGCGAATG | 28. | GGGTGGT | 172. | GGG | 9 | 0.6131 |
| | | high | | 005920.3 | 000001.11 | | sense | AGTAGACT | | GAGCGAA | | | | |
| | | | | | | | | | | TGAGTAG | | | | |
| | | | | | | | | | | ACTGGGA | | | | |
| | | | | | | | | | | GA | | | | |
| UBP1 | CTLA4 | CTLA4 | 7342 | NM_ | NC_ | 33425595 | sense | ACGCTTACTTATTT | 29. | TGAAACGC | 173. | AGG | 3 | 0.6109 |
| | | high | | 001128161. | 000003.12 | | | GAACCA | | TTACTTAT | | | | |
| | | | | 1 | | | | | | TTGAACCA | | | | |
| | | | | | | | | | | AGGTTA | | | | |
| UBP1 | CTLA4 | CTLA4 | 7342 | NM_ | NC_ | 33411659 | sense | CTGTGGGAATAAT | 30. | ATGTCTGT | 174. | AGG | 6 | 0.6499 |
| | | high | | 001128161. | 000003.12 | | | TGACACA | | GGGAATA | | | | |
| | | | | 1 | | | | | | ATTGACAC | | | | |
| | | | | | | | | | | AAGGACG | | | | |
| UBP1 | CTLA4 | CTLA4 | 7342 | NM_ | NC_ | 33408700 | sense | GGTGATTCTCTGG | 31. | CTACGGTG | 175. | AGG | 9 | 0.6695 |
| | | high | | 001128161. | 000003.12 | | | CAAAGCG | | ATTCTCTG | | | | |
| | | | | 1 | | | | | | GCAAAGC | | | | |
| | | | | | | | | | | GAGGCAG | | | | |
| UBP1 | CTLA4 | CTLA4 | 7342 | NM_ | NC_ | 33409564 | sense | TTTACTCCACGGA | 32. | AGAATTTA | 176. | AGG | 7 | 0.7811 |
| | | high | | 001128161. | 000003.12 | | | AGCACGG | | CTCCACGG | | | | |
| | | | | 1 | | | | | | AAGCACG | | | | |
| | | | | | | | | | | GAGGTGA | | | | |
| MYC | CTLA4 | CTLA4 | 4609 | NM_ | NC_ | 1.28E+08 | sense | AGAGTGCATCGA | 33. | CCTCAGAG | 177. | TGG | 2 | 0.6543 |
| | | high | | 002467.4 | 000008.11 | | | CCCCTCGG | | TGCATCGA | | | | |
| | | | | | | | | | | CCCCTCGG | | | | |
| | | | | | | | | | | TGGTCT | | | | |
| MYC | CTLA4 | CTLA4 | 4609 | NM_ | NC_ | 1.28E+08 | anti- | CTGCGGGGAGGÅ | 34. | TGCCCTGC | 178. | AGG | 2 | 0.6832 |
| | | high | | 002467.4 | 000008.11 | | sense | CTCCGTCG | | GGGGAGG | | | | |
| | | | | | | | | | | ACTCCGTC | | | | |
| | | | | | | | | | | GAGGAGA | | | | |
| MYC | CTLA4 | CTLA4 | 4609 | NM_ | NC_. | 1.28E+08 | sense | CTTCGGGGAGAC | 35. | CTCCCTTC | 179. | CGG | 2 | 0.6919 |
| | | high | | 002467.4 | 00000811 | | | AACGACGG | | GGGGAGA | | | | |
| | | | | | | | | | | CAACGAC | | | | |
| | | | | | | | | | | GGCGGTG | | | | |
| | | | | | | | | | | G | | | | |
| MYC | CTLA4 | CTLA4 | 4609 | NM_ | NC_ | 1.28E+08 | anti- | GCTGCACCGAGTC | 36. | TACGGCTG | 180. | AGG | 2 | 0.6842 |
| | | high | | 002467.4 | 000008.11 | | sense | GTAGTCG | | CACCGAGT | | | | |
| | | | | | | | | | | CGTAGTCG | | | | |
| | | | | | | | | | | AGGTCA | | | | |
| NR2C2 | CTLA4 | CTLA4 | 7182 | NM_ | NC_ | 15030406 | sense | CCAGTOGACACCC | 37. | GAGACCA | 181. | AGG | 10 | 0.6976 |
| | | high | | 003298.4 | 000003.12 | | | ATCATTG | | GTCGACAC | | | | |
| | | | | | | | | | | CCATCATT | | | | |
| | | | | | | | | | | GAGGTTG | | | | |
| NR2C2 | CTLA4 | CTLA4 | 7182 | NM_ | NC_ | 15016174 | anti- | CCCCAGTAAACGC | 38. | TCTTCCCC | 182. | AGG | 5 | 0.7308 |
| | | high | | 003298.4 | 000003.12 | | sense | TCCACAG | | AGTAAAC | | | | |
| | | | | | | | | | | GCTCCACA | | | | |
| | | | | | | | | | | GAGGCAG | | | | |
| NR2C2 | CTLA4 | CTLA4 | 7182 | NM_ | NC_ | 15024201 | anti- | GAACGTCACCTTA | 39. | TAGAGAA | 183. | TGG | 8 | 0.6168 |
| | | high | | 003298.4 | 000003.12 | | sense | GAATCCG | | CGTCACCT | | | | |
| | | | | | | | | | | TAGAATCC | | | | |
| | | | | | | | | | | GTGGCCA | | | | |
| NR2C2 | CTLA4 | CTLA4 | 7182 | NM_ | NC_ | 15023322 | anti- | TCTTTGTCTGCCA | 40. | TCCATCTT | 184. | GGG | 7 | 0.6618 |
| | | high | | 003298.4 | 000003.12 | | sense | CAAACGT | | TGTCTGCC | | | | |
| | | | | | | | | | | ACAAACGT | | | | |
| | | | | | | | | | | GGGAGT | | | | |
| HINFP | CTLA4 | CTLA4 | 25988 | NM_ | NC_ | 1.19E+08 | sense | CACACCAAGCTGA | 41. | CTACCACA | 185. | GGG | 4 | 0.6982 |
| | | high | | 015517.4 | 000011.10 | | | AACAGTG | | CCAAGCTG | | | | |
| | | | | | | | | | | AAACAGT | | | | |
| | | | | | | | | | | GGGGGCT | | | | |
| HINFP | CTLA4 | CTLA4 | 25988 | NM_ | NC_ | 1.19E+08 | sense | CATGCGCTTTCGT | 42. | ACCACATG | 186. | AGG | 8 | 0.7351 |
| | | high | | 015517.4 | 000011.10 | | | CACAGTG | | CGCTTTCG | | | | |
| | | | | | | | | | | TCACAGTG | | | | |
| | | | | | | | | | | AGGACC | | | | |
| HINFP | CTLA4 | CTLA4 | 25988 | NM_ | NC_ | 1.19E+08 | anti- | GGTGCTCTCGAAG | 43. | CGGAGGT | 187. | CGG | 6 | 0.7508 |
| | | high | | 015517.4 | 000011.10 | | sense | TTTACTG | | GCTCTCGA | | | | |
| | | | | | | | | | | AGTTTACT | | | | |
| | | | | | | | | | | GCGGTCC | | | | |
| HINFP | CTLA4 | CTLA4 | 25988 | NM_ | NC_ | 1.19E+08 | anti- | TGACTACTTACGA | 44 | CCTCTGAC | 188. | AGG | 6 | 0.7145 |
| | | high | | 015517.4 | 000011.10 | | sense | TCCAATG | | TACTTACG | | | | |
| | | | | | | | | | | ATCCAATG | | | | |
| | | | | | | | | | | AGGTCT | | | | |
| ZNF235 | CTLA4 | CTLA4 | 9310 | NM_ | NC_ | 44298866 | sense | ATATGATATCCCA | 45. | CCAGATAT | 189. | AGG | 4 | 0.6116 |
| | | high | | 004234.4 | 000019.10 | | | GTTGGAG | | GATATCCC | | | | |
| | | | | | | | | | | AGTTGGA | | | | |
| | | | | | | | | | | GAGGGAA | | | | |
| ZNF235 | CTLA4 | CTLA4 | 9310 | NM_ | NC_ | 44288951 | sense | CTGTCTAGTGAAT | 46. | ACAACTGT | 190. | GGG | 5 | 0.7225 |
| | | high | | 004234.4 | 000019.10 | | | CACATAG | | CTAGTGAA | | | | |
| | | | | | | | | | | TCACATAG | | | | |
| | | | | | | | | | | GGGATC | | | | |
| ZNF235 | CTLA4 | CTLA4 | 9310 | NM_ | NC_ | 44288764 | anti- | GCACTATATTATC | 47. | TTGTGCAC | 191. | TGG | 5 | 0.7311 |
| | | high | | 004234.4 | 000019.10 | | sense | ATCATGG | | TATATTAT | | | | |
| | | | | | | | | | | CATCATGG | | | | |
| | | | | | | | | | | TGGTGT | | | | |
| ZNF235 | CTLA4 | CTLA4 | 9310 | NM_ | NC_ | 44288559 | anti- | TTCTCATGTGTAC | 48. | GTCCTTCT | 192. | TGG | 5 | 0.6489 |
| | | high | | 004234.4 | 000019.10 | | sense | TACACGC | | CATGTGTA | | | | |
| | | | | | | | | | | CTACACGC | | | | |
| | | | | | | | | | | TGGAGA | | | | |
| NFIL3 | CTLA4 | CTLA4 | 4783 | NM_ | NC_ | 91410555 | sense | ACAAATCTTCTGC | 49 | AAGAACA | 193. | AGG | 2 | 0.6396 |
| | | high | | 001289999. | 000009.12 | | | ATGTCGG | | AATCTTCT | | | | |
| | | | | 1 | | | | | | GCATGTCG | | | | |
| | | | | | | | | | | GAGGAAA | | | | |
| NFIL3 | CTLA4 | CTLA4 | 4783 | NM_ | NC_ | 91410227 | anti- | ACAACTACTTGAC | 50. | AAATACAA | 194. | AGG | 2 | 0.7129 |
| | | high | | 001289999. | 000009.12 | | sense | ACCATCG | | CTACTTGA | | | | |
| | | | | 1 | | | | | | CACCATCG | | | | |
| | | | | | | | | | | AGGGTT | | | | |
| NFIL3 | CTLA4 | CTLA4 | 4783 | NM_ | NC_ | 91410443 | sense | GAAGCGTCGACT | 51. | GTGAGAA | 195. | TGG | 2 | 0.6849 |
| | | high | | 001289999. | 000009.12 | | | GAATGACC | | GCGTCGAC | | | | |
| | | | | 1 | | | | | | TGAATGAC | | | | |
| | | | | | | | | | | CTGGTTT | | | | |
| NFIL3 | CTLA4 | CTLA4 | 4783 | NM_ | NC_ | 91410050 | sense | GGAATTAGAGAG | 52. | CGATGGA | 196. | GGG | 2 | 0.7312 |
| | | high | | 001289999. | 000009.12 | | | CTACACAA | | ATTAGAGA | | | | |
| | | | | 1 | | | | | | GCTACACA | | | | |
| | | | | | | | | | | AGGGAGC | | | | |
| IL2 | CTLA4 | CTLA4 | 3558 | NM_ | NC_ | 1.22E+08 | anti- | AAACTTAAATGTG | 53. | TGTAAAAC | 197. | TGG | 2 | 0.6039 |
| | | high | | 000586.3 | 000004.12 | | sense | AGCATCC | | TTAAATGT | | | | |
| | | | | | | | | | | GAGCATCC | | | | |
| | | | | | | | | | | TGGTGA | | | | |
| IL2 | CTLA4 | CTLA4 | 3558 | NM_ | NC_ | 1.22E+08 | sense | ACAACTGGAGCAT | 54. | AGCTACAA | 198. | TGG | 1 | 0.531 |
| | | high | | 000586.3 | 000004.12 | | | TTACTGC | | CTGGAGC | | | | |
| | | | | | | | | | | ATTTACTG | | | | |
| | | | | | | | | | | CTGGATT | | | | |
| IL2 | CTLA4 | CTLA4 | 3558 | NM_ | NC_ | 1.22E+08 | sense | AGAAGAAGAACT | 55. | GTCTAGAA | 199. | TGG | 3 | 0.4893 |
| | | high | | 000586.3 | 000004.12 | | | CAAACCTC | | GAAGAAC | | | | |
| | | | | | | | | | | TCAAACCT | | | | |
| | | | | | | | | | | CTGGAGG | | | | |
| IL2 | CTLA4 | CTLA4 | 3558 | NM_ | NC_ | 1.22E+08 | anti- | TTCTTTGTAGAAC | 56. | TGTTTTCTT | 200. | AGG | 1 | 0.5452 |
| | | high | | 000586.3 | 000004.12 | | sense | TTGAAGT | | TGTAGAAC | | | | |
| | | | | | | | | | | TTGAAGTA | | | | |
| | | | | | | | | | | GGTGC | | | | |
| VEZF1 | CTLA4 | CTLA4 | 7716 | NM_ | NC_ | 57980604 | sense | AACAAGGCATCA | 57. | TGTAAACA | 201. | AGG | 4 | 0.5923 |
| | | high | | 007146.2 | 000017.11 | | | GTAAAAGT | | AGGCATCA | | | | |
| | | | | | | | | | | GTAAAAGT | | | | |
| | | | | | | | | | | AGGTGG | | | | |
| VEZF1 | CTLA4 | CTLA4 | 7716 | NM_ | NC_ | 57982767 | sense | ACCGGATGACTTA | 58. | AAGGACC | 202. | AGG | 2 | 0.6541 |
| | | high | | 007146.2 | 000017.11 | | | CCATGTG | | GGATGACT | | | | |
| | | | | | | | | | | TACCATGT | | | | |
| | | | | | | | | | | GAGGTCT | | | | |
| VEZF1 | CTLA4 | CTLA4 | 7716 | NM_ | NC_ | 57983069 | sense | CCTTATCTCTACCA | 59 | TTCCCCTT | 203. | GGG | 2 | 0.6992 |
| | | high | | 007146.2 | 000017.11 | | | TCGCTG | | ATCTCTAC | | | | |
| | | | | | | | | | | CATCGCTG | | | | |
| | | | | | | | | | | GGGACA | | | | |
| VEZF1 | CTLA4 | CTLA4 | 7716 | NM_ | NC_ | 57980754 | sense | TTGCCACCAAAGA | 60. | GCCTTTGC | 204. | CGG | 4 | 0.692 |
| | | high | | 007146.2 | 000017.11 | | | CAGACTG | | CACCAAAG | | | | |
| | | | | | | | | | | ACAGACTG | | | | |
| | | | | | | | | | | CGGACA | | | | |
| ATXN7L3 | CTLA4 | CTLA4 | 56970 | NM_ | NC_ | 44197610 | sense | CACGGACCCTGAT | 61. | ACGACAC | 205. | LAGG | 2 | 0.6328 |
| | | high | | 001098833. | 000017.11 | | | AGCATGA | | GGACCCTG | | | | |
| | | | | 1 | | | | | | ATAGCATG | | | | |
| | | | | | | | | | | AAGGATT | | | | |
| ATXN7L3 | CTLA4 | CTLA4 | 56970 | NM_ | NC_ | 44197712 | sense | CATCGCTCAGGAG | 62. | AGGCCATC | 206. | CGG | 2 | 0.7491 |
| | | high | | 001098833. | 000017.11 | | | ATATACG | | GCTCAGG | | | | |
| | | | | 1 | | | | | | AGATATAC | | | | |
| | | | | | | | | | | GCGGACC | | | | |
| ATXN7L3 | CTLA4 | CTLA4 | 56970 | NM_ | NC_ | 44197233 | sense | GCAGCCGAATCG | 63. | AACAGCA | 207. | CGG | 3 | 0.6135 |
| | | high | | 001098833. | 000017.11 | | | CCAACCGC | | GCCGAATC | | | | |
| | | | | 1 | | | | | | GCCAACCG | | | | |
| | | | | | | | | | | CCGGTGA | | | | |
| ATXN7L3 | CTLA4 | CTLA4 | 56970 | NM_ | NC_ | 44195424 | sense | GCTTCGCAGCCTG | 64. | AGGAGCTT | 208. | CGG | 8 | 0.6566 |
| | | high | | 001098833. | 000017.11 | | | CTAACCA | | CGCAGCCT | | | | |
| | | | | 1 | | | | | | GCTAACCA | | | | |
| | | | | | | | | | | CGGTGA | | | | |
| TFDP1 | CTLA4 | CTLA4 | 7027 | NM_ | NC_ | 1.14E+08 | sense | ACCGGCAGCGTC | 65. | AGAGACC | 209. | TGG | 5 | 0.6315 |
| | | high | | 007111.4 | 000013.11 | | | AAACACCC | | GGCAGCG | | | | |
| | | | | | | | | | | TCAAACAC | | | | |
| | | | | | | | | | | CCTGGTGG | | | | |
| TFDP1 | CTLA4 | CTLA4 | 7027 | NM_ | NC_ | 1.14E+08 | sense | ATGACCAGAAAA | 66. | GCTTATGA | 210. | CGG | 7 | 0.6204 |
| | | high | | 007111.4 | 000013.11 | | | ACATAAGA | | CCAGAAA | | | | |
| | | | | | | | | | | AACATAAG | | | | |
| | | | | | | | | | | ACGGCGC | | | | |
| TFDP1 | CTLA4 | CTLA4 | 7027 | NM_ | NC_ | 1.14E+08 | anti- | CCTTCATGGAGAA | 67. | CAGACCTT | 211. | AGG | 6 | 0.6851 |
| | | high | | 007111.4 | 000013.11 | | sense | ATGCCGT | | CATGGAG | | | | |
| | | | | | | | | | | AAATGCCG | | | | |
| | | | | | | | | | | TAGGCCC | | | | |
| TFDP1 | CTLA4 | CTLA4 | 7027 | NM_ | NC_ | 1.14E+08 | sense | GGTGCAGAGAAA | 68. | ACCTGGTG | 212. | CGG | 9 | 0.6402 |
| | | high | | 007111.4 | 000013.11 | | | CCGGCATG | | CAGAGAA | | | | |
| | | | | | | | | | | ACCGGCAT | | | | |
| | | | | | | | | | | GCGGAGC | | | | |
| NSD1 | CTLA4 | CTLA4 | 64324 | NM_ | NC_ | 1.77E+08 | sense | AAGCACATAAAG | 69. | TTTGAAGC | 213. | AGG | 5 | 0.7285 |
| | | high | | 022455.4 | 000005.10 | | | ATGAACGG | | ACATAAAG | | | | |
| | | | | | | | | | | ATGAACG | | | | |
| | | | | | | | | | | GAGGGGA | | | | |
| NSD1 | CTLA4 | CTLA4 | 64324 | NM_ | NC_ | 1.77E+08 | sense | GAATTGCTAGTTA | 70. | TGAGGAA | 214. | AGG | 7 | 0.6674 |
| | | high | | 022455.4 | 000005.10 | | | AAACGCC | | TTGCTAGT | | | | |
| | | | | | | | | | | TAAAACGC | | | | |
| | | | | | | | | | | CAGGTAA | | | | |
| NSD1 | CTLA4 | CTLA4 | 64324 | NM_ | NC_ | 1.77E+08 | sense | GCCCTATCGGCAG | 71. | GGAGGCC | 215. | TGG | 4 | 0.6824 |
| | | high | | 022455.4 | 000005.10 | | | TACTACG | | CTATCGGC | | | | |
| | | | | | | | | | | AGTACTAC | | | | |
| | | | | | | | | | | GTGGAGG | | | | |
| NSD1 | CTLA4 | CTLA4 | 64324 | NM_ | NC_ | 1.77E+08 | sense | TATGCATGATAGT | 72. | AAGATATG | 216. | AGG | 5 | 0.6849 |
| | | high | | 022455.4 | 000005.10 | | | AAGACGA | | CATGATAG | | | | |
| | | | | | | | | | | TAAGACG | | | | |
| | | | | | | | | | | AAGGAGC | | | | |
| VPS52 | CTLA4 | CTLA4 | 6293 | NM_ | NC_ | 33267957 | sense | CAATGAACGAGC | 73. | TGGGCAAT | 217. | AGG | 8 | 0.6103 |
| | | high | | 001289174. | 000006.12 | | | AACAGCAA | | GAACGAG | | | | |
| | | | | 1 | | | | | | CAACAGCA | | | | |
| | | | | | | | | | | AAGGAGA | | | | |
| VPS52 | CTLA4 | CTLA4 | 6293 | NM_ | NC_ | 33266567 | sense | CCGTACACTCAGC | 74. | TGGGCCGT | 218. | TGG | 11 | 0.6309 |
| | | high | | 001289174. | 000006.12 | | | ATGACCC | | ACACTCAG | | | | |
| | | | | 1 | | | | | | CATGACCC | | | | |
| | | | | | | | | | | TGGTAA | | | | |
| VPS52 | CTLA4 | CTLA4 | 6293 | NM_ | NC_ | 33269070 | sense | GAAATCGCCAGG | 75. | CTTCGAAA | 219. | GGG | 5 | 0.7039 |
| | | high | | 001289174. | 000006.12 | | | CAGTTCGG | | TCGCCAGG | | | | |
| | | | | 1 | | | | | | CAGTTCGG | | | | |
| | | | | | | | | | | GGGAAA | | | | |
| VPS52 | CTLA4 | CTLA4 | 6293 | NM_ | NC_ | 33264461 | anti- | TCCAGGATCAGTT | 76. | CATCTCCA | 220. | TGG | 13 | 0.6852 |
| | | high | | 001289174. | 000006.12 | | sense | CAAACCG | | GGATCAGT | | | | |
| | | | | 1 | | | | | | TCAAACCG | | | | |
| | | | | | | | | | | TGGCCA | | | | |
| TP53 | CTLA4 | CTLA4 | 7157 | NM_ | NC_ | 7676227 | anti- | CCATTGTTCAATA | 77. | TGAACCAT | 221. | GGG | 4 | 0.6899 |
| | | high | | 000546.5 | 000017.11 | | sense | TCGTCCG | | TGTTCAAT | | | | |
| | | | | | | | | | | ATCGTCCG | | | | |
| | | | | | | | | | | GGGACA | | | | |
| TP53 | CTLA4 | CTLA4 | 7157 | NM_ | NC_ | 7675058 | sense | GAGCGCTGCTCA | 78. | CCATGAGC | 222. | TGG | 5 | 0.6542 |
| | | high | | 000546.5 | 000017.11 | | | GATAGCGA | | GCTGCTCA | | | | |
| | | | | | | | | | | GATAGCG | | | | |
| | | | | | | | | | | ATGGTGA | | | | |
| TP53 | CTLA4 | CTLA4 | 7157 | NM_ | NC_ | 7676527 | anti- | GATCCACTCACAG | 79. | AATGGATC | 223. | AGG | 2 | 0.6108 |
| | | high | | 000546.5 | 000017.11 | | sense | TTTCCAT | | CACTCACA | | | | |
| | | | | | | | | | | GTTTCCAT | | | | |
| | | | | | | | | | | AGGTCT | | | | |
| TP53 | CTLA4 | CTLA4 | 7157 | NM_ | NC_ | 7674864 | sense | GGTGCCCTATGAG | 80. | TGGTGGT | 224. | AGG | 6 | 0.6259 |
| | | high | | 000546.5 | 000017.11 | | | CCGCCTG | | GCCCTATG | | | | |
| | | | | | | | | | | AGCCGCCT | | | | |
| | | | | | | | | | | GAGGTCT | | | | |
| TGIF1 | CTLA4 | CTLA4 | 7050 | NM_ | NC_ | 3457612 | anti- | ACGAGCCAAAACT | 81. | ATGGACG | 225. | GGG | 4 | 0.6815 |
| | | high | | 001278684. | 000018.10 | | sense | GATCCCG | | AGCCAAA | | | | |
| | | | | 1 | | | | | | ACTGATCC | | | | |
| | | | | | | | | | | CGGGGAT | | | | |
| | | | | | | | | | | G | | | | |
| TGIF1 | CTLA4 | CTLA4 | 7050 | NM_ | NC_ | 3456514 | anti- | CTGAAGGATAGG | 82. | TGCTCTGA | 226. | CGG | 3 | 0.6508 |
| | | high | | 001278684. | 000018.10 | | sense | CATTGTAA | | AGGATAG | | | | |
| | | | | 1 | | | | | | GCATTGTA | | | | |
| | | | | | | | | | | ACGGTGC | | | | |
| TGIF1 | CTLA4 | CTLA4 | 7050 | NM_ | NC_ | 3457459 | sense | GTTCACAATTTCC | 83. | ATCAGTTC | 227. | GGG | 4 | 0.6788 |
| | | high | | 001278684. | 000018.10 | | | CGCCGTG | | ACAATTTC | | | | |
| | | | | 1 | | | | | | CCGCCGTG | | | | |
| | | | | | | | | | | GGGCCA | | | | |
| TGIF1 | CTLA4 | CTLA4 | 7050 | NM_ | NC_ | 3456466 | anti- | TCTGCACAGACTC | 84. | AGAATCTG | 228. | AGG | 3 | 0.6745 |
| | | high | | 001278684. | 000018.10 | | sense | CTTGGGT | | CACAGACT | | | | |
| | | | | 1 | | | | | | CCTTGGGT | | | | |
| | | | | | | | | | | AGGTTG | | | | |
| ZBTB7B | CTLA4 | CTLA4 | 51043 | NM_ | NC_ | 1.55E+08 | sense | AGCAAACCACCTA | 85. | CCAGAGC | 229. | AGG | 2 | 0.6906 |
| | | high | | 001256455. | 000001.11 | | | GTCCCTG | | AAACCACC | | | | |
| | | | | 1 | | | | | | TAGTCCCT | | | | |
| | | | | | | | | | | GAGGTGC | | | | |
| ZBTB7B | CTLA4 | CTLA4 | 51043 | NM_ | NC_ | 1.55E+08 | sense | CAGAGCTACGAAC | 86. | TCCCCAGA | 230. | GGG | 2 | 0.6187 |
| | | high | | 001256455. | 000001.11 | | | CCTATGA | | GCTACGAA | | | | |
| | | | | 1 | | | | | | CCCTATGA | | | | |
| | | | | | | | | | | GGGTGA | | | | |
| ZBTB7B | CTLA4 | CTLA4 | 51043 | NM_ | NC_ | 1.55E+08 | anti- | TCCGGATGGTGA | 87. | TGCGTCCG | 231. | AGG | 2 | 0.6762 |
| | | high | | 001256455. | 000001.11 | | sense | GGTCACAT | | GATGGTG | | | | |
| | | | | 1 | | | | | | AGGTCACA | | | | |
| | | | | | | | | | | TAGGTGG | | | | |
| ZBTB7B | CTLA4 | CTLA4 | 51043 | NM_ | NC_ | 1.55E+08 | anti- | TGTATAGGCAAAT | 88. | TGGCTGTA | 232. | GGG | 2 | 0.6181 |
| | | high | | 001256455. | 000001.11 | | sense | TCAAGGA | | TAGGCAA | | | | |
| | | | | 1 | | | | | | ATTCAAGG | | | | |
| | | | | | | | | | | AGGGCGC | | | | |
| USF2 | CTLA4 | CTLA4 | 7392 | NM_ | NC_ | 35270541 | anti- | AGCCGTAGTATCT | 89. | ACACAGCC | 233. | TGG | 5 | 0.6444 |
| | | high | | 003367.2 | 000019.10 | | sense | CCCACAC | | GTAGTATC | | | | |
| | | | | | | | | | | TOCCACAC | | | | |
| | | | | | | | | | | TGGACG | | | | |
| USF2 | CTLA4 | CTLA4 | 7392 | NM_ | NC_ | 35269463 | sense | CCACGACAAGGG | 90. | GCAGCCAC | 234. | CGG | 2 | 0.6052 |
| | | high | | 003367.2 | 000019.10 | | | ACCCGAGG | | GACAAGG | | | | |
| | | | | | | | | | | GACCCGA | | | | |
| | | | | | | | | | | GGCGGAG | | | | |
| | | | | | | | | | | G | | | | |
| USF2 | CTLA4 | CTLA4 | 7392 | NM_ | NC_ | 35270800 | anti- | CCTGCACATACGG | 91. | GTCCCCTG | 235. | GGG | 6 | 0.6405 |
| | | high | | 003367.2 | 000019.10 | | sense | AGAGTAA | | CACATACG | | | | |
| | | | | | | | | | | GAGAGTA | | | | |
| | | | | | | | | | | AGGGTGT | | | | |
| USF2 | CTLA4 | CTLA4 | 7392 | NM_ | NC_ | 35269690 | sense | TTCCGCACAGAGA | 92. | CCAGTTCC | 236. | AGG | 3 | 0.6997 |
| | | high | | 003367.2 | 1000019.0 | | | CAAATGG | | GCACAGA | | | | |
| | | | | | | | | | | GACAAAT | | | | |
| | | | | | | | | | | GGAGGAC | | | | |
| | | | | | | | | | | A | | | | |
| TBX21 | CTLA4 | CTLA4 | 30009 | NM_ | NC_ | 47744217 | anti- | CCGGGGCTGGTA | 93. | GCAGCCG | 237. | GGG | 4 | 0.6246 |
| | | high | | 013351.1 | 000017.11 | | sense | CTTATGGA | | GGGCTGG | | | | |
| | | | | | | | | | | TACTTATG | | | | |
| | | | | | | | | | | GAGGGAC | | | | |
| | | | | | | | | | | T | | | | |
| TBX21 | CTLA4 | CTLA4 | 30009 | NM_ | NC_ | 47733630 | sense | CCTGGGGTCTCCC | 94. | GCAGCCTG | 238. | GGG | 1 | 0.6294 |
| | | high | | 013351.1 | 000017.11 | | | TACCCGG | | GGGTCTCC | | | | |
| | | | | | | | | | | CTACCCGG | | | | |
| | | | | | | | | | | GGGGCG | | | | |
| TBX21 | CTLA4 | CTLA4 | 30009 | NM_ | NC_ | 47742726 | sense | GCGGTACCAGAG | 95. | ACTGGCG | 239. | GGG | 2 | 0.6321 |
| | | high | | 013351.1 | 000017.11 | | | CGGCAAGT | | GTACCAGA | | | | |
| | | | | | | | | | | GCGGCAA | | | | |
| | | | | | | | | | | GTGGGTG | | | | |
| | | | | | | | | | | C | | | | |
| TBX21 | CTLA4 | CTLA4 | 30009 | NM_ | NC_ | 47733889 | anti- | TAAACTTGGACCA | 96. | TGATTAAA | 240. | TGG | 1 | 0.6344 |
| | | high | | 013351.1 | 000017.11 | | sense | CAACAGG | | CTTGGACC | | | | |
| | | | | | | | | | | ACAACAG | | | | |
| | | | | | | | | | | GTGGTTG | | | | |
| JUN | CTLA4 | CTLA4 | 3725 | NM_ | NC_ | 58782886 | anti- | GCCCCACGTCGG | 97. | AGCAGCCC | 241. | AGG | 1 | 0.56 |
| | | high | | 002228.3 | 000001.11 | | sense | GCGAGGTG | | CACGTCGG | | | | |
| | | | | | | | | | | GCGAGGT | | | | |
| | | | | | | | | | | GAGGAGG | | | | |
| JUN | CTLA4 | CTLA4 | 3725 | NM_ | NC_ | 58783019 | anti- | GCTCTCGGACGG | 98. | GTCCGCTC | 242. | AGG | 1 | 0.6332 |
| | | high | | 002228.3 | 000001.11 | | sense | GAGGAACG | | TCGGACG | | | | |
| | | | | | | | | | | GGAGGAA | | | | |
| | | | | | | | | | | CGAGGCG | | | | |
| | | | | | | | | | | T | | | | |
| JUN | CTLA4 | CTLA4 | 3725 | NM_ | NC_ | 58782659 | sense | GGCGGCGCAGCC | 99. | CGTCGGC | 243. | GGG | 1 | 0.7008 |
| | | high | | 002228.3 | 000001.11 | | | GGTCAACG | | GGCGCAG | | | | |
| | | | | | | | | | | CCGGTCAA | | | | |
| | | | | | | | | | | CGGGGCA | | | | |
| | | | | | | | | | | G | | | | |
| JUN | CTLA4 | CTLA4 | 3725 | NM_ | NC_ | 58782936 | sense | TGAACCTGGCCGA | 100. | ACCCTGAA | 244. | GGG | 1 | 0.6651 |
| | | high | | 002228.3 | 000001.11 | | | CCCAGTG | | CCTGGCCG | | | | |
| | | | | | | | | | | ACCCAGTG | | | | |
| | | | | | | | | | | GGGAGC | | | | |
| EGR2 | CTLA4 | CTLA4 | 1959 | NM_ | NC_ | 62815887 | anti- | CTCCGTTCATCTG | 101. | GCCACTCC | 245. | GGG | 1 | 0.6291 |
| | | high | | 000399.3 | 000010.11 | | sense | GTCAAAG | | GTTCATCT | | | | |
| | | | | | | | | | | GGTCAAA | | | | |
| | | | | | | | | | | GGGGCCT | | | | |
| EGR2 | CTLA4 | CTLA4 | 1959 | NM_ | NC_ | 62815873 | sense | CTTTGACCAGATG | 102. | GCCCCTTT | 246. | TGG | 1 | 0.7256 |
| | | high | | 000399.3 | 000010.11 | | | AACGGAG | | GACCAGAT | | | | |
| | | | | | | | | | | GAACGGA | | | | |
| | | | | | | | | | | GTGGCCG | | | | |
| EGR2 | CTLA4 | CTLA4 | 1959 | NM_ | NC_ | 62815927 | anti- | GGGAAAGATGGT | 103. | CATTGGGA | 247. | TGG | 1 | 0.6771 |
| | | high | | 000399.3 | 000010.11 | | sense | CACCGACG | | AAGATGG | | | | |
| | | | | | | | | | | TCACCGAC | | | | |
| | | | | | | | | | | GTGGCGG | | | | |
| EGR2 | CTLA4 | CTLA4 | 1959 | NM_ | NC_ | 62815951 | sense | GTCTGACAACATC | 104. | AGCTGTCT | 248. | TGG | 1 | 0.6887 |
| | | high | | 000399.3 | 000010.11 | | | TACCCGG | | GACAACAT | | | | |
| | | | | | | | | | | CTACCCGG | | | | |
| | | | | | | | | | | TGGAGG | | | | |
| NFATC2 | CTLA4 | CTLA4 | 4773 | NM_ | NC_ | 51474071 | sense | ACATTGGAAGAA | 105. | ACGGACAT | 249. | CGG | 5 | 0.7428 |
| | | high | | 001258297. | 000020.11 | | | AGAACACG | | TGGAAGA | | | | |
| | | | | 1 | | | | | | AAGAACA | | | | |
| | | | | | | | | | | CGCGGGT | | | | |
| | | | | | | | | | | G | | | | |
| NFATC2 | CTLA4 | CTLA4 | 4773 | NM_ | NC_ | 51454601 | anti- | ATGTAAAGTTCTG | 106. | TCGGATGT | 250. | AGG | 6 | 0.7057 |
| | | high | | 001258297. | 000020.11 | | sense | CCCCGTG | | AAAGTTCT | | | | |
| | | | | 1 | | | | | | GCCCCGTG | | | | |
| | | | | | | | | | | AGGATC | | | | |
| NFATC2 | CTLA4 | CTLA4 | 4773 | NM_ | NC_ | 51523157 | sense | GCAGGGCGAGAG | 107. | GCGAGCA | 251. | CGG | 2 | 0.6354 |
| | | high | | 001258297. | 000020.11 | | | GAGAAACT | | GGGCGAG | | | | |
| | | | | 1 | | | | | | AGGAGAA | | | | |
| | | | | | | | | | | ACTCGGCT | | | | |
| | | | | | | | | | | C | | | | |
| NFATC2 | CTLA4 | CTLA4 | 4773 | NM_ | NC_ | 51523397 | sense | GCCGCAGCCCTCA | 108. | CCTCGCCG | 252. | TGG | 2 | 0.6587 |
| | | high | | 001258297. | 000020.11 | | | TCTCACG | | CAGCCCTC | | | | |
| | | | | 1 | | | | | | ATCTCACG | | | | |
| | | | | | | | | | | TGGCAC | | | | |
| RUNX3 | CTLA4 | CTLA4 | 864 | NM_ | NC_ | 24927597 | anti- | CACTGCGGCCCAC | 109. | CGCCCACT | 253. | AGG | 2 | 0.6012 |
| | | high | | 004350.2 | 000001.11 | | sense | GAAGCGA | | GCGGCCC | | | | |
| | | | | | | | | | | ACGAAGC | | | | |
| | | | | | | | | | | GAAGGTC | | | | |
| | | | | | | | | | | G | | | | |
| RUNX3 | CTLA4 | CTLA4 | 864 | NM_ | NC_ | 24902559 | sense | CCCCAGGATGCAT | 110. | CAGACCCC | 254. | GGG | 5 | 0.642 |
| | | high | | 004350.2 | 000001.11 | | | TATCCCG | | AGGATGC | | | | |
| | | | | | | | | | | ATTATCCC | | | | |
| | | | | | | | | | | GGGGCCA | | | | |
| RUNX3 | CTLA4 | CTLA4 | 864 | NM_ | NC_ | 24907265 | anti- | CCGTGCCGTACCT | 111. | CCCTCCGT | 255. | GGG | 4 | 0.6118 |
| | | high | | 004350.2 | 000001.11 | | sense | TGGATTG | | GCCGTACC | | | | |
| | | | | | | | | | | TTGGÅTTG | | | | |
| | | | | | | | | | | GGGTCT | | | | |
| RUNX3 | CTLA4 | CTLA4 | 864 | NM_ | NC_ | 24919297 | anti- | TCGGTGGTAGGTC | 112. | TGGCTCGG | 256. | GGG | 3 | 0.5939 |
| | | high | | 004350.2 | 000001.11 | | sense | GCCACTT | | TGGTAGGT | | | | |
| | | | | | | | | | | CGCCACTT | | | | |
| | | | | | | | | | | GGGTGG | | | | |
| SATB1 | CTLA4 | CTLA4 | 6304 | NM_ | NC_ | 18415117 | anti- | ATGCTAAGTACCT | 113. | TTCTATGC | 257. | GGG | 5 | 0.6378 |
| | | high | | 002971.4 | 000003.12 | | sense | GTGAAAG | | TAAGTACC | | | | |
| | | | | | | | | | | TGTGAAA | | | | |
| | | | | | | | | | | GGGGGCA | | | | |
| SATB1 | CTLA4 | CTLA4 | 6304 | NM_ | NC_ | 18417016 | sense | CATTGAATATGAT | 114 | ACGCCATT | 258. | AGG | 3 | 0.7747 |
| | | high | | 002971.4 | 000003.12 | | | TGCAAGG | | GAATATGA | | | | |
| | | | | | | | | | | TTGCAAGG | | | | |
| | | | | | | | | | | AGGAGC | | | | |
| SATB1 | CTLA4 | CTLA4 | 6304 | NM_ | NC_ | 18394751 | anti- | TAGGTGTTGATAC | 115. | CTGATAGG | 259. | GGG | 7 | 0.6656 |
| | | high | | 002971.4 | 000003.12 | | sense | GAGCCCA | | TGTTGATA | | | | |
| | | | | | | | | | | CGAGCCCA | | | | |
| | | | | | | | | | | GGGTGC | | | | |
| SATB1 | CTLA4 | CTLA4 | 6304 | NM_ | NC_ | 18394610 | anti- | TATTCATAGATCT | 116. | GGCTTATT | 260. | GGG | 7 | 0.6676 |
| | | high | | 002971.4 | 000003.12 | | sense | ACTGACA | | CATAGATC | | | | |
| | | | | | | | | | | TACTGACA | | | | |
| | | | | | | | | | | GGGGGA | | | | |
| FOS | CTLA4 | CTLA4 | 2353 | NM_ | NC_ | 75280663 | sense | GCTGACTGATACA | 117. | GGGAGCT | 261. | CGG | 3 | 0.7252 |
| | | high | | 005252.3 | 000014.9 | | | CTCCAAG | | GACTGATA | | | | |
| | | | | | | | | | | CACTCCAA | | | | |
| | | | | | | | | | | GCGGTAG | | | | |
| FOS | CTLA4 | CTLA4 | 2353 | NM_ | NC_ | 75280868 | sense | GGAAAAACTAGA | 118. | AGAAGGA | 262. | TGG | 4 | 0.6049 |
| | | high | | 005252.3 | 000014.9 | | | GTTCATCC | | AAAACTAG | | | | |
| | | | | | | | | | | AGTTCATC | | | | |
| | | | | | | | | | | CTGGCAG | | | | |
| FOS | CTLA4 | CTLA4 | 2353 | NM_ | NC_ | 75279056 | anti- | GTAGTAAGAGAG | 119. | AGTGGTA | 263. | CGG | 1 | 0.6017 |
| | | high | | 005252.3 | 000014.9 | | sense | GCTATCCC | | GTAAGAG | | | | |
| | | | | | | | | | | AGGCTATC | | | | |
| | | | | | | | | | | CCCGGCCG | | | | |
| FOS | CTLA4 | CTLA4 | 2353 | NM_ | NC_ | 75279925 | anti- | GTCGAGATGGCA | 120. | ACTGGTCG | 264. | GGG | 2 | 0.6244 |
| | | high | | 005252.3 | 000014.9 | | sense | GTGACCGT | | AGATGGC | | | | |
| | | | | | | | | | | AGTGACC | | | | |
| | | | | | | | | | | GTGGGAA | | | | |
| | | | | | | | | | | T | | | | |
| ZNF217 | CTLA4 | CTLA4 | 7764 | NM_ | NC_ | 53581993 | sense | CAAAATCTCACCC | 121. | AGACCAA | 265. | GGG | 1 | 0.6608 |
| | | high | | 006526.2 | 000020.11 | | | TGAAACG | | AATCTCAC | | | | |
| | | | | | | | | | | CCTGAAAC | | | | |
| | | | | | | | | | | GGGGAAG | | | | |
| ZNF217 | CTLA4 | CTLA4 | 7764 | NM_ | NC_ | 53581749 | sense | CCACGGCGAAGC | 122. | ACTCCCAC | 266. | TGG | 1 | 0.6177 |
| | | high | | 006526.2 | 000020.11 | | | GCCCTCCG | | GGCGAAG | | | | |
| | | | | | | | | | | CGCCCTCC | | | | |
| | | | | | | | | | | GTGGACG | | | | |
| ZNF217 | CTLA4 | CTLA4 | 7764 | NM_ | NC_ | 53582284 | sense | GGACACATAATG | 123. | ATGCGGA | 267. | GGG | 1 | 0.7154 |
| | | high | | 006526.2 | 000020.11 | | | GCAAATCG | | CACATAAT | | | | |
| | | | | | | | | | | GGCAAATC | | | | |
| | | | | | | | | | | GGGGGCC | | | | |
| ZNF217 | CTLA4 | CTLA4 | 7764 | NM_ | NC_ | 53576811 | anti- | TGGGTGGTACTGC | 124. | GTTATGGG | 268. | AGG | 3 | 0.6707 |
| | | high | | 006526.2 | 000020.11 | | sense | CATCCGG | | TGGTACTG | | | | |
| | | | | | | | | | | CCATCCGG | | | | |
| | | | | | | | | | | AGGAGG | | | | |
| FOXK1 | CTLA4 | CTLA4 | 221937 | NM_ | NC_ | 4757132 | sense | AACAGGCATTOCG | 125. | GTGGAAC | 269. | AGG | 5 | 0.6681 |
| | | high | | 001037165. | 000007.14 | | | GAAACGG | | AGGCATTC | | | | |
| | | | | 1 | | | | | | CGGAAAC | | | | |
| | | | | | | | | | | GGAGGCA | | | | |
| | | | | | | | | | | G | | | | |
| FOXK1 | CTLA4 | CTLA4 | 221937 | NM_ | NC_ | 4754516 | anti- | AGGTCACGTTCTG | 126. | TCCGAGGT | 270. | CGG | 3 | 0.734 |
| | | high | | 001037165. | 000007.14 | | sense | CACAAAG | | CACGTTCT | | | | |
| | | | | 1 | | | | | | GCACAAA | | | | |
| | | | | | | | | | | GCGGTAA | | | | |
| FOXK1 | CTLA4 | CTLA4 | 221937 | NM_ | NC_ | 4759162 | anti- | CTAACTTGGACCC | 127. | GAAGCTA | 271. | GGG | 6 | 0.6672 |
| | | high | | 001037165. | 000007.14 | | sense | AAACTCA | | ACTTGGAC | | | | |
| | | | | 1 | | | | | | CCAAACTC | | | | |
| | | | | | | | | | | AGGGTCG | | | | |
| FOXK1 | CTLA4 | CTLA4 | 221937 | NM_ | NC_ | 4755361 | sense | GCATTACCCCTAC | 128. | CCAAGCAT | 272. | CGG | 4 | 0.7131 |
| | | high | | 001037165. | 000007.14 | | | TACCGGA | | TACCCCTA | | | | |
| | | | | 1 | | | | | | CTACCGGA | | | | |
| | | | | | | | | | | CGGCCG | | | | |
| SMARCB1 | CTLA4 | CTLA4 | 6598 | NM_ | NC_ | 23791773 | anti- | GAGAACCTCGGA | 129. | TACAGAG | 273. | AGG | 2 | 0.731 |
| | | high | | 003073.3 | 000022.11 | | sense | ACATACGG | | AACCTCGG | | | | |
| | | | | | | | | | | AACATACG | | | | |
| | | | | | | | | | | GAGGTAG | | | | |
| SMARCB1 | CTLA4 | CTLA4 | 6598 | NM_ | NC_ | 23816887 | sense | GCAGATCGAGTCC | 130. | GACAGCA | 274. | CGG | 6 | 0.6894 |
| | | high | | 003073.3 | 000022.11 | | | TACCCCA | | GATCGAGT | | | | |
| | | | | | | | | | | CCTACCCC | | | | |
| | | | | | | | | | | ACGGACA | | | | |
| SMARCB1 | CTLA4 | CTLA4 | 6598 | NM_ | NC_ | 23801049 | anti- | TCTTCTTGTCTCG | 131. | GTTCTCTT | 275. | CGG | 4 | 0.6806 |
| | | high | | 003073.3 | 000022.11 | | sense | GCCCATG | | CTTGTCTC | | | | |
| | | | | | | | | | | GGCCCATG | | | | |
| | | | | | | | | | | CGGTTC | | | | |
| SMARCB1 | CTLA4 | CTLA4 | 6598 | NM_ | NC_ | 23803342 | sense | TGAGAACGCATCT | 132. | TCCATGAG | 276. | AGG | 5 | 0.6804 |
| | | high | | 003073.3 | 000022.11 | | | CAGCCCG | | AACGCATC | | | | |
| | | | | | | | | | | TCAGCCCG | | | | |
| | | | | | | | | | | AGGTGC | | | | |
| FLI1 | CTLA4 | CTLA4 | 2313 | NM_ | NC_ | 1.29E+08 | anti- | ACTCAATCGTGAG | 133. | TGACACTC | 277. | CGG | 5 | 0.6162 |
| | | high | | 002017.4 | 000011.10 | | sense | GATTGGT | | AATCGTGA | | | | |
| | | | | | | | | | | GGATTGGT | | | | |
| | | | | | | | | | | CGGTGT | | | | |
| FLI1 | CTLA4 | CTLA4 | 2313 | NM_ | NC_ | 1.29E+08 | sense | ACTGTGTAAAATG | 134. | AGGAACT | 278. | AGG | 4 | 0.8003 |
| | | high | | 002017.4 | 000011.10 | | | AACAAGG | | GTGTAAAA | | | | |
| | | | | | | | | | | TGAACAA | | | | |
| | | | | | | | | | | GGAGGAC | | | | |
| | | | | | | | | | | T | | | | |
| FLI1 | CTLA4 | CTLA4 | 2313 | NM_ | NC_ | 1.29E+08 | sense | CCAACATGACCAC | 135. | CCTCCCAA | 279. | AGG | 3 | 0.6923 |
| | | high | | 002017.4 | 000011.10 | | | CAACGAG | | CATGACCA | | | | |
| | | | | | | | | | | CCAACGA | | | | |
| | | | | | | | | | | GAGGAGA | | | | |
| FLI1 | CTLA4 | CTLA4 | 2313 | NM_ | NC_ | 1.29E+08 | anti- | CTTACCTGGATCC | 136. | TGAGCTTA | 280. | TGG | 2 | 0.7136 |
| | | high | | 002017.4 | 000011.10 | | sense | ATTCATG | | CCTGGATC | | | | |
| | | | | | | | | | | CATTCATG | | | | |
| | | | | | | | | | | TGGTCA | | | | |
| MYB | CTLA4 | CTLA4 | 4602 | NM_ | NC_ | 1.35E+08 | sense | ACCAGGCACACAA | 137. | ATTTACCA | 281. | GGG | 5 | 0.6859 |
| | | high | | 005375.2 | 000006.12 | | | GAGACTG | | GGCACAC | | | | |
| | | | | | | | | | | AAGAGAC | | | | |
| | | | | | | | | | | TGGGGAA | | | | |
| | | | | | | | | | | C | | | | |
| MYB | CTLA4 | CTLA4 | 4602 | NM_ | NC_ | 1.35E+08 | sense | AGAAATACGGTCC | 138. | GTACAGA | 282. | TGG | 5 | 0.6009 |
| | | high | | 005375.2 | 000006.12 | | | GAAACGT | | AATACGGT | | | | |
| | | | | | | | | | | CCGAAAC | | | | |
| | | | | | | | | | | GTTGGTCT | | | | |
| MYB | CTLA4 | CTLA4 | 4602 | NM_ | NC_ | 1.35E+08 | sense | AGTCTGGAAAGC | 139. | CCCAAGTC | 283. | GGG | 2 | 0.7063 |
| | | high | | 005375.2 | 000006.12 | | | GTCACTTG | | TGGAAAG | | | | |
| | | | | | | | | | | CGTCACTT | | | | |
| | | | | | | | | | | GGGGAAA | | | | |
| MYB | CTLA4 | CTLA4 | 4602 | NM_ | NC_ | 1.35E+08 | anti- | TATTTACATGTAA | 140. | ACTATATT | 284. | GGG | 7 | 0.6157 |
| | | high | | 005375.2 | 000006.12 | | sense | CGCTACA | | TACATGTA | | | | |
| | | | | | | | | | | ACGCTACA | | | | |
| | | | | | | | | | | GGGTAT | | | | |
| CBFB | CTLA4 | CTLA4 | 865 | NM_ | NC_ | 67036720 | anti- | AAGTCGACATACT | 141. | TTCTAAGT | 285. | AGG | 3 | 0.5993 |
| | | high | | 001755.2 | 000016.10 | | sense | CTCGGCT | | CGACATAC | | | | |
| | | | | | | | | | | TCTCGGCT | | | | |
| | | | | | | | | | | AGGTGT | | | | |
| CBFB | CTLA4 | CTLA4 | 865 | NM_ | NC_ | 67029479 | anti- | CCTGCCTCACCTC | 142. | CCCGCCTG | 286. | CGG | 1 | 0.6743 |
| | | high | | 001755.2 | 000016.10 | | sense | ACACTCG | | CCTCACCT | | | | |
| | | | | | | | | | | CACACTCG | | | | |
| | | | | | | | | | | CGGCTC | | | | |
| CBFB | CTLA4 | CTLA4 | 865 | NM_ | NC_ | 67029807 | anti- | GCCGACTTACGAT | 143. | GCCAGCC | 287. | CGG | 2 | 0.7383 |
| | | high | | 001755.2 | 000016.10 | | sense | TTCCGAG | | GACTTACG | | | | |
| | | | | | | | | | | ATTTCCGA | | | | |
| | | | | | | | | | | GCGGCCG | | | | |
| CBFB | CTLA4 | CTLA4 | 865 | NM_ | NC_ | 67066729 | sense | GGAGTCTGTGTTA | 144. | GAATGGA | 288. | AGG | 4 | 0.5938 |
| | | high | | 001755.2 | 000016.10 | | | TCTGGAA | | GTCTGTGT | | | | |
| | | | | | | | | | | TATCTGGA | | | | |
| | | | | | | | | | | AAGGCTG | | | | |

TABLE 2
Nuclear factors that can be inhibited to decrease CTLA4 expression or overexpressed to increase CTLA4 expression

| | | | | | | Position of | | | | | | | | Rule |
|---|---|---|---|---|---|---|---|---|---|---|---|---|---|---|
| | | | Target | | | Base After | | sgRNA | | | | | | Set |
| gene_ | screen_ | screen_ | Gene | Target | Genomic | Cut (1- | | Target | SEQ ID | | SEQ ID | PAM | Exon | 2 |
| id | target | direction | ID | Transcript | Sequence | based) | Strand | Sequence | NO: | Target Context Sequence | NO. | Sequence | Number | score |
| CTLA4 | CTLA4 | CTLA4 | 1493 | NM_005214.4 | NC_000002.12 | 2.04E+08 | sense | CCTCACTA | 289. | TGAACCTCACTATCCAAGGACTG | 381. | GGG | 2 | 0.6385 |
| | | low | | | | | | TCCAAGG | | AGGGCCA | | | | |
| | | | | | | | | ACTGA | | | | | | |
| CTLA4 | CTLA4 | CTLA4 | 1493 | NM_005214.4 | NC_000002.12 | 2.04E+08 | antisense | GCAGATG | 290. | CCGTGCAGATGGAATCATCTAG | 382. | AGG | 2 | 0.6184 |
| | | low | | | | | | GAATCAT | | GAAGGTCA | | | | |
| | | | | | | | | CTAGGA | | | | | | |
| CTLA4 | CTLA4 | CTLA4 | 1493 | NM_005214.4 | NC_000002.12 | 2.04E+08 | sense | TACCCACC | 291. | CATGTACCCACCGCCATACTACC | 383. | GGG | 2 | 0.6501 |
| | | low | | | | | | GCCATAC | | TGGGCAT | | | | |
| | | | | | | | | TACCT | | | | | | |
| CTLA4 | CTLA4 | CTLA4 | 1493 | NM_005214.4 | NC_000002.12 | 2.04E+08 | sense | TTCCATGC | 292. | GCTTTTCCATGCTAGCAATGCAC | 384. | TGG | 2 | 0.642 |
| | | low | | | | | | TAGCAAT | | GTGGCCC | | | | |
| | | | | | | | | GCACG | | | | | | |
| FOXP1 | CTLA4 | CTLA4 | 27086 | NM_032682.5 | NC_000003.12 | 71041428 | antisense | AGAGGAG | 293. | GTGCAGAGGAGGAGACACATGT | 385. | TGG | 11 | 0.6926 |
| | | low | | | | | | GAGACAC | | CGTGGTCA | | | | |
| | | | | | | | | ATGTCG | | | | | | |
| FOXP1 | CTLA4 | CTLA4 | 27086 | NM_032682.5 | NC_000003.12 | 71015617 | antisense | CATACACC | 294. | CTTGCATACACCATGTCCATAGA | 386. | AGG | 12 | 0.6547 |
| | | low | | | | | | ATGTCCAT | | GAGGATG | | | | |
| | | | | | | | | AGAG | | | | | | |
| FOXP1 | CTLA4 | CTLA4 | 27086 | NM_032682.5 | NC_000003.12 | 71046982 | sense | GCCTTCTG | 295. | CAAGGCCTTCTGACAATTCAGCC | 387. | GGG | 10 | 0.5683 |
| | | low | | | | | | ACAATTCA | | CGGGCAG | | | | |
| | | | | | | | | GCCC | | | | | | |
| FOXP1 | CTLA4 | CTLA4 | 27086 | NM_032682.5 | NC_000003.12 | 70988031 | antisense | GTTCTGTA | 296. | TTGGGTTCTGTAGACTTCACATG | 388. | AGG | 14 | 0.5925 |
| | | low | | | | | | GACTTCA | | CAGGTGG | | | | |
| | | | | | | | | CATGC | | | | | | |
| PTEN | CTLA4 | CTLA4 | 5728 | NM_001304718.1 | NC_000010.11 | 87961027 | sense | AGAGCGT | 297. | GTATAGAGCGTGCAGATAATGA | 389. | AGG | 8 | 0.7179 |
| | | low | | | | | | GCAGATA | | CAAGGAAT | | | | |
| | | | | | | | | ATGACA | | | | | | |
| PTEN | CTLA4 | CTLA4 | 5728 | NM_001304718.1 | NC_000010.11 | 87957861 | antisense | AGCTGGC | 298. | CTTTAGCTGGCAGACCACAAACT | 390. | AGG | 7 | 0.6493 |
| | | low | | | | | | AGACCAC | | GAGGATC | | | | |
| | | | | | | | | AAACTG | | | | | | |
| PTEN | CTLA4 | CTLA4 | 5728 | NM_001304718.1 | NC_000010.11 | 87960940 | sense | ATTCTTCA | 299. | ATACATTCTTCATACCAGGACCA | 391. | AGG | 8 | 0.6697 |
| | | low | | | | | | TACCAGG | | GAGGAAA | | | | |
| | | | | | | | | ACCAG | | | | | | |
| PTEN | CTLA4 | CTLA4 | 5728 | NM_001304718.1 | NC_000010.11 | 87957915 | sense | CCAATTCA | 300. | TCCTCCAATTCAGGACCCACACG | 392. | CGG | 7 | 0.6752 |
| | | low | | | | | | GGACCCA | | ACGGGAA | | | | |
| | | | | | | | | CACGA | | | | | | |
| FOXO1 | CTLA4 | CTLA4 | 2308 | NM_002015.3 | NC_000013.11 | 40665740 | antisense | ACAGGTT | 301. | TAGGACAGGTTGCCCCACGOGTT | 393. | CGG | 1 | 0.547 |
| | | low | | | | | | GCCCCAC | | GCGGCGG | | | | |
| | | | | | | | | GCGTTG | | | | | | |
| FOXO1 | CTLA4 | CTLA4 | 2308 | NM_002015.3 | NC_000013.11 | 40666107 | sense | GGAGTTT | 302. | GGCCGGAGTTTAGCCAGTCCAA | 394. | CGG | 1 | 0.629 |
| | | low | | | | | | AGCCAGT | | CTCGGCCA | | | | |
| | | | | | | | | CCAACT | | | | | | |
| FOXO1 | CTLA4 | CTLA4 | 2308 | NM_002015.3 | NC_000013.11 | 40560279 | antisense | GGTGGCG | 303. | GTTTGGTGGCGCAAACGAGTAG | 395. | CGG | 2 | 0.6741 |
| | | low | | | | | | CAAACGA | | CACGGCGT | | | | |
| | | | | | | | | GTAGCA | | | | | | |
| FOXO1 | CTLA4 | CTLA4 | 2308 | NM_002015.3 | NC_000013.11 | 40560544 | antisense | TAGCATTT | 304. | GTACTAGCATTTGAGCTAGTTCG | 396. | GGG | 2 | 0.6665 |
| | | low | | | | | | GAGCTAG | | AGGGCGA | | | | |
| | | | | | | | | TTCGA | | | | | | |
| MED12 | CTLA4 | CTLA4 | 9968 | NM_005120.2 | NC_000023.11 | 71130165 | sense | ACATCGA | 305. | ATCCACATCGACTGCTGGACAAT | 397. | AGG | 28 | 0.6457 |
| | | low | | | | | | CTGCTGG | | GAGGATG | | | | |
| | | | | | | | | ACAATG | | | | | | |
| MED12 | CTLA4 | CTLA4 | 9968 | NM_005120.2 | NC_000023.11 | 71122231 | antisense | CAGTGAG | 306. | CAGTCAGTGAGTAGTGCCAAAC | 398. | AGG | 8 | 0.688 |
| | | low | | | | | | TAGTGCC | | CAAGGCAC | | | | |
| | | | | | | | | AAACCA | | | | | | |
| MED12 | CTLA4 | CTLA4 | 9968 | NM_005120.2 | NC_000023.11 | 71125111 | antisense | GTGGCGT | 307. | ATGGGTGGCGTACTGCACGTGT | 399. | TGG | 15 | 0.6661 |
| | | low | | | | | | ACTGCAC | | CGTGGCTG | | | | |
| | | | | | | | | GTGTCG | | | | | | |
| MED12 | CTLA4 | CTLA4 | 9968 | NM_005120.2 | NC_000023.11 | 71126138 | sense | TTCACATT | 308. | ACCTTTCACATTATGACCAACAC | 400. | AGG | 18 | 0.6594 |
| | | low | | | | | | ATGACCA | | CAGGTCA | | | | |
| | | | | | | | | ACACC | | | | | | |
| IRF4 | CTLA4 | CTLA4 | 3662 | NM_001195286.1 | NC_000006.12 | 394977 | sense | CAGACCC | 309. | ATCTCAGACCCGTACAAAGTGTA | 401. | AGG | 3 | 0.5822 |
| | | low | | | | | | GTACAAA | | CAGGATT | | | | |
| | | | | | | | | GTGTAC | | | | | | |
| IRF4 | CTLA4 | CTLA4 | 3662 | NM_001195286.1 | NC_000006.12 | 397215 | sense | CCCATGA | 310. | ATGTCCCATGACGTTTGGACCCC | 402. | CGG | 5 | 0.7031 |
| | | low | | | | | | CGTTTGG | | GCGGCCA | | | | |
| | | | | | | | | ACCCCG | | | | | | |
| IRF4 | CTLA4 | CTLA4 | 3662 | NM_001195286.1 | NC_000006.12 | 401466 | sense | CTACCGG | 311. | TGTACTACCGGGAAATCCTCGTG | 403. | AGG | 7 | 0.6428 |
| | | low | | | | | | GAAATCC | | AAGGAGC | | | | |
| | | | | | | | | TCGTGA | | | | | | |
| IRF4 | CTLA4 | CTLA4 | 3662 | NM_001195286.1 | NC_000006.12 | 393251 | sense | CTGATCG | 312. | GTGGCTGATCGACCAGATCGAC | 404. | CGG | 2 | 0.6887 |
| | | low | | | | | | ACCAGAT | | AGCGGCAA | | | | |
| | | | | | | | | CGACAG | | | | | | |
| KMT2A | CTLA4 | CTLA4 | 4297 | NM_005933.3 | NC_000011.10 | 1.19E+08 | antisense | AAGATCA | 313. | ATTCAAGATCAGTAGCGGTCCCG | 405. | TGG | 27 | 0.7172 |
| | | low | | | | | | GTAGCGG | | GTGGTGG | | | | |
| | | | | | | | | TCCCGG | | | | | | |
| KMT2A | CTLA4 | CTLA4 | 4297 | NM_005933.3 | NC_000011.10 | 1.18E+08 | sense | AGAAAGG | 314. | GTAAAGAAAGGACGTCGATCGA | 406. | CGG | 5 | 0.6636 |
| | | low | | | | | | ACGTCGA | | GGCGGTGT | | | | |
| | | | | | | | | TCGAGG | | | | | | |
| KMT2A | CTLA4 | CTLA4 | 4297 | NM_005933.3 | NC_000011.10 | 1.18E+08 | antisense | AGGGGTC | 315. | GCCGAGGGGTCTTAATGATCCG | 407. | AGG | 3 | 0.757 |
| | | low | | | | | | TTAATGAT | | CGAGGAGA | | | | |
| | | | | | | | | CCGCG | | | | | | |
| KMT2A | CTLA4 | CTLA4 | 4297 | NM_005933.3 | NC_000011.10 | 1.18E+08 | sense | TTGACCAT | 316 | TCACTTGACCATAATTATGCTCA | 408. | TGG | 19 | 0.7229 |
| | | low | | | | | | AATTATG | | GTGGCAG | | | | |
| | | | | | | | | CTCAG | | | | | | |
| IKZFS | CTLA4 | CTLA4 | 22806 | NM_012481.4 | NC_000017.11 | 39792732 | sense | AAGATGA | 317. | TGGAAAGATGAACTGCGATGTG | 409. | TGG | 4 | 0.7182 |
| | | low | | | | | | ACTGCGA | | TGTGGATT | | | | |
| | | | | | | | | TGTGTG | | | | | | |
| IKZF3 | CTLA4 | CTLA4 | 22806 | NM_012481.4 | NC_000017.11 | 39788318 | sense | CAAGCAG | 318. | GTTACAAGCAGAGAAGTTCCCTT | 410. | AGG | 6 | 0.6635 |
| | | low | | | | | | AGAAGTT | | GAGGAGC | | | | |
| | | | | | | | | CCCTTG | | | | | | |
| IKZF3 | CTLA4 | CTLA4 | 22806 | NM_012481.4 | NC_000017.11 | 39766413 | sense | GCTCATA | 319. | GTGAGCTCATACAGACCCGCAT | 411. | TGG | 8 | 0.6101 |
| | | low | | | | | | CAGACCC | | GATGGACC | | | | |
| | | | | | | | | GCATGA | | | | | | |
| IKZF3 | CTLA4 | CTLA4 | 22806 | NM_012481.4 | NC_000017.11 | 39777693 | sense | GGACAGA | 320. | TACTGGACAGATTAGCAAGCAA | 412. | TGG | 7 | 0.7336 |
| | | low | | | | | | TTAGCAA | | TGTGGCAA | | | | |
| | | | | | | | | GCAATG | | | | | | |
| MED30 | CTLA4 | CTLA4 | 90390 | NM_080651.3 | NC_000008.11 | 1.18E+08 | sense | ACACTGG | 321. | TACCACACTGGAACATATCAAGA | 413. | CGG | 2 | 0.6317 |
| | | low | | | | | | AACATATC | | CCGGTTA | | | | |
| | | | | | | | | AAGAC | | | | | | |
| MED30 | CTLA4 | CTLA4 | 90390 | NM_080651.3 | NC_000008.11 | 1.18E+08 | sense | GACAAAT | 322. | ATATGACAAATGCAATGAAAACT | 414. | TGG | 2 | 0.7364 |
| | | low | | | | | | GCAATGA | | GTGGTGG | | | | |
| | | | | | | | | AAACTG | | | | | | |
| MED30 | CTLA4 | CTLA4 | 90390 | NM_080651.3 | NC_000008.11 | 1.18E+08 | sense | GGACATC | 323. | TGCAGGACATCGTGTACCGCACC | 415. | TGG | 1 | 0.6878 |
| | | low | | | | | | GTGTACC | | ATGGAGA | | | | |
| | | | | | | | | GCACCA | | | | | | |
| MED30 | CTLA4 | CTLA4 | 90390 | NM_080651.3 | NC_000008.11 | 1.18E+08 | sense | GGCCGCC | 324. | AGCAGGCCGCCCGGGAAGTCAA | 416. | CGG | 1 | 0.5967 |
| | | low | | | | | | CGGGAÅG | | CACGGCGT | | | | |
| | | | | | | | | TCAACA | | | | | | |
| IRF1 | CTLA4 | CTLA4 | 3659 | NM_002198.2 | NC_000005.10 | 1.32E+08 | sense | GAACTCC | 325. | CCATGAACTCCCTGCCAGATATC | 417. | AGG | 4 | 0.6297 |
| | | low | | | | | | CTGCCAG | | GAGGAGG | | | | |
| | | | | | | | | ATATCG | | | | | | |
| IRF1 | CTLA4 | CTLA4 | 3659 | NM_002198.2 | NC_000005.10 | 1.32E+08 | sense | TCTAGGC | 326. | GCCCTCTAGGCCGATACAAAGC | 418. | GGG | 4 | 0.718 |
| | | low | | | | | | CGATACA | | AGGGGAAA | | | | |
| | | | | | | | | AAGCAG | | | | | | |
| IRF1 | CTLA4 | CTLA4 | 3659 | NM_002198.2 | NC_000005.10 | 1.32E+08 | sense | TCTCCCTC | 327. | GCTTTCTCCCTCGACAGTCATGT | 419. | GGG | 6 | 0.6137 |
| | | low | | | | | | GACAGTC | | GGGGATT | | | | |
| | | | | | | | | ATGTG | | | | | | |
| IRF1 | CTLA4 | CTLA4 | 3659 | NM_002198.2 | NC_000005.10 | 1.32E+08 | sense | TTAATTCC | 328. | CAGATTAATTCCAACCAAATCCC | 420. | GGG | 2 | 0.7113 |
| | | low | | | | | | AACCAAA | | GGGGCTC | | | | |
| | | | | | | | | TCCCG | | | | | | |
| RELA | CTLA4 | CTLA4 | 5970 | NM_001243984.1 | NC_000011.10 | 65659757 | sense | ACTACGA | 329. | GGGGACTACGACCTGAATGCTG | 421. | CGG | 6 | 0.7259 |
| | | low | | | | | | CCTGAAT | | TGCGGCTC | | | | |
| | | | | | | | | GCTGTG | | | | | | |
| RELA | CTLA4 | CTLA4 | 5970 | NM_001243984.1 | NC_000011.10 | 65662009 | sense | GCTTCCG | 330. | ATGCGCTTCCGCTACAAGTGCGA | 422. | GGG | 3 | 0.7137 |
| | | low | | | | | | CTACAAG | | GGGGCGC | | | | |
| | | | | | | | | TGCGAG | | | | | | |
| RELA | CTLA4 | CTLA4 | 5970 | NM_001243984.1 | NC_000011.10 | 65658759 | antisense | GGAAGAT | 331. | AGTAGGAAGATCTCATCCCCACC | 423. | AGG | 7 | 0.7554 |
| | | low | | | | | | CTCATCCC | | GAGGCAG | | | | |
| | | | | | | | | CACCG | | | | | | |
| RELA | CTLA4 | CTLA4 | 5970 | NM_001243984.1 | NC_000011.10 | 65661818 | sense | TCAATGG | 332. | CAGATCAATGGCTACACAGGAC | 424. | GGG | 4 | 0.7164 |
| | | low | | | | | | CTACACA | | CAGGGACA | | | | |
| | | | | | | | | GGACCA | | | | | | |
| MED14 | CTLA4 | CTLA4 | 9282 | NM_004229.3 | NC_000023.11 | 40692233 | antisense | ATCACAC | 333. | TTGTATCACACATAGCGACGAAG | 425. | GGG | 15 | 0.6711 |
| | | low | | | | | | ATAGCGA | | TGGGCTA | | | | |
| | | | | | | | | CGAAGT | | | | | | |
| MED14 | CTLA4 | CTLA4 | 9282 | NM_004229.3 | NC_000023.11 | 40714644 | antisense | CAGAGCA | 334. | GGACCAGAGCATCTCTAGCTAAC | 426. | AGG | 4 | 0.6206 |
| | | low | | | | | | TCTCTAGC | | GAGGCCA | | | | |
| | | | | | | | | TAACG | | | | | | |
| MED14 | CTLA4 | CTLA4 | 9282 | NM_004229.3 | NC_000023.11 | 40682898 | antisense | CTAACTCT | 335. | AACACTAACTCTGCTACCCAAGT | 427. | CGG | 17 | 0.7032 |
| | | low | | | | | | GCTACCC | | GCGGTTA | | | | |
| | | | | | | | | AAGTG | | | | | | |
| MED14 | ICTLA4 | CTLA4 | 9282 | NM_004229.3 | NC_000023.11 | 40711237 | sense | TAATGTTA | 336. | ACTCTAATGTTAATCCGAGAACG | 428. | TGG | 8 | 0.6368 |
| | | low | | | | | | ATCCGAG | | GTGGGGA | | | | |
| | | | | | | | | AACGG | | | | | | |
| TAF5L | CTLA4 | CTLA4 | 27097 | NM_014409.3 | NC_000001.11 | 2.3E+08 | antisense | CGGGACA | 337. | GATGCGGGACACGTCTACTTGG | 429. | GGG | 4 | 0.6222 |
| | | low | | | | | | CGTCTACT | | TGGGGCTC | | | | |
| | | | | | | | | TGGTG | | | | | | |
| TAF5L | CTLA4 | \|CTLA4 | 27097 | NM_014409.3 | NC_000001.11 | 2.3E+08 | sense | GCAGAAC | 338. | TTCTGCAGAACGAGGCTGCCCTA | 430. | AGG | 4 | 0.6216 |
| | | low | | | | | | GAGGCTG | | GAGGTCT | | | | |
| | | | | | | | | CCCTAG | | | | | | |
| TAF5L | CTLA4 | CTLA4 | 27097 | NM_014409.3 | NC_000001.11 | 2.3E+08 | sense | GCGGACC | 339. | CACTGCGGACCAGTGTACAGCA | 431. | AGG | 5 | 0.7066 |
| | | low | | | | | | AGTGTAC | | CGAGGTTC | | | | |
| | | | | | | | | AGCACG | | | | | | |
| TAF5L | CTLA4 | CTLA4 | 27097 | NM_014409.3 | NC_000001.11 | 2.3E+08 | antisense | TAAGGTG | 340. | TATGTAAGGTGAGGACTTTGCAC | 432. | GGG | 4 | 0.6402 |
| | | low | | | | | | AGGACTT | | AGGGCAG | | | | |
| | | | | | | | | TGCACA | | | | | | |
| MTF1 | CTLA4 | CTLA4 | 4520 | NM_005955.2 | NC_000001.11 | 37835092 | sense | AATGCAC | 341. | ATACAATGCACTTCCACAACACA | 433. | TGG | 6 | 0.6619 |
| | | low | | | | | | TTCCACAA | | ATGGATC | | | | |
| | | | | | | | | CACAA | | | | | | |
| MTF1 | CTLA4 | CTLA4 | 4520 | NM_005955.2 | NC_000001.11 | 37857385 | antisense | AATGTGC | 342. | AGATAATGTGCTGCACATAACCC | 434. | GGG | 2 | 0.6361 |
| | | low | | | | | | TGCACAT | | TGGGACA | | | | |
| | | | | | | | | AACCCT | | | | | | |
| MTF1 | CTLA4 | CTLA4 | 4520 | NM_005955.2 | NC_000001.11 | 37839984 | sense | CCACGTG | 343. | GGATCCACGTGCGAGTGCACAC | 435. | AGG | 3 | 0.7358 |
| | | low | | | | | | CGAGTGC | | GAAGGAGA | | | | |
| | | | | | | | | ACACGA | | | | | | |
| MTF1 | CTLA4 | CTLA4 | 4520 | NM_005955.2 | NC_000001.11 | 37838644 | sense | GCACATT | 344. | GGAAGCACATTCGAACTCATACA | 436. | GGG | 4 | 0.7005 |
| | | low | | | | | | CGAACTC | | GGGGAAA | | | | |
| | | | | | | | | ATACAG | | | | | | |
| IKZF1 | CTLA4 | CTLA4 | 10320 | NM_006060.5 | NC_000007.14 | 50376659 | sense | GAAAATG | 345. | GAGAGAAAATGAATGGCTCCCA | 437. | GGG | 4 | 0.66 |
| | | low | | | | | | AATGGCT | | CAGGGACC | | | | |
| | | | | | | | | CCCACA | | | | | | |
| IKZF1 | CTLA4 | CTLA4 | 10320 | NM_006060.5 | NC_000007.14 | 50399996 | antisense | GATGGCT | 346. | TGTTGATGGCTTGGTCCATCACG | 438. | GGG | 8 | 0.7379 |
| | | low | | | | | | TGGTCCA | | TGGGACT | | | | |
| | | | | | | | | TCACGT | | | | | | |
| IKZF1 | CTLA4 | CTLA4 | 10320 | NM_006060.5 | NC_000007.14 | 50382586 | sense | GGGGCCT | 347 | GTGCGGGGCCTCATTCACCCAG | 439. | GGG | 5 | 0.6196 |
| | | low | | | | | | CATTCACC | | AAGGGCAA | | | | |
| | | | | | | | | CAGAA | | | | | | |
| IKZF1 | CTLA4 | CTLA4 | 10320 | NM_006060.5 | NC_000007.14 | 50327753 | sense | TCCAAGA | 348. | AAGCTCCAAGAGTGACAGAGTC | 440. | GGG | 3 | 0.617 |
| | | low | | | | | | GTGACAG | | GTGGGTAA | | | | |
| | | | | | | | | AGTCGT | | | | | | |
| BCL11B | CTLA4 | CTLA4 | 64919 | NM_001282237.1 | NC_000014.9 | 99175544 | sense | AGCAAGT | 349. | CAAGAGCAAGTCGTGCGAGTTC | 441. | CGG | 4 | 0.6246 |
| | | low | | | | | | CGTGCGA | | TGCGGCAA | | | | |
| | | | | | | | | GTTCTG | | | | | | |
| BCL11B | CTLA4 | CTLA4 | 64919 | NM_001282237.1 | NC_000014.9 | 99176056 | sense | CCAGCAG | 350. | CCGGCCAGCAGCTCGCTCACGCC | 442. | CGG | 4 | 0.6152 |
| | | low | | | | | | CTCGCTCA | | GCGGCTC | | | | |
| | | | | | | | | CGCCG | | | | | | |
| BCL11B | CTLA4 | CTLA4 | 64919 | NM_001282237.1 | NC_000014.9 | 99175744 | sense | CCGCCAT | 351. | TCGCCCGCCATGGACTTCTCGCG | 443. | CGG | 4 | 0.7423 |
| | | low | | | | | | GGACTTC | | GCGGCTC | | | | |
| | | | | | | | | TCGCGG | | | | | | |
| BCL11B | CTLA4 | CTLA4 | 64919 | NM_001282237.1 | NC_000014.9 | 99231381 | sense | TCAGGGT | 352. | GGACTCAGGGTGAGGGTCAGAC | 444 | AGG | 3 | 0.6195 |
| | | low | | | | | | GAGGGTC | | GGAGGCTC | | | | |
| | | | | | | | | AGACGG | | | | | | |
| STAT3 | CTLA4 | CTLA4 | 6774 | NM_139276.2 | NC_000017.11 | 42329588 | sense | AACATGG | 353. | GATGAACATGGAAGAATCCAAC | 445. | CGG | 13 | 0.6983 |
| | | low | | | | | | AAGAATC | | AACGGCAG | | | | |
| | | | | | | | | CAACAA | | | | | | |
| STAT3 | CTLA4 | CTLA4 | 6774 | NM_139276.2 | NC_000017.11 | 42333711 | antisense | ACGCCGG | 354. | CTGGACGCCGGTCTTGATGACG | 446. | GGG | 10 | 0.6587 |
| | | low | | | | | | TCTTGATG | | AGGGGCCG | | | | |
| | | | | | | | | ACGAG | | | | | | |
| STAT3 | CTLA4 | CTLA4 | 6774 | NM_139276.2 | NC_000017.11 | 42326141 | sense | GAGACCG | 355. | CTTTGAGACCGAGGTGTATCACC | 447. | AGG | 15 | 0.7276 |
| | | low | | | | | | AGGTGTA | | AAGGCCT | | | | |
| | | | | | | | | TCACCA | | | | | | |
| STAT3 | CTLA4 | CTLA4 | 6774 | NM_139276.2 | NC_000017.11 | 42333954 | antisense | TCGGCCG | 356. | AGCATCGGCCGGTGCTGTACAA | 448. | GGG | 9 | 0.6901 |
| | | low | | | | | | GTGCTGT | | TGGGGTCC | | | | |
| | | | | | | | | ACAATG | | | | | | |
| ZNF384 | CTLA4 | CTLA4 | 171017 | NM_001135734.2 | NC_000012.12 | 6678985 | sense | CGTTACCC | 357. | CGTCCGTTACCCAGAATATCACG | 449. | TGG | 4 | 0.835 |
| | | low | | | | | | AGAATAT | | GTGGTCC | | | | |
| | | | | | | | | CACGG | | | | | | |
| ZNF384 | CTLA4 | CTLA4 | 171017 | NM_001135734.2 | NC_000012.12 | 6673292 | sense | GCACATC | 358. | CCCAGCACATCCGTATACACTCA | 450. | GGG | 7 | 0.7689 |
| | | low | | | | | | CGTATAC | | GGGGCTA | | | | |
| | | | | | | | | ACTCAG | | | | | | |
| ZNF384 | CTLA4 | CTLA4 | 171017 | NM_001135734.2 | NC_000012.12 | 6678296 | sense | GGTAGCA | 359. | AGAAGGTAGCATCGACCCTAAC | 451. | AGG | 6 | 0.7069 |
| | | low | | | | | | TCGACCCT | | CGAGGAAG | | | | |
| | | | | | | | | AACCG | | | | | | |
| ZNF384 | CTLA4 | CTLA4 | 171017 | NM_001135734.2 | NC_000012.12 | 6672434 | antisense | TGGCCCC | 360. | GGCTTGGCCCCCGAGTGGATAC | 452. | AGG | 8 | 0.6923 |
| | | low | | | | | | CGAGTGG | | GGAGGTGC | | | | |
| | | | | | | | | ATACGG | | | | | | |
| BPTF | CTLA4 | CTLA4 | 2186 | NM_182641.3 | NC_000017.11 | 67912848 | sense | AAAGGAG | 361. | CTGTAAAGGAGCAGAGCAAAAC | 453. | TGG | 11 | 0.7483 |
| | | low | | | | | | CAGAGCA | | CGTGGTCA | | | | |
| | | | | | | | | AAACCG | | | | | | |
| BPTF | CTLA4 | CTLA4 | 2186 | NM_182641.3 | NC_000017.11 | 67911774 | antisense | ACTGCTAT | 362. | CTTCACTGCTATCCTGAATAGAC | 454. | CGG | 11 | 0.7291 |
| | | low | | | | | | CCTGAAT | | ACGGTAT | | | | |
| | | | | | | | | AGACA | | | | | | |
| BPTF | CTLA4 | \|CTLA4 | 2186 | NM_182641.3 | NC_000017.11 | 67854008 | antisense | GAGGACT | 363. | CTCAGAGGACTTGGGAAATTCA | 455. | GGG | 2 | 0.6802 |
| | | low | | | | | | TGGGAAA | | AGGGGCGG | | | | |
| | | | | | | | | TTCAAG | | | | | | |
| BPTF | CTLA4 | CTLA4 | 2186 | NM_182641.3 | NC_000017.11 | 67922881 | sense | GCCTTCG | 364. | AAAGGCCTTCGATCAAGTGCACT | 456. | CGG | 14 | 0.7554 |
| | | low | | | | | | ATCAAGT | | GCGGCCA | | | | |
| | | | | | | | | GCACTG | | | | | | |
| IL1R1 | CTLA4 | CTLA4 | 3554 | NM_000877.3 | NC_000002.12 | 1.02E+08 | antisense | AATAGTC | 365. | CTGTAATAGTCTTCCCCTAGCAC | 457. | GGG | 8 | 0.5943 |
| | | low | | | | | | TTCCCCTA | | TGGGTCA | | | | |
| | | | | | | | | GCACT | | | | | | |
| IL1R1 | CTLA4 | CTLA4 | 3554 | NM_000877.3 | NC_000002.12 | 1.02E+08 | antisense | ATTACAG | 366. | TGACATTACAGATCAATTGTATC | 458. | GGG | 8 | 0.5573 |
| | | low | | | | | | ATCAATT | | TGGGATC | | | | |
| | | | | | | | | GTATCT | | | | | | |
| IL1R1 | CTLA4 | CTLA4 | 3554 | NM_000877.3 | NC_000002.12 | 1.02E+08 | sense | CAAGCAA | 367. | TGGGCAAGCAATATCCTATTACC | 459. | GGG | 6 | 0.6217 |
| | | low | | | | | | TATCCTAT | | CGGGTAA | | | | |
| | | | | | | | | TACCC | | | | | | |
| IL1R1 | CTLA4 | CTLA4 | 3554 | NM_000877.3 | NC_000002.12 | 1.02E+08 | antisense | TTTGTGTT | 368. | TCTCTTTGTGTTGATGAATCCTG | 460. | AGG | 4 | 0.6689 |
| | | low | | | | | | GATGAAT | | GAGGCTT | | | | |
| | | | | | | | | CCTGG | | | | | | |
| MED11 | CTLA4 | CTLA4 | 400569 | NM_001001683.3 | NC_000017.11 | 4731567 | antisense | ATCCCGA | 369. | TCGCATCCCGAACCTGCATTCTG | 461. | AGG | 1 | 0.567 |
| | | low | | | | | | ACCTGCA | | AAGGATG | | | | |
| | | | | | | | | TTCTGA | | | | | | |
| MED11 | CTLA4 | CTLA4 | 400569 | NM_001001683.3 | NC_000017.11 | 4731863 | sense | CACCGCTT | 370. | CCTTCACCGCTTCAGTGCAACAC | 462. | TGG | 2 | 0.6458 |
| | | low | | | | | | CAGTGCA | | GTGGAGG | | | | |
| | | | | | | | | ACACG | | | | | | |
| MED11 | CTLA4 | CTLA4 | 400569 | NM_001001683.3 | NC_000017.11 | 4731814 | sense | CCAAGGA | 371. | TTGTCCAAGGAAAAAACTAACG | 463. | CGG | 2 | 0.6925 |
| | | low | | | | | | AAAAACT | | AGCGGCTC | | | | |
| | | | | | | | | AACGAG | | | | | | |
| MED11 | CTLA4 | CTLA4 | 400569 | NM_001001683.3 | NC_000017.11 | 4731833 | sense | GCGGCTC | 372. | ACGAGCGGCTCCTAGACCGGCA | 464. | CGG | 2 | 0.6487 |
| | | low | | | | | | CTAGACC | | GGCGGCGG | | | | |
| | | | | | | | | GGCAGG | | | | | | |
| KLF6 | CTLA4 | CTLA4 | 1316 | NM_001160124.1 | NC_000010.11 | 3781951 | antisense | TCGGAGG | 373. | GGGGTCGGAGGTAAACTTGGCC | 465. | GGG | 2 | 0.6468 |
| | | low | | | | | | TAAACTT | | GTGGGAGA | | | | |
| | | | | | | | | GGCCGT | | | | | | |
| KLF6 | CTLA4 | CTLA4 | 1316 | NM_001160124.1 | NC_000010.11 | 3782035 | antisense | TCTAAGTT | 374. | GGTCTCTAAGTTGTAACAAAAGC | 466. | CGG | 2 | 0.5276 |
| | | low | | | | | | GTAACAA | | TCGGGCT | | | | |
| | | | | | | | | AAGCT | | | | | | |
| KLF6 | CTLA4 | CTLA4 | 1316 | NM_001160124.1 | NC_000010.11 | 3782173 | antisense | TCTGAGG | 375. | GATTTCTGAGGCTGAAACATAGC | 467. | GGG | 2 | 0.671 |
| | | low | | | | | | CTGAAAC | | AGGGCTC | | | | |
| | | | | | | | | ATAGCA | | | | | | |
| KLF6 | CTLA4 | CTLA4 | 1316 | NM_001160124.1 | NC_000010.11 | 3784934 | antisense | TGCCAGT | 376. | CTGTTGCCAGTACTCCTCCAGAG | 468. | CGG | 1 | 0.6381 |
| | | low | | | | | | ACTCCTCC | | ACGGCAG | | | | |
| | | | | | | | | AGAGA | | | | | | |
| CREB1 | CTLA4 | CTLA4 | 1385 | NM_004379.3 | NC_000002.12 | 2.08E+08 | antisense | AGCTGTA | 377. | GGGCAGCTGTACTAGAGTTACG | 469. | GGG | 3 | 0.6415 |
| | | low | | | | | | CTAGAGT | | GTGGGAGC | | | | |
| | | | | | | | | TACGGT | | | | | | |
| CREB1 | CTLA4 | CTLA4 | 1385 | NM_004379.3 | NC_000002.12 | 2.08E+08 | sense | GGCTAAC | 378. | AGCTGGCTAACAATGGTACCGA | 470. | GGG | 6 | 0.7053 |
| | | low | | | | | | AATGGTA | | TGGGGTAC | | | | |
| | | | | | | | | CCGATG | | | | | | |
| CREB1 | CTLA4 | CTLA4 | 1385 | NM_004379.3 | NC_000002.12 | 2.08E+08 | antisense | TGGAGTT | 379. | AAATTGGAGTTGGCACCGTTACA | 471. | TGG | 5 | 0.6189 |
| | | low | | | | | | GGCACCG | | GTGGTGA | | | | |
| | | | | | | | | TTACAG | | | | | | |
| CREB1 | CTLA4 | CTLA4 | 1385 | NM_004379.3 | NC_000002.12 | 2.08E+08 | antisense | TGTGGAG | 380. | GACTTGTGGAGACTGAATAACT | 472. | TGG | 3 | 0.6776 |
| | | low | | | | | | ACTGAAT | | GATGGCTG | | | | |
| | | | | | | | | AACTGA | | | | | | |

TABLE 3
Nuclear factors that can be inhibited to increase FOXP3 expression or overexpressed to decrease FOXP3 expression

| | | | | | | Position of | | | | | | | | |
|---|---|---|---|---|---|---|---|---|---|---|---|---|---|---|
| | | | Target | | | Base After | | sgRNA | | Target | | | | |
| gene_ | screen_ | screen_ | Gene | Target | Genomic | Cut (1- | | Target | SEQ ID | Context | SEQ ID | PAM | Exon | Rule Set 2 |
| id | target | direction | ID | Transcript | Sequence | based) | Strand | Sequence | NO: | Sequence | NO. | Sequence | Number | score |
| TERF2 | FOXP3 | FOXP3 | 7014 | NM_005652.4 | NC_000016.10 | 69370578 | antisense | AAAGTTCT | 473. | ATGAAAA | 549. | GGG | 5 | 0.6289 |
| | | high | | | | | | GGATAAC | | GTTCTGGA | | | | |
| | | | | | | | | AGGAT | | TAACAGG | | | | |
| | | | | | | | | | | ATGGGCC | | | | |
| | | | | | | | | | | A | | | | |
| TERF2 | FOXP3 | FOXP3 | 7014 | NM_005652.4 | NC_000016.10 | 69385848 | sense | CGCGCGG | 474. | AGGGCGC | 550. | TGG | 1 | 0.6198 |
| | | high | | | | | | CGATCGG | | GCGGCGA | | | | |
| | | | | | | | | ACACGA | | TCGGACA | | | | |
| | | | | | | | | | | CGATGGC | | | | |
| | | | | | | | | | | GG | | | | |
| TERF2 | FOXP3 | FOXP3 | 7014 | NM_005652.4 | NC_000016.10 | 69385419 | sense | GGGTTAT | 475. | CTGCGGG | 551. | CGG | 2 | 0.6807 |
| | | high | | | | | | GCAGTGT | | TTATGCAG | | | | |
| | | | | | | | | CTGTCG | | TGTCTGTC | | | | |
| | | | | | | | | | | GCGGATT | | | | |
| TERF2 | FOXP3 | FOXP3 | 7014 | NM_005652.4 | NC_000016.10 | 69385628 | antisense | TCTGTCTG | 476. | CGGATCT | 552. | CGG | 1 | 0.6089 |
| | | high | | | | | | AAGTCCCC | | GTCTGAA | | | | |
| | | | | | | | | GTAC | | GTCCCCGT | | | | |
| | | | | | | | | | | ACCGGCT | | | | |
| | | | | | | | | | | A | | | | |
| FLI1 | FOXP3 | FOXP3 | 2313 | NM_002017.4 | NC_000011.10 | 1.29E+08 | antisense | ACTCAATC | 477. | TGACACTC | 553. | CGG | 5 | 0.6162 |
| | | high | | | | | | GTGAGGA | | AATCGTG | | | | |
| | | | | | | | | TTGGT | | AGGATTG | | | | |
| | | | | | | | | | | GTCGGTG | | | | |
| | | | | | | | | | | T | | | | |
| FLI1 | FOXP3 | FOXP3 | 2313 | NM_002017.4 | NC_000011.10 | 1.29E+08 | sense | ACTGTGTA | 478. | AGGAACT | 554. | AGG | 4 | 0.8003 |
| | | high | | | | | | AAATGAA | | GTGTAAA | | | | |
| | | | | | | | | CAAGG | | ATGAACA | | | | |
| | | | | | | | | | | AGGAGGA | | | | |
| | | | | | | | | | | CT | | | | |
| FLI1 | FOXP3 | FOXP3 | 2313 | NM_002017.4 | NC_000011.10 | 1.29E+08 | sense | CCAACATG | 479. | CCTCCCAA | 555. | AGG | 3 | 0.6923 |
| | | high | | | | | | ACCACCAA | | CATGACCA | | | | |
| | | | | | | | | CGAG | | CCAACGA | | | | |
| | | | | | | | | | | GAGGAGA | | | | |
| FLI1 | FOXP3 | FOXP3 | 2313 | NM_002017.4 | NC_000011.10 | 1.29E+08 | antisense | CTTACCTG | 480 | TGAGCTTA | 556. | TGG | 2 | 0.7136 |
| | | high | | | | | | GATCCATT | | CCTGGATC | | | | |
| | | | | | | | | CATG | | CATTCATG | | | | |
| | | | | | | | | | | TGGTCA | | | | |
| HMGA1 | FOXP3 | FOXP3 | 3159 | NM_145899.2 | NC_000006.12 | 34240847 | sense | AAAAGGA | 481. | CAGGAAA | 557. | CGG | 3 | 0.555 |
| | | high | | | | | | CGGCACT | | AGGACGG | | | | |
| | | | | | | | | GAGAAG | | CACTGAG | | | | |
| | | | | | | | | | | AAGCGGG | | | | |
| | | | | | | | | | | GC | | | | |
| HMGA1 | FOXP3 | FOXP3 | 3159 | NM_145899.2 | NC_000006.12 | 34240914 | sense | AGCGCTG | 482. | GGACAGC | 558. | TGG | 3 | 0.5867 |
| | | high | | | | | | GTAGGGA | | GCTGGTA | | | | |
| | | | | | | | | GTCAGG | | GGGAGTC | | | | |
| | | | | | | | | | | AGGTGGG | | | | |
| | | | | | | | | | | TG | | | | |
| HMGA1 | FOXP3 | FOXP3 | 3159 | NM_145899.2 | NC_000006.12 | 34242747 | sense | CCAACACC | 483. | AGTGCCA | 559. | GGG | 4 | 0.7288 |
| | | high | | | | | | TAAGAGA | | ACACCTAA | | | | |
| | | | | | | | | CCTCG | | GAGACCT | | | | |
| | | | | | | | | | | CGGGGCC | | | | |
| | | | | | | | | | | G | | | | |
| HMGA1 | FOXP3 | FOXP3 | 3159 | NM_145899.2 | NC_000006.12 | 34240883 | antisense | GCTGTCCC | 484. | CAGCGCT | 560. | AGG | 3 | 0.6102 |
| | | high | | | | | | GGGACTC | | GTCCCGG | | | | |
| | | | | | | | | ACCGG | | GACTCACC | | | | |
| | | | | | | | | | | GGAGGCT | | | | |
| | | | | | | | | | | G | | | | |
| KLF1 | FOXP3 | FOXP3 | 10661 | NM_006563.3 | NC_000019.10 | 12885695 | antisense | CCGCGGG | 485. | CGGTCCG | 561. | AGG | 2 | 0.6423 |
| | | high | | | | | | AAGTAGC | | CGGGAAG | | | | |
| | | | | | | | | CACCCG | | TAGCCACC | | | | |
| | | | | | | | | | | CGAGGAG | | | | |
| | | | | | | | | | | C | | | | |
| KLF1 | FOXP3 | FOXP3 | 10661 | NM_006563.3 | NC_000019.10 | 12885920 | antisense | CGGCGGC | 486. | CGGGCGG | 562. | CGG | 2 | 0.6145 |
| | | high | | | | | | GGATATT | | CGGCGGA | | | | |
| | | | | | | | | GCGCCC | | TATTGCGC | | | | |
| | | | | | | | | | | CCCGGAG | | | | |
| | | | | | | | | | | G | | | | |
| KLF1 | FOXP3 | FOXP3 | 10661 | NM_006563.3 | NC_000019.10 | 12885616 | antisense | GAGGCGC | 487. | TACTGAG | 563. | GGG | 2 | 0.6739 |
| | | high | | | | | | CGGGTAC | | GCGCCGG | | | | |
| | | | | | | | | ATCGCG | | GTACATCG | | | | |
| | | | | | | | | | | CGGGGTA | | | | |
| | | | | | | | | | | C | | | | |
| KLF1 | FOXP3 | FOXP3 | 10661 | NM_006563.3 | NC_000019.10 | 12885524 | antisense | GGGTCCC | 488. | TCCCGGGT | 564. | AGG | 2 | 0.6507 |
| | | high | | | | | | AAACAACT | | CCCAAACA | | | | |
| | | | | | | | | CAGGA | | ACTCAGG | | | | |
| | | | | | | | | | | AAGGAGG | | | | |
| MYBL2 | FOXP3 | FOXP3 | 4605 | NM_002466.3 | NC_000020.11 | 43682870 | sense | CAAGGGG | 489. | TTGTCAAG | 565. | AGG | 4 | 0.6606 |
| | | high | | | | | | CCATGGA | | GGGCCAT | | | | |
| | | | | | | | | CCAAAG | | GGACCAA | | | | |
| | | | | | | | | | | AGAGGAA | | | | |
| | | | | | | | | | | G | | | | |
| MYBL2 | FOXP3 | FOXP3 | 4605 | NM_002466.3 | NC_000020.11 | 43692255 | antisense | CTCCAGCA | 490. | CGAGCTCC | 566. | GGG | 6 | 0.6859 |
| | | high | | | | | | GCAAGTA | | AGCAGCA | | | | |
| | | | | | | | | CACTG | | AGTACACT | | | | |
| | | | | | | | | | | GGGGGCT | | | | |
| MYBL2 | FOXP3 | FOXP3 | 4605 | NM_002466.3 | NC_000020.11 | 43699865 | antisense | GCGTCCA | 491. | CACTGCGT | 567. | GGG | 7 | 0.6074 |
| | | high | | | | | | GATCTGTA | | CCAGATCT | | | | |
| | | | | | | | | CCGAT | | GTACCGAT | | | | |
| | | | | | | | | | | GGGCTC | | | | |
| MYBL2 | FOXP3 | FOXP3 | 4605 | NM_002466.3 | NC_000020.11 | 43702498 | antisense | TCAGGTCA | 492. | TTACTCAG | 568. | GGG | 8 | 0.6578 |
| | | high | | | | | | CACCAAG | | GTCACACC | | | | |
| | | | | | | | | CATCA | | AAGCATC | | | | |
| | | | | | | | | | | AGGGTCC | | | | |
| ZBTB1 | FOXP3 | FOXP3 | 22890 | NM_001123329.1 | NC_000014.9 | 64522022 | sense | AAATACAC | 493. | CGGTAAA | 569. | AGG | 2 | 0.6809 |
| | | high | | | | | | CACATAAT | | TACACCAC | | | | |
| | | | | | | | | AGAG | | ATAATAG | | | | |
| | | | | | | | | | | AGAGGCT | | | | |
| | | | | | | | | | | G | | | | |
| ZBTB1 | FOXP3 | FOXP3 | 22890 | NM_001123329.1 | NC_000014.9 | 64522356 | sense | GCACAGA | 494. | TTCTGCAC | 570. | AGG | 2 | 0.7303 |
| | | high | | | | | | CGGACAA | | AGACGGA | | | | |
| | | | | | | | | ATACAG | | CAAATACA | | | | |
| | | | | | | | | | | GAGGAGA | | | | |
| ZBTB1 | FOXP3 | FOXP3 | 22890 | NM_001123329.1 | NC_000014.9 | 64522796 | sense | GTGTGGA | 495. | AGCTGTGT | 571. | AGG | 2 | 0.7293 |
| | | high | | | | | | CTTACAAT | | GGACTTAC | | | | |
| | | | | | | | | AACCG | | AATAACC | | | | |
| | | | | | | | | | | GAGGAGG | | | | |
| ZBTB1 | FOXP3 | FOXP3 | 22890 | NM_001123329.1 | NC_000014.9 | 64522141 | sense | GTGTGTCA | 496. | GAACGTG | 572. | CGG | 2 | 0.6916 |
| | | high | | | | | | AGACGCTT | | TGTCAAG | | | | |
| | | | | | | | | TGGG | | ACGCTTTG | | | | |
| | | | | | | | | | | GGCGGAG | | | | |
| | | | | | | | | | | T | | | | |
| E2F4 | FOXP3 | FOXP3 | 1874 | NM_001950.3 | NC_000016.10 | 67193119 | sense | ACTAGACC | 497. | AAGAACT | 573. | GGG | 3 | 0.658 |
| | | high | | | | | | AGCACAA | | AGACCAG | | | | |
| | | | | | | | | GGTGT | | CACAAGG | | | | |
| | | | | | | | | | | TGTGGGT | | | | |
| | | | | | | | | | | GC | | | | |
| E2F4 | FOXP3 | FOXP3 | 1874 | NM_001950.3 | NC_000016.10 | 67193038 | antisense | CAGTTTGT | 498. | CAATCAGT | 574. | GGG | 3 | 0.6002 |
| | | high | | | | | | CAGCAATC | | TTGTCAGC | | | | |
| | | | | | | | | CCC | | AATCTCCC | | | | |
| | | | | | | | | | | GGGTAT | | | | |
| E2F4 | FOXP3 | FOXP3 | 1874 | NM_001950.3 | NC_000016.10 | 67194741 | sense | GAGTGTG | 499. | TGAAGAG | 575. | AGG | 6 | 0.6509 |
| | | high | | | | | | AGTGGTC | | TGTGAGT | | | | |
| | | | | | | | | CCATTG | | GGTCCCAT | | | | |
| | | | | | | | | | | TGAGGTTC | | | | |
| E2F4 | FOXP3 | FOXP3 | 1874 | NM_001950.3 | NC_000016.10 | 67193483 | antisense | GATGTCCT | 500. | TGCAGAT | 576. | AGG | 4 | 0.6736 |
| | | high | | | | | | CATGAGT | | GTCCTCAT | | | | |
| | | | | | | | | GACGT | | GAGTGAC | | | | |
| | | | | | | | | | | GTAGGCC | | | | |
| | | | | | | | | | | A | | | | |
| HINFP | FOXP3 | FOXP3 | 25988 | NM_015517.4 | NC_000011.10 | 1.19E+08 | sense | CACACCAA | 501. | CTACCACA | 577. | GGG | 4 | 0.6982 |
| | | high | | | | | | GCTGAAA | | CCAAGCT | | | | |
| | | | | | | | | CAGTG | | GAAACAG | | | | |
| | | | | | | | | | | TGGGGGC | | | | |
| | | | | | | | | | | T | | | | |
| HINFP | FOXP3 | FOXP3 | 25988 | NM_015517,4 | NC_000011.10 | 1.19E+08 | sense | CATGCGCT | 502. | ACCACATG | 578. | AGG | 8 | 0.7351 |
| | | high | | | | | | TTCGTCAC | | CGCTTTCG | | | | |
| | | | | | | | | AGTG | | TCACAGTG | | | | |
| | | | | | | | | | | AGGACC | | | | |
| HINFP | FOXP3 | FOXP3 | 25988 | NM_015517.4 | NC_000011.10 | 1.19E+08 | antisense | GGTGCTCT | 503. | CGGAGGT | 579. | CGG | 6 | 0.7508 |
| | | high | | | | | | CGAAGTTT | | GCTCTCGA | | | | |
| | | | | | | | | ACTG | | AGTTTACT | | | | |
| | | | | | | | | | | GCGGTCC | | | | |
| HINFP | FOXP3 | FOXP3 | 25988 | NM_015517.4 | NC_000011.10 | 1.19E+08 | antisense | TGACTACT | 504. | CCTCTGAC | 580. | AGG | 6 | 0.7145 |
| | | high | | | | | | TACGATCC | | TACTTACG | | | | |
| | | | | | | | | AATG | | ATCCAATG | | | | |
| | | | | | | | | | | AGGTCT | | | | |
| SP1 | FOXP3 | FOXP3 | 6667 | NM_001251825.1 | NC_000012.12 | 53382598 | sense | CAACAGA | 505. | AAACCAA | 581. | AGG | 3 | 0.6198 |
| | | high | | | | | | TTATCACA | | CAGATTAT | | | | |
| | | | | | | | | AATCG | | CACAAATC | | | | |
| | | | | | | | | | | GAGGAAG | | | | |
| SP1 | FOXP3 | FOXP3 | 6667 | NM_001251825.1 | NC_000012.12 | 53383311 | sense | CATCATCC | 506. | CCATCATC | 582. | TGG | 3 | 0.649 |
| | | high | | | | | | GGACACC | | ATCCGGA | | | | |
| | | | | | | | | AACAG | | CACCAACA | | | | |
| | | | | | | | | | | GTGGGGC | | | | |
| SP1 | FOXP3 | FOXP3 | 6667 | NM_001251825.1 | NC_000012.12 | 53382717 | sense | GTATGTG | 507. | CTCAGTAT | 583. | TGG | 3 | 0.6661 |
| | | high | | | | | | ACCAATGT | | GTGACCA | | | | |
| | | | | | | | | ACCAG | | ATGTACCA | | | | |
| | | | | | | | | | | GTGGCCC | | | | |
| SP1 | FOXP3 | FOXP3 | 6667 | NM_001251825.1 | NC_000012.12 | 53382986 | sense | TTACTACC | 508. | AACTTTAC | 584. | GGG | 3 | 0.7044 |
| | | high | | | | | | AGTGGAT | | TACCAGTG | | | | |
| | | | | | | | | CATCA | | GATCATCA | | | | |
| | | | | | | | | | | GGGACC | | | | |
| TP53 | FOXP3 | FOXP3 | 7157 | NM_000546.5 | NC_000017.11 | 7676227 | antisense | CCATTGTT | 509. | TGAACCAT | 585. | GGG | 4 | 0.6899 |
| | | high | | | | | | CAATATCG | | TGTTCAAT | | | | |
| | | | | | | | | TCCG | | ATCGTCCG | | | | |
| | | | | | | | | | | GGGACA | | | | |
| TP53 | FOXP3 | FOXP3 | 7157 | NM_000546.5 | NC_000017.11 | 7675058 | sense | GAGCGCT | 510. | CCATGAG | 586. | TGG | 5 | 0.6542 |
| | | high | | | | | | GCTCAGAT | | CGCTGCTC | | | | |
| | | | | | | | | AGCGA | | AGATAGC | | | | |
| | | | | | | | | | | GATGGTG | | | | |
| TP53 | FOXP3 | FOXP3 | 7157 | NM_000546.5 | NC_000017.11 | 7676527 | antisense | GATCCACT | 511. | AATGGAT | 587. | AGG | 2 | 0.6108 |
| | | high | | | | | | CACAGTTT | | CCACTCAC | | | | |
| | | | | | | | | CCAT | | AGTTTCCA | | | | |
| | | | | | | | | | | TAGGTCT | | | | |
| TP53 | FOXP3 | FOXP3 | 7157 | NM_000546.5 | NC_000017.11 | 7674864 | sense | GGTGCCCT | 512. | TGGTGGT | 588. | AGG | 6 | 0.6259 |
| | | high | | | | | | ATGAGCC | | GCCCTATG | | | | |
| | | | | | | | | GCCTG | | AGCCGCCT | | | | |
| | | | | | | | | | | GAGGTCT | | | | |
| MAF | FOXP3 | FOXP3 | 4094 | NM_005360.4 | NC_000016.10 | 79599622 | sense | GAAGACT | 513. | CCTGGAA | 589. | CGG | 1 | 0.5494 |
| | | high | | | | | | ACTACTGG | | GACTACTA | | | | |
| | | | | | | | | ATGAC | | CTGGATG | | | | |
| | | | | | | | | | | ACCGGCT | | | | |
| | | | | | | | | | | A | | | | |
| MAF | FOXP3 | FOXP3 | 4094 | NM_005360.4 | NC_000016.10 | 79599416 | antisense | GATCACG | 514. | CGGCGAT | 590. | CGG | 1 | 0.7152 |
| | | high | | | | | | GCGGACA | | CACGGCG | | | | |
| | | | | | | | | CCACGG | | GACACCA | | | | |
| | | | | | | | | | | CGGCGGC | | | | |
| | | | | | | | | | | GG | | | | |
| MAF | FOXP3 | FOXP3 | 4094 | NM_005360.4 | NC_000016.10 | 79599725 | antisense | GCTGCAC | 515. | CCGAGCT | 591. | GGG | 1 | 0.7019 |
| | | high | | | | | | GGCGTGC | | GCACGGC | | | | |
| | | | | | | | | TCATGG | | GTGCTCAT | | | | |
| | | | | | | | | | | GGGGGTG | | | | |
| | | | | | | | | | | G | | | | |
| MAF | FOXP3 | FOXP3 | 4094 | NM_005360.4 | NC_000016.10 | 79599797 | sense | TGAAGTG | 516. | AGTTTGAA | 592. | TGG | 1 | 0.6793 |
| | | high | | | | | | AAAAAGG | | GTGAAAA | | | | |
| | | | | | | | | AACCGG | | AGGAACC | | | | |
| | | | | | | | | | | GGTGGAG | | | | |
| | | | | | | | | | | A | | | | |
| MBD2 | FOXP3 | FOXP3 | 8932 | NM_003927.4 | NC_000018.10 | 54224170 | sense | AGCCGGT | 517. | CGGGAGC | 593. | GGG | 1 | 0.5949 |
| | | high | | | | | | CCCTTTCC | | CGGTCCCT | | | | |
| | | | | | | | | CGTCG | | TTCCCGTC | | | | |
| | | | | | | | | | | GGGGAGC | | | | |
| MBD2 | FOXP3 | FOXP3 | 8932 | NM_003927.4 | NC_000018.10 | 54205113 | sense | CCTCAGTT | 518. | CAAGCCTC | 594. | GGG | 2 | 0.5818 |
| | | high | | | | | | GGCAAGG | | AGTTGGC | | | | |
| | | | | | | | | TACCT | | AAGGTAC | | | | |
| | | | | | | | | | | CTGGGAA | | | | |
| | | | | | | | | | | A | | | | |
| MBD2 | FOXP3 | FOXP3 | 8932 | NM_003927.4 | NC_000018.10 | 54204999 | sense | CCTCTCAA | 519. | CGATCCTC | 595. | TGG | 2 | 0.5401 |
| | | high | | | | | | TCAAAATA | | TCAATCAA | | | | |
| | | | | | | | | AGGT | | AATAAGG | | | | |
| | | | | | | | | | | TTGGTTA | | | | |
| MBD2 | FOXP3 | FOXP3 | 8932 | NM_003927.4 | NC_000018.10 | 54224048 | sense | CGAAAAT | 520. | GATCCGA | 596. | TGG | 1 | 0.494 |
| | | high | | | | | | CTGGGCT | | AAATCTG | | | | |
| | | | | | | | | AAGTGC | | GGCTAAG | | | | |
| | | | | | | | | | | TGCTGGC | | | | |
| | | | | | | | | | | AA | | | | |
| SMARCB1 | FOXP3 | FOXP3 | 6598 | NM_003073.3 | NC_000022.11 | 23791773 | antisense | GAGAACC | 521. | TACAGAG | 597. | AGG | 2 | 0.731 |
| | | high | | | | | | TCGGAAC | | AACCTCG | | | | |
| | | | | | | | | ATACGG | | GAACATA | | | | |
| | | | | | | | | | | CGGAGGT | | | | |
| | | | | | | | | | | AG | | | | |
| SMARCB1 | FOXP3 | FOXP3 | 6598 | NM_003073.3 | NC_000022.11 | 23816887 | sense | GCAGATC | 522. | GACAGCA | 598. | CGG | 6 | 0.6894 |
| | | high | | | | | | GAGTCCTA | | GATCGAG | | | | |
| | | | | | | | | CCCCA | | TCCTACCC | | | | |
| | | | | | | | | | | CACGGAC | | | | |
| | | | | | | | | | | A | | | | |
| SMARCB1 | FOXP3 | FOXP3 | 6598 | NM_003073.3 | NC_000022.11 | 23801049 | antisense | TCTTCTTG | 523. | GTTCTCTT | 599. | CGG | 4 | 0.6806 |
| | | high | | | | | | TCTCGGCC | | CTTGTCTC | | | | |
| | | | | | | | | CATG | | GGCCCAT | | | | |
| | | | | | | | | | | GCGGTTC | | | | |
| SMARCB1 | FOXP3 | FOXP3 | 6598 | NM_003073.3 | NC_000022.11 | 23803342 | sense | TGAGAAC | 524. | TCCATGAG | 600. | AGG | 5 | 0.6804 |
| | | high | | | | | | GCATCTCA | | AACGCATC | | | | |
| | | | | | | | | GCCCG | | TCAGCCCG | | | | |
| | | | | | | | | | | AGGTGC | | | | |
| ETS1 | FOXP3 | FOXP3 | 2113 | NM_005238.3 | NC_000022.11 | 1.28E+08 | antisense | CTTACTAA | 525. | TGAACTTA | 601. | AGG | 4 | 0.6808 |
| | | high | | | | | | TGAAGTA | | CTAATGAA | | | | |
| | | | | | | | | ATCCG | | GTAATOCG | | | | |
| | | | | | | | | | | AGGTAT | | | | |
| ETS1 | FOXP3 | FOXP3 | 2113 | NM_005238.3 | NC_000022.11 | 1.28E+08 | antisense | GAGAAAG | 526. | GCTCGAG | 602. | GGG | 3 | 0.6487 |
| | | high | | | | | | CAGTCTTT | | AAAGCAG | | | | |
| | | | | | | | | ACCCA | | TCTTTACC | | | | |
| | | | | | | | | | | CAGGGCG | | | | |
| | | | | | | | | | | C | | | | |
| ETS1 | FOXP3 | FOXP3 | 2113 | NM_005238.3 | NC_000022.11 | 1.28E+08 | antisense | GGTCTCG | 527. | AGAGGGT | 603. | GGG | 5 | 0.7649 |
| | | high | | | | | | GAGAATG | | CTCGGAG | | | | |
| | | | | | | | | ACCGAG | | AATGACC | | | | |
| | | | | | | | | | | GAGGGGT | | | | |
| | | | | | | | | | | AG | | | | |
| ETS1 | FOXP3 | FOXP3 | 2113 | NM_005238.3 | NC_000022.11 | 1.28E+08 | sense | TGCATGG | 528. | CATGTGCA | 604. | TGG | 5 | 0.6503 |
| | | high | | | | | | GGAGGAC | | TGGGGAG | | | | |
| | | | | | | | | CAGTCG | | GACCAGT | | | | |
| | | | | | | | | | | CGTGGTA | | | | |
| | | | | | | | | | | G | | | | |
| ZBTB7A | FOXP3 | FOXP3 | 51341 | NM_015898.2 | NC_000019.10 | 4054925 | sense | ACCGTCA | 529. | GCTCACCG | 605. | GGG | 2 | 0.6826 |
| | | high | | | | | | GCACAGC | | TCAGCACA | | | | |
| | | | | | | | | CAACGT | | GCCAACG | | | | |
| | | | | | | | | | | TGGGTGA | | | | |
| ZBTB7A | FOXP3 | FOXP3 | 51341 | NM_015898.2 | NC_000019.10 | 4054671 | antisense | ATCATCGG | 530. | GGTCATCA | 606. | CGG | 2 | 0.6019 |
| | | high | | | | | | ACGCCCCA | | TCGGACG | | | | |
| | | | | | | | | AAGG | | CCCCAAA | | | | |
| | | | | | | | | | | GGCGGAC | | | | |
| | | | | | | | | | | C | | | | |
| ZBTB7A | FOXP3 | FOXP3 | 51341 | NM_015898.2 | NC_000019.10 | 4054199 | sense | GAGTCGC | 531. | CGAGGAG | 607. | GGG | 2 | 0.6111 |
| | | high | | | | | | GGGCCGA | | TCGCGGG | | | | |
| | | | | | | | | CGACAA | | CCGACGA | | | | |
| | | | | | | | | | | CAAGGGC | | | | |
| | | | | | | | | | | GT | | | | |
| ZBTB7A | FOXP3 | FOXP3 | 51341 | NM_015898.2 | NC_000019.10 | 4054431 | antisense | GCCGTAG | 532. | CGCGGCC | 608. | TGG | 2 | 0.6268 |
| | | high | | | | | | TGGCCGTT | | GTAGTGG | | | | |
| | | | | | | | | CTGCG | | CCGTTCTG | | | | |
| | | | | | | | | | | CGTGGCG | | | | |
| | | | | | | | | | | G | | | | |
| MYB | FOXP3 | FOXP3 | 4602 | NM_005375.2 | NC_000006.12 | 1.35E+08 | sense | ACCAGGC | 533. | ATTTACCA | 609. | GGG | 5 | 0.6859 |
| | | high | | | | | | ACACAAG | | GGCACAC | | | | |
| | | | | | | | | AGACTG | | AAGAGAC | | | | |
| | | | | | | | | | | TGGGGAA | | | | |
| | | | | | | | | | | C | | | | |
| MYB | FOXP3 | FOXP3 | 4602 | NM_005375.2 | NC_000006.12 | 1.35E+08 | sense | AGAAATA | 534. | GTACAGA | 610. | TGG | 5 | 0.6009 |
| | | high | | | | | | CGGTCCG | | AATACGG | | | | |
| | | | | | | | | AAACGT | | TCCGAAAC | | | | |
| | | | | | | | | | | GTTGGTCT | | | | |
| MYB | FOXP3 | FOXP3 | 4602 | NM_005375.2 | NC_000006.12 | 1.35E+08 | sense | AGTCTGG | 535. | CCCAAGTC | 611. | GGG | 2 | 0.7063 |
| | | high | | | | | | AAAGCGT | | TGGAAAG | | | | |
| | | | | | | | | CACTTG | | CGTCACTT | | | | |
| | | | | | | | | | | GGGGAAA | | | | |
| MYB | FOXP3 | FOXP3 | 4602 | NM_005375.2 | NC_000006.12 | 1.35E+08 | antisense | TATTTACA | 536. | ACTATATT | 612. | GGG | 7 | 0.6157 |
| | | high | | | | | | TGTAACGC | | TACATGTA | | | | |
| | | | | | | | | TACA | | ACGCTACA | | | | |
| | | | | | | | | | | GGGTAT | | | | |
| SATB1 | FOXP3 | FOXP3 | 6304 | NM_002971.4 | NC_000006.12 | 18415117 | antisense | ATGCTAA | 537. | TTCTATGC | 613. | GGG | 5 | 0.6378 |
| | | high | | | | | | GTACCTGT | | TAAGTACC | | | | |
| | | | | | | | | GAAAG | | TGTGAAA | | | | |
| | | | | | | | | | | GGGGGCA | | | | |
| SATB1 | FOXP3 | FOXP3 | 6304 | NM_002971.4 | NC_000006.12 | 18417016 | sense | CATTGAAT | 538. | ACGCCATT | 614. | AGG | 3 | 0.7747 |
| | | high | | | | | | ATGATTGC | | GAATATG | | | | |
| | | | | | | | | AAGG | | ATTGCAA | | | | |
| | | | | | | | | | | GGAGGAG | | | | |
| | | | | | | | | | | C | | | | |
| SATB1 | FOXP3 | FOXP3 | 6304 | NM_002971.4 | NC_000006.12 | 18394751 | antisense | TAGGTGTT | 539. | CTGATAG | 615. | GGG | 7 | 0.6656 |
| | | high | | | | | | GATACGA | | GTGTTGAT | | | | |
| | | | | | | | | GCCCA | | ACGAGCC | | | | |
| | | | | | | | | | | CAGGGTG | | | | |
| | | | | | | | | | | C | | | | |
| SATB1 | FOXP3 | FOXP3 | 6304 | NM_002971.4 | NC_000006.12 | 18394610 | antisense | TATTCATA | 540. | GGCTTATT | 616. | GGG | 7 | 0.6676 |
| | | high | | | | | | GATCTACT | | CATAGATC | | | | |
| | | | | | | | | GACA | | TACTGACA | | | | |
| | | | | | | | | | | GGGGGA | | | | |
| TFDP1 | FOXP3 | FOXP3 | 7027 | NM_007111.4 | NC_000013.11 | 1.14E+08 | sense | ACCGGCA | 541. | AGAGACC | 617. | TGG | 5 | 0.6315 |
| | | high | | | | | | GCGTCAA | | GGCAGCG | | | | |
| | | | | | | | | ACACCC | | TCAAACAC | | | | |
| | | | | | | | | | | CCTGGTG | | | | |
| | | | | | | | | | | G | | | | |
| TFDP1 | FOXP3 | FOXP3 | 7027 | NM_007111.4 | NC_000013.11 | 1.14E+08 | sense | ATGACCA | 542. | GCTTATGA | 618. | CGG | 7 | 0.6204 |
| | | high | | | | | | GAAAAAC | | CCAGAAA | | | | |
| | | | | | | | | ATAAGA | | AACATAA | | | | |
| | | | | | | | | | | GACGGCG | | | | |
| | | | | | | | | | | C | | | | |
| TFDP1 | FOXP3 | FOXP3 | 7027 | NM_007111.4 | NC_000013.11 | 1.14E+08 | antisense | CCTTCATG | 543. | CAGACCTT | 619. | AGG | 6 | 0.6851 |
| | | high | | | | | | GAGAAAT | | CATGGAG | | | | |
| | | | | | | | | GCCGT | | AAATGCC | | | | |
| | | | | | | | | | | GTAGGCC | | | | |
| | | | | | | | | | | C | | | | |
| TFDP1 | FOXP3 | FOXP3 | 7027 | NM_007111.4 | NC_000013.11 | 1.14E+08 | sense | GGTGCAG | 544. | ACCTGGT | 620. | CGG | 9 | 0.6402 |
| | | high | | | | | | AGAAACC | | GCAGAGA | | | | |
| | | | | | | | | GGCATG | | AACCGGC | | | | |
| | | | | | | | | | | ATGCGGA | | | | |
| | | | | | | | | | | GC | | | | |
| DNMT1 | FOXP3 | FOXP3 | 1786 | NM_001379.2 | NC_000019.10 | 10146475 | sense | GAGGCAA | 545. | AGATGAG | 621. | GGG | 27 | 0.7285 |
| | | high | | | | | | AAAGAAA | | GCAAAAA | | | | |
| | | | | | | | | TCCCCA | | GAAATCCC | | | | |
| | | | | | | | | | | CAGGGTC | | | | |
| | | | | | | | | | | C | | | | |
| DNMT1 | FOXP3 | FOXP3 | 1786 | NM_001379.2 | NC_000019.10 | 10162681 | sense | GATTTCTG | 546. | CACAGATT | 622. | AGG | 12 | 0.7345 |
| | | high | | | | | | ATGAAAA | | TCTGATGA | | | | |
| | | | | | | | | AGACG | | AAAAGAC | | | | |
| | | | | | | | | | | GAGGATG | | | | |
| DNMT1 | FOXP3 | FOXP3 | 1786 | NM_001379.2 | NC_000019.10 | 10142150 | sense | GCTCTACT | 547. | ACCTGCTC | 623. | AGG | 29 | 0.6656 |
| | | high | | | | | | GGAGCGA | | TACTGGA | | | | |
| | | | | | | | | CGAGG | | GCGACGA | | | | |
| | | | | | | | | | | GGAGGCC | | | | |
| | | | | | | | | | | G | | | | |
| DNMT1 | FOXP3 | FOXP3 | 1786 | NM_001379.2 | NC_000019.10 | 10151459 | sense | TCACCCAA | 548. | GCCGTCAC | 624. | GGG | 23 | 0.7089 |
| | | high | | | | | | AAAAATG | | CCAAAAA | | | | |
| | | | | | | | | CACCA | | AATGCACC | | | | |
| | | | | | | | | | | AGGGGAA | | | | |

TABLE 4
Nuclear factors that can be inhibited to decrease FOXP3 expression or overexpressed to increase FOXP3 expression

| | | | | | | Position | | | | | | | | |
|---|---|---|---|---|---|---|---|---|---|---|---|---|---|---|
| | | screen_ | | | | of Base | | sgRNA | SEQ | Target | SEQ | | | Rule |
| gene_ | screen_ | di- | Target | Target | Genomic | After Cut | | Target | ID | Context | ID | PAM | Exon | Set 2 |
| id | target | rection | Gene ID | Transcript | Sequence | (1-based) | Strand | Sequence | NO: | Sequence | NO: | Sequence | Number | score |
| PTEN | FOXP3 | FOXP3 low | 5728 | NM_ | NC_ | 87961027 | sense | AGAGCGT | 625. | GTATAGA | 693. | AGG | 8 | 0.7179 |
| | | | | 001304718.1 | 000010.11 | | | GCAGATA | | GCGTGCA | | | | |
| | | | | | | | | ATGACA | | GATAATG | | | | |
| | | | | | | | | | | ACAAGGA | | | | |
| | | | | | | | | | | AT | | | | |
| PTEN | FOXP3 | FOXP3 low | 5728 | NM_ | NC_ | 87957861 | anti- | AGCTGGC | 626. | CTTTAGCT | 694. | AGG | 7 | 0.6493 |
| | | | | 001304718.1 | 000010.11 | | sense | AGACCAC | | GGCAGAC | | | | |
| | | | | | | | | AAACTG | | CACAAACT | | | | |
| | | | | | | | | | | GAGGATC | | | | |
| PTEN | FOXP3 | FOXP3 low | 5728 | NM_ | NC_ | 87960940 | sense | ATTCTTCA | 627. | ATACATTC | 695. | AGG | 8 | 0.6697 |
| | | | | 001304718.1 | 000010.11 | | | TACCAGG | | TTCATACC | | | | |
| | | | | | | | | ACCAG | | AGGACCA | | | | |
| | | | | | | | | | | GAGGAAA | | | | |
| PTEN | FOXP3 | FOXP3 low | 5728 | NM_ | NC_ | 87957915 | sense | CCAATTCA | 628. | TCCTCCAA | 696. | CGG | 7 | 0.6752 |
| | | | | 001304718.1 | 000010.11 | | | GGACCCA | | TTCAGGAC | | | | |
| | | | | | | | | CACGA | | CCACACG | | | | |
| | | | | | | | | | | ACGGGAA | | | | |
| GATA3 | FOXP3 | FOXP3 low | 2625 | NM_ | NC_ | 8055892 | sense | AGGTACC | 629. | GCAGAGG | 697. | CGG | 2 | 0.6857 |
| | | | | 002051.2 | 000010.11 | | | CTCCGACC | | TACCCTCC | | | | |
| | | | | | | | | CACCA | | GACCCACC | | | | |
| | | | | | | | | | | ACGGTGA | | | | |
| GATA3 | FOXP3 | FOXP3 low | 2625 | NM_ | NC | 8064014 | sense | CAGGGAG | 630. | AAGGCAG | 698. | GGG | 4 | 0.737 |
| | | | | 002051.2 | 000010.11 | | | TGTGTGA | | GGAGTGT | | | | |
| | | | | | | | | ACTGTG | | GTGAACT | | | | |
| | | | | | | | | | | GTGGGGC | | | | |
| | | | | | | | | | | AA | | | | |
| GATA3 | FOXP3 | FOXP3 low | 2625 | NM_ | NC_ | 8058740 | anti- | GGAGCTG | 631. | GTCCGGA | 699. | AGG | 3 | 0.6273 |
| | | | | 002051.2 | 000010.11 | | sense | TACTCGG | | GCTGTACT | | | | |
| | | | | | | | | GCACGT | | CGGGCAC | | | | |
| | | | | | | | | | | GTAGGGC | | | | |
| | | | | | | | | | | G | | | | |
| GATA3 | FOXP3 | FOXP3 low | 2625 | NM_ | NC_ | 8058432 | sense | TCCAAGAC | 632. | CTTCTCCA | 700. | CGG | 3 | 0.7243 |
| | | | | 002051.2 | 000010.11 | | | GTCCATCC | | AGACGTC | | | | |
| | | | | | | | | ACCA | | CATCCACC | | | | |
| | | | | | | | | | | ACGGCTC | | | | |
| STAT5B | FOXP3 | FOXP3 low | 6777 | NM_ | NC_ | 42216055 | sense | CAGCCAG | 633. | ATGGCAG | 701. | CGG | 12 | 0.6375 |
| | | | | 401248.3 | 000017.11 | | | GACAACA | | CCAGGAC | | | | |
| | | | | | | | | ATGCGA | | AACAATG | | | | |
| | | | | | | | | | | CGACGGC | | | | |
| | | | | | | | | | | CA | | | | |
| STAT5B | FOXP3 | FOXP3 low | 6777 | NM_ | NC_ | 42227658 | anti- | GTGGCCTT | 634. | CTGGGTG | 702. | TGG | 3 | 0.6157 |
| | | | | 012448.3 | 000017.11 | | sense | AATGTTCT | | GCCTTAAT | | | | |
| | | | | | | | | CCTG | | GTTCTCCT | | | | |
| | | | | | | | | | | GTGGATT | | | | |
| STAT5B | FOXP3 | FOXP3 low | 6777 | NM_ | NC_ | 42224822 | anti- | GTTCATTG | 635. | CTCTGTTC | 703. | CGG | 4 | 0.6583 |
| | | | | 012448.3 | 000017.11 | | sense | TACAATAT | | ATTGTACA | | | | |
| | | | | | | | | ATGG | | ATATATGG | | | | |
| | | | | | | | | | | CGGATG | | | | |
| STAT5B | FOXP3 | FOXP3 low | 6777 | NM_ | NC_ | 42217252 | sense | TAAGAGG | 636. | GAATTAA | 704. | GGG | 11 | 0.7097 |
| | | | | 012448.3 | 000017.11 | | | TCAGACC | | GAGGTCA | | | | |
| | | | | | | | | GTCGTG | | GACCGTC | | | | |
| | | | | | | | | | | GTGGGGC | | | | |
| | | | | | | | | | | AG | | | | |
| MED30 | FOXP3 | FOXP3 low | 90390 | NM_ | NC_ | 1.18E+08 | sense | ACACTGG | 637. | TACCACAC | 705. | CGG | 2 | 0.6317 |
| | | | | 080651.3 | 000008.11 | | | AACATATC | | TGGAACA | | | | |
| | | | | | | | | AAGAC | | TATCAAGA | | | | |
| | | | | | | | | | | CCGGTTA | | | | |
| MED30 | FOXP3 | FOXP3 low | 90390 | NM_ | NC_ | 1.18E+08 | sense | GACAAAT | 638. | ATATGACA | 706. | TGG | 2 | 0.7364 |
| | | | | 080651.3 | 000008.11 | | | GCAATGA | | AATGCAAT | | | | |
| | | | | | | | | AAACTG | | GAAAACT | | | | |
| | | | | | | | | | | GTGGTGG | | | | |
| MED30 | FOXP3 | FOXP3 low | 90390 | NM_ | NC_ | 1.18E+08 | sense | GGACATC | 639. | TGCAGGA | 707. | TGG | 1 | 0.6878 |
| | | | | 080651.3 | 000008.11 | | | GTGTACC | | CATCGTGT | | | | |
| | | | | | | | | GCACCA | | ACCGCACC | | | | |
| | | | | | | | | | | ATGGAGA | | | | |
| MED30 | FOXP3 | FOXP3 low | 90390 | NM_ | NC_ | 1.18E+08 | sense | GGCCGCC | 640. | AGCAGGC | 708. | CGG | 1 | 0.5967 |
| | | | | 080651.3 | 000008.11 | | | CGGGAAG | | CGCCCGG | | | | |
| | | | | | | | | TCAACA | | GAAGTCA | | | | |
| | | | | | | | | | | ACACGGC | | | | |
| | | | | | | | | | | GT | | | | |
| FOXP1 | FOXP3 | FOXP3 low | 27086 | NM_ | NC_ | 71041428 | anti- | AGAGGAG | 641. | GTGCAGA | 709. | TGG | 11 | 0.6926 |
| | | | | 032682.5 | 000003.12 | | sense | GAGACAC | | GGAGGAG | | | | |
| | | | | | | | | ATGTCG | | ACACATGT | | | | |
| | | | | | | | | | | CGTGGTC | | | | |
| | | | | | | | | | | A | | | | |
| FOXP1 | FOXP3 | FOXP3 low | 27086 | NM_ | NC_ | 71015617 | anti- | CATACACC | 642. | CTTGCATA | 710. | AGG | 12 | 0.6547 |
| | | | | 032682.5 | 000003.12 | | sense | ATGTCCAT | | CACCATGT | | | | |
| | | | | | | | | AGAG | | CCATAGA | | | | |
| | | | | | | | | | | GAGGATG | | | | |
| FOXP1 | FOXP3 | FOXP3 low | 27086 | NM_ | NC_ | 71046982 | sense | GCCTTCTG | 643. | CAAGGCC | 711. | GGG | 10 | 0.5683 |
| | | | | 032682.5 | 000003.12 | | | ACAATTCA | | TTCTGACA | | | | |
| | | | | | | | | GCCC | | ATTCAGCC | | | | |
| | | | | | | | | | | CGGGCAG | | | | |
| FOXP1 | FOXP3 | FOXP3 low | 27086 | NM_ | NC_ | 70988031 | anti- | GTTCTGTA | 644. | TTGGGTTC | 712. | AGG | 14 | 0.5925 |
| | | | | 032682.5 | 000003.12 | | sense | GACTTCAC | | TGTAGACT | | | | |
| | | | | | | | | ATGC | | TCACATGC | | | | |
| | | | | | | | | | | AGGTGG | | | | |
| FOXP3 | FOXP3 | FOXP3 low | 50943 | NM_ | NC_ | 49254057 | sense | ACCCAGG | 645. | CCTCACCC | 713. | GGG | 9 | 0.6666 |
| | | | | 014009.3 | 000023.11 | | | CATCATCC | | AGGCATC | | | | |
| | | | | | | | | GACAA | | ATCCGACA | | | | |
| | | | | | | | | | | AGGGCTC | | | | |
| FOXP3 | FOXP3 | FOXP3 low | 50943 | NM_ | NC_ | 49257007 | sense | CCCACCCA | 646. | TGTCCCCA | 714. | TGG | 5 | 0.7429 |
| | | | | 001409.3 | 000023.11 | | | CAGGGAT | | CCCACAG | | | | |
| | | | | | | | | CAACG | | GGATCAA | | | | |
| | | | | | | | | | | CGTGGCC | | | | |
| | | | | | | | | | | A | | | | |
| FOXP3 | FOXP3 | FOXP3 low | 50943 | NM_ | NC_ | 49255795 | sense | CCTACTTA | 647. | TCTCCCTA | 715. | CGG | 7 | 0.6738 |
| | | | | 001409.3 | 000023.11 | | | GGCACTG | | CTTAGGCA | | | | |
| | | | | | | | | CCAGG | | CTGCCAG | | | | |
| | | | | | | | | | | GCGGACC | | | | |
| FOXP3 | FOXP3 | FOXP3 low | 50943 | NM_ | NC_ | 49257751 | anti- | GAGGGTG | 648. | CCCGGAG | 716. | GGG | 3 | 0.6447 |
| | | | | 014009.3 | 000023.11 | | sense | CCACCATG | | GGTGCCA | | | | |
| | | | | | | | | ACTAG | | CCATGACT | | | | |
| | | | | | | | | | | AGGGGCA | | | | |
| | | | | | | | | | | G | | | | |
| CBFB | FOXP3 | FOXP3 low | 865 | NM_ | NC_ | 67036720 | anti- | AAGTCGA | 649. | TTCTAAGT | 717. | AGG | 3 | 0.5993 |
| | | | | 001755.2 | 000016.10 | | sense | CATACTCT | | CGACATAC | | | | |
| | | | | | | | | CGGCT | | TCTCGGCT | | | | |
| | | | | | | | | | | AGGTGT | | | | |
| CBFB | FOXP3 | FOXP3 low | 865 | NM_ | NC_ | 67029479 | anti- | CCTGCCTC | 650. | CCCGCCTG | 718. | CGG | 1 | 0.6743 |
| | | | | 001755.2 | 600001.10 | | sense | ACCTCACA | | CCTCACCT | | | | |
| | | | | | | | | CTCG | | CACACTCG | | | | |
| | | | | | | | | | | CGGCTC | | | | |
| CBFB | FOXP3 | FOXP3 low | 865 | NM_ | NC_ | 167029807 | anti- | GCCGACTT | 651. | GCCAGCC | 719. | CGG | 2 | 0.7383 |
| | | | | 001755.2 | 000016.10 | | sense | ACGATTTC | | GACTTACG | | | | |
| | | | | | | | | CGAG | | ATTTCCGA | | | | |
| | | | | | | | | | | GCGGCCG | | | | |
| CBFB | FOXP3 | FOXP3 low | 865 | NM_ | NC_ | 67066729 | sense | GGAGTCT | 652. | GAATGGA | 720. | AGG | 4 | 0.5938 |
| | | | | 001755.2 | 000016.10 | | | GTGTTATC | | GTCTGTGT | | | | |
| | | | | | | | | TGGAA | | TATCTGGA | | | | |
| | | | | | | | | | | AAGGCTG | | | | |
| FOXO1 | FOXP3 | FOXP3 low | 2308 | NM_ | NC_ | 40665740 | anti- | ACAGGTT | 653. | TAGGACA | 721. | CGG | 1 | 0.547 |
| | | | | 002015.3 | 000013.11 | | sense | GCCCCAC | | GGTTGCCC | | | | |
| | | | | | | | | GCGTTG | | CACGCGTT | | | | |
| | | | | | | | | | | GCGGCGG | | | | |
| FOX01 | FOXP3 | FOXP3 low | 2308 | NM_ | NC_ | 40666107 | sense | GGAGTTT | 654. | GGCCGGA | 722. | CGG | 1 | 0.629 |
| | | | | 002015.3 | 000013.11 | | | AGCCAGT | | GTTTAGCC | | | | |
| | | | | | | | | CCAACT | | AGTCCAAC | | | | |
| | | | | | | | | | | TCGGCCA | | | | |
| FOX01 | FOXP3 | FOXP3 low | 2308 | NM_ | NC_ | 40560279 | anti- | GGTGGCG | 655. | GTTTGGTG | 723. | CGG | 2 | 0.6741 |
| | | | | 002015.3 | 000013.11 | | sense | CAAACGA | | GCGCAAA | | | | |
| | | | | | | | | GTAGCA | | CGAGTAG | | | | |
| | | | | | | | | | | CACGGCG | | | | |
| | | | | | | | | | | T | | | | |
| FOX01 | FOXP3 | FOXP3 low | 2308 | NM_ | NC_ | 40560544 | anti- | TAGCATTT | 656. | GTACTAGC | 724. | GGG | 2 | 0.6665 |
| | | | | 002015.3 | 000013.11 | | sense | GAGCTAG | | ATTTGAGC | | | | |
| | | | | | | | | TTCGA | | TAGTTCGA | | | | |
| | | | | | | | | | | GGGCGA | | | | |
| SS18 | FOXP3 | FOXP3 low | 6760 | NM_ | NC_ | 26052686 | sense | AATCAGAT | 657. | ACAGAAT | 725. | GGG | 5 | 0.6566 |
| | | | | 001007559.1 | 800001.10 | | | GACAATG | | CAGATGA | | | | |
| | | | | | | | | AGTCA | | CAATGAG | | | | |
| | | | | | | | | | | TCAGGGA | | | | |
| | | | | | | | | | | CA | | | | |
| SS18 | FOXP3 | FOXP3 low | 6760 | NM_ | NC_ | 26039408 | sense | CAATACAA | 658. | TCAGCAAT | 726. | AGG | 6 | 0.7278 |
| | | | | 001007559.1 | 000018.10 | | | TATGCCAC | | ACAATATG | | | | |
| | | | | | | | | AGGG | | CCACAGG | | | | |
| | | | | | | | | | | GAGGCGG | | | | |
| SS18 | FOXP3 | FOXP3 low | 6760 | NM_ | NC_ | 26052827 | sense | CCTAACCA | 659. | AGGGCCT | 727. | GGG | 5 | 0.6709 |
| | | | | 000107559.1 | 000018.10 | | | TATGCCTA | | AACCATAT | | | | |
| | | | | | | | | TGCA | | GCCTATGC | | | | |
| | | | | | | | | | | AGGGACC | | | | |
| SS18 | FOXP3 | FOXP3 low | 6760 | NM_ | NC_ | 26057677 | anti- | GGCATGTT | 660. | TGAAGGC | 728. | AGG | 4 | 0.7849 |
| | | | | 001007559.1 | 000018.10 | | sense | GTGAGAG | | ATGTTGTG | | | | |
| | | | | | | | | CGTGG | | AGAGCGT | | | | |
| | | | | | | | | | | GGAGGTG | | | | |
| | | | | | | | | | | G | | | | |
| MED14 | FOXP3 | FOXP3 low | 9282 | NM_ | NC_ | 40692233 | anti- | ATCACACA | 661. | TTGTATCA | 729. | GGG | 15 | 0.6711 |
| | | | | 004229.3 | 000023.11 | | sense | TAGCGAC | | CACATAGC | | | | |
| | | | | | | | | GAAGT | | GACGAAG | | | | |
| | | | | | | | | | | TGGGCTA | | | | |
| MED14 | FOXP3 | FOXP3 low | 9282 | NM_ | NC_ | 40714644 | anti- | CAGAGCA | 662. | GGACCAG | 730. | AGG | 4 | 0.6206 |
| | | | | 004229.3 | 000023.11 | | sense | TCTCTAGC | | AGCATCTC | | | | |
| | | | | | | | | TAACG | | TAGCTAAC | | | | |
| | | | | | | | | | | GAGGCCA | | | | |
| MED14 | FOXP3 | FOXP3 low | 9282 | NM_ | NC_ | 40682898 | anti- | CTAACTCT | 663. | AACACTAA | 731. | CGG | 17 | 0.7032 |
| | | | | 004229.3 | 000023.11 | | sense | GCTACCCA | | CTCTGCTA | | | | |
| | | | | | | | | AGTG | | CCCAAGT | | | | |
| | | | | | | | | | | GCGGTTA | | | | |
| MED14 | FOXP3 | FOXP3 low | 9282 | NM_ | NC_ | 40711237 | sense | TAATGTTA | 664. | ACTCTAAT | 732. | TGG | 8 | 0.6368 |
| | | | | 004229.3 | 000023.11 | | | ATCCGAG | | GTTAATCC | | | | |
| | | | | | | | | AACGG | | GAGAACG | | | | |
| | | | | | | | | | | GTGGGGA | | | | |
| DDX39B | FOXP3 | FOXP3 low | 7919 | NM_ | NC_ | 31540429 | sense | CCTGCCAA | 665. | GGCCCCT | 733. | GGG | 2 | 0.6194 |
| | | | | 080598.5 | 000006.12 | | | GAAGGAT | | GCCAAGA | | | | |
| | | | | | | | | GTCAA | | AGGATGT | | | | |
| | | | | | | | | | | CAAGGGC | | | | |
| | | | | | | | | | | TC | | | | |
| DDX39B | FOXP3 | FOXP3 low | 7919 | NM_ | NC_ | 31540352 | anti- | CTCAAAGC | 666. | GATGCTCA | 734. | TGG | 2 | 0.6078 |
| | | | | 080598.5 | 000006.12 | | sense | CACAGTC | | AAGCCAC | | | | |
| | | | | | | | | GACAA | | AGTCGAC | | | | |
| | | | | | | | | | | AATGGCC | | | | |
| | | | | | | | | | | C | | | | |
| DDX39B | FOXP3 | FOXP3 low | 7919 | NM_ | NC_ | 31536578 | anti- | GAGGCTC | 667. | GGTTGAG | 735. | GGG | 5 | 0.6386 |
| | | | | 080598.5 | 000006.12 | | sense | TTATTTCG | | GCTCTTAT | | | | |
| | | | | | | | | AGCCA | | TTCGAGCC | | | | |
| | | | | | | | | | | AGGGCTA | | | | |
| DDX39B | FOXP3 | FOXP3 low | 7919 | NM_ | NC_ | 31536505 | sense | GATAAGA | 668. | ATGTGATA | 736. | CGG | 5 | 0.7042 |
| | | | | 908058.5 | 000006.12 | | | TGCTTGÅA | | AGATGCTT | | | | |
| | | | | | | | | CAGCT | | GAACAGC | | | | |
| | | | | | | | | | | TCGGTGA | | | | |
| SOCS3 | FOXP | FOXP3 low | 9021 | NM_ | NC_ | 78358663 | sense | ACCTACTG | 669. | CGCCACCT | 737. | AGG | 2 | 0.6512 |
| | | | | 003955.4 | 000017.11 | | | AACCCTCC | | ACTGAACC | | | | |
| | | | | | | | | TCCG | | CTCCTCCG | | | | |
| | | | | | | | | | | AGGTGC | | | | |
| SOCS3 | FOXP3 | FOXP3 low | 9021 | NM_ | NC_ | 78358900 | anti- | GCGGATC | 670. | TGTCGCG | 738. | CGG | 2 | 0.6181 |
| | | | | 003955.4 | 000017.11 | | sense | AGAAAGG | | GATCAGA | | | | |
| | | | | | | | | TGCCGG | | AAGGTGC | | | | |
| | | | | | | | | | | CGGCGGG | | | | |
| | | | | | | | | | | CT | | | | |
| SOCS3 | FOXP3 | FOXP3 low | 9021 | NM_ | NC_ | 78358835 | sense | TCAGOGTC | 671. | ACGCTCA | 739. | GGG | 2 | 0.5946 |
| | | | | 003955.4 | 000017.11 | | | AAGACCC | | GCGTCAA | | | | |
| | | | | | | | | AGTCT | | GACCCAG | | | | |
| | | | | | | | | | | TCTGGGA | | | | |
| | | | | | | | | | | CC | | | | |
| SOCS3 | FOXP3 | FOXP3 low | 9021 | NM_ | NC_ | 78358748 | anti- | TTGAGCAC | 672. | CAGCTTGA | 740. | GGG | 2 | 0.5968 |
| | | | | 003955.4 | 000017.11 | | sense | GCAGTCG | | GCACGCA | | | | |
| | | | | | | | | AAGCG | | GTCGAAG | | | | |
| | | | | | | | | | | CGGGGCA | | | | |
| | | | | | | | | | | C | | | | |
| SP2 | FOXP3 | FOXP3 low | 6668 | NM_ | NC_ | 47922963 | anti- | AGGCGTG | 673. | CGGAAGG | 741. | GGG | 4 | 0.6481 |
| | | | | 003110.5 | 000017.11 | | sense | CGGATGT | | CGTGCGG | | | | |
| | | | | | | | | AGACCT | | ATGTAGA | | | | |
| | | | | | | | | | | CCTGGGA | | | | |
| | | | | | | | | | | GG | | | | |
| SP2 | FOXP3 | FOXP3 low | 6668 | NM_ | NC_ | 47916790 | anti- | CTTGTTAG | 674. | CTTTCTTG | 742. | GGG | 3 | 0.7011 |
| | | | | 003110.5 | 000017.11 | | sense | TCTTAGAC | | TTAGTCTT | | | | |
| | | | | | | | | AGCG | | AGACAGC | | | | |
| | | | | | | | | | | GGGGTTG | | | | |
| SP2 | FOXP3 | FOXP3 low | 6668 | NM_ | NC_ | 47916469 | sense | TGCCAATA | 675. | CAAATGCC | 743. | CGG | 3 | 0.7354 |
| | | | | 003110.5 | 000017.11 | | | TCCAGTAC | | AATATCCA | | | | |
| | | | | | | | | CAGG | | GTACCAG | | | | |
| | | | | | | | | | | GCGGTCC | | | | |
| SP2 | FOXP3 | FOXP3 low | 6668 | NM_ | NC_ | 47916267 | anti- | TTGATAG | 676. | AGGTTTG | 744. | CGG | 3 | 0.7038 |
| | | | | 003110.5 | 000017.11 | | sense | GGACAAG | | ATAGGGA | | | | |
| | | | | | | | | TTTCCG | | CAAGTTTC | | | | |
| | | | | | | | | | | CGCGGTG | | | | |
| | | | | | | | | | | T | | | | |
| STATSA | FOXP3 | FOXP3 low | 6776 | NM_ | NC_ | 42292035 | sense | ACATTCTG | 677. | CGGCACA | 745. | AGG | 5 | 0.7341 |
| | | | | 500312.3 | 000017.11 | | | TACAATGA | | TTCTGTAC | | | | |
| | | | | | | | | ACAG | | AATGAAC | | | | |
| | | | | | | | | | | AGAGGCT | | | | |
| | | | | | | | | | | G | | | | |
| STAT5A | FOXP3 | FOXP3 low | 6776 | NM_ | NC_ | 42304559 | sense | ATCAAGC | 678. | GAGGATC | 746. | GGG | 12 | 0.6531 |
| | | | | 003152.3 | 000017.11 | | | GTGCTGA | | AAGCGTG | | | | |
| | | | | | | | | CCGGCG | | CTGACCG | | | | |
| | | | | | | | | | | GCGGGGT | | | | |
| | | | | | | | | | | GC | | | | |
| STAT5A | FOXP3 | FOXP3 low | 6776 | NM_ | NC_ | 42305662 | sense | CAGCCAG | 679. | ACGGCAG | 747. | CGG | 13 | 0.6255 |
| | | | | 003152.3 | 000017.11 | | | GACCACA | | CCAGGAC | | | | |
| | | | | | | | | ATGCCA | | CACAATGC | | | | |
| | | | | | | | | | | CACGGCT | | | | |
| | | | | | | | | | | Å | | | | |
| STAT5A | FOXP3 | FOXP3 low | 6776 | NM_ | NC_ | 42301385 | sense | CGTGCAC | 680. | TGAACGT | 748. | AGG | 10 | 0.5812 |
| | | | | 003152.3 | 000017.11 | | | ATGAATCC | | GCACATG | | | | |
| | | | | | | | | CCCCC | | AATCCCCC | | | | |
| | | | | | | | | | | CCAGGTG | | | | |
| | | | | | | | | | | A | | | | |
| TAF5L | FOXP3 | FOXP3 low | 27097 | NM_ | NC_ | 2.3E+08 | anti- | CGGGACA | 681. | GATGCGG | 749. | GGG | 4 | 0.6222 |
| | | | | 014409.3 | 000001.11 | | sense | CGTCTACT | | GACACGT | | | | |
| | | | | | | | | TGGTG | | CTACTTGG | | | | |
| | | | | | | | | | | TGGGGCT | | | | |
| | | | | | | | | | | C | | | | |
| TAF5L | FOXP3 | FOXP3 low | 27097 | NM_ | NC_ | 2.3E+08 | sense | GCAGAAC | 682. | TTCTGCAG | 750. | AGG | 4 | 0.6216 |
| | | | | 014409.3 | 000001.11 | | | GAGGCTG | | AACGAGG | | | | |
| | | | | | | | | CCCTAG | | CTGCCCTA | | | | |
| | | | | | | | | | | GAGGTCT | | | | |
| TAF5L | FOXP3 | FOXP3 low | 27097 | NM_ | NC_ | 2.3E+08 | sense | GCGGACC | 683. | CACTGCG | 751. | AGG | 5 | 0.7066 |
| | | | | 014409.3 | 000001.11 | | | AGTGTAC | | GACCAGT | | | | |
| | | | | | | | | AGCACG | | GTACAGC | | | | |
| | | | | | | | | | | ACGAGGT | | | | |
| | | | | | | | | | | TC | | | | |
| TAF5L | FOXP3 | FOXP3 low | 27097 | NM_ | NC_ | 2.3E+08 | anti- | TAAGGTG | 684. | TATGTAAG | 752. | GGG | 4 | 0.6402 |
| | | | | 014409.3 | 000001.11 | | sense | AGGACTTT | | GTGAGGA | | | | |
| | | | | | | | | GCACA | | CTTTGCAC | | | | |
| | | | | | | | | | | AGGGCAG | | | | |
| SETDB1 | FOXP3 | FOXP3 low | 9869 | NM_ | NC_ | 1.51E+08 | sense | AAGGAAA | 685. | AACAAAG | 753. | GGG | 7 | 0.663 |
| | | | | 012432.3 | 000001.11 | | | GAGTCTAC | | GAAAGAG | | | | |
| | | | | | | | | TGTCG | | TCTACTGT | | | | |
| | | | | | | | | | | CGGGGAA | | | | |
| | | | | | | | | | | C | | | | |
| SETDB1 | FOXP3 | FOXP3 low | 9869 | NM_ | NC_ | 1.51E+08 | sense | AGATGTG | 686. | CAAAAGA | 754. | AGG | 10 | 0.6692 |
| | | | | 012432.3 | 000001.11 | | | AGTGGAT | | TGTGAGT | | | | |
| | | | | | | | | CTATCG | | GGATCTAT | | | | |
| | | | | | | | | | | CGAGGCT | | | | |
| | | | | | | | | | | C | | | | |
| SETDB1 | FOXP3 | FOXP3 low | 9869 | NM_ | NC_ | 1.51E+08 | anti- | CCTTACCT | 687. | CATCCCTT | 755. | AGG | 3 | 0.723 |
| | | | | 012432.3 | 000001.11 | | sense | GAATCAAT | | ACCTGAAT | | | | |
| | | | | | | | | ACTG | | CAATACTG | | | | |
| | | | | | | | | | | AGGACA | | | | |
| SETDB1 | FOXP3 | FOXP3 low | 9869 | NM_ | NC_ | 1.51E+08 | sense | GTTATCTA | 688. | TCATGTTA | 756. | TGG | 13 | 0.6888 |
| | | | | 012432.3 | 000001.11 | | | TAAGACA | | TCTATAAG | | | | |
| | | | | | | | | CCTTG | | ACACCTTG | | | | |
| | | | | | | | | | | TGGTCT | | | | |
| RNF20 | FOXP3 | FOXP3 low | 56254 | NM_ | NC_ | 1.02E+08 | sense | ACTTCGGC | 689. | AGAAACTT | 757. | AGG | 9 | 0.6981 |
| | | | | 019592.6 | 000009.12 | | | AAGACTTT | | CGGCAAG | | | | |
| | | | | | | | | GAGG | | ACTTTGAG | | | | |
| | | | | | | | | | | GAGGTCA | | | | |
| RNF20 | FOXP3 | FOXP3 low | 56254 | NM_ | NC_ | 1.02E+08 | sense | GCATCGC | 690. | AAAAGCA | 758. | AGG | 6 | 0.6768 |
| | | | | 019592.6 | 000009.12 | | | ACCATGTC | | TCGCACCA | | | | |
| | | | | | | | | TCAGG | | TGTCTCAG | | | | |
| | | | | | | | | | | GAGGTAC | | | | |
| RNF20 | FOXP3 | FOXP3 low | 56254 | NM_ | NC_ | 1.02E+08 | anti- | GGAGGGC | 691 | TGCAGGA | 759. | AGG | 13 | 0.6635 |
| | | | | 019592.6 | 000009.12 | | sense | ACTACCAC | | GGGCACT | | | | |
| | | | | | | | | TACGC | | ACCACTAC | | | | |
| | | | | | | | | | | GCAGGCG | | | | |
| | | | | | | | | | | T | | | | |
| RNF20 | FOXP3 | FOXP3 low | 56254 | NM_ | NC_ | 1.02E+08 | anti- | TCGGTTGA | 692. | AGTATCG | 760. | AGG | 3 | 0.658 |
| | | | | 019592.6 | 000009.12 | | sense | CAATCAAT | | GTTGACA | | | | |
| | | | | | | | | AGTG | | ATCAATAG | | | | |
| | | | | | | | | | | TGAGGCA | | | | |
| | | | | | | | | | | T | | | | |

TABLE 5
Nuclear factors that can be inhibited to increase IL-2 expression or overexpressed to decrease IL-2 expression

| | | | | | | Position | | | | | | | | |
|---|---|---|---|---|---|---|---|---|---|---|---|---|---|---|
| | | | | | | of Base | | sgRNA | SEQ | Target | SEQ | | | Rule |
| gene_ | screen_ | screen_ | Target | Target | Genomic | After Cut | | Target | ID | Context | ID | PAM | Exon | Set 2 |
| id | target | direction | Gene ID | Transcript | Sequence | (1-based) | Strand | Sequence | NO: | Sequence | NO: | Sequence | Number | score |
| TBX21 | IL2 | IL2 high | 30009 | NM_ | NC_ | 47744217 | anti- | CCGGGGC | 761. | GCAGCCG | 837. | GGG | 4 | 0.6246 |
| | | | | 013351.1 | 000017.11 | | sense | TGGTACTT | | GGGCTGG | | | | |
| | | | | | | | | ATGGA | | TACTTATG | | | | |
| | | | | | | | | | | GAGGGAC | | | | |
| | | | | | | | | | | T | | | | |
| TBX21 | IL2 | IL2 high | 30009 | NM_ | NC_00001 | 47733630 | sense | CCTGGGG | 762. | GCAGCCTG | 838. | GGG | 1 | 0.6294 |
| | | | | 013351.1 | 7.11 | | | TCTCCCTA | | GGGTCTCC | | | | |
| | | | | | | | | CCCGG | | CTACCCGG | | | | |
| | | | | | | | | | | GGGGCG | | | | |
| TBX21 | IL2 | IL2 high | 30009 | NM_ | NC_00001 | 47742726 | sense | GCGGTACC | 763. | ACTGGCG | 839. | GGG | 2 | 0.6321 |
| | | | | 013351.1 | 7.11 | | | AGAGCGG | | GTACCAGA | | | | |
| | | | | | | | | CAAGT | | GCGGCAA | | | | |
| | | | | | | | | | | GTGGGTG | | | | |
| | | | | | | | | | | C | | | | |
| TBX21 | IL2 | IL2 high | 30009 | NM_ | NC_ | 47733889 | anti- | TAAACTTG | 764. | TGATTAAA | 840. | TGG | 1 | 0.6344 |
| | | | | 013351.1 | 000017.11 | | sense | GACCACAA | | CTTGGACC | | | | |
| | | | | | | | | CAGG | | ACAACAG | | | | |
| | | | | | | | | | | GTGGTTG | | | | |
| ARID3A | IL2 | IL2 high | 1820 | NM_ | NC_ | 929887 | anti- | AACTCACA | 765. | CCCCAACT | 841. | AGG | 2 | 0.5991 |
| | | | | 005224.2 | 000019.10 | | sense | TGTCCTCG | | CACATGTC | | | | |
| | | | | | | | | TCGG | | CTCGTCGG | | | | |
| | | | | | | | | | | AGGCCA | | | | |
| ARID3A | IL2 | IL2 high | 1820 | NM_ | NC_ | 960105 | sense | ACAGCTCT | 766. | CCTCACAG | 842. | GGG | 4 | 0.7347 |
| | | | | 005224.2 | 000019.10 | | | ACGAACTC | | CTCTACGA | | | | |
| | | | | | | | | GACG | | ACTCGACG | | | | |
| | | | | | | | | | | GGGACC | | | | |
| ARID3A | IL2 | IL2 high | 1820 | NM_ | NC_ | 964923 | sense | ATAGACA | 767. | AGCCATAG | 843. | GGG | 6 | 0.6087 |
| | | | | 005224.2 | 000019.10 | | | GCAACCG | | ACAGCAAC | | | | |
| | | | | | | | | ACGGGA | | CGACGGG | | | | |
| | | | | | | | | | | AGGGCCG | | | | |
| ARID3A | IL2 | IL2 high | 1820 | NM_ | NC_ | 964374 | sense | GCTGTGG | 768. | AGAAGCT | 844. | AGG | 5 | 0.776 |
| | | | | 005224.2 | 000019.10 | | | CGTGAGAT | | GTGGCGT | | | | |
| | | | | | | | | CACCA | | GAGATCAC | | | | |
| | | | | | | | | | | CAAGGGC | | | | |
| | | | | | | | | | | C | | | | |
| GMEB1 | IL2 | IL2 high | 10691 | NM_ | NC_ | 28693028 | sense | AAGAGAG | 769. | CTGGAAG | 845. | TGG | 5 | 0.6435 |
| | | | | 006582.3 | 000001.11 | | | CTATTCGT | | AGAGCTAT | | | | |
| | | | | | | | | CTGGG | | TCGTCTGG | | | | |
| | | | | | | | | | | GTGGGAT | | | | |
| GMEB1 | IL2 | IL2 high | 10691 | NM_ | NC_ | 28690130 | sense | AGCTGGG | 770. | ATGAAGCT | 846. | CGG | 3 | 0.6723 |
| | | | | 006582.3 | 000001.11 | | | TCGGAGA | | GGGTCGG | | | | |
| | | | | | | | | ACAACA | | AGAACAA | | | | |
| | | | | | | | | | | CACGGCA | | | | |
| | | | | | | | | | | G | | | | |
| GMEB1 | IL2 | IL2 high | 10691 | NM_ | NC_ | 28697055 | sense | GCCAGGA | 771. | CAGTGCCA | 847. | TGG | 6 | 0.6728 |
| | | | | 006582.3 | 100000.11 | | | CAGCAGA | | GGACAGC | | | | |
| | | | | | | | | CAAGTG | | AGACAAG | | | | |
| | | | | | | | | | | TGTGGTGC | | | | |
| GMEB1 | IL2 | IL2 high | 10691 | NM_ | NC_ | 28691639 | sense | TGCTTACC | 772. | AAATTGCT | 848. | GGG | 4 | 0.6198 |
| | | | | 006582.3 | 000001.11 | | | CCATAACT | | TACCCCAT | | | | |
| | | | | | | | | TGTG | | AACTTGTG | | | | |
| | | | | | | | | | | GGGAGA | | | | |
| TP53 | IL2 | IL2 high | 7157 | NM_ | NC_ | 7676227 | anti- | CCATTGTT | 773. | TGAACCAT | 849. | GGG | 4 | 0.6899 |
| | | | | 600054.5 | 000017.11 | | sense | CAATATCG | | TGTTCAAT | | | | |
| | | | | | | | | TCCG | | ATCGTCCG | | | | |
| | | | | | | | | | | GGGACA | | | | |
| TP53 | IL2 | IL2 high | 7157 | NM_ | NC_ | 7675058 | sense | GAGCGCT | 774. | CCATGAGC | 850. | TGG | 5 | 0.6542 |
| | | | | 000546.5 | 000017.11 | | | GCTCAGAT | | GCTGCTCA | | | | |
| | | | | | | | | AGCGA | | GATAGCG | | | | |
| | | | | | | | | | | ATGGTGA | | | | |
| TP53 | IL2 | IL2 high | 7157 | NM_ | NC_ | 7676527 | anti- | GATCCACT | 775. | AATGGATC | 851. | AGG | 2 | 0.6108 |
| | | | | 000546.5 | 000017.11 | | sense | CACAGTTT | | CACTCACA | | | | |
| | | | | | | | | CCAT | | GTTTCCAT | | | | |
| | | | | | | | | | | AGGTCT | | | | |
| TP53 | IL2 | IL2 high | 7157 | NM_ | NC_ | 7674864 | sense | GGTGCCCT | 776. | TGGTGGT | 852. | AGG | 6 | 0.6259 |
| | | | | 600054.5 | 000017.11 | | | ATGAGCC | | GCCCTATG | | | | |
| | | | | | | | | GCCTG | | AGCCGCCT | | | | |
| | | | | | | | | | | GAGGTCT | | | | |
| CREB1 | IL2 | IL2 high | 1385 | NM_ | NC_ | 2.08E+08 | anti- | AGCTGTAC | 777. | GGGCAGC | 853. | GGG | 3 | 0.6415 |
| | | | | 004379.3 | 000002.12 | | sense | TAGAGTTA | | TGTACTAG | | | | |
| | | | | | | | | CGGT | | AGTTACGG | | | | |
| | | | | | | | | | | TGGGAGC | | | | |
| CREB1 | IL2 | IL2 high | 1385 | NM_ | NC_ | 2.08E+08 | sense | GGCTAACA | 778. | AGCTGGCT | 854. | GGG | 6 | 0.7053 |
| | | | | 004379.3 | 000002.12 | | | ATGGTACC | | AACAATG | | | | |
| | | | | | | | | GATG | | GTACCGAT | | | | |
| | | | | | | | | | | GGGGTAC | | | | |
| CREB1 | IL2 | IL2 high | 1385 | NM_ | NC_ | 2.08E+08 | anti- | TGGAGTT | 779. | AAATTGGA | 855. | TGG | 5 | 0.6189 |
| | | | | 004379.3 | 000002.12 | | sense | GGCACCGT | | GTTGGCAC | | | | |
| | | | | | | | | TACAG | | CGTTACAG | | | | |
| | | | | | | | | | | TGGTGA | | | | |
| CREB1 | IL2 | IL2 high | 1385 | NM_ | NC_ | 2.08E+08 | anti- | TGTGGAG | 780. | GACTTGTG | 856. | TGG | 3 | 0.6776 |
| | | | | 004379.3 | 000002.12 | | sense | ACTGAATA | | GAGACTG | | | | |
| | | | | | | | | ACTGA | | AATAACTG | | | | |
| | | | | | | | | | | ATGGCTG | | | | |
| KLF13 | IL2 | IL2 high | 51621 | NM_ | NC_ | 31327702 | sense | CCGACCTC | 781. | CGCGCCG | 857. | AGG | 1 | 0.7221 |
| | | | | 015995.3 | 000015.10 | | | GAGTCCCC | | ACCTCGAG | | | | |
| | | | | | | | | GCAG | | TCCCCGCA | | | | |
| | | | | | | | | | | GAGGAAG | | | | |
| KLF13 | IL2 | IL2 high | 51621 | NM_ | NC_ | 31327415 | sense | CGTGGTG | 782. | TCTTCGTG | 858. | CGG | 1 | 0.6798 |
| | | | | 015995.3 | 000015.10 | | | GCGCGGA | | GTGGCGC | | | | |
| | | | | | | | | TCCTAG | | GGATCCTA | | | | |
| | | | | | | | | | | GCGGACC | | | | |
| KLF13 | IL2 | IL2 high | 51621 | NM_ | NC_ | 31327274 | sense | CGTGTCCA | 783. | GCCTCGTG | 859. | CGG | 1 | 0.7 |
| | | | | 015995.3 | 000015.10 | | | TGTCGAGC | | TCCATGTC | | | | |
| | | | | | | | | CGCG | | GAGCCGC | | | | |
| | | | | | | | | | | GCGGTCG | | | | |
| KLF13 | IL2 | IL2 high | 51621 | NM_ | NC_ | 31327767 | anti- | GAGTTCTC | 784. | GTGTGAG | 860. | AGG | 1 | 0.592 |
| | | | | 015995.3 | 000015.10 | | sense | AGGTGCG | | TTCTCAGG | | | | |
| | | | | | | | | CCTTG | | TGCGCCTT | | | | |
| | | | | | | | | | | GAGGTGC | | | | |
| TAF5L | IL2 | IL2 high | 27097 | NM_ | NC_ | 2.3E+08 | anti- | CGGGACA | 785. | GATGCGG | 861. | GGG | 4 | 0.6222 |
| | | | | 014409.3 | 000001.11 | | sense | CGTCTACT | | GACACGTC | | | | |
| | | | | | | | | TGGTG | | TÅCTTGGT | | | | |
| | | | | | | | | | | GGGGCTC | | | | |
| TAF5L | IL2 | IL2 high | 27097 | NM_ | NC_ | 2.3E+08 | sense | GCAGAAC | 786. | TTCTGCAG | 862. | AGG | 4 | 0.6216 |
| | | | | 014409.3 | 000001.11 | | | GAGGCTG | | AACGAGG | | | | |
| | | | | | | | | CCCTAG | | CTGCCCTA | | | | |
| | | | | | | | | | | GAGGTCT | | | | |
| TAF5L | IL2 | IL2 high | 27097 | NM_ | NC_ | 2.3E+08 | sense | GCGGACC | 787. | CACTGCGG | 863. | AGG | 5 | 0.7066 |
| | | | | 014409.3 | 000001.11 | | | AGTGTACA | | ACCAGTGT | | | | |
| | | | | | | | | GCACG | | ACAGCAC | | | | |
| | | | | | | | | | | GAGGTTC | | | | |
| TAF5L | IL2 | IL2 high | 27097 | NM_ | NC_ | 2.3E+08 | anti- | TAAGGTG | 788. | TATGTAAG | 864. | GGG | 4 | 0.6402 |
| | | | | 014409.3 | 000001.11 | | sense | AGGACTTT | | GTGAGGA | | | | |
| | | | | | | | | GCACA | | CTTTGCAC | | | | |
| | | | | | | | | | | AGGGCAG | | | | |
| ZBTB43 | IL2 | IL2 high | 23099 | NM_ | NC_ | 1.27E+08 | anti- | AGTACCTG | 789. | CAGGAGT | 865. | GGG | 3 | 0.6089 |
| | | | | 014007.3 | 000009.12 | | sense | GTCACAAA | | ACCTGGTC | | | | |
| | | | | | | | | AGTA | | ACAAAAGT | | | | |
| | | | | | | | | | | AGGGTGA | | | | |
| ZBTB43 | IL2 | IL2 high | 23099 | NM_ | NC_ | 1.27E+08 | sense | CTTCCTCC | 790. | CAAGCTTC | 866. | TGG | 3 | 0.6684 |
| | | | | 014007.3 | 000009.12 | | | AGATGTG | | CTCCAGAT | | | | |
| | | | | | | | | GCATG | | GTGGCAT | | | | |
| | | | | | | | | | | GTGGTAG | | | | |
| ZBTB43 | IL2 | IL2 high | 23099 | NM_ | NC_ | 1.27E+08 | sense | GCGCCGA | 791. | GACAGCG | 867. | CGG | 3 | 0.6365 |
| | | | | 014007.3 | 000009.12 | | | GTTCCACT | | CCGAGTTC | | | | |
| | | | | | | | | ACACC | | CACTACAC | | | | |
| | | | | | | | | | | CCGGCCC | | | | |
| ZBTB43 | IL2 | IL2 high | 23099 | NM_ | NC_ | 1.27E+08 | anti- | GCTGGGC | 792. | AGTTGCTG | 868. | CGG | 3 | 0.5735 |
| | | | | 014007.3 | 000009.12 | | sense | AGGTATTC | | GGCAGGT | | | | |
| | | | | | | | | GTGCT | | ATTCGTGC | | | | |
| | | | | | | | | | | TCGGTGA | | | | |
| RNF20 | IL2 | IL2 high | 56254 | NM_ | NC_ | 1.02E+08 | sense | ACTTCGGC | 793. | AGAAACTT | 869. | AGG | 9 | 0.6981 |
| | | | | 019592.6 | 000009.12 | | | AAGACTTT | | CGGCAAG | | | | |
| | | | | | | | | GAGG | | ACTTTGAG | | | | |
| | | | | | | | | | | GAGGTCA | | | | |
| RNF20 | IL2 | IL2 high | 56254 | NM_ | NC_ | 1.02E+08 | sense | GCATCGCA | 794. | AAAAGCAT | 870. | AGG | 6 | 0.6768 |
| | | | | 019592.6 | 000009.12 | | | CCATGTCT | | CGCACCAT | | | | |
| | | | | | | | | CAGG | | GTCTCAGG | | | | |
| | | | | | | | | | | AGGTAC | | | | |
| RNF20 | IL2 | IL2 high | 56254 | NM_ | NC_ | 1.02E+08 | anti- | GGAGGGC | 795. | TGCAGGA | 871. | AGG | 13 | 0.6635 |
| | | | | 019592.6 | 000009.12 | | sense | ACTACCAC | | GGGCACT | | | | |
| | | | | | | | | TACGC | | ACCACTAC | | | | |
| | | | | | | | | | | GCAGGCG | | | | |
| | | | | | | | | | | T | | | | |
| RNF20 | IL2 | IL2 high | 56254 | NM_ | NC | 1.02E+08 | anti- | TCGGTTGA | 796. | AGTATCGG | 872. | AGG | 3 | 0.658 |
| | | | | 019592.6 | 000009.12 | | sense | CAATCAAT | | TTGACAAT | | | | |
| | | | | | | | | AGTG | | CAATAGTG | | | | |
| | | | | | | | | | | AGGCAT | | | | |
| TFDP1 | IL2 | IL2 high | 7027 | NM_ | NC_ | 1.14E+08 | sense | ACCGGCA | 797. | AGAGACC | 873. | TGG | 5 | 0.6315 |
| | | | | 007111.4 | 000013.11 | | | GCGTCAAA | | GGCAGCG | | | | |
| | | | | | | | | CACCC | | TCAAACAC | | | | |
| | | | | | | | | | | CCTGGTG | | | | |
| | | | | | | | | | | G | | | | |
| TFDP1 | IL2 | IL2 high | 7027 | NM_ | NC_ | 1.14E+08 | sense | ATGACCAG | 798. | GCTTATGA | 874. | CGG | 7 | 0.6204 |
| | | | | 007111.4 | 000013.11 | | | AAAAACAT | | CCAGAAA | | | | |
| | | | | | | | | AAGA | | AACATAAG | | | | |
| | | | | | | | | | | ACGGCGC | | | | |
| TFDP1 | IL2 | IL2 high | 7027 | NM_ | NC_ | 1.14E+08 | anti- | CCTTCATG | 799. | CAGACCTT | 875. | AGG | 6 | 0.6851 |
| | | | | 007111.4 | 000013.11 | | sense | GAGAAAT | | CATGGAG | | | | |
| | | | | | | | | GCCGT | | AAATGCCG | | | | |
| | | | | | | | | | | TAGGCCC | | | | |
| TFDP1 | IL2 | IL2 high | 7027 | NM_ | NC_ | 1.14E+08 | sense | GGTGCAG | 800. | ACCTGGTG | 876. | CGG | 9 | 0.6402 |
| | | | | 007111.4 | 000013.11 | | | AGAAACC | | CAGAGAA | | | | |
| | | | | | | | | GGCATG | | ACCGGCAT | | | | |
| | | | | | | | | | | GCGGAGC | | | | |
| RFX7 | IL2 | IL2 high | 64864 | NM_ | NC_ | 56098123 | anti- | ACAACGAT | 801. | TGCCACAA | 877. | AGG | 8 | 0.6173 |
| | | | | 022841.5 | 000015.10 | | sense | ACCAATAG | | CGATACCA | | | | |
| | | | | | | | | GTTG | | ATAGGTTG | | | | |
| | | | | | | | | | | AGGAGA | | | | |
| RFX7 | IL2 | IL2 high | 64864 | NM_ | NC_ | 56095516 | anti- | AGCTGAAT | 802. | CCAAAGCT | 878. | GGG | 9 | 0.6523 |
| | | | | 022841.5 | 000015.10 | | sense | CACTGATA | | GAATCACT | | | | |
| | | | | | | | | ACAA | | GATAACAA | | | | |
| | | | | | | | | | | GGGCAG | | | | |
| RFX7 | IL2 | IL2 high | 64864 | NM_ | NC_ | 56142833 | sense | CTGGATTC | 803. | TTTCCTGG | 879. | AGG | 4 | 0.6867 |
| | | | | 022841.5 | 000015.10 | | | GGAATACC | | ATTCGGAA | | | | |
| | | | | | | | | CTAG | | TACCCTAG | | | | |
| | | | | | | | | | | AGGAAC | | | | |
| RFX7 | IL2 | IL2 high | 64864 | NM_ | NC_ | 56101446 | anti- | GAAGCGG | 804. | CAAGGAA | 880. | CGG | 7 | 0.6903 |
| | | | | 022841.5 | 000015.10 | | sense | GCTAATTC | | GCGGGCT | | | | |
| | | | | | | | | CAAGA | | AATTCCAA | | | | |
| | | | | | | | | | | GACGGTG | | | | |
| | | | | | | | | | | T | | | | |
| RUNX3 | IL2 | IL2 high | 864 | NM_ | NC_ | 24927597 | anti- | CACTGCGG | 805. | CGCCCACT | 881. | AGG | 2 | 0.6012 |
| | | | | 004350.2 | 000001.11 | | sense | CCCACGAA | | GCGGCCC | | | | |
| | | | | | | | | GCGA | | ACGAAGC | | | | |
| | | | | | | | | | | GAAGGTC | | | | |
| | | | | | | | | | | G | | | | |
| RUNX3 | IL2 | IL2 high | 864 | NM_ | NC_ | 24902559 | sense | CCCCAGGA | 806. | CAGACCCC | 882. | GGG | 5 | 0.642 |
| | | | | 004350.2 | 000001.11 | | | TGCATTAT | | AGGATGC | | | | |
| | | | | | | | | CCCG | | ATTATCCC | | | | |
| | | | | | | | | | | GGGGCCA | | | | |
| RUNX3 | IL2 | IL2 high | 864 | NM_ | NC_ | 24907265 | anti- | CCGTGCCG | 807 | CCCTCCGT | 883. | GGG | 4 | 0.6118 |
| | | | | 004350.2 | 000001.11 | | sense | TACCTTGG | | GCCGTACC | | | | |
| | | | | | | | | ATTG | | TTGGATTG | | | | |
| | | | | | | | | | | GGGTCT | | | | |
| RUNX3 | IL2 | IL2 high | 864 | NM_ | NC_ | 24919297 | anti- | TCGGTGGT | 808. | TGGCTCG | 884. | GGG | 3 | 0.5939 |
| | | | | 004350.2 | 000001.11 | | sense | AGGTCGCC | | GTGGTAG | | | | |
| | | | | | | | | ACTT | | GTCGCCAC | | | | |
| | | | | | | | | | | TTGGGTG | | | | |
| | | | | | | | | | | G | | | | |
| CXXC1 | IL2 | IL2 high | 30827 | NM_ | NC_ | 50285885 | sense | AAACGGTC | 809 | GATCAAAC | 885. | TGG | 5 | 0.6028 |
| | | | | 014593.3 | 000018.10 | | | AGCCCGCA | | GGTCAGCC | | | | |
| | | | | | | | | TGTG | | CGCATGTG | | | | |
| | | | | | | | | | | TGGTGA | | | | |
| CXXC1 | IL2 | IL2 high | 30827 | NM_ | NC_ | 50284548 | sense | AGAAGGA | 810. | CAACAGA | 886. | CGG | 9 | 0.6935 |
| | | | | 014593.3 | 000018.10 | | | GGAGCGA | | AGGAGGA | | | | |
| | | | | | | | | TACAAG | | GCGATACA | | | | |
| | | | | | | | | | | AGCGGCA | | | | |
| | | | | | | | | | | T | | | | |
| CXXC1 | IL2 | IL2 high | 30827 | NM_ | NC_ | 50285170 | anti- | ATGCGCCC | 811. | ACGGATG | 887. | TGG | 7 | 0.5917 |
| | | | | 014593.3 | 000018.10 | | sense | TAACTTCT | | CGCCCTAA | | | | |
| | | | | | | | | GTGA | | CTTCTGTG | | | | |
| | | | | | | | | | | ATGGCTG | | | | |
| CXXC1 | IL2 | IL2 high | 30827 | NM_ | NC_ | 50286232 | sense | CCAAGCTA | 812. | GACCCCAA | 888. | CGG | 4 | 0.6313 |
| | | | | 014593.3 | 000018.10 | | | GAGATTCG | | GCTAGAG | | | | |
| | | | | | | | | CTAT | | ATTCGCTA | | | | |
| | | | | | | | | | | TCGGCAC | | | | |
| IKZF3 | IL2 | IL2 high | 22806 | NM_ | NC_ | 39792732 | sense | AAGATGA | 813. | TGGAAAG | 889. | TGG | 4 | 0.7182 |
| | | | | 012481.4 | 000017.11 | | | ACTGCGAT | | ATGAACTG | | | | |
| | | | | | | | | GTGTG | | CGATGTGT | | | | |
| | | | | | | | | | | GTGGATT | | | | |
| IKZF3 | IL2 | IL2 high | 22806 | NM_ | NC_ | 39788318 | sense | CAAGCAG | 814. | GTTACAAG | 890. | AGG | 6 | 0.6635 |
| | | | | 012481.4 | 000017.11 | | | AGAAGTTC | | CAGAGAA | | | | |
| | | | | | | | | CCTTG | | GTTCCCTT | | | | |
| | | | | | | | | | | GAGGAGC | | | | |
| IKZF3 | IL2 | IL2 high | 22806 | NM_ | NC_ | 39766413 | sense | GCTCATAC | 815. | GTGAGCTC | 891 | TGG | 8 | 0.6101 |
| | | | | 012481.4 | 000017.11 | | | AGACCCGC | | ATACAGAC | | | | |
| | | | | | | | | ATGA | | CCGCATGA | | | | |
| | | | | | | | | | | TGGACC | | | | |
| IKZF3 | IL2 | IL2 high | 22806 | NM_ | NC_ | 39777693 | sense | GGACAGA | 816. | TACTGGAC | 892. | TGG | 7 | 0.7336 |
| | | | | 012481.4 | 000017.11 | | | TTAGCAAG | | AGATTAGC | | | | |
| | | | | | | | | CAATG | | AAGCAAT | | | | |
| | | | | | | | | | | GTGGCAA | | | | |
| PRDM1 | IL2 | IL2 high | 639 | NM_ | NC_ | 1.06E+08 | sense | AGGATGC | 817. | ATGGAGG | 893. | TGG | 2 | 0.7731 |
| | | | | 001198.3 | 000006.12 | | | GGATATG | | ATGCGGAT | | | | |
| | | | | | | | | ACTCTG | | ATGACTCT | | | | |
| | | | | | | | | | | GTGGACA | | | | |
| PRDM1 | IL2 | IL2 high | 639 | NM_ | NC_ | 1.06E+08 | anti- | GGACGCG | 818. | CGTAGGA | 894. | AGG | 5 | 0.6665 |
| | | | | 001198.3 | 000006.12 | | sense | TTCAAGTA | | CGCGTTCA | | | | |
| | | | | | | | | AGCGT | | AGTAAGC | | | | |
| | | | | | | | | | | GTAGGAG | | | | |
| | | | | | | | | | | T | | | | |
| PRDM1 | IL2 | IL2 high | 639 | NM_ | NC_ | 1.06E+08 | anti- | GGGGAGC | 819. | AATGGGG | 895. | GGG | 5 | 0.6871 |
| | | | | 001198.3 | 000006.12 | | sense | GAGTGAT | | GAGCGAG | | | | |
| | | | | | | | | GTACGT | | TGATGTAC | | | | |
| | | | | | | | | | | GTGGGTCT | | | | |
| PRDM1 | IL2 | IL2 high | 639 | NM_ | NC_ | 1.06E+08 | sense | TTTGGACA | 820. | CTCTTTTG | 896. | AGG | 4 | 0.6708 |
| | | | | 001198.3 | 000006.12 | | | GATCTATT | | GACAGATC | | | | |
| | | | | | | | | CCAG | | TATTCCAG | | | | |
| | | | | | | | | | | AGGGGA | | | | |
| IKZF1 | IL2 | IL2 high | 10320 | NM_ | NC 00000 | 50376659 | sense | GAAAATG | 821. | GAGAGAA | 897. | GGG | 4 | 0.66 |
| | | | | 006060.5 | 7.14 | | | AATGGCTC | | AATGAATG | | | | |
| | | | | | | | | CCACA | | GCTCCCAC | | | | |
| | | | | | | | | | | AGGGACC | | | | |
| IKZF1 | IL2 | IL2 high | 10320 | NM_ | NC_ | 50399996 | anti- | GATGGCTT | 822. | TGTTGATG | 898. | GGG | 8 | 0.7379 |
| | | | | 006060.5 | 000007.14 | | sense | GGTCCATC | | GCTTGGTC | | | | |
| | | | | | | | | ACGT | | CATCACGT | | | | |
| | | | | | | | | | | GGGACT | | | | |
| IKZF1 | IL2 | IL2 high | 10320 | NM_ | NC_ | 50382586 | sense | GGGGCCT | 823. | GTGCGGG | 899. | GGG | 5 | 0.6196 |
| | | | | 006060.5 | 000007.14 | | | CATTCACC | | GCCTCATT | | | | |
| | | | | | | | | CAGAA | | CACCCAGA | | | | |
| | | | | | | | | | | AGGGCAA | | | | |
| IKZF1 | IL2 | IL2 high | 10320 | NM _ | NC_ | 50327753 | sense | TCCAAGAG | 824. | AAGCTCCA | 900. | GGG | 3 | 0.617 |
| | | | | 006060.5 | 000007.14 | | | TGACAGA | | AGAGTGA | | | | |
| | | | | | | | | GTCGT | | CAGAGTC | | | | |
| | | | | | | | | | | GTGGGTA | | | | |
| | | | | | | | | | | A | | | | |
| FOXP1 | IL2 | IL2 high | 27086 | NM_ | NC_ | 71041428 | anti- | AGAGGAG | 825. | GTGCAGA | 901. | TGG | 11 | 0.6926 |
| | | | | 032682.5 | 000003.12 | | sense | GAGACAC | | GGAGGAG | | | | |
| | | | | | | | | ATGTCG | | ACACATGT | | | | |
| | | | | | | | | | | CGTGGTCA | | | | |
| FOXP1 | IL2 | IL2 high | 27086 | NM_ | NC_ | 71015617 | anti- | CATACACC | 826. | CTTGCATA | 902. | AGG | 12 | 0.6547 |
| | | | | 032682.5 | 000003.12 | | sense | ATGTCCAT | | CACCATGT | | | | |
| | | | | | | | | AGAG | | CCATAGAG | | | | |
| | | | | | | | | | | AGGATG | | | | |
| FOXP1 | IL2 | IL2 high | 27086 | NM_ | NC_ | 71046982 | sense | GCCTTCTG | 827. | CAAGGCCT | 903. | GGG | 10 | 0.5683 |
| | | | | 032682.5 | 000003.12 | | | ACAATTCA | | TCTGACAA | | | | |
| | | | | | | | | GCCC | | TTCAGCCC | | | | |
| | | | | | | | | | | GGGCAG | | | | |
| FOXP1 | IL2 | IL2 high | 27086 | NM_ | NC_ | 70988031 | anti- | GTTCTGTA | 828 | TTGGGTTC | 904. | AGG | 14 | 0.5925 |
| | | | | 032682.5 | 000003.12 | | sense | GACTTCAC | | TGTAGACT | | | | |
| | | | | | | | | ATGC | | TCACATGC | | | | |
| | | | | | | | | | | AGGTGG | | | | |
| PTEN | IL2 | IL2 high | 5728 | NM_ | NC_ | 87961027 | sense | AGAGCGT | 829. | GTATAGA | 905. | AGG | 8 | 0.7179 |
| | | | | 001304718. | 000010.11 | | | GCAGATA | | GCGTGCA | | | | |
| | | | | 1 | | | | ATGACA | | GATAATGA | | | | |
| | | | | | | | | | | CAAGGAA | | | | |
| | | | | | | | | | | T | | | | |
| PTEN | IL2 | IL2 high | 5728 | NM_ | NC_ | 87957861 | anti- | AGCTGGC | 830. | CTTTAGCT | 906. | AGG | 7 | 0.6493 |
| | | | | 001304718. | 000010.11 | | sense | AGACCACA | | GGCAGAC | | | | |
| | | | | 1 | | | | AACTG | | CACAAACT | | | | |
| | | | | | | | | | | GAGGATC | | | | |
| PTEN | IL2 | IL2 high | 5728 | NM_ | NC_ | 87960940 | sense | ATTCTTCA | 831. | ATACATTC | 907. | AGG | 8 | 0.6697 |
| | | | | 001304718. | 000010.11 | | | TACCAGGA | | TTCATACC | | | | |
| | | | | 1 | | | | CCAG | | AGGACCA | | | | |
| | | | | | | | | | | GAGGAAA | | | | |
| PTEN | IL2 | IL2 high | 5728 | NM_ | NC_ | 87957915 | sense | CCAATTCA | 832. | TCCTCCAA | 908. | CGG | 7 | 0.6752 |
| | | | | 001304718. | 000010.11 | | | GGACCCAC | | TTCAGGAC | | | | |
| | | | | 1 | | | | ACGA | | CCACACGA | | | | |
| | | | | | | | | | | CGGGAA | | | | |
| MED12 | IL2 | IL2 high | 9968 | NM_ | NC_ | 71130165 | sense | ACATCGAC | 833. | ATCCACAT | 909. | AGG | 28 | 0.6457 |
| | | | | 005120.2 | 000023.11 | | | TGCTGGAC | | CGACTGCT | | | | |
| | | | | | | | | AATG | | GGACAAT | | | | |
| | | | | | | | | | | GAGGATG | | | | |
| MED12 | IL2 | IL2 high | 9968 | NM_ | NC_ | 71122231 | anti- | CAGTGAGT | 834. | CAGTCAGT | 910. | AGG | 8 | 0.688 |
| | | | | 005120.2 | 000023.11 | | sense | AGTGCCAA | | GAGTAGT | | | | |
| | | | | | | | | ACCA | | GCCAAACC | | | | |
| | | | | | | | | | | AAGGCAC | | | | |
| MED12 | IL2 | IL2 high | 9968 | NM_ | NC_ | 71125111 | anti- | GTGGCGT | 835. | ATGGGTG | 911. | TGG | 15 | 0.6661 |
| | | | | 005120.2 | 000023.11 | | sense | ACTGCACG | | GCGTACTG | | | | |
| | | | | | | | | TGTCG | | CACGTGTC | | | | |
| | | | | | | | | | | GTGGCTG | | | | |
| MED12 | IL2 | IL2 high | 9968 | NM_ | NC_ | 71126138 | sense | TTCACATT | 836. | ACCTTTCA | 912. | AGG | 18 | 0.6594 |
| | | | | 005120.2 | 000023.11 | | | ATGACCAA | | CATTATGA | | | | |
| | | | | | | | | CACC | | CCAACACC | | | | |
| | | | | | | | | | | AGGTCA | | | | |

TABLE 6
Nuclear factors that can be inhibited to decrease IL-2 expression or overexpressed to increase IL-2 expression

| | | | | | | Position | | | | | | | | |
|---|---|---|---|---|---|---|---|---|---|---|---|---|---|---|
| | | | | | | of Base | | sgRNA | SEQ | Target | SEQ | | | Rule |
| gene_ | screen_ | screen_ | Target | Target | Genomic | After Cut | | Target | ID | Context | ID | PAM | Exon | Set 2 |
| id | target | direction | Gene ID | Transcript | Sequence | (1-based) | Strand | Sequence | NO: | Sequence | NO: | Sequence | Number | score |
| SMARCB1 | IL2 | IL2 low | 6598 | NM_ | NC_ | 23791773 | anti- | GAGAACCT | 913. | TACAGAG | 1101. | AGG | 2 | 0.731 |
| | | | | 003073.3 | 000022.11 | | sense | CGGAACAT | | AACCTCGG | | | | |
| | | | | | | | | ACGG | | AACATACG | | | | |
| | | | | | | | | | | GAGGTAG | | | | |
| SMARCB1 | IL2 | IL2 low | 6598 | NM_ | NC_ | 23816887 | sense | GCAGATC | 914. | GACAGCA | 1102. | CGG | 6 | 0.6894 |
| | | | | 003073.3 | 000022.11 | | | GAGTCCTA | | GATCGAGT | | | | |
| | | | | | | | | CCCCA | | CCTACCCC | | | | |
| | | | | | | | | | | ACGGACA | | | | |
| SMARCB1 | IL2 | IL2 low | 6598 | NM_ | NC_ | 23801049 | anti- | TCTTCTTG | 915. | GTTCTCTT | 1103. | CGG | 4 | 0.6806 |
| | | | | 003073.3 | 000022.11 | | sense | TCTCGGCC | | CTTGTCTC | | | | |
| | | | | | | | | CATG | | GGCCCATG | | | | |
| | | | | | | | | | | CGGTTC | | | | |
| SMARCB1 | IL2 | IL2 low | 6598 | NM_ | NC_ | 23803342 | sense | TGAGAAC | 916. | TCCATGAG | 1104. | AGG | 5 | 0.6804 |
| | | | | 003073.3 | 000022.11 | | | GCATCTCA | | AACGCATC | | | | |
| | | | | | | | | GCCCG | | TCAGCCCG | | | | |
| | | | | | | | | | | AGGTGC | | | | |
| GTF2B | IL2 | IL2 low | 2959 | NM_ | NC_ | 88864042 | sense | ACAAAAG | 917. | AGCAACA | 1105. | TGG | 3 | 0.6003 |
| | | | | 001514.5 | 000001.11 | | | ATCCATCT | | AAAGATCC | | | | |
| | | | | | | | | CGAGT | | ATCTCGAG | | | | |
| | | | | | | | | | | TTGGAGA | | | | |
| GTF2B | IL2 | IL2 low | 2959 | NM_ | NC_ | 88857407 | anti- | AGTTGTAA | 918. | CCCCAGTT | 1106. | TGG | 6 | 0.6617 |
| | | | | 001514.5 | 000001.11 | | sense | TCAAATCC | | GTAATCAA | | | | |
| | | | | | | | | ACAC | | ATCCACAC | | | | |
| | | | | | | | | | | TGGTTT | | | | |
| GTF2B | IL2 | IL2 low | 2959 | NM_ | NC_ | 88887278 | sense | GATATGAT | 919. | CGGTGATA | 1107. | TGG | 2 | 0.627 |
| | | | | 001514.5 | 000001.11 | | | CTGTCCTG | | TGATCTGT | | | | |
| | | | | | | | | AATG | | CCTGAATG | | | | |
| | | | | | | | | | | TGGCTT | | | | |
| GTF2B | IL2 | IL2 low | 2959 | NM_ | NC_ | 88863986 | sense | TTTGTCTA | 920. | GAGATTTG | 1108. | AGG | 3 | 0.609 |
| | | | | 001514.5 | 000001.11 | | | CCATGATT | | TCTACCAT | | | | |
| | | | | | | | | GGCA | | GATTGGCA | | | | |
| | | | | | | | | | | AGGTAA | | | | |
| MTF1 | IL2 | IL2 low | 4520 | NM_ | NC_ | 37835092 | sense | AATGCACT | 921. | ATACAATG | 1109. | TGG | 6 | 0.6619 |
| | | | | 005955.2 | 000001.11 | | | TCCACAAC | | CACTTCCA | | | | |
| | | | | | | | | ACAA | | CAACACAA | | | | |
| | | | | | | | | | | TGGATC | | | | |
| MTF1 | IL2 | IL2 low | 4520 | NM_ | NC_ | 37857385 | anti- | AATGTGCT | 922. | AGATAATG | 1110. | GGG | 2 | 0.6361 |
| | | | | 005955.2 | 000001.11 | | sense | GCACATAA | | TGCTGCAC | | | | |
| | | | | | | | | CCCT | | ATAACCCT | | | | |
| | | | | | | | | | | GGGACA | | | | |
| MTF1 | IL2 | IL2 low | 4520 | NM_ | NC_ | 37839984 | sense | CCACGTGC | 923. | GGATCCAC | 1111. | AGG | 3 | 0.7358 |
| | | | | 005955.2 | 000001.11 | | | GAGTGCA | | GTGCGAG | | | | |
| | | | | | | | | CACGA | | TGCACACG | | | | |
| | | | | | | | | | | AAGGAGA | | | | |
| MTF1 | IL2 | IL2 low | 4520 | NM_ | NC_ | 37838644 | sense | GCACATTC | 924. | GGAAGCA | 1112. | GGG | 4 | 0.7005 |
| | | | | 005955.2 | 000001.11 | | | GAACTCAT | | CATTCGAA | | | | |
| | | | | | | | | ACAG | | CTCATACA | | | | |
| | | | | | | | | | | GGGGAAA | | | | |
| IL2 | IL2 | IL2 low | 3558 | NM_ | NC_ | 1.22E+08 | anti- | AAACTTAA | 925. | TGTAAAAC | 1113. | TGG | 2 | 0.6039 |
| | | | | 000586.3 | 000004.12 | | sense | ATGTGAGC | | TTAAATGT | | | | |
| | | | | | | | | ATCC | | GAGCATCC | | | | |
| | | | | | | | | | | TGGTGA | | | | |
| IL2 | IL2 | IL2 low | 3558 | NM_ | NC_ | 1.22E+08 | sense | ACAACTGG | 926. | AGCTACAA | 1114. | TGG | 1 | 0.531 |
| | | | | 000586.3 | 000004.12 | | | AGCATTTA | | CTGGAGC | | | | |
| | | | | | | | | CTGC | | ATTTACTG | | | | |
| | | | | | | | | | | CTGGATT | | | | |
| IL2 | IL2 | IL2 low | 3558 | NM_ | NC_ | 1.22E+08 | sense | AGAAGAA | 927. | GTCTAGAA | 1115. | TGG | 3 | 0.4893 |
| | | | | 000586.3 | 000004.12 | | | GAACTCAA | | GAAGAAC | | | | |
| | | | | | | | | ACCTC | | TCAAACCT | | | | |
| | | | | | | | | | | CTGGAGG | | | | |
| IL2 | IL2 | IL2 low | 3558 | NM_ | NC_ | 1.22E+08 | anti- | TTCTTTGT | 928. | TGTTTTCT | 1116. | AGG | 1 | 0.5452 |
| | | | | 000586.3 | 000004.12 | | sense | AGAACTTG | | TTGTAGAA | | | | |
| | | | | | | | | AAGT | | CTTGAAGT | | | | |
| | | | | | | | | | | AGGTGC | | | | |
| NFKB1 | IL2 | IL2 low | 4790 | NM_ | NC_ | 1.03E+08 | anti- | AAGTAGG | 929. | CCATAAGT | 1117. | GGG | 13 | 0.608 |
| | | | | 003998.3 | 000004.12 | | sense | AAATCCAT | | AGGAAAT | | | | |
| | | | | | | | | AGTGT | | CCATAGTG | | | | |
| | | | | | | | | | | TGGGAAG | | | | |
| NFKB1 | IL2 | IL2 low | 4790 | NM_ | NC_ | 1.03E+08 | anti- | GGCACCA | 930. | TAGAGGC | 1118. | GGG | 5 | 0.5997 |
| | | | | 003998.3 | 000004.12 | | sense | GGTAGTCC | | ACCAGGTA | | | | |
| | | | | | | | | ACCAT | | GTCCACCA | | | | |
| | | | | | | | | | | TGGGATG | | | | |
| NFKB1 | IL2 | IL2 low | 4790 | NM_ | NC_ | 1.03E+08 | anti- | TAGATGGC | 931. | GTCATAGA | 1119. | GGG | 8 | 0.6614 |
| | | | | 003998.3 | 000004.12 | | sense | GTCTGATA | | TGGCGTCT | | | | |
| | | | | | | | | CCAC | | GATACCAC | | | | |
| | | | | | | | | | | GGGTTC | | | | |
| NFKB1 | IL2 | IL2 low | 4790 | NM_ | NC_ | 1.03E+08 | anti- | TTGTCTAT | 932. | TTACTTGT | 1120. | GGG | 10 | 0.6663 |
| | | | | 003998.3 | 000004.12 | | sense | GAACATCT | | CTATGAAC | | | | |
| | | | | | | | | GTGG | | ATCTGTGG | | | | |
| | | | | | | | | | | GGGAAA | | | | |
| DNMT1 | IL2 | IL2 low | 1786 | NM_ | NC_ | 10146475 | sense | GAGGCAA | 933. | AGATGAG | 1121. | GGG | 27 | 0.7285 |
| | | | | 001379.2 | 000019.10 | | | AAAGAAA | | GCAAAAA | | | | |
| | | | | | | | | TCCCCA | | GAAATCCC | | | | |
| | | | | | | | | | | CAGGGTCC | | | | |
| DNMT1 | IL2 | IL2 low | 1786 | NM_ | NC_ | 10162681 | sense | GATTTCTG | 934. | CACAGATT | 1122. | AGG | 12 | 0.7345 |
| | | | | 001379.2 | 900001.10 | | | ATGAAAA | | TCTGATGA | | | | |
| | | | | | | | | AGACG | | AAAAGAC | | | | |
| | | | | | | | | | | GAGGATG | | | | |
| DNMT1 | IL2 | IL2 low | 1786 | NM_ | NC_ | 10142150 | sense | GCTCTACT | 935. | ACCTGCTC | 1123. | AGG | 29 | 0.6656 |
| | | | | 001379.2 | 000019.10 | | | GGAGCGA | | TACTGGAG | | | | |
| | | | | | | | | CGAGG | | CGACGAG | | | | |
| | | | | | | | | | | GAGGCCG | | | | |
| DNMT1 | IL2 | IL2 low | 1786 | NM_ | NC_ | 10151459 | sense | TCACCCAA | 936. | GCCGTCAC | 1124. | GGG | 23 | 0.7089 |
| | | | | 001379.2 | 000019.10 | | | AAAAATGC | | CCAAAAAA | | | | |
| | | | | | | | | ACCA | | ATGCACCA | | | | |
| | | | | | | | | | | GGGGAA | | | | |
| RELA | IL2 | IL2 low | 5970 | NM_ | NC_ | 65659757 | sense | ACTACGAC | 937. | GGGGACT | 1125. | CGG | 6 | 0.7259 |
| | | | | 001243984. | 000011.10 | | | CTGAATGC | | ACGACCTG | | | | |
| | | | | 1 | | | | TGTG | | AATGCTGT | | | | |
| | | | | | | | | | | GCGGCTC | | | | |
| RELA | IL2 | IL2 low | 5970 | NM_ | NC_ | 65662009 | sense | GCTTCCGC | 938. | ATGCGCTT | 1126. | GGG | 3 | 0.7137 |
| | | | | 001243984. | 000011.10 | | | TACAAGTG | | CCGCTACA | | | | |
| | | | | 1 | | | | CGAG | | AGTGCGA | | | | |
| | | | | | | | | | | GGGGCGC | | | | |
| RELA | IL2 | IL2 low | 5970 | NM_ | NC_ | 65658759 | anti- | GGAAGAT | 939. | AGTAGGA | 1127. | AGG | 7 | 0.7554 |
| | | | | 001243984. | 000011.10 | | sense | CTCATCCC | | AGATCTCA | | | | |
| | | | | 1 | | | | CACCG | | TCCCCACC | | | | |
| | | | | | | | | | | GAGGCAG | | | | |
| RELA | IL2 | IL2 low | 5970 | NM_ | NC_ | 65661818 | sense | TCAATGGC | 940. | CAGATCAA | 1128. | GGG | 4 | 0.7164 |
| | | | | 001243984. | 000011.10 | | | TACACAGG | | TGGCTACA | | | | |
| | | | | 1 | | | | ACCA | | CAGGACC | | | | |
| | | | | | | | | | | AGGGACA | | | | |
| MAF | IL2 | IL2 low | 4094 | NM_ | NC_ | 79599622 | sense | GAAGACT | 941. | CCTGGAA | 1129. | CGG | 1 | 0.5494 |
| | | | | 005360.4 | 000016.10 | | | ACTACTGG | | GACTACTA | | | | |
| | | | | | | | | ATGAC | | CTGGATGA | | | | |
| | | | | | | | | | | CCGGCTA | | | | |
| MAF | IL2 | IL2 low | 4094 | NM_ | NC_ | 79599416 | anti- | GATCACG | 942. | CGGCGATC | 1130. | CGG | 1 | 0.7152 |
| | | | | 005360.4 | 000016.10 | | sense | GCGGACA | | ACGGCGG | | | | |
| | | | | | | | | CCACGG | | ACACCACG | | | | |
| | | | | | | | | | | GCGGCGG | | | | |
| MAF | IL2 | IL2 low | 4094 | NM_ | NC_ | 79599725 | anti- | GCTGCACG | 943. | CCGAGCTG | 1131. | GGG | 1 | 0.7019 |
| | | | | 005360.4 | 000016.10 | | sense | GCGTGCTC | | CACGGCGT | | | | |
| | | | | | | | | ATGG | | GCTCATGG | | | | |
| | | | | | | | | | | GGGTGG | | | | |
| MAF | IL2 | IL2 low | 4094 | NM_ | NC_ | 79599797 | sense | TGAAGTG | 944. | AGTTTGAA | 1132. | TGG | 1 | 0.6793 |
| | | | | 005360.4 | 000016.10 | | | AAAAAGG | | GTGAAAA | | | | |
| | | | | | | | | AACCGG | | AGGAACC | | | | |
| | | | | | | | | | | GGTGGAG | | | | |
| | | | | | | | | | | A | | | | |
| XBP1 | IL2 | IL2 low | 7494 | NM_ | NC_ | 28797122 | sense | AGGAGTT | 945. | AACCAGG | 1133. | GGG | 3 | 0.5997 |
| | | | | 005080.3 | 000022.11 | | | AAGACAG | | AGTTAAGA | | | | |
| | | | | | | | | CGCTTG | | CAGCGCTT | | | | |
| | | | | | | | | | | GGGGATG | | | | |
| XBP1 | IL2 | IL2 low | 7494 | NM_ | NC_ | 28800395 | sense | CATGGTGC | 946. | CGCTCATG | 1134. | GGG | 1 | 0.6268 |
| | | | | 005080.3 | 000022.11 | | | CAGCCCAG | | GTGCCAGC | | | | |
| | | | | | | | | AGAG | | CCAGAGA | | | | |
| | | | | | | | | | | GGGGCCA | | | | |
| XBP1 | IL2 | IL2 low | 7494 | NM_ | NC_ | 28796184 | sense | CCTCCCAG | 947. | CTTTCCTC | 1135. | AGG | 4 | 0.6692 |
| | | | | 005080.3 | 000022.11 | | | GGGAATG | | CCAGGGG | | | | |
| | | | | | | | | AAGTG | | AATGAAGT | | | | |
| | | | | | | | | | | GAGGCCA | | | | |
| XBP1 | IL2 | IL2 low | 7494 | NM_ | NC_ | 28800465 | sense | CTAAAGTT | 948. | ACCCCTAA | 1136. | GGG | 1 | 0.5959 |
| | | | | 005080.3 | 000022.11 | | | CTGCTTCT | | AGTTCTGC | | | | |
| | | | | | | | | GTCG | | TTCTGTCG | | | | |
| | | | | | | | | | | GGGCAG | | | | |
| BCL11B | IL2 | IL2 low | 64919 | NM_ | NC_ | 99175544 | sense | AGCAAGTC | 949. | CAAGAGC | 1137. | CGG | 4 | 0.6246 |
| | | | | 200128237. | 000014.9 | | | GTGCGAG | | AAGTCGTG | | | | |
| | | | | 1 | | | | TTCTG | | CGAGTTCT | | | | |
| | | | | | | | | | | GCGGCAA | | | | |
| BCL11B | IL2 | IL2 low | 64919 | NM_ | NC_ | 99176056 | sense | CCAGCAGC | 950. | CCGGCCA | 1138. | CGG | 4 | 0.6152 |
| | | | | 001282237. | 000014.9 | | | TCGCTCAC | | GCAGCTCG | | | | |
| | | | | 1 | | | | GCCG | | CTCACGCC | | | | |
| | | | | | | | | | | GCGGCTC | | | | |
| BCL11B | IL2 | IL2 low | 64919 | NM_ | NC_ | 99175744 | sense | CCGCCATG | 951. | TCGCCCGC | 1139. | CGG | 4 | 0.7423 |
| | | | | 001282237. | 000014.9 | | | GACTTCTC | | CATGGACT | | | | |
| | | | | 1 | | | | GCGG | | TCTCGCGG | | | | |
| | | | | | | | | | | CGGCTC | | | | |
| BCL11B | IL2 | IL2 low | 64919 | NM_ | NC_ | 99231381 | sense | TCAGGGT | 952. | GGACTCA | 1140. | AGG | 3 | 0.6195 |
| | | | | 001282237. | 000014.9 | | | GAGGGTC | | GGGTGAG | | | | |
| | | | | 1 | | | | AGACGG | | GGTCAGA | | | | |
| | | | | | | | | | | CGGAGGC | | | | |
| | | | | | | | | | | TC | | | | |
| ETS1 | IL2 | IL2 low | 2113 | NM_ | NC_ | 1.28E+08 | anti- | CTTACTAA | 953. | TGAACTTA | 1141. | AGG | 4 | 0.6808 |
| | | | | 005238.3 | 000011.10 | | sense | TGAAGTAA | | CTAATGAA | | | | |
| | | | | | | | | TCCG | | GTAATCCG | | | | |
| | | | | | | | | | | AGGTAT | | | | |
| ETS1 | IL2 | IL2 low | 2113 | NM_ | NC_ | 1.28E+08 | anti- | GAGAAAG | 954. | GCTCGAG | 1142. | GGG | 3 | 0.6487 |
| | | | | 005238.3 | 000011.10 | | sense | CAGTCTTT | | AAAGCAG | | | | |
| | | | | | | | | ACCCA | | TCTTTACC | | | | |
| | | | | | | | | | | CAGGGCG | | | | |
| | | | | | | | | | | C | | | | |
| ETS1 | IL2 | IL2 low | 2113 | NM_ | NC_ | 1.28E+08 | anti- | GGTCTCG | 955. | AGAGGGT | 1143. | GGG | 5 | 0.7649 |
| | | | | 005238.3 | 000011.10 | | sense | GAGAATG | | CTCGGAG | | | | |
| | | | | | | | | ACCGAG | | AATGACCG | | | | |
| | | | | | | | | | | AGGGGTA | | | | |
| | | | | | | | | | | G | | | | |
| ETS1 | IL2 | IL2 low | 2113 | NM_ | NC_ | 1.28E+08 | sense | TGCATGG | 956. | CATGTGCA | 1144. | TGG | 5 | 0.6503 |
| | | | | 005238.3 | 000011.10 | | | GGAGGAC | | TGGGGAG | | | | |
| | | | | | | | | CAGTCG | | GACCAGTC | | | | |
| | | | | | | | | | | GTGGTAG | | | | |
| ZBTB7B | IL2 | IL2 low | 51043 | NM_ | NC_ | 1.55E+08 | sense | AGCAAACC | 957. | CCAGAGC | 1145. | AGG | 2 | 0.6906 |
| | | | | 001256455. | 000001.11 | | | ACCTAGTC | | AAACCACC | | | | |
| | | | | 1 | | | | CCTG | | TAGTCCCT | | | | |
| | | | | | | | | | | GAGGTGC | | | | |
| ZBTB7B | IL2 | IL2 low | 51043 | NM_ | NC_ | 1.55E+08 | sense | CAGAGCTA | 958. | TCCCCAGA | 1146. | GGG | 2 | 0.6187 |
| | | | | 001256455. | 000001.11 | | | CGAACCCT | | GCTACGAA | | | | |
| | | | | 1 | | | | ATGA | | CCCTATGA | | | | |
| | | | | | | | | | | GGGTGA | | | | |
| ZBTB7B | IL2 | IL2 low | 51043 | NM_ | NC_ | 1.55E+08 | anti- | TCCGGATG | 959. | TGCGTCCG | 1147. | AGG | 2 | 0.6762 |
| | | | | 001256455. | 000001.11 | | sense | GTGAGGT | | GATGGTG | | | | |
| | | | | 1 | | | | CACAT | | AGGTCACA | | | | |
| | | | | | | | | | | TAGGTGG | | | | |
| ZBTB7B | IL2 | IL2 low | 51043 | NM_ | NC_ | 1.55E+08 | anti- | TGTATAGG | 960. | TGGCTGTA | 1148. | GGG | 2 | 0.6181 |
| | | | | 001256455. | 000001.11 | | sense | CAAATTCA | | TAGGCAA | | | | |
| | | | | 1 | | | | AGGA | | ATTCAAGG | | | | |
| | | | | | | | | | | AGGGCGC | | | | |
| MED30 | IL2 | IL2 low | 90390 | NM_ | NC_ | 1.18E+08 | sense | ACACTGGA | 961. | TACCACAC | 1149. | CGG | 2 | 0.6317 |
| | | | | 080651.3 | 000008.11 | | | ACATATCA | | TGGAACAT | | | | |
| | | | | | | | | AGAC | | ATCAAGAC | | | | |
| | | | | | | | | | | CGGTTA | | | | |
| MED30 | IL2 | IL2 low | 90390 | NM_ | NC_ | 1.18E+08 | sense | GACAAAT | 962. | ATATGACA | 1150. | TGG | 2 | 0.7364 |
| | | | | 080651.3 | 000008.11 | | | GCAATGA | | AATGCAAT | | | | |
| | | | | | | | | AAACTG | | GAAAACT | | | | |
| | | | | | | | | | | GTGGTGG | | | | |
| MED30 | IL2 | IL2 low | 90390 | NM_ | NC_ | 1.18E+08 | sense | GGACATC | 963. | TGCAGGA | 1151. | TGG | 1 | 0.6878 |
| | | | | 080651.3 | 000008.11 | | | GTGTACCG | | CATCGTGT | | | | |
| | | | | | | | | CACCA | | ACCGCACC | | | | |
| | | | | | | | | | | ATGGAGA | | | | |
| MED30 | IL2 | IL2 low | 90390 | NM_ | NC_ | 1.18E+08 | sense | GGCCGCCC | 964. | AGCAGGC | 1152. | CGG | 1 | 0.5967 |
| | | | | 080651.3 | 000008.11 | | | GGGAAGT | | CGCCCGG | | | | |
| | | | | | | | | CAACA | | GAAGTCA | | | | |
| | | | | | | | | | | ACACGGC | | | | |
| | | | | | | | | | | GT | | | | |
| YBX1 | IL2 | IL2 low | 4904 | NM_ | NC_ | 42696740 | sense | AGACGCTA | 965. | TTATAGAC | 1153. | GGG | 5 | 0.6497 |
| | | | | 004559.3 | 000001.11 | | | TCCACGTC | | GCTATCCA | | | | |
| | | | | | | | | GTAG | | CGTCGTAG | | | | |
| | | | | | | | | | | GGGTCC | | | | |
| YBX1 | IL2 | IL2 low | 4904 | NM_ | NC_ | 42696671 | sense | GCAAATGT | 966. | GGCAGCA | 1154. | TGG | 5 | 0.6973 |
| | | | | 004559.3 | 000001.11 | | | TACAGGTC | | AATGTTAC | | | | |
| | | | | | | | | CTGG | | AGGTCCTG | | | | |
| | | | | | | | | | | GTGGTGT | | | | |
| YBX1 | IL2 | IL2 low | 4904 | NM_ | NC_ | 42682727 | sense | GGCGGGG | 967. | TGCCGGC | 1155. | CGG | 1 | 0.5977 |
| | | | | 004559.3 | 000001.11 | | | ACAAGAA | | GGGGACA | | | | |
| | | | | | | | | GGTCAT | | AGAAGGT | | | | |
| | | | | | | | | | | CATCGGTG | | | | |
| | | | | | | | | | | A | | | | |
| YBX1 | IL2 | IL2 low | 4904 | NM_ | NC_ | 42693498 | sense | GTCTTGCA | 968. | TTCTGTCT | 1156. | AGG | 3 | 0.6955 |
| | | | | 004559.3 | 000001.11 | | | GGAATGA | | TGCAGGA | | | | |
| | | | | | | | | CACCA | | ATGACACC | | | | |
| | | | | | | | | | | AAGGAAG | | | | |
| ZBTB7A | IL2 | IL2 low | 51341 | NM_ | NC_ | 4054925 | sense | ACCGTCAG | 969. | GCTCACCG | 1157. | GGG | 2 | 0.6826 |
| | | | | 015898.2 | 000019.10 | | | CACAGCCA | | TCAGCACA | | | | |
| | | | | | | | | ACGT | | GCCAACGT | | | | |
| | | | | | | | | | | GGGTGA | | | | |
| ZBTB7A | IL2 | IL2 low | 51341 | NM_ | NC_ | 4054671 | anti- | ATCATCGG | 970. | GGTCATCA | 1158. | CGG | 2 | 0.6019 |
| | | | | 015898.2 | 000019.10 | | sense | ACGCCCCA | | TCGGACGC | | | | |
| | | | | | | | | AAGG | | CCCAAAG | | | | |
| | | | | | | | | | | GCGGACC | | | | |
| ZBTB7A | IL2 | IL2 low | 51341 | NM_ | NC_ | 4054199 | sense | GAGTCGC | 971. | CGAGGAG | 1159. | GGG | 2 | 0.6111 |
| | | | | 015898.2 | 000019.10 | | | GGGCCGA | | TCGCGGG | | | | |
| | | | | | | | | CGACAA | | CCGACGAC | | | | |
| | | | | | | | | | | AAGGGCG | | | | |
| | | | | | | | | | | T | | | | |
| ZBTB7A | IL2 | IL2 low | 51341 | NM_ | NC_ | 4054431 | anti- | GCCGTAGT | 972. | CGCGGCC | 1160. | TGG | 2 | 0.6268 |
| | | | | 015898.2 | 000019.10 | | sense | GGCCGTTC | | GTAGTGG | | | | |
| | | | | | | | | TGCG | | CCGTTCTG | | | | |
| | | | | | | | | | | CGTGGCG | | | | |
| | | | | | | | | | | G | | | | |
| ATXN7L3 | IL2 | IL2 low | 56970 | NM_ | NC_ | 44197610 | sense | CACGGACC | 973. | ACGACAC | 1161. | AGG | 2 | 0.6328 |
| | | | | 001098833. | 000017.11 | | | CTGATAGC | | GGACCCTG | | | | |
| | | | | 1 | | | | ATGA | | ATAGCATG | | | | |
| | | | | | | | | | | AAGGATT | | | | |
| ATXN7L3 | IL2 | IL2 low | 56970 | NM_ | NC_ | 44197712 | sense | CATCGCTC | 974. | AGGCCATC | 1162. | CGG | 2 | 0.7491 |
| | | | | 001098833. | 000017.11 | | | AGGAGAT | | GCTCAGG | | | | |
| | | | | 1 | | | | ATACG | | AGATATAC | | | | |
| | | | | | | | | | | GCGGACC | | | | |
| ATXN7L3 | IL2 | IL2 low | 56970 | NM_ | NC_ | 44197233 | sense | GCAGCCG | 975. | AACAGCA | 1163. | CGG | 3 | 0.6135 |
| | | | | 001098833. | 000017.11 | | | AATCGCCA | | GCCGAATC | | | | |
| | | | | 1 | | | | ACCGC | | GCCAACCG | | | | |
| | | | | | | | | | | CCGGTGA | | | | |
| ATXN7L3 | IL2 | IL2 low | 56970 | NM_ | NC_ | 44195424 | sense | GCTTCGCA | 976. | AGGAGCTT | 1164. | CGG | 8 | 0.6566 |
| | | | | 001098833. | 000017.11 | | | GCCTGCTA | | CGCAGCCT | | | | |
| | | | | 1 | | | | ACCA | | GCTAACCA | | | | |
| | | | | | | | | | | CGGTGA | | | | |
| SRF | IL2 | IL2 low | 6722 | NM_ | NC_ | 43175724 | anti- | AGGTTGG | 977. | CGGCAGG | 1165. | CGG | 3 | 0.6646 |
| | | | | 003131.3 | 000006.12 | | sense | TGACTGTG | | TTGGTGAC | | | | |
| | | | | | | | | AACGC | | TGTGAACG | | | | |
| | | | | | | | | | | CCGGCTT | | | | |
| SRF | IL2 | IL2 low | 6722 | NM_ | NC_ | 43172119 | sense | AGTTCATC | 978. | ATGGAGTT | 1166. | CGG | 1 | 0.7054 |
| | | | | 003131.3 | 000006.12 | | | GACAACA | | CATCGACA | | | | |
| | | | | | | | | AGCTG | | ACAAGCTG | | | | |
| | | | | | | | | | | CGGCGC | | | | |
| SRF | IL2 | IL2 low | 6722 | NM_ | NC_ | 43175844 | anti- | GGGCTGA | 979. | ACTGGGG | 1167. | TGG | 3 | 0.65 |
| | | | | 003131.3 | 000006.12 | | sense | CACTAGCA | | CTGACACT | | | | |
| | | | | | | | | GACAC | | AGCAGAC | | | | |
| | | | | | | | | | | ACTGGTGC | | | | |
| SRF | IL2 | IL2 low | 6722 | NM_ | NC_ | 43174015 | anti- | TCTGTTGT | 980. | CTGGTCTG | 1168. | GGG | 2 | 0.605 |
| | | | | 003131.3 | 000006.12 | | sense | GGGGTCT | | TTGTGGG | | | | |
| | | | | | | | | GAACG | | GTCTGAAC | | | | |
| | | | | | | | | | | GGGGTGG | | | | |
| YY1 | IL2 | IL2 low | 7528 | NM_ | NC_ | 1E+08 | sense | AGATATTG | 981. | AAAAAGA | 1169. | TGG | 2 | 0.667 |
| | | | | 003403.4 | 000014.9 | | | ACCATGAG | | TATTGACC | | | | |
| | | | | | | | | ACAG | | ATGAGAC | | | | |
| | | | | | | | | | | AGTGGTT | | | | |
| | | | | | | | | | | G | | | | |
| YY1 | IL2 | IL2 low | 7528 | NM_ | NC_ | 1E+08 | sense | GGAGACC | 982 | CGGTGGA | 1170. | ITGG | 1 | 0.7975 |
| | | | | 003403.4 | 000014.9 | | | ATCGAGAC | | GACCATCG | | | | |
| | | | | | | | | CACAG | | AGACCACA | | | | |
| | | | | | | | | | | GTGGTGG | | | | |
| YY1 | IL2 | IL2 low | 7528 | NM_ | NC_ | 1E+08 | sense | GGTCACCG | 983. | CGCTGGTC | 1171. | AGG | 1 | 0.6314 |
| | | | | 003403.4 | 000014.9 | | | ACGACCCG | | ACCGACG | | | | |
| | | | | | | | | ACCC | | ACCCGACC | | | | |
| | | | | | | | | | | CAGGTGC | | | | |
| YY1 | IL2 | IL2 low | 7528 | NM_ | NC_ | 1E+08 | sense | TGAACAAA | 984. | ACATTGAA | 1172. | TGG | 1 | 0.7093 |
| | | | | 003403.4 | 000014.9 | | | CGCTGGTC | | CAAACGCT | | | | |
| | | | | | | | | ACCG | | GGTCACCG | | | | |
| | | | | | | | | | | TGGCGG | | | | |
| HINFP | IL2 | IL2 low | 25988 | NM_ | NC_ | 1.19E+08 | sense | CACACCAA | 985. | CTACCACA | 1173. | GGG | 4 | 0.6982 |
| | | | | 015517.4 | 000011.10 | | | GCTGAAAC | | CCAAGCTG | | | | |
| | | | | | | | | AGTG | | AAACAGT | | | | |
| | | | | | | | | | | GGGGGCT | | | | |
| HINFP | IL2 | IL2 low | 25988 | NM_ | NC_ | 1.19E+08 | sense | CATGCGCT | 986. | ACCACATG | 1174. | AGG | 8 | 0.7351 |
| | | | | 015517.4 | 000011.10 | | | TTCGTCAC | | CGCTTTCG | | | | |
| | | | | | | | | AGTG | | TCACAGTG | | | | |
| | | | | | | | | | | AGGACC | | | | |
| HINFP | IL2 | IL2 low | 25988 | NM_ | NC_ | 1.19E+08 | anti- | GGTGCTCT | 987. | CGGAGGT | 1175. | CGG | 6 | 0.7508 |
| | | | | 015517.4 | 000011.10 | | sense | CGAAGTTT | | GCTCTCGA | | | | |
| | | | | | | | | ACTG | | AGTTTACT | | | | |
| | | | | | | | | | | GCGGTCC | | | | |
| HINFP | IL2 | IL2 low | 25988 | NM_ | NC_ | 1.19E+08 | anti- | TGACTACT | 988. | CCTCTGAC | 1176. | AGG | 6 | 0.7145 |
| | | | | 015517.4 | 000011.10 | | sense | TACGATCC | | TACTTACG | | | | |
| | | | | | | | | AATG | | ATCCAATG | | | | |
| | | | | | | | | | | AGGTCT | | | | |
| MED14 | IL2 | IL2 low | 9282 | NM_ | NC_ | 40692233 | anti- | ATCACACA | 989. | TTGTATCA | 1177. | GGG | 15 | 0.6711 |
| | | | | 004229.3 | 000023.11 | | sense | TAGCGAC | | CACATAGC | | | | |
| | | | | | | | | GAAGT | | GACGAAG | | | | |
| | | | | | | | | | | TGGGCTA | | | | |
| MED14 | IL2 | IL2 low | 9282 | NM_ | NC_ | 40714644 | anti- | CAGAGCAT | 990. | GGACCAG | 1178. | AGG | 4 | 0.6206 |
| | | | | 004229.3 | 000023.11 | | sense | CTCTAGCT | | AGCATCTC | | | | |
| | | | | | | | | AACG | | TAGCTAAC | | | | |
| | | | | | | | | | | GAGGCCA | | | | |
| MED14 | IL2 | IL2 low | 9282 | NM_ | NC_ | 40682898 | anti- | CTAACTCT | 991. | AACACTAA | 1179. | CGG | 17 | 0.7032 |
| | | | | 004229.3 | 000023.11 | | sense | GCTACCCA | | CTCTGCTA | | | | |
| | | | | | | | | AGTG | | CCCAAGTG | | | | |
| | | | | | | | | | | CGGTTA | | | | |
| MED14 | IL2 | IL2 low | 9282 | NM_ | NC_ | 40711237 | sense | TAATGTTA | 992. | ACTCTAAT | 1180. | TGG | 8 | 0.6368 |
| | | | | 004229.3 | 300002.11 | | | ATCCGAGA | | GTTAATCC | | | | |
| | | | | | | | | ACGG | | GAGAACG | | | | |
| | | | | | | | | | | GTGGGGA | | | | |
| TUBB | IL2 | IL2 low | 203068 | NM_ | NC_ | 30722938 | anti- | AGATCCAC | 993. | TTCTAGAT | 1181. | AGG | 3 | 0.7005 |
| | | | | 178014.3 | 000006.12 | | sense | CAGGATG | | CCACCAGG | | | | |
| | | | | | | | | GCACG | | ATGGCAC | | | | |
| | | | | | | | | | | GAGGAAC | | | | |
| TUBB | IL2 | IL2 low | 203068 | NM_ | NC_ | 30722589 | sense | CCCCACCG | 994. | TCGACCCC | 1182. | GGG | 2 | 0.7034 |
| | | | | 178014.3 | 000006.12 | | | GCACCTAC | | ACCGGCAC | | | | |
| | | | | | | | | CACG | | CTACCACG | | | | |
| | | | | | | | | | | GGGACA | | | | |
| TUBB | IL2 | IL2 low | 203068 | NM_ | NC_ | 30723346 | sense | CTGCATTC | 995. | CTGACTGC | 1183. | GGG | 4 | 0.6146 |
| | | | | 178014.3 | 000006.12 | | | CAGGTCA | | ATTCCAGG | | | | |
| | | | | | | | | GTCTG | | TCAGTCTG | | | | |
| | | | | | | | | | | GGGCAG | | | | |
| TUBB | IL2 | IL2 low | 203068 | NM_ | NC_ | 30723727 | sense | GCTGACCA | 996. | TGAAGCTG | 1184. | GGG | 4 | 0.7623 |
| | | | | 178014.3 | 000006.12 | | | CACCAACC | | ACCACACC | | | | |
| | | | | | | | | TACG | | AACCTACG | | | | |
| | | | | | | | | | | GGGATC | | | | |
| MED11 | IL2 | IL2 low | 400569 | NM_ | NC_ | 4731567 | anti- | ATCCCGAA | 997. | TCGCATCC | 1185. | AGG | 1 | 0.567 |
| | | | | 001001683. | 000017.11 | | sense | CCTGCATT | | CGAACCTG | | | | |
| | | | | 3 | | | | CTGA | | CATTCTGA | | | | |
| | | | | | | | | | | AGGATG | | | | |
| MED11 | IL2 | IL2 low | 400569 | NM_ | NC_ | 4731863 | sense | CACCGCTT | 998. | CCTTCACC | 1186. | TGG | 2 | 0.6458 |
| | | | | 001001683. | 000017.11 | | | CAGTGCAA | | GCTTCAGT | | | | |
| | | | | | | | | CACG | | GCAACAC | | | | |
| | | | | | | | | | | GTGGAGG | | | | |
| MED11 | IL2 | IL2 low | 400569 | NM_ | NC_ | 4731814 | sense | CCAAGGA | 999. | TTGTCCAA | 1187. | CGG | 2 | 0.6925 |
| | | | | 3001001683. | 000017.11 | | | AAAAACTA | | GGAAAAA | | | | |
| | | | | 3 | | | | ACGAG | | ACTAACGA | | | | |
| | | | | | | | | | | GCGGCTC | | | | |
| MED11 | IL2 | IL2 low | 400569 | NM_ | NC_ | 4731833 | sense | GCGGCTCC | 1000. | ACGAGCG | 1188. | CGG | 2 | 0.6487 |
| | | | | 001001683. | 000017.11 | | | TAGACCG | | GCTCCTAG | | | | |
| | | | | 3 | | | | GCAGG | | ACCGGCA | | | | |
| | | | | | | | | | | GGCGGCG | | | | |
| | | | | | | | | | | G | | | | |
| FOXD4L5 | IL2 | IL2 low | 653427 | NM_ | NC_ | 65283612 | anti- | AGCGTAA | 1001. | GCAGAGC | 1189. | GGG | 1 | 0.68 |
| | | | | 001126334. | 000009.12 | | sense | GGGCATCT | | GTAAGGG | | | | |
| | | | | 1 | | | | CCCGG | | CATCTCCC | | | | |
| | | | | | | | | | | GGGGGCG | | | | |
| | | | | | | | | | | G | | | | |
| FOXD4L5 | IL2 | IL2 low | 653427 | NM_ | NC_ | 65284136 | anti- | CAAACTCT | 1002. | GTGCCAAA | 1190. | GGG | 1 | 0.6038 |
| | | | | 001126334. | 000009.12 | | sense | GAGGGGT | | CTCTGAGG | | | | |
| | | | | 1 | | | | CACTC | | GGTCACTC | | | | |
| | | | | | | | | | | GGGCCG | | | | |
| FOXD4L5 | IL2 | IL2 low | 653427 | NM_ | NC_ | 65284210 | sense | TAGAGCA | 1003. | TTCCTAGA | 1191. | GGG | 1 | 0.5918 |
| | | | | 001126334. | 000009.12 | | | GTCACTCC | | GCAGTCAC | | | | |
| | | | | 1 | | | | AGCCG | | TCCAGCCG | | | | |
| | | | | | | | | | | GGGCTG | | | | |
| FOXD4L5 | IL2 | IL2 low | 653427 | NM_ | NC_ | 65283953 | anti- | TGCGGCG | 1004. | AACTTGCG | 1192. | CGG | 1 | 0.5887 |
| | | | | 001126334. | 000009.12 | | sense | GTAGTATG | | GCGGTAG | | | | |
| | | | | 1 | | | | GGAÅG | | TATGGGA | | | | |
| | | | | | | | | | | AGCGGCC | | | | |
| | | | | | | | | | | A | | | | |
| MBD2 | IL2 | IL2 low | 8932 | NM_ | NC_ | 54224170 | sense | AGCCGGTC | 1005. | CGGGAGC | 1193. | GGG | 1 | 0.5949 |
| | | | | 003927.4 | 000018.10 | | | CCTTTCCC | | CGGTCCCT | | | | |
| | | | | | | | | GTCG | | TTCCCGTC | | | | |
| | | | | | | | | | | GGGGAGC | | | | |
| MBD2 | IL2 | IL2 low | 8932 | NM_ | NC_ | 54205113 | sense | CCTCAGTT | 1006. | CAAGCCTC | 1194. | GGG | 2 | 0.5818 |
| | | | | 003927.4 | 000018.10 | | | GGCAAGG | | AGTTGGCA | | | | |
| | | | | | | | | TACCT | | AGGTACCT | | | | |
| | | | | | | | | | | GGGAAA | | | | |
| MBD2 | IL2 | IL2 low | 8932 | NM_ | NC_ | 54204999 | sense | CCTCTCAA | 1007. | CGATCCTC | 1195. | TGG | 2 | 0.5401 |
| | | | | 003927.4 | 000018.10 | | | TCAAAATA | | TCAATCAA | | | | |
| | | | | | | | | AGGT | | AATAAGGT | | | | |
| | | | | | | | | | | TGGTTA | | | | |
| MBD2 | IL2 | IL2 low | 8932 | NM_ | NC_ | 54224048 | sense | CGAAAATC | 1008. | GATCCGAA | 1196. | ITGG | 1 | 0.494 |
| | | | | 003927.4 | 000018.10 | | | TGGGCTAA | | AATCTGGG | | | | |
| | | | | | | | | GTGC | | CTAAGTGC | | | | |
| | | | | | | | | | | TGGCAA | | | | |
| DMAP1 | IL2 | IL2 low | 55929 | NM_ | NC_ | 44218708 | sense | ATGCTGG | 1009. | TTTGATGC | 1197. | CGG | 6 | 0.6518 |
| | | | | 001034023. | 000001.11 | | | GCACGAA | | TGGGCAC | | | | |
| | | | | 1 | | | | CGACGG | | GAACGAC | | | | |
| | | | | | | | | | | GGCGGAA | | | | |
| | | | | | | | | | | G | | | | |
| DMAP1 | IL2 | IL2 low | 55929 | NM_ | NC_ | 44218427 | anti- | CATGGATA | 1010. | CGGTCATG | 1198. | AGG | 5 | 0.7087 |
| | | | | 001034023. | 000001.11 | | sense | ACAACAAA | | GATAACAA | | | | |
| | | | | 1 | | | | ACGC | | CAAAACGC | | | | |
| | | | | | | | | | | AGGTCA | | | | |
| DMAP1 | IL2 | IL2 low | 55929 | NM_ | NC_ | 44219225 | sense | GAAGCTAC | 1011. | AAAAGAA | 1199. | AGG | 7 | 0.693 |
| | | | | 001034023. | 000001.11 | | | CCCAGAAA | | GCTACCCC | | | | |
| | | | | 1 | | | | AAGG | | AGAAAAA | | | | |
| | | | | | | | | | | GGAGGCT | | | | |
| | | | | | | | | | | G | | | | |
| DMAP1 | IL2 | IL2 low | 55929 | NM_ | NC_ | 44213854 | sense | GGACATTA | 1012. | AGAAGGA | 1200. | AGG | 2 | 0.6418 |
| | | | | 001034023. | 000001.11 | | | TCAACCCG | | CATTATCA | | | | |
| | | | | 1 | | | | GACA | | ACCCGGAC | | | | |
| | | | | | | | | | | AAGGTAG | | | | |
| ABCF1 | IL2 | IL2 low | 23 | NM_ | NC_ | 30584469 | sense | CTGCAGA | 1013. | GCAGCTGC | 1201. | CGG | 14 | 0.6725 |
| | | | | 001025091. | 000006.12 | | | GGCCAAA | | AGAGGCC | | | | |
| | | | | 1 | | | | GCACGG | | AAAGCAC | | | | |
| | | | | | | | | | | GGCGGAT | | | | |
| | | | | | | | | | | C | | | | |
| ABCF1 | IL2 | IL2 low | 23 | NM_ | NC_ | 30583082 | sense | GATGGAG | 1014. | CTCAGATG | 1202. | TGG | 10 | 0.7118 |
| | | | | 001025091. | 000006.12 | | | TATGAGCG | | GAGTATG | | | | |
| | | | | 1 | | | | CCAAG | | AGCGCCA | | | | |
| | | | | | | | | | | AGTGGCTT | | | | |
| ABCF1 | IL2 | IL2 low | 23 | NM_ | NC_ | 30583862 | anti- | GCAACACA | 1015. | CACAGCAA | 1203. | GGG | 12 | 0.6289 |
| | | | | 001025091. | 000006.12 | | sense | TCAATGTT | | CACATCAA | | | | |
| | | | | 1 | | | | GGGA | | TGTTGGG | | | | |
| | | | | | | | | | | AGGGATG | | | | |
| ABCF1 | IL2 | IL2 low | 23 | NM_ | NC_ | 30585666 | anti- | TGTAATTG | 1016. | TTACTGTA | 1204. | TGG | 16 | 0.5878 |
| | | | | 001025091. | 000006.12 | | sense | CCCCTATA | | ATTGCCCC | | | | |
| | | | | 1 | | | | GTAG | | TATAGTAG | | | | |
| | | | | | | | | | | TGGAGC | | | | |
| ATF6B | IL2 | IL2 low | 1388 | NM_ | NC_ | 32121301 | sense | ATGTTCTT | 1017. | CTCCATGT | 1205. | AGG | 6 | 0.6359 |
| | | | | 004381.4 | 000006.12 | | | CCGTCAAC | | TCTTCCGT | | | | |
| | | | | | | | | TCTG | | CAACTCTG | | | | |
| | | | | | | | | | | AGGCCT | | | | |
| ATF6B | IL2 | IL2 low | 1388 | NM_ | NC_ | 32127108 | sense | CTCCACAG | 1018. | GTCTCTCC | 1206. | AGG | 4 | 0.7161 |
| | | | | 004381.4 | 000006.12 | | | AGCCATCC | | ACAGAGC | | | | |
| | | | | | | | | AGCG | | CATCCAGC | | | | |
| | | | | | | | | | | GAGGTGA | | | | |
| ATF6B | IL2 | IL2 low | 1388 | NM_ | NC_ | 32117995 | sense | CTCTCCTC | 1019. | CCATCTCT | 1207. | GGG | 12 | 0.7174 |
| | | | | 004381.4 | 000006.12 | | | GGATGAÅ | | CCTCGGAT | | | | |
| | | | | | | | | CAAGG | | GAACAAG | | | | |
| | | | | | | | | | | GGGGAGC | | | | |
| ATF6B | IL2 | IL2 low | 1388 | NM_ | NC_ | 32127466 | anti- | GATCGGC | 1020. | GGAAGAT | 1208. | GGG | 3 | 0.6296 |
| | | | | 004381.4 | 000006.12 | | sense | AGGAGTTC | | CGGCAGG | | | | |
| | | | | | | | | CCATG | | AGTTCCCA | | | | |
| | | | | | | | | | | TGGGGGC | | | | |
| | | | | | | | | | | T | | | | |
| GATA3 | IL2 | IL2 low | 2625 | NM_ | NC_ | 8055892 | sense | AGGTACCC | 1021. | GCAGAGG | 1209. | CGG | 2 | 0.6857 |
| | | | | 002051.2 | 000010.11 | | | TCCGACCC | | TACCCTCC | | | | |
| | | | | | | | | ACCA | | GACCCACC | | | | |
| | | | | | | | | | | ACGGTGA | | | | |
| GATA3 | IL2 | IL2 low | 2625 | NM_ | NC_ | 8064014 | sense | CAGGGAG | 1022. | AAGGCAG | 1210. | GGG | 4 | 0.737 |
| | | | | 002051.2 | 000010.11 | | | TGTGTGAA | | GGAGTGT | | | | |
| | | | | | | | | CTGTG | | GTGAACTG | | | | |
| | | | | | | | | | | TGGGGCA | | | | |
| | | | | | | | | | | A | | | | |
| GATA3 | IL2 | IL2 low | 2625 | NM_ | NC_ | 8058740 | anti- | GGAGCTG | 1023. | GTCCGGA | 1211. | AGG | 3 | 0.6273 |
| | | | | 002051.2 | 000010.11 | | sense | TACTCGGG | | GCTGTACT | | | | |
| | | | | | | | | CACGT | | CGGGCAC | | | | |
| | | | | | | | | | | GTAGGGC | | | | |
| | | | | | | | | | | G | | | | |
| GATA3 | IL2 | IL2 low | 2625 | NM_ | NC_ | 8058432 | sense | TCCAAGAC | 1024. | CTTCTCCA | 1212. | CGG | 3 | 0.7243 |
| | | | | 002051.2 | 000010.11 | | | GTCCATCC | | AGACGTCC | | | | |
| | | | | | | | | ACCA | | ATCCACCA | | | | |
| | | | | | | | | | | CGGCTC | | | | |
| IRF2 | IL2 | IL2 low | 3660 | NM_ | NC_ | 1.84E+08 | sense | ACCTGATC | 1025. | ATAAACCT | 1213. | AGG | 4 | 0.6049 |
| | | | | 002199.3 | 000004.12 | | | CCAAAACA | | GATCCCAA | | | | |
| | | | | | | | | TGGA | | AACATGG | | | | |
| | | | | | | | | | | AAGGCGA | | | | |
| IRF2 | IL2 | IL2 low | 3660 | NM_ | NC_ | 1.84E+08 | anti- | CAGCATTC | 1026. | GGGGCAG | 1214. | AGG | 4 | 0.6028 |
| | | | | 002199.3 | 000004.12 | | sense | GGTAGAC | | CATTCGGT | | | | |
| | | | | | | | | CCTGA | | AGACCCTG | | | | |
| | | | | | | | | | | AAGGCAT | | | | |
| IRF2 | IL2 | IL2 low | 3660 | NM_ | NC_ | 1.84E+08 | sense | GGATGCAT | 1027. | CCCTGGAT | 1215. | GGG | 3 | 0.6231 |
| | | | | 002199.3 | 000004.12 | | | GCGGCTA | | GCATGCG | | | | |
| | | | | | | | | GACAT | | GCTAGACA | | | | |
| | | | | | | | | | | TGGGTGG | | | | |
| IRF2 | IL2 | IL2 low | 3660 | NM_ | NC_ | 1.84E+08 | anti- | TACCTGCA | 1028. | CGCTTACC | 1216. | GGG | 7 | 0.7207 |
| | | | | 002199.3 | 000004.12 | | sense | TAGGAAG | | TGCATAGG | | | | |
| | | | | | | | | ACACG | | AAGACAC | | | | |
| | | | | | | | | | | GGGGGAG | | | | |
| NFATC2 | IL2 | IL2 low | 4773 | NM_ | NC_ | 51474071 | anti- | ACATTGGA | 1029. | ACGGACAT | 1217. | CGG | 5 | 0.7428 |
| | | | | 001258297. | 000020.11 | | sense | AGAAAGA | | TGGAAGA | | | | |
| | | | | 1 | | | | ACACG | | AAGAACA | | | | |
| | | | | | | | | | | CGCGGGT | | | | |
| | | | | | | | | | | G | | | | |
| NFATC2 | IL2 | IL2 low | 4773 | NM_ | NC_ | 51454601 | anti- | ATGTAAAG | 1030. | TCGGATGT | 1218. | AGG | 6 | 0.7057 |
| | | | | 001258297. | 000020.11 | | sense | TTCTGCCC | | AAAGTTCT | | | | |
| | | | | 1 | | | | CGTG | | GCCCCGTG | | | | |
| | | | | | | | | | | AGGATC | | | | |
| NFATC2 | IL2 | IL2 low | 4773 | NM_ | NC_ | 51523157 | sense | GCAGGGC | 1031. | GCGAGCA | 1219. | CGG | 2 | 0.6354 |
| | | | | 001258297. | 000020.11 | | | GAGAGGA | | GGGCGAG | | | | |
| | | | | 1 | | | | GAAACT | | AGGAGAA | | | | |
| | | | | | | | | | | ACTCGGCT | | | | |
| | | | | | | | | | | C | | | | |
| NFATC2 | IL2 | IL2 low | 4773 | NM_ | NC_ | 51523397 | sense | GCCGCAG | 1032. | CCTCGCCG | 1220. | TGG | 2 | 0.6587 |
| | | | | 001258297. | 000020.11 | | | CCCTCATC | | CAGCCCTC | | | | |
| | | | | 1 | | | | TCACG | | ATCTCACG | | | | |
| | | | | | | | | | | TGGCAC | | | | |
| ZEB1 | IL2 | IL2 low | 6935 | NM_ | NC_ | 31520685 | sense | AATGCTTC | 1033. | AATCAATG | 1221. | AGG | 7 | 0.7295 |
| | | | | 030751.5 | 000010.11 | | | ACCCATAC | | CTTCACCC | | | | |
| | | | | | | | | AACA | | ATACAACA | | | | |
| | | | | | | | | | | AGGTGG | | | | |
| ZEB1 | IL2 | IL2 low | 6935 | NM_ | NC_ | 31461183 | sense | CAGACCA | 1034. | CCAACAGA | 1222. | GGG | 2 | 0.6782 |
| | | | | 030751.5 | 000010.11 | | | GACAGTGT | | CCAGACA | | | | |
| | | | | | | | | TACCA | | GTGTTACC | | | | |
| | | | | | | | | | | AGGGAGG | | | | |
| ZEB1 | IL2 | IL2 low | 6935 | NM_ | NC_ | 31520903 | sense | GAAGGAC | 1035. | CTGAGAA | 1223. | GGG | 7 | 0.669 |
| | | | | 030751.5 | 000010.11 | | | AAAAGCTT | | GGACAAA | | | | |
| | | | | | | | | TGAAG | | AGCTTTGA | | | | |
| | | | | | | | | | | AGGGGGG | | | | |
| | | | | | | | | | | G | | | | |
| ZEB1 | IL2 | IL2 low | 6935 | NM_ | NC_ | 31521504 | sense | GGTTACTT | 1036. | ACAGGGTT | 1224. | GGG | 7 | 0.66 |
| | | | | 030751.5 | 000010.11 | | | GTACACAG | | ACTTGTAC | | | | |
| | | | | | | | | CTGA | | ACAGCTGA | | | | |
| | | | | | | | | | | GGGTGC | | | | |
| PKNOX1 | IL2 | IL2 low | 5316 | NM_ | NC_ | 43013196 | sense | AACAGTG | 1037. | AATGAACA | 1225. | TGG | 5 | 0.6274 |
| | | | | 004571.4 | 000021.9 | | | AAACTCTG | | GTGAAACT | | | | |
| | | | | | | | | TTGAG | | CTGTTGAG | | | | |
| | | | | | | | | | | TGGAGA | | | | |
| PKNOX1 | IL2 | IL2 low | 5316 | NM_ | NC_ | 43016996 | sense | GGGAAAC | 1038. | AGCAGGG | 1226. | CGG | 6 | 0.6554 |
| | | | | 004571.4 | 000021.9 | | | GTAGCCAT | | AAACGTA | | | | |
| | | | | | | | | GGCGA | | GCCATGGC | | | | |
| | | | | | | | | | | GACGGTG | | | | |
| | | | | | | | | | | G | | | | |
| PKNOX1 | IL2 | IL2 low | 5316 | NM_ | NC_ | 43021368 | sense | GGTTCATC | 1039. | TGATGGTT | 1227. | GGG | 8 | 0.6957 |
| | | | | 004571.4 | 000021.9 | | | TAAGAACA | | CATCTAAG | | | | |
| | | | | | | | | AGAG | | AACAAGA | | | | |
| | | | | | | | | | | GGGGCGT | | | | |
| PKNOX1 | IL2 | IL2 low | 5316 | NM_ | NC_ | 43007550 | sense | TGCTCTGA | 1040. | AAACTGCT | 1228. | AGG | 3 | 0.583 |
| | | | | 004571.4 | 000021.9 | | | ACCCGATG | | CTGAACCC | | | | |
| | | | | | | | | CAGA | | GATGCAG | | | | |
| | | | | | | | | | | AAGGAGT | | | | |
| USP22 | IL2 | IL2 low | 23326 | NM_ | NC_ | 21015837 | sense | ACCTGGTG | 1041. | CTGCACCT | 1229. | AGG | 6 | 0.6275 |
| | | | | 015276.1 | 000017.11 | | | TGGACCCA | | GGTGTGG | | | | |
| | | | | | | | | CGCG | | ACCCACGC | | | | |
| | | | | | | | | | | GAGGCAC | | | | |
| USP22 | IL2 | IL2 low | 23326 | NM_ | NC_ | 21019085 | sense | CCTCGAAC | 1042. | ATCACCTC | 1230. | GGG | 4 | 0.6518 |
| | | | | 015276.1 | 000017.11 | | | TGCACCAT | | GAACTGCA | | | | |
| | | | | | | | | AGGT | | CCATAGGT | | | | |
| | | | | | | | | | | GGGTGG | | | | |
| USP22 | IL2 | IL2 low | 23326 | NM_ | NC_ | 21021211 | sense | GCCATTGA | 1043. | CTCAGCCA | 1231. | AGG | 3 | 0.6813 |
| | | | | 015276.1 | 000017.11 | | | TCTGATGT | | TTGATCTG | | | | |
| | | | | | | | | ACGG | | ATGTACGG | | | | |
| | | | | | | | | | | AGGCAT | | | | |
| USP22 | IL2 | IL2 low | 23326 | NM_ | NC_ | 21018000 | anti- | TGGGGCT | 1044. | GAGCTGG | 1232. | CGG | 5 | 0.6752 |
| | | | | 601527.1 | 700001.11 | | sense | CTGCATCT | | GGCTCTGC | | | | |
| | | | | | | | | CACAG | | ATCTCACA | | | | |
| | | | | | | | | | | GCGGTGC | | | | |
| ZBTB11 | IL2 | IL2 low | 27107 | NM_ | NC_ | 1.02E+08 | anti- | AATAAGAT | 1045. | GACGAAT | 1233. | AGG | 6 | 0.6273 |
| | | | | 501441.3 | 000003.12 | | sense | GTGCTCGC | | AAGATGT | | | | |
| | | | | | | | | AAAG | | GCTCGCAA | | | | |
| | | | | | | | | | | AGAGGCA | | | | |
| | | | | | | | | | | C | | | | |
| ZBTB11 | IL2 | IL2 low | 27107 | NM_ | NC_ | 1.02E+08 | sense | CAGGACTT | 1046. | CACCCAGG | 1234. | TGG | 4 | 0.605 |
| | | | | 014415.3 | 000003.12 | | | ACGAGTAC | | ACTTACGA | | | | |
| | | | | | | | | AGAA | | GTACAGA | | | | |
| | | | | | | | | | | ATGGAGG | | | | |
| ZBTB11 | IL2 | IL2 low | 27107 | NM_ | NC_ | 1.02E+08 | sense | GGCATATA | 1047. | AAGGGGC | 1235. | AGG | 4 | 0.5992 |
| | | | | 014415.3 | 000003.12 | | | TTCGACTA | | ATATATTC | | | | |
| | | | | | | | | CACA | | GACTACAC | | | | |
| | | | | | | | | | | AAGGGAA | | | | |
| ZBTB11 | IL2 | IL2 low | 27107 | NM_ | NC_ | 1.02E+08 | anti- | TATACATC | 1048. | CAGCTATA | 1236. | GGG | 4 | 0.6653 |
| | | | | 014415.3 | 000003.12 | | sense | CTACTGAT | | CATCCTAC | | | | |
| | | | | | | | | GACA | | TGATGACA | | | | |
| | | | | | | | | | | GGGCAG | | | | |
| TBP | IL2 | IL2 low | 6908 | NM_ | NC_ | 1.71E+08 | sense | ACGTCCCA | 1049. | GTCAACGT | 1237. | GGG | 3 | 0.6765 |
| | | | | 003194.4 | 000006.12 | | | GCAGGCA | | CCCAGCAG | | | | |
| | | | | | | | | ACACA | | GCAACACA | | | | |
| | | | | | | | | | | GGGAAC | | | | |
| TBP | IL2 | IL2 low | 6908 | NM_ | NC_ | 1.71E+08 | sense | CCAATGAT | 1050. | TAGTCCAA | 1238. | TGG | 3 | 0.4983 |
| | | | | 003194.4 | 000006.12 | | | GCCTTATG | | TGATGCCT | | | | |
| | | | | | | | | GCAC | | TATGGCAC | | | | |
| | | | | | | | | | | TGGACT | | | | |
| TBP | IL2 | IL2 low | 6908 | NM_ | NC_ | 1.71E+08 | sense | GATAAGA | 1051. | TGAGGAT | 1239. | CGG | 5 | 0.6043 |
| | | | | 003194.4 | 000006.12 | | | GAGCCAC | | AAGAGAG | | | | |
| | | | | | | | | GAACCA | | CCACGAAC | | | | |
| | | | | | | | | | | CACGGCAC | | | | |
| TBP | IL2 | IL2 low | 6908 | NM_ | NC_ | 1.71E+08 | anti- | GTTTCGGG | 1052. | CGGCGTTT | 1240. | TGG | 4 | 0.5851 |
| | | | | 003194.4 | 000006.12 | | sense | CACGAAGT | | CGGGCAC | | | | |
| | | | | | | | | GCAA | | GAAGTGC | | | | |
| | | | | | | | | | | AATGGTCT | | | | |
| JAK3 | IL2 | IL2 low | 3718 | NM_ | NC_ | 17835098 | anti- | ACTCTCCA | 1053. | ACTTACTC | 1241. | CGG | 15 | 0.7301 |
| | | | | 000215.3 | 000019.10 | | sense | GGCTTAAC | | TCCAGGCT | | | | |
| | | | | | | | | ACAG | | TAACACAG | | | | |
| | | | | | | | | | | CGGGGC | | | | |
| JAK3 | IL2 | IL2 low | 3718 | NM_ | NC_ | 17839577 | anti- | AGCTCTCG | 1054. | CAGGAGC | 1242. | GGG | 10 | 0.7109 |
| | | | | 000215.3 | 000019.10 | | sense | AAGACTGC | | TCTCGAAG | | | | |
| | | | | | | | | TGTG | | ACTGCTGT | | | | |
| | | | | | | | | | | GGGGTCG | | | | |
| JAK3 | IL2 | IL2 low | 3718 | NM_ | NC_ | 17836043 | anti- | GTGTACAA | 1055. | CCAGGTGT | 1243. | TGG | 14 | 0.6764 |
| | | | | 000215.3 | 000019.10 | | sense | ATTCCTGC | | ACAAATTC | | | | |
| | | | | | | | | ACCA | | CTGCACCA | | | | |
| | | | | | | | | | | TGGTGC | | | | |
| JAK3 | IL2 | IL2 low | 3718 | NM_ | NC_ | 17842538 | sense | TGACGCG | 1056. | TTCGTGAC | 1244. | AGG | 6 | 0.6476 |
| | | | | 500021.3 | 000019.10 | | | GAGGCGT | | GCGGAGG | | | | |
| | | | | | | | | ATTCGG | | CGTATTCG | | | | |
| | | | | | | | | | | GAGGACG | | | | |
| PRRC2A | IL2 | IL2 low | 7916 | NM_ | NC_ | 31631224 | anti- | AGAGGAA | 1057. | CTCCAGAG | 1245. | GGG | 16 | 0.6995 |
| | | | | 080686.2 | 000006.12 | | sense | AGCGAGA | | GAAAGCG | | | | |
| | | | | | | | | GATTGG | | AGAGATT | | | | |
| | | | | | | | | | | GGGGGCC | | | | |
| | | | | | | | | | | C | | | | |
| PRRC2A | IL2 | IL2 low | 7916 | NM_ | NC_ | 31632445 | sense | ATAAACCG | 1058. | CAGGATA | 1246. | AGG | 16 | 0.6561 |
| | | | | 608068.2 | 000006.12 | | | CCTCGTTT | | AACCGCCT | | | | |
| | | | | | | | | CCGG | | CGTTTCCG | | | | |
| | | | | | | | | | | GAGGCTG | | | | |
| PRRC2A | IL2 | IL2 low | 7916 | NM_ | NC_ | 31629740 | anti- | CGGGGAT | 1059. | CCATCGGG | 1247. | GGG | 14 | 0.6731 |
| | | | | 080686.2 | 000006.12 | | sense | CAAAGTTC | | GATCAAA | | | | |
| | | | | | | | | ATTGG | | GTTCATTG | | | | |
| | | | | | | | | | | GGGGCAT | | | | |
| PRRC2A | IL2 | IL2 low | 7916 | NM_ | NC_ | 31625834 | sense | CTCCGCCC | 1060. | TTCCCTCC | 1248. | GGG | 8 | 0.721 |
| | | | | 080686.2 | 000006.12 | | | TATGGACC | | GCCCTATG | | | | |
| | | | | | | | | CCAG | | GACCCCAG | | | | |
| | | | | | | | | | | GGGCCT | | | | |
| RBPJ | IL2 | IL2 low | 3516 | NM_ | NC_ | 26415547 | sense | AAAGAAC | 1061. | AAAAAAA | 1249. | TGG | 5 | 0.6336 |
| | | | | 005349.3 | 000004.12 | | | AAATGGA | | GAACAAAT | | | | |
| | | | | | | | | ACGCGA | | GGAACGC | | | | |
| | | | | | | | | | | GATGGTT | | | | |
| | | | | | | | | | | G | | | | |
| RBPJ | IL2 | IL2 low | 3516 | NM_ | NC_ | 26386378 | anti- | CACCTAGT | 1062. | TACTCACC | 1250. | TGG | 3 | 0.6435 |
| | | | | 005349.3 | 000004.12 | | sense | AAGTOGTT | | TAGTAAGT | | | | |
| | | | | | | | | TAGG | | CGTTTAGG | | | | |
| | | | | | | | | | | TGGAGG | | | | |
| RBPJ | IL2 | IL2 low | 3516 | NM_ | NC_ | 26424454 | sense | CATGCCAG | 1063. | TTTTCATG | 1251. | GGG | 7 | 0.6844 |
| | | | | 005349.3 | 000004.12 | | | TTCACAGC | | CCAGTTCA | | | | |
| | | | | | | | | AGTG | | CAGCAGT | | | | |
| | | | | | | | | | | GGGGAGC | | | | |
| RBPJ | IL2 | IL2 low | 3516 | NM_ | NC_ | 26424363 | sense | CATTGCCT | 1064. | TATGCATT | 1252. | TGG | 7 | 0.65 |
| | | | | 005349.3 | 000004.12 | | | CAGGAAC | | GCCTCAGG | | | | |
| | | | | | | | | AAAGG | | AACAAAG | | | | |
| | | | | | | | | | | GTGGCTC | | | | |
| STAT5B | IL2 | IL2 low | 6777 | NM_ | NC_ | 42216055 | sense | CAGCCAG | 1065. | ATGGCAG | 1253. | CGG | 12 | 0.6375 |
| | | | | 012448.3 | 000017.11 | | | GACAACA | | CCAGGAC | | | | |
| | | | | | | | | ATGCGA | | AACAATGC | | | | |
| | | | | | | | | | | GACGGCC | | | | |
| | | | | | | | | | | A | | | | |
| STAT5B | IL2 | IL2 low | 6777 | NM_ | NC_ | 42227658 | anti- | GTGGCCTT | 1066. | CTGGGTG | 1254. | TGG | 3 | 0.6157 |
| | | | | 012448.3 | 000017.11 | | sense | AATGTTCT | | GCCTTAAT | | | | |
| | | | | | | | | CCTG | | GTTCTCCT | | | | |
| | | | | | | | | | | GTGGATT | | | | |
| STAT5B | IL2 | IL2 low | 6777 | NM_ | NC_ | 42224822 | anti- | GTTCATTG | 1067. | CTCTGTTC | 1255. | CGG | 4 | 0.6583 |
| | | | | 012448.3 | 000017.11 | | sense | TACAATAT | | ATTGTACA | | | | |
| | | | | | | | | ATGG | | ATATATGG | | | | |
| | | | | | | | | | | CGGATG | | | | |
| STAT5B | IL2 | IL2 low | 6777 | NM_ | NC_ | 42217252 | sense | TAAGAGG | 1068. | GAATTAAG | 1256. | GGG | 11 | 0.7097 |
| | | | | 012448.3 | 000017.11 | | | TCAGACCG | | AGGTCAG | | | | |
| | | | | | | | | TCGTG | | ACCGTCGT | | | | |
| | | | | | | | | | | GGGGCAG | | | | |
| PRKRIR | IL2 | IL2 low | 5612 | NM_ | NC_ | 76351762 | sense | AGATGGA | 1069. | TATTAGAT | 1257. | TGG | 5 | 0.5452 |
| | | | | 004705.3 | 000011.10 | | | ATAACTAT | | GGAATAA | | | | |
| | | | | | | | | ATAGC | | CTATATAG | | | | |
| | | | | | | | | | | CTGGCCG | | | | |
| PRKRIR | IL2 | IL2 low | 5612 | NM_ | NC_ | 76352722 | sense | CAGAACCC | 1070. | TGCTCAGA | 1258. | GGG | 5 | 0.6252 |
| | | | | 004705.3 | 000011.10 | | | CAGCGAA | | ACCCCAGC | | | | |
| | | | | | | | | GAAGA | | GAAGAAG | | | | |
| | | | | | | | | | | AGGGTGA | | | | |
| PRKRIR | IL2 | IL2 low | 5612 | NM_ | NC_ | 76352601 | anti- | TCAGCCTC | 1071. | TTCATCAG | 1259. | AGG | 5 | 0.6814 |
| | | | | 004705.3 | 000011.10 | | sense | ATGTCCAT | | CCTCATGT | | | | |
| | | | | | | | | CCAG | | CCATCCAG | | | | |
| | | | | | | | | | | AGGTAT | | | | |
| PRKRIR | IL2 | IL2 low | 5612 | NM_ | NC_ | 76352282 | sense | TGAATCTC | 1072. | TTGATGAA | 1260. | AGG | 5 | 0.6598 |
| | | | | 500470.3 | 000011.10 | | | ATAACCTA | | TCTCATAA | | | | |
| | | | | | | | | AGAG | | CCTAAGAG | | | | |
| | | | | | | | | | | AGGAAT | | | | |
| CDC5L | IL2 | IL2 low | 988 | NM_ | NC_ | 44408511 | sense | AATTGCAC | 1073. | GTGAAATT | 1261. | AGG | 8 | 0.6775 |
| | | | | 001253.3 | 000006.12 | | | GTCAAACT | | GCACGTCA | | | | |
| | | | | | | | | GCCG | | AACTGCCG | | | | |
| | | | | | | | | | | AGGAAT | | | | |
| CDC5L | IL2 | IL2 low | 988 | NM_ | NC_ | 44392761 | sense | AGTTGATG | 1074. | GCCAAGTT | 1262. | AGG | 3 | 0.6555 |
| | | | | 001253.3 | 000006.12 | | | CCAACTCA | | GATGCCAA | | | | |
| | | | | | | | | GTGG | | CTCAGTGG | | | | |
| | | | | | | | | | | AGGACC | | | | |
| CDCSL | IL2 | IL2 low | 988 | NM_ | NC_ | 44393543 | sense | ATCCAGAA | 1075. | CCAAATCC | 1263. | CGG | 4 | 0.7584 |
| | | | | 001253.3 | 000006.12 | | | ACAAAACC | | AGAAACA | | | | |
| | | | | | | | | AGCG | | AAACCAGC | | | | |
| | | | | | | | | | | GCGGCCT | | | | |
| CDC5L | IL2 | IL2 low | 988 | NM_ | NC_ | 44419516 | anti- | GAAGTCAC | 1076. | CTGAGAA | 1264. | GGG | 9 | 0.742 |
| | | | | 001253.3 | 000006.12 | | sense | TCTCATGC | | GTCACTCT | | | | |
| | | | | | | | | AATG | | CATGCAAT | | | | |
| | | | | | | | | | | GGGGTAT | | | | |
| TERF2 | IL2 | IL2 low | 7014 | NM_ | NC_ | 69370578 | anti- | AAAGTTCT | 1077. | ATGAAAA | 1265. | GGG | 5 | 0.6289 |
| | | | | 005652.4 | 000016.10 | | sense | GGATAAC | | GTTCTGGA | | | | |
| | | | | | | | | AGGAT | | TAACAGG | | | | |
| | | | | | | | | | | ATGGGCC | | | | |
| | | | | | | | | | | A | | | | |
| TERF2 | IL2 | IL2 low | 7014 | NM_ | NC_ | 69385848 | sense | CGCGCGG | 1078. | AGGGCGC | 1266. | TGG | 1 | 0.6198 |
| | | | | 005652.4 | 000016.10 | | | CGATCGG | | GCGGCGA | | | | |
| | | | | | | | | ACACGA | | TCGGACAC | | | | |
| | | | | | | | | | | GATGGCG | | | | |
| | | | | | | | | | | G | | | | |
| TERF2 | IL2 | IL2 low | 7014 | NM_ | NC_ | 69385419 | sense | GGGTTAT | 1079. | CTGCGGG | 1267. | CGG | 2 | 0.6807 |
| | | | | 005652.4 | 000016.10 | | | GCAGTGTC | | TTATGCAG | | | | |
| | | | | | | | | TGTCG | | TGTCTGTC | | | | |
| | | | | | | | | | | GCGGATT | | | | |
| TERF2 | IL2 | IL2 low | 7014 | NM_ | NC_ | 69385628 | anti- | TCTGTCTG | 1080. | CGGATCTG | 1268. | CGG | 1 | 0.6089 |
| | | | | 005652.4 | 000016.10 | | sense | AAGTCCCC | | TCTGAAGT | | | | |
| | | | | | | | | GTAC | | CCCCGTAC | | | | |
| | | | | | | | | | | CGGCTA | | | | |
| PBX2 | IL2 | IL2 low | 5089 | NM_ | NC_ | 32188361 | anti- | AGGGGAC | 1081. | TGTCAGG | 1269. | AGG | 3 | 0.6149 |
| | | | | 002586.4 | 000006.12 | | sense | ACACCACC | | GGACACA | | | | |
| | | | | | | | | ACCAG | | CCACCACC | | | | |
| | | | | | | | | | | AGAGGCT | | | | |
| | | | | | | | | | | G | | | | |
| PBX2 | IL2 | IL2 low | 5089 | NM_ | NC_ | 32188279 | anti- | CATACTTC | 1082. | TGCTCATA | 1270. | TGG | 3 | 0.7077 |
| | | | | 002586.4 | 000006.12 | | sense | TCCAGCTC | | CTTCTCCA | | | | |
| | | | | | | | | CGAG | | GCTCCGAG | | | | |
| | | | | | | | | | | TGGTAT | | | | |
| PBX2 | IL2 | IL2 low | 5089 | NM_ | NC_ | 32189820 | anti- | CCGGGGT | 1083. | CCCACCGG | 1271. | AGG | 1 | 0.6787 |
| | | | | 002586.4 | 000006.12 | | sense | CTCCGCCA | | GGTCTCCG | | | | |
| | | | | | | | | CCGGG | | CCACCGG | | | | |
| | | | | | | | | | | GAGGCTC | | | | |
| PBX2 | IL2 | IL2 low | 5089 | NM_ | NC_ | 32187751 | sense | CTTCAGCA | 1084. | GTAACTTC | 1272. | AGG | 5 | 0.6791 |
| | | | | 002586.4 | 000006.12 | | | AACAGGC | | AGCAAAC | | | | |
| | | | | | | | | CACTG | | AGGCCACT | | | | |
| | | | | | | | | | | GAGGTCC | | | | |
| CIC | IL2 | IL2 low | 23152 | NM_ | NC_ | 42291060 | sense | ACTGTCAC | 1085. | TGCCACTG | 1273. | GGG | 10 | 0.6591 |
| | | | | 015125.4 | 000019.10 | | | TAACCTAC | | TCACTAAC | | | | |
| | | | | | | | | TGGT | | CTACTGGT | | | | |
| | | | | | | | | | | GGGCAC | | | | |
| CIC | IL2 | IL2 low | 23152 | NM_ | NC_ | 42292311 | anti- | CCCGCCCG | 1086. | CGTGCCCG | 1274. | AGG | 13 | 0.6689 |
| | | | | 015125.4 | 000019.10 | | sense | CTGACTGC | | CCCGCTGA | | | | |
| | | | | | | | | ACAT | | CTGCACAT | | | | |
| | | | | | | | | | | AGGTGA | | | | |
| CIC | IL2 | IL2 low | 23152 | NM_ | NC_ | 42287372 | sense | CTCTACCG | 1087. | TTGCCTCT | 1275. | CGG | 4 | 0.6842 |
| | | | | 015125.4 | 000019.10 | | | CCCGGAA | | ACCGCCCG | | | | |
| | | | | | | | | AACGT | | GAAAACG | | | | |
| | | | | | | | | | | TCGGACC | | | | |
| CIC | IL2 | IL2 low | 23152 | NM_ | NC_ | 42289198 | anti- | TTGGGCCA | 1088. | GGCTTTGG | 1276. | GGG | 8 | 0.6248 |
| | | | | 015125.4 | 000019.10 | | sense | GAGTACG | | GCCAGAG | | | | |
| | | | | | | | | ATGCA | | TACGATGC | | | | |
| | | | | | | | | | | AGGGCCA | | | | |
| SS18 | IL2 | IL2 low | 6760 | NM_ | NC_ | 26052686 | sense | AATCAGAT | 1089. | ACAGAATC | 1277. | GGG | 5 | 0.6566 |
| | | | | 001007559. | 000018.10 | | | GACAATG | | AGATGAC | | | | |
| | | | | 1 | | | | AGTCA | | AATGAGTC | | | | |
| | | | | | | | | | | AGGGACA | | | | |
| SS18 | IL2 | IL2 low | 6760 | NM_ | NC_ | 26039408 | sense | CAATACAA | 1090. | TCAGCAAT | 1278. | AGG | 6 | 0.7278 |
| | | | | 001007559. | 000018.10 | | | TATGCCAC | | ACAATATG | | | | |
| | | | | 1 | | | | AGGG | | CCACAGG | | | | |
| | | | | | | | | | | GAGGCGG | | | | |
| SS18 | IL2 | IL2 low | 6760 | NM_ | NC_ | 26052827 | sense | CCTAACCA | 1091. | AGGGCCT | 1279. | GGG | 5 | 0.6709 |
| | | | | 001007559. | 000018.10 | | | TATGCCTA | | AACCATAT | | | | |
| | | | | 1 | | | | TGCA | | GCCTATGC | | | | |
| | | | | | | | | | | AGGGACC | | | | |
| SS18 | IL2 | IL2 low | 6760 | NM_ | NC_ | 26057677 | anti- | GGCATGTT | 1092. | TGAAGGC | 1280. | AGG | 4 | 0.7849 |
| | | | | 001007559. | 000018.10 | | sense | GTGAGAG | | ATGTTGTG | | | | |
| | | | | 1 | | | | CGTGG | | AGAGCGT | | | | |
| | | | | | | | | | | GGAGGTG | | | | |
| | | | | | | | | | | G | | | | |
| HMGA1 | IL2 | IL2 low | 3159 | NM_ | NC_ | 34240847 | sense | AAAAGGA | 1093. | CAGGAAA | 1281. | CGG | 3 | 0.555 |
| | | | | 145899.2 | 000006.12 | | | CGGCACTG | | AGGACGG | | | | |
| | | | | | | | | AGAAG | | CACTGAGA | | | | |
| | | | | | | | | | | AGCGGGG | | | | |
| | | | | | | | | | | C | | | | |
| HMGA1 | IL2 | IL2 low | 3159 | NM_ | NC_ | 34240914 | sense | AGCGCTG | 1094. | GGACAGC | 1282. | TGG | 3 | 0.5867 |
| | | | | 145899.2 | 000006.12 | | | GTAGGGA | | GCTGGTA | | | | |
| | | | | | | | | GTCAGG | | GGGAGTC | | | | |
| | | | | | | | | | | AGGTGGG | | | | |
| | | | | | | | | | | TG | | | | |
| HMGA1 | IL2 | IL2 low | 3159 | NM_ | NC_ | 34242747 | sense | CCAACACC | 1095. | AGTGCCAA | 1283. | GGG | 4 | 0.7288 |
| | | | | 145899.2 | 000006.12 | | | TAAGAGA | | CACCTAAG | | | | |
| | | | | | | | | CCTCG | | AGACCTCG | | | | |
| | | | | | | | | | | GGGCCG | | | | |
| HMGA1 | IL2 | IL2 low | 3159 | NM_ | NC_ | 34240883 | anti- | GCTGTCCC | 1096. | CAGCGCTG | 1284. | AGG | 3 | 0.6102 |
| | | | | 145899.2 | 000006.12 | | sense | GGGACTC | | TCCCGGGA | | | | |
| | | | | | | | | ACCGG | | CTCACCGG | | | | |
| | | | | | | | | | | AGGCTG | | | | |
| DUX4 | IL2 | IL2 low | 1E+08 | NM_ | NC_ | 1.9E+08 | sense | ACACCGGC | 1097. | GCCCACAC | 1285. | GGG | 1 | 0.5159 |
| | | | | 001293798. | 000004.12 | | | GCGTGGG | | CGGCGCG | | | | |
| | | | | 1 | | | | GAACG | | TGGGGAA | | | | |
| | | | | | | | | | | CGGGGCT | | | | |
| | | | | | | | | | | T | | | | |
| DUX4 | IL2 | IL2 low | 1E+08 | NM_ | NC_ | 1.9E+08 | sense | GGCAGGC | 1098. | CGCAGGC | 1286. | CGG | 1 | 0.617 |
| | | | | 001293798. | 000004.12 | | | GGCCTGT | | AGGCGGC | | | | |
| | | | | 1 | | | | GCAGCG | | CTGTGCAG | | | | |
| | | | | | | | | | | CGCGGCCC | | | | |
| DUX4 | IL2 | IL2 low | 1E+08 | NM_ | NC_ | 1.9E+08 | sense | GGGGATC | 1099. | CAGAGGG | 1287. | CGG | 1 | 0.5181 |
| | | | | 001293798. | 000004.12 | | | TCCCAACC | | GATCTCCC | | | | |
| | | | | 1 | | | | TGCCC | | AACCTGCC | | | | |
| | | | | | | | | | | CCGGCGC | | | | |
| DUX4 | IL2 | IL2 low | 1E+08 | NM_ | NC_ | 1.9E+08 | sense | TCCAGGCA | 1100. | GCTTTCCA | 1288. | AGG | 1 | 0.556 |
| | | | | 001293798. | 000004.12 | | | TCGCCGCC | | GGCATCGC | | | | |
| | | | | 1 | | | | CGGG | | CGCCCGG | | | | |
| | | | | | | | | | | GAGGAGC | | | | |

TABLE 7
Nuclear factors that can be inhibited to increase IL2RA
expression or overexpressed to decrease IL2RA expression

| | | | | | | Position | | | | | | | | |
|---|---|---|---|---|---|---|---|---|---|---|---|---|---|---|
| | | | Target | | | of Base | | sgRNA | SEQ | Target | SEQ | | | Rule |
| gene_ | screen_ | screen_ | Gene | Target | Genomic | After Cut | | Target | ID | Context | ID | PAM | Exon | Set 2 |
| id | target | direction | ID | Transcript | Sequence | (1-based) | Strand | Sequence | NO: | Sequence | NO: | Sequence | Number | score |
| CTCF | IL2RA | IL2RA high | 10664 | NM_006565.3 | NC_000016.10 | 67612001 | antisense | CGATCCAA | 1289. | GTGACGAT | 1381. | TGG | 4 | 0.6364 |
| | | | | | | | | ATTTGAAC | | CCAAATTT | | | | |
| | | | | | | | | GCCG | | GAACGCC | | | | |
| | | | | | | | | | | GTGGACA | | | | |
| CTCF | IL2RA | IL2RA high | 10664 | NM_006565.3 | NC_000016.10 | 67611476 | sense | GAGCAAA | 1290. | AAAAGAG | 1382. | AGG | 3 | 0.6793 |
| | | | | | | | | CTGCGTTA | | CAAACTGC | | | | |
| | | | | | | | | TACAG | | GTTATACA | | | | |
| | | | | | | | | | | GAGGAGG | | | | |
| CTCF | IL2RA | IL2RA high | 10664 | NM_006565.3 | NC_000016.10 | 67610967 | sense | TTACCCCA | 1291. | CCACTTAC | 1383. | TGG | 3 | 0.6378 |
| | | | | | | | | GAACCAG | | CCCAGAAC | | | | |
| | | | | | | | | ACGGA | | CAGACGG | | | | |
| | | | | | | | | | | ATGGGGG | | | | |
| CTCF | IL2RA | IL2RA high | 10664 | NM_006565.3 | NC_000016.10 | 67620773 | sense | TTTGTGCA | 1292. | GCAGTTTG | 1384. | GGG | 6 | 0.6444 |
| | | | | | | | | GTTATGCC | | TGCAGTTA | | | | |
| | | | | | | | | AGCA | | TGCCAGCA | | | | |
| | | | | | | | | | | GGGACA | | | | |
| PTPRC | IL2RA | IL2RA high | 5788 | NM_002838.4 | NC_000001.11 | 1.99E+08 | antisense | AGCATTAT | 1293. | CACTAGCA | 1385. | GGG | 4 | 0.6692 |
| | | | | | | | | CCAAAGA | | TTATCCAA | | | | |
| | | | | | | | | GTCCG | | AGAGTCC | | | | |
| | | | | | | | | | | GGGGATA | | | | |
| PTPRC | IL2RA | IL2RA high | 5788 | NM_002838.4 | NC_000001.11 | 1.99E+08 | antisense | GGAAACTT | 1294. | TATAGGAA | 1386. | CGG | 19 | 0.6482 |
| | | | | | | | | GCTGAACA | | ACTTGCTG | | | | |
| | | | | | | | | CCCG | | AACACCCG | | | | |
| | | | | | | | | | | CGGGAT | | | | |
| PTPRC | IL2RA | IL2RA high | 5788 | NM_002838.4 | NC_000001.11 | 1.99E+08 | antisense | TCCAAATG | 1295. | AACTTCCA | 1387. | GGG | 14 | 0.6167 |
| | | | | | | | | GTAACGTT | | AATGGTAA | | | | |
| | | | | | | | | CATG | | CGTTCATG | | | | |
| | | | | | | | | | | GGGGCC | | | | |
| PTPRC | IL2RA | IL2RA high | 5788 | NM_002838.4 | NC_000001.11 | 1.99E+08 | sense | TGTGGATT | 1296. | TCACTGTG | 1388. | AGG | 9 | 0.6937 |
| | | | | | | | | ACTTATAT | | GATTACTT | | | | |
| | | | | | | | | AACA | | ATATAACA | | | | |
| | | | | | | | | | | AGGAAA | | | | |
| NR2C2 | IL2RA | IL2RA high | 7182 | NM_003298.4 | NC_000003.12 | 15030406 | sense | CCAGTCGA | 1297. | GAGACCA | 1389. | AGG | 10 | 0.6976 |
| | | | | | | | | CACCCATC | | GTCGACAC | | | | |
| | | | | | | | | ATTG | | CCATCATT | | | | |
| | | | | | | | | | | GAGGTTG | | | | |
| NR2C2 | IL2RA | IL2RA high | 7182 | NM_003298.4 | NC_000003.12 | 15016174 | antisense | CCCCAGTA | 1298. | TCTTCCCC | 1390. | AGG | 5 | 0.7308 |
| | | | | | | | | AACGCTCC | | AGTAAAC | | | | |
| | | | | | | | | ACAG | | GCTCCACA | | | | |
| | | | | | | | | | | GAGGCAG | | | | |
| NR2C2 | IL2RA | IL2RA high | 7182 | NM_003298.4 | NC_000003.12 | 15024201 | antisense | GAACGTCA | 1299. | TAGAGAA | 1391. | TGG | 8 | 0.6168 |
| | | | | | | | | CCTTAGAA | | CGTCACCT | | | | |
| | | | | | | | | TCCG | | TAGAATCC | | | | |
| | | | | | | | | | | GTGGCCA | | | | |
| NR2C2 | IL2RA | IL2RA high | 7182 | NM_003298.4 | NC_000003.12 | 15023322 | antisense | TCTTTGTC | 1300. | TCCATCTT | 1392 | GGG | 7 | 0.6618 |
| | | | | | | | | TGCCACAA | | TGTCTGCC | | | | |
| | | | | | | | | ACGT | | ACAAACGT | | | | |
| | | | | | | | | | | GGGAGT | | | | |
| TP53 | IL2RA | IL2RA high | 7157 | NM_000546.5 | NC_000017.11 | 7676227 | antisense | CCATTGTT | 1301. | TGAACCAT | 1393. | GGG | 4 | 0.6899 |
| | | | | | | | | CAATATCG | | TGTTCAAT | | | | |
| | | | | | | | | TCCG | | ATCGTCCG | | | | |
| | | | | | | | | | | GGGACA | | | | |
| TP53 | IL2RA | IL2RA high | 7157 | NM_000546.5 | NC_000017.11 | 7675058 | sense | GAGCGCT | 1302. | CCATGAGC | 1394. | TGG | 5 | 0.6542 |
| | | | | | | | | GCTCAGAT | | GCTGCTCA | | | | |
| | | | | | | | | AGCGA | | GATAGCG | | | | |
| | | | | | | | | | | ATGGTGA | | | | |
| TP53 | IL2RA | IL2RA high | 7157 | NM_000546.5 | NC_000017.11 | 7676527 | antisense | GATCCACT | 1303. | AATGGATC | 1395. | AGG | 2 | 0.6108 |
| | | | | | | | | CACAGTTT | | CACTCACA | | | | |
| | | | | | | | | CCAT | | GTTTCCAT | | | | |
| | | | | | | | | | | AGGTCT | | | | |
| TP53 | IL2RA | IL2RA high | 7157 | NM_000546.5 | NC_000017.11 | 7674864 | sense | GGTGCCCT | 1304. | TGGTGGT | 1396. | AGG | 6 | 0.6259 |
| | | | | | | | | ATGAGCC | | GCCCTATG | | | | |
| | | | | | | | | GCCTG | | AGCCGCCT | | | | |
| | | | | | | | | | | GAGGTCT | | | | |
| ATXN7L3 | IL2RA | IL2RA high | 56970 | NM_001098833.1 | NC_000017.11 | 44197610 | sense | CACGGACC | 1305. | ACGACAC | 1397. | AGG | 2 | 0.6328 |
| | | | | | | | | CTGATAGC | | GGACCCTG | | | | |
| | | | | | | | | ATGA | | ATAGCATG | | | | |
| | | | | | | | | | | AAGGATT | | | | |
| ATXN7L3 | IL2RA | IL2RA high | 56970 | NM_001098833.1 | NC_000017.11 | 44197712 | sense | CATCGCTC | 1306. | AGGCCATC | 1398. | CGG | 2 | 0.7491 |
| | | | | | | | | AGGAGAT | | GCTCAGG | | | | |
| | | | | | | | | ATACG | | AGATATAC | | | | |
| | | | | | | | | | | GCGGACC | | | | |
| ATXN7L3 | IL2RA | IL2RA high | 56970 | NM_001098833.1 | NC_000017.11 | 44197233 | sense | GCAGCCG | 1307. | AACAGCA | 1399. | CGG | 3 | 0.6135 |
| | | | | | | | | AATCGCCA | | GCCGAATC | | | | |
| | | | | | | | | ACCGC | | GCCAACCG | | | | |
| | | | | | | | | | | CCGGTGA | | | | |
| ATXN7L3 | IL2RA | IL2RA high | 56970 | NM_001098833.1 | NC_000017.11 | 44195424 | sense | GCTTCGCA | 1308. | AGGAGCTT | 1400. | CGG | 8 | 0.6566 |
| | | | | | | | | GCCTGCTA | | CGCAGCCT | | | | |
| | | | | | | | | ACCA | | GCTAACCA | | | | |
| | | | | | | | | | | CGGTGA | | | | |
| TFDP1 | IL2RA | IL2RA high | 7027 | NM_007111.4 | NC_000013.11 | 1.14E+08 | sense | ACCGGCA | 1309. | AGAGACC | 1401. | TGG | 5 | 0.6315 |
| | | | | | | | | GCGTCAAA | | GGCAGCG | | | | |
| | | | | | | | | CACCC | | TCAAACAC | | | | |
| | | | | | | | | | | CCTGGTG | | | | |
| | | | | | | | | | | G | | | | |
| TFDP1 | IL2RA | IL2RA high | 7027 | NM_007111.4 | NC_000013.11 | 1.14E+08 | sense | ATGACCAG | 1310. | GCTTATGA | 1402. | CGG | 7 | 0.6204 |
| | | | | | | | | AAAAACAT | | CCAGAAA | | | | |
| | | | | | | | | AAGA | | AACATAAG | | | | |
| | | | | | | | | | | ACGGCGC | | | | |
| TFDP1 | IL2RA | IL2RA high | 7027 | NM_007111.4 | NC_000013.11 | 1.14E+08 | antisense | CCTTCATG | 1311. | CAGACCTT | 1403. | AGG | 6 | 0.6851 |
| | | | | | | | | GAGAAAT | | CATGGAG | | | | |
| | | | | | | | | GCCGT | | AAATGCCG | | | | |
| | | | | | | | | | | TAGGCCC | | | | |
| TFDP1 | IL2RA | IL2RA high | 7027 | NM_007111.4 | NC_000013.11 | 1.14E+08 | sense | GGTGCAG | 1312. | ACCTGGTG | 1404. | CGG | 9 | 0.6402 |
| | | | | | | | | AGAAACC | | CAGAGAA | | | | |
| | | | | | | | | GGCATG | | ACCGGCAT | | | | |
| | | | | | | | | | | GCGGAGC | | | | |
| HNRNPK | IL2RA | IL2RA high | 3190 | NM_002140.3 | NC_000009.12 | 83972098 | sense | ATGATGTT | 1313. | TACAATGA | 1405. | CGG | 11 | 0.7754 |
| | | | | | | | | TGATGACC | | TGTTTGAT | | | | |
| | | | | | | | | GTCG | | GACCGTCG | | | | |
| | | | | | | | | | | CGGACG | | | | |
| HNRNPK | IL2RA | IL2RA high | 3190 | NM_002140.3 | NC_000009.12 | 83975465 | antisense | CTGTTGGG | 1314. | TAAACTGT | 1406. | GGG | 6 | 0.6273 |
| | | | | | | | | ACATACCG | | TGGGACAT | | | | |
| | | | | | | | | CTCG | | ACCGCTCG | | | | |
| | | | | | | | | | | GGGCCA | | | | |
| HNRNPK | IL2RA | IL2RA high | 3190 | NM_002140.3 | NC_000009.12 | 83971978 | sense | GATGATAT | 1315. | TTATGATG | 1407. | AGG | 11 | 0.7149 |
| | | | | | | | | GAGCCCTC | | ATATGAGC | | | | |
| | | | | | | | | GTCG | | CCTCGTCG | | | | |
| | | | | | | | | | | AGGACC | | | | |
| HNRNPK | IL2RA | IL2RA high | 3190 | NM_002140.3 | NC_000009.12 | 83973291 | sense | TAAAATCA | 1316. | GTGCTAAA | 1408. | AGG | 9 | 0.6608 |
| | | | | | | | | AAGAACTT | | ATCAAAGA | | | | |
| | | | | | | | | CGAG | | ACTTCGAG | | | | |
| | | | | | | | | | | AGGTAA | | | | |
| NFKB2 | IL2RA | IL2RA high | 4791 | NM_001077494.3 | NC_000010.11 | 1.02E+08 | sense | ACTCGACT | 1317. | CCCTACTC | 1409. | CGG | 14 | 0.7089 |
| | | | | | | | | ACGGCGTC | | GACTACG | | | | |
| | | | | | | | | ACCG | | GCGTCACC | | | | |
| | | | | | | | | | | GCGGACG | | | | |
| NFKB2 | IL2RA | IL2RA high | 4791 | NM_001077494.3 | NC_000010.11 | 1.02E+08 | sense | CCCACTCC | 1318. | ATATCCCA | 1410. | GGG | 9 | 0.6913 |
| | | | | | | | | ATAGAATC | | CTCCATAG | | | | |
| | | | | | | | | TCCG | | AATCTCCG | | | | |
| | | | | | | | | | | GGGGCA | | | | |
| NFKB2 | IL2RA | IL2RA high | 4791 | NM_001077494.3 | NC_000010.11 | 1.02E+08 | sense | CTGCAACT | 1319. | GTTTCTGC | 1411. | AGG | 11 | 0.6401 |
| | | | | | | | | GAAACGC | | AACTGAAA | | | | |
| | | | | | | | | AAGCG | | CGCAAGC | | | | |
| | | | | | | | | | | GAGGAGG | | | | |
| NFKB2 | IL2RA | IL2RA high | 4791 | NM_001077494.3 | NC_000010.11 | 1.02E+08 | sense | GGGACCA | 1320. | ACGAGGG | 1412. | TGG | 6 | 0.7003 |
| | | | | | | | | GCCAAGAT | | ACCAGCCA | | | | |
| | | | | | | | | CGAGG | | AGATCGA | | | | |
| | | | | | | | | | | GGTGGAC | | | | |
| | | | | | | | | | | C | | | | |
| PRDM1 | IL2RA | IL2RA high | 639 | NM_001198.3 | NC_000006.12 | 1.06E+08 | sense | AGGATGC | 1321. | ATGGAGG | 1413. | TGG | 2 | 0.7731 |
| | | | | | | | | GGATATG | | ATGCGGAT | | | | |
| | | | | | | | | ACTCTG | | ATGACTCT | | | | |
| | | | | | | | | | | GTGGACA | | | | |
| PRDM1 | IL2RA | IL2RA high | 639 | NM_001198.3 | NC_000006.12 | 1.06E+08 | antisense | GGACGCG | 1322. | CGTAGGA | 1414. | AGG | 5 | 0.6665 |
| | | | | | | | | TTCAAGTA | | CGCGTTCA | | | | |
| | | | | | | | | AGCGT | | AGTAAGC | | | | |
| | | | | | | | | | | GTAGGAG | | | | |
| | | | | | | | | | | T | | | | |
| PRDM1 | IL2RA | IL2RA high | 639 | NM_001198.3 | NC_000006.12 | 1.06E+08 | antisense | GGGGAGC | 1323. | AATGGGG | 1415. | GGG | 5 | 0.6871 |
| | | | | | | | | GAGTGAT | | GAGCGAG | | | | |
| | | | | | | | | GTACGT | | TGATGTAC | | | | |
| | | | | | | | | | | GTGGGTCT | | | | |
| PRDM1 | IL2RA | IL2RA high | 639 | NM_001198.3 | NC_000006.12 | 1.06E+08 | sense | TTTGGACA | 1324. | CTCTTTTG | 1416. | AGG | 4 | 0.6708 |
| | | | | | | | | GATCTATT | | GACAGATC | | | | |
| | | | | | | | | CCAG | | TATTCCAG | | | | |
| | | | | | | | | | | AGGGGA | | | | |
| MYC | IL2RA | IL2RA high | 4609 | NM_002467.4 | NC_000008.11 | 1.28E+08 | sense | AGAGTGC | 1325. | CCTCAGAG | 1417. | TGG | 2 | 0.6543 |
| | | | | | | | | ATCGACCC | | TGCATCGA | | | | |
| | | | | | | | | CTCGG | | CCCCTCGG | | | | |
| | | | | | | | | | | TGGTCT | | | | |
| MYC | IL2RA | IL2RA high | 4609 | NM_002467.4 | NC_000008.11 | 1.28E+08 | antisense | CTGCGGG | 1326. | TGCCCTGC | 1418. | AGG | 2 | 0.6832 |
| | | | | | | | | GAGGACT | | GGGGAGG | | | | |
| | | | | | | | | CCGTCG | | ACTCCGTC | | | | |
| | | | | | | | | | | GAGGAGA | | | | |
| MYC | IL2RA | IL2RA high | 4609 | NM_002467.4 | NC_000008.11 | 1.28E+08 | sense | CTTCGGG | 1327. | CTCCCTTC | 1419. | CGG | 2 | 0.6919 |
| | | | | | | | | GAGACAA | | GGGGAGA | | | | |
| | | | | | | | | CGACGG | | CAACGAC | | | | |
| | | | | | | | | | | GGCGGTG | | | | |
| | | | | | | | | | | G | | | | |
| MYC | IL2RA | IL2RA high | 4609 | NM_002467.4 | NC_000008.11 | 1.28E+08 | antisense | GCTGCACC | 1328. | TACGGCTG | 1420. | AGG | 2 | 0.6842 |
| | | | | | | | | GAGTCGTA | | CACCGAGT | | | | |
| | | | | | | | | GTCG | | CGTAGTCG | | | | |
| | | | | | | | | | | AGGTCA | | | | |
| SMARCB1 | IL2RA | IL2RA high | 6598 | NM_003073.3 | NC_000022.11 | 23791773 | antisense | GAGAACCT | 1329. | TACAGAG | 1421. | AGG | 2 | 0.731 |
| | | | | | | | | CGGAACAT | | AACCTCGG | | | | |
| | | | | | | | | ACGG | | AACATACG | | | | |
| | | | | | | | | | | GAGGTAG | | | | |
| SMARCB1 | IL2RA | IL2RA high | 6598 | NM_003073.3 | NC_000022.11 | 23816887 | sense | GCAGATC | 1330. | GACAGCA | 1422. | CGG | 6 | 0.6894 |
| | | | | | | | | GAGTCCTA | | GATCGAGT | | | | |
| | | | | | | | | CCCCA | | CCTACCCC | | | | |
| | | | | | | | | | | ACGGACA | | | | |
| SMARCB1 | IL2RA | IL2RA high | 6598 | NM_003073.3 | NC_000022.11 | 23801049 | antisense | TCTTCTTG | 1331. | GTTCTCTT | 1423. | CGG | 4 | 0.6806 |
| | | | | | | | | TCTCGGCC | | CTTGTCTC | | | | |
| | | | | | | | | CATG | | GGCCCATG | | | | |
| | | | | | | | | | | CGGTTC | | | | |
| SMARCB1 | IL2RA | IL2RA high | 6598 | NM_003073.3 | NC_000022.11 | 23803342 | sense | TGAGAAC | 1332. | TCCATGAG | 1424. | AGG | 5 | 0.6804 |
| | | | | | | | | GCATCTCA | | AACGCATC | | | | |
| | | | | | | | | GCCCG | | TCAGCCCG | | | | |
| | | | | | | | | | | AGGTGC | | | | |
| KLF13 | IL2RA | IL2RA high | 51621 | NM_015995.3 | NC_000015.10 | 31327702 | sense | CCGACCTC | 1333. | CGCGCCG | 1425. | AGG | 1 | 0.7221 |
| | | | | | | | | GAGTCCCC | | ACCTCGAG | | | | |
| | | | | | | | | GCAG | | TCCCCGCA | | | | |
| | | | | | | | | | | GAGGAAG | | | | |
| KLF13 | IL2RA | IL2RA high | 51621 | NM_015995.3 | NC_000015.10 | 31327415 | sense | CGTGGTG | 1334. | TCTTCGTG | 1426. | CGG | 1 | 0.6798 |
| | | | | | | | | GCGCGGA | | GTGGCGC | | | | |
| | | | | | | | | TCCTAG | | GGATCCTA | | | | |
| | | | | | | | | | | GCGGACC | | | | |
| KLF13 | IL2RA | IL2RA high | 51621 | NM_015995.3 | NC_000015.10 | 31327274 | sense | CGTGTCCA | 1335. | GCCTCGTG | 1427 | CGG | 1 | 0.7 |
| | | | | | | | | TGTCGAGC | | TCCATGTC | | | | |
| | | | | | | | | CGCG | | GAGCCGC | | | | |
| | | | | | | | | | | GCGGTCG | | | | |
| KLF13 | IL2RA | IL2RA high | 51621 | NM_015995.3 | NC_000015.10 | 31327767 | antisense | GAGTTCTC | 1336. | GTGTGAG | 1428. | AGG | 1 | 0.592 |
| | | | | | | | | AGGTGCG | | TTCTCAGG | | | | |
| | | | | | | | | CCTTG | | TGCGCCTT | | | | |
| | | | | | | | | | | GAGGTGC | | | | |
| IRF1 | IL2RA | IL2RA high | 3659 | NM_002198.2 | NC_000005.10 | 1.32E+08 | sense | GAACTCCC | 1337. | CCATGAAC | 1429. | AGG | 4 | 0.6297 |
| | | | | | | | | TGCCAGAT | | TCCCTGCC | | | | |
| | | | | | | | | ATCG | | AGATATCG | | | | |
| | | | | | | | | | | AGGAGG | | | | |
| IRF1 | IL2RA | IL2RA high | 3659 | NM_002198.2 | NC_000005.10 | 1.32E+08 | sense | TCTAGGCC | 1338. | GCCCTCTA | 1430. | GGG | 4 | 0.718 |
| | | | | | | | | GATACAAA | | GGCCGAT | | | | |
| | | | | | | | | GCAG | | ACAAAGC | | | | |
| | | | | | | | | | | AGGGGAA | | | | |
| | | | | | | | | | | A | | | | |
| IRF1 | IL2RA | IL2RA high | 3659 | NM_002198.2 | NC_000005.10 | 1.32E+08 | sense | TCTCCCTC | 1339. | GCTTTCTC | 1431. | GGG | 6 | 0.6137 |
| | | | | | | | | GACAGTCA | | CCTCGACA | | | | |
| | | | | | | | | TGTG | | GTCATGTG | | | | |
| | | | | | | | | | | GGGATT | | | | |
| IRF1 | IL2RA | IL2RA high | 3659 | NM_002198.2 | NC_000005.10 | 1.32E+08 | sense | TTAATTCC | 1340. | CAGATTAA | 1432. | GGG | 2 | 0.7113 |
| | | | | | | | | AACCAAAT | | TTCCAACC | | | | |
| | | | | | | | | CCCG | | AAATCCCG | | | | |
| | | | | | | | | | | GGGCTC | | | | |
| FOXO1 | IL2RA | IL2RA high | 2308 | NM_002015.3 | NC_000013.11 | 40665740 | antisense | ACAGGTTG | 1341. | TAGGACA | 1433. | CGG | 1 | 0.547 |
| | | | | | | | | CCCCACGC | | GGTTGCCC | | | | |
| | | | | | | | | GTTG | | CACGCGTT | | | | |
| | | | | | | | | | | GCGGCGG | | | | |
| FOXO1 | IL2RA | IL2RA high | 2308 | NM_002015.3 | NC_000013.11 | 40666107 | sense | GGAGTTTA | 1342. | GGCCGGA | 1434. | CGG | 1 | 0.629 |
| | | | | | | | | GCCAGTCC | | GTTTAGCC | | | | |
| | | | | | | | | AACT | | AGTCCAAC | | | | |
| | | | | | | | | | | TCGGCCA | | | | |
| FOXO1 | IL2RA | IL2RA high | 2308 | NM_002015.3 | NC_000013.11 | 40560279 | antisense | GGTGGCG | 1343. | GTTTGGTG | 1435. | CGG | 2 | 0.6741 |
| | | | | | | | | CAAACGA | | GCGCAAA | | | | |
| | | | | | | | | GTAGCA | | CGAGTAG | | | | |
| | | | | | | | | | | CACGGCGT | | | | |
| FOXO1 | IL2RA | IL2RA high | 2308 | NM_002015.3 | NC_000013.11 | 40560544 | antisense | TAGCATTT | 1344. | GTACTAGC | 1436. | GGG | 2 | 0.6665 |
| | | | | | | | | GAGCTAGT | | ATTTGAGC | | | | |
| | | | | | | | | TCGA | | TAGTTCGA | | | | |
| | | | | | | | | | | GGGCGA | | | | |
| IRF2 | IL2RA | IL2RA high | 3660 | NM_002199.3 | NC_000004.12 | 1.84E+08 | sense | ACCTGATC | 1345. | ATAAACCT | 1437. | AGG | 4 | 0.6049 |
| | | | | | | | | CCAAAACA | | GATCCCAA | | | | |
| | | | | | | | | TGGA | | AACATGG | | | | |
| | | | | | | | | | | AAGGCGA | | | | |
| IRF2 | IL2RA | IL2RA high | 3660 | NM_002199.3 | NC_000004.12 | 1.84E+08 | antisense | CAGCATTC | 1346. | GGGGCAG | 1438. | AGG | 4 | 0.6028 |
| | | | | | | | | GGTAGAC | | CATTCGGT | | | | |
| | | | | | | | | CCTGA | | AGACCCTG | | | | |
| | | | | | | | | | | AAGGCAT | | | | |
| IRF2 | IL2RA | IL2RA high | 3660 | NM_002199.3 | NC_000004.12 | 1.84E+08 | sense | GGATGCAT | 1347. | CCCTGGAT | 1439. | GGG | 3 | 0.6231 |
| | | | | | | | | GCGGCTA | | GCATGCG | | | | |
| | | | | | | | | GACAT | | GCTAGACA | | | | |
| | | | | | | | | | | TGGGTGG | | | | |
| IRF2 | IL2RA | IL2RA high | 3660 | NM_002199.3 | NC_000004.12 | 1.84E+08 | antisense | TACCTGCA | 1348. | CGCTTACC | 1440. | GGG | 7 | 0.7207 |
| | | | | | | | | TAGGAAG | | TGCATAGG | | | | |
| | | | | | | | | ACACG | | AAGACAC | | | | |
| | | | | | | | | | | GGGGGAG | | | | |
| KLF2 | IL2RA | IL2RA high | 10365 | NM_016270.2 | NC_000019.10 | 16325729 | antisense | AAACCAG | 1349. | GCCGAAA | 1441. | CGG | 2 | 0.5997 |
| | | | | | | | | GGCCACC | | CCAGGGC | | | | |
| | | | | | | | | GAAAGG | | CACCGAAA | | | | |
| | | | | | | | | | | GGCGGCG | | | | |
| | | | | | | | | | | G | | | | |
| KLF2 | IL2RA | IL2RA high | 10365 | NM_016270.2 | NC_000019.10 | 16325576 | antisense | CCCTCGCG | 1350. | GGCGCCCT | 1442. | CGG | 2 | 0.5868 |
| | | | | | | | | CTTGAGGC | | CGCGCTTG | | | | |
| | | | | | | | | CGCG | | AGGCCGC | | | | |
| | | | | | | | | | | GCGGTCC | | | | |
| KLF2 | IL2RA | IL2RA high | 10365 | NM_016270.2 | NC_000019.10 | 16325811 | sense | CTTCGGTC | 1351. | CAGCCTTC | 1443. | CGG | 2 | 0.7252 |
| | | | | | | | | TCTTCGAC | | GGTCTCTT | | | | |
| | | | | | | | | GACG | | CGACGAC | | | | |
| | | | | | | | | | | GCGGCCG | | | | |
| KLF2 | IL2RA | IL2RA high | 10365 | NM_016270.2 | NC_000019.10 | 16325354 | antisense | TCGGGGT | 1352. | GGGTTCG | 1444. | CGG | 2 | 0.6967 |
| | | | | | | | | AATAGAAC | | GGGTAAT | | | | |
| | | | | | | | | GCAGG | | AGAACGC | | | | |
| | | | | | | | | | | AGGCGGC | | | | |
| | | | | | | | | | | GG | | | | |
| ZNF217 | IL2RA | IL2RA high | 7764 | NM_006526.2 | NC_000020.11 | 53581993 | sense | CAAAATCT | 1353. | AGACCAA | 1445. | GGG | 1 | 0.6608 |
| | | | | | | | | CACCCTGA | | AATCTCAC | | | | |
| | | | | | | | | AACG | | CCTGAAAC | | | | |
| | | | | | | | | | | GGGGAAG | | | | |
| ZNF217 | IL2RA | IL2RA high | 7764 | NM_006526.2 | NC_000020.11 | 53581749 | sense | CCACGGC | 1354. | ACTCCCAC | 1446. | TGG | 1 | 0.6177 |
| | | | | | | | | GAAGCGC | | GGCGAAG | | | | |
| | | | | | | | | CCTCCG | | CGCCCTCC | | | | |
| | | | | | | | | | | GTGGACG | | | | |
| ZNF217 | IL2RA | IL2RA high | 7764 | NM_006526.2 | NC_000020.11 | 53582284 | sense | GGACACAT | 1355. | ATGCGGA | 1447. | GGG | 1 | 0.7154 |
| | | | | | | | | AATGGCA | | CACATAAT | | | | |
| | | | | | | | | AATCG | | GGCAAATC | | | | |
| | | | | | | | | | | GGGGGCC | | | | |
| ZNF217 | IL2RA | IL2RA high | 7764 | NM_006526.2 | NC_000020.11 | 53576811 | antisense | TGGGTGG | 1356. | GTTATGG | 1448. | AGG | 3 | 0.6707 |
| | | | | | | | | TACTGCCA | | GTGGTACT | | | | |
| | | | | | | | | TCCGG | | GCCATCCG | | | | |
| | | | | | | | | | | GAGGAGG | | | | |
| TNFAIP3 | IL2RA | IL2RA high | 7128 | NM_001270507.1 | NC_000006.12 | 1.38E+08 | sense | CCACTTGT | 1357. | TGTTCCAC | 1449. | GGG | 6 | 0.6901 |
| | | | | | | | | TAACAGA | | TTGTTAAC | | | | |
| | | | | | | | | GACCG | | AGAGACC | | | | |
| | | | | | | | | | | GGGGAAG | | | | |
| TNFAIP3 | IL2RA | IL2RA high | 7128 | NM_001270507.1 | NC_000006.12 | 1.38E+08 | sense | CTTGTGGC | 1358. | GAAGCTTG | 1450. | CGG | 2 | 0.6709 |
| | | | | | | | | GCTGAAA | | TGGCGCT | | | | |
| | | | | | | | | ACGAA | | GAAAACG | | | | |
| | | | | | | | | | | AACGGTA | | | | |
| | | | | | | | | | | A | | | | |
| TNFAIP3 | IL2RA | IL2RA high | 7128 | NM_001270507.1 | NC_000006.12 | 1.38E+08 | sense | TATGCCAT | 1359. | CCTTTATG | 1451. | AGG | 7 | 0.7533 |
| | | | | | | | | GAGTGCTC | | CCATGAGT | | | | |
| | | | | | | | | AGAG | | GCTCAGA | | | | |
| | | | | | | | | | | GAGGCGG | | | | |
| TNFAIP3 | IL2RA | IL2RA high | 7128 | NM_001270507.1 | NC_000006.12 | 1.38E+08 | antisense | TGAGAGA | 1360. | GATTTGAG | 1452. | CGG | 3 | 0.6393 |
| | | | | | | | | CTCCAGTT | | AGACTCCA | | | | |
| | | | | | | | | GCCAG | | GTTGCCAG | | | | |
| | | | | | | | | | | CGGAAT | | | | |
| FOXK1 | IL2RA | IL2RA high | 221937 | NM_001037165.1 | NC_000007.14 | 4757132 | sense | AACAGGC | 1361. | GTGGAAC | 1453. | AGG | 5 | 0.6681 |
| | | | | | | | | ATTCCGGA | | AGGCATTC | | | | |
| | | | | | | | | AACGG | | CGGAAAC | | | | |
| | | | | | | | | | | GGAGGCA | | | | |
| | | | | | | | | | | G | | | | |
| FOXK1 | IL2RA | IL2RA high | 221937 | NM_001037165.1 | NC_000007.14 | 4754516 | antisense | AGGTCAC | 1362. | TCCGAGGT | 1454. | CGG | 3 | 0.734 |
| | | | | | | | | GTTCTGCA | | CACGTTCT | | | | |
| | | | | | | | | CAAAG | | GCACAAA | | | | |
| | | | | | | | | | | GCGGTAA | | | | |
| FOXK1 | IL2RA | IL2RA high | 221937 | NM_001037165.1 | NC_000007.14 | 4759162 | antisense | CTAACTTG | 1363. | GAAGCTA | 1455. | GGG | 6 | 0.6672 |
| | | | | | | | | GACCCAAA | | ACTTGGAC | | | | |
| | | | | | | | | CTCA | | CCAAACTC | | | | |
| | | | | | | | | | | AGGGTCG | | | | |
| FOXK1 | IL2RA | IL2RA high | 221937 | NM_001037165.1 | NC_000007.14 | 4755361 | sense | GCATTACC | 1364. | CCAAGCAT | 1456. | CGG | 4 | 0.7131 |
| | | | | | | | | CCTACTAC | | TACCCCTA | | | | |
| | | | | | | | | CGGA | | CTACCGGA | | | | |
| | | | | | | | | | | CGGCCG | | | | |
| MYB | IL2RA | IL2RA high | 4602 | NM_005375.2 | NC_000006.12 | 1.35E+08 | sense | ACCAGGC | 1365. | ATTTACCA | 1457. | GGG | 5 | 0.6859 |
| | | | | | | | | ACACAAG | | GGCACAC | | | | |
| | | | | | | | | AGACTG | | AAGAGAC | | | | |
| | | | | | | | | | | TGGGGAA | | | | |
| | | | | | | | | | | C | | | | |
| MYB | IL2RA | IL2RA high | 4602 | NM_005375.2 | NC_000006.12 | 1.35E+08 | sense | AGAAATAC | 1366. | GTACAGA | 1458. | TGG | 5 | 0.6009 |
| | | | | | | | | GGTCCGA | | AATACGGT | | | | |
| | | | | | | | | AACGT | | CCGAAAC | | | | |
| | | | | | | | | | | GTTGGTCT | | | | |
| MYB | IL2RA | IL2RA high | 4602 | NM_005375.2 | NC_000006.12 | 1.35E+08 | sense | AGTCTGGA | 1367. | CCCAAGTC | 1459. | GGG | 2 | 0.7063 |
| | | | | | | | | AAGCGTCA | | TGGAAAG | | | | |
| | | | | | | | | CTTG | | CGTCACTT | | | | |
| | | | | | | | | | | GGGGAAA | | | | |
| MYB | IL2RA | IL2RA high | 4602 | NM_005375.2 | NC_000006.12 | 1.35E+08 | antisense | TATTTACA | 1368. | ACTATATT | 1460 | GGG | 7 | 0.6157 |
| | | | | | | | | TGTAACGC | | TACATGTA | | | | |
| | | | | | | | | TACA | | ACGCTACA | | | | |
| | | | | | | | | | | GGGTAT | | | | |
| CBFB | IL2RA | IL2RA high | 865 | NM_001755.2 | NC_000016.10 | 67036720 | antisense | AAGTCGAC | 1369. | TTCTAAGT | 1461. | AGG | 3 | 0.5993 |
| | | | | | | | | ATACTCTC | | CGACATAC | | | | |
| | | | | | | | | GGCT | | TCTCGGCT | | | | |
| | | | | | | | | | | AGGTGT | | | | |
| CBFB | IL2RA | IL2RA high | 865 | NM_001755.2 | NC_000016.10 | 67029479 | antisense | CCTGCCTC | 1370. | CCCGCCTG | 1462. | CGG | 1 | 0.6743 |
| | | | | | | | | ACCTCACA | | CCTCACCT | | | | |
| | | | | | | | | CTCG | | CACACTCG | | | | |
| | | | | | | | | | | CGGCTC | | | | |
| CBFB | IL2RA | IL2RA high | 865 | NM_001755.2 | NC_000016.10 | 67029807 | antisense | GCCGACTT | 1371. | GCCAGCC | 1463. | CGG | 2 | 0.7383 |
| | | | | | | | | ACGATTTC | | GACTTACG | | | | |
| | | | | | | | | CGAG | | ATTTCCGA | | | | |
| | | | | | | | | | | GCGGCCG | | | | |
| CBFB | IL2RA | IL2RA high | 865 | NM_001755.2 | NC_000016.10 | 67066729 | sense | GGAGTCT | 1372. | GAATGGA | 1464. | AGG | 4 | 0.5938 |
| | | | | | | | | GTGTTATC | | GTCTGTGT | | | | |
| | | | | | | | | TGGAA | | TATCTGGA | | | | |
| | | | | | | | | | | AAGGCTG | | | | |
| HIVEP2 | IL2RA | IL2RA high | 3097 | NM_006734.3 | NC_000006.12 | 1.43E+08 | antisense | CCCTGGAT | 1373. | CGGTCCCT | 1465. | CGG | 5 | 0.6747 |
| | | | | | | | | AGAATACA | | GGATAGA | | | | |
| | | | | | | | | TCGT | | ATACATCG | | | | |
| | | | | | | | | | | TCGGAAC | | | | |
| HIVEP2 | IL2RA | IL2RA high | 3097 | NM_006734.3 | NC_000006.12 | 1.43E+08 | sense | GACAAGA | 1374. | TTCAGACA | 1466. | GGG | 5 | 0.7382 |
| | | | | | | | | TGTCAGAC | | AGATGTCA | | | | |
| | | | | | | | | CTAGG | | GACCTAG | | | | |
| | | | | | | | | | | GGGGCAG | | | | |
| HIVEP2 | IL2RA | IL2RA high | 3097 | NM_006734.3 | NC_000006.12 | 1.43E+08 | antisense | GAGGTGG | 1375. | GTCAGAG | 1467. | GGG | 5 | 0.6922 |
| | | | | | | | | AAGGTAA | | GTGGAAG | | | | |
| | | | | | | | | ACACAA | | GTAAACAC | | | | |
| | | | | | | | | | | AAGGGGA | | | | |
| | | | | | | | | | | T | | | | |
| HIVEP2 | IL2RA | IL2RA high | 3097 | NM_006734.3 | NC_000006.12 | 1.43E+08 | antisense | TTCTAGGA | 1376. | TTTTTTCTA | 1468. | TGG | 5 | 0.71 |
| | | | | | | | | TAACCACC | | GGATAACC | | | | |
| | | | | | | | | ACTG | | ACCACTGT | | | | |
| | | | | | | | | | | GGCCA | | | | |
| MED12 | IL2RA | IL2RA high | 9968 | NM_005120.2 | NC_000023.11 | 71130165 | sense | ACATCGAC | 1377. | ATCCACAT | 1469. | AGG | 28 | 0.6457 |
| | | | | | | | | TGCTGGAC | | CGACTGCT | | | | |
| | | | | | | | | AATG | | GGACAAT | | | | |
| | | | | | | | | | | GAGGATG | | | | |
| MED12 | IL2RA | IL2RA high | 9968 | NM_005120.2 | NC_000023.11 | 71122231 | antisense | CAGTGAGT | 1378. | CAGTCAGT | 1470. | AGG | 8 | 0.688 |
| | | | | | | | | AGTGCCAA | | GAGTAGT | | | | |
| | | | | | | | | ACCA | | GCCAAACC | | | | |
| | | | | | | | | | | AAGGCAC | | | | |
| MED12 | IL2RA | IL2RA high | 9968 | NM_005120.2 | NC_000023.11 | 71125111 | antisense | GTGGCGT | 1379. | ATGGGTG | 1471. | TGG | 15 | 0.6661 |
| | | | | | | | | ACTGCACG | | GCGTACTG | | | | |
| | | | | | | | | TGTCG | | CACGTGTC | | | | |
| | | | | | | | | | | GTGGCTG | | | | |
| MED12 | IL2RA | IL2RA high | 9968 | NM_005120.2 | NC_000023.11 | 71126138 | sense | TTCACATT | 1380. | ACCTTTCA | 1472. | AGG | 18 | 0.6594 |
| | | | | | | | | ATGACCAA | | CATTATGA | | | | |
| | | | | | | | | CACC | | CCAACACC | | | | |
| | | | | | | | | | | AGGTCA | | | | |

TABLE 8
Nuclear factors that can be inhibited to decrease IL2RA expression or overexpressed to increase IL2RA expression

| | | | | | | Position of | | | | | | | |
|---|---|---|---|---|---|---|---|---|---|---|---|---|---|
| | | | Target | | | Base After | | sgRNA | SEQ | Target | SEQ | | |
| gene_ | screen_ | screen_ | Gene | Target | Genomic | Cut (1- | | Target | ID | Context | ID | PAM | Exon |
| id | target | direction | ID | Transcript | Sequence | based) | Strand | Sequence | NO: | Sequence | NO: | Sequence | Number |
| IL2RA | IL2RA | IL2RA low | 3559 | NM_000417.2 | NC_000010.11 | 6021582 | sense | GGATACAGG | 1473. | CCAGGGATACA | 1584 | AGG | 4 |
| | | | | | | | | GCTCTACAC | | GGGCTCTACAC | | | |
| | | | | | | | | AG | | AGAGGTCC | | | |
| IL2RA | IL2RA | IL2RA low | 3559 | NM_000417.2 | NC_000010.11 | 6025992 | antisense | TGGCTTTGA | 1474. | GCCATGGCTTT | 1585 | GGG | 2 |
| | | | | | | | | ATGTGGCGT | | GAATGTGGCGT | | | |
| | | | | | | | | GT | | GTGGGATC | | | |
| IL2RA | IL2RA | IL2RA low | 3559 | NM_000417.2 | NC_000010.11 | 6024349 | antisense | TTGTTTCGTT | 1475. | TCACTTGTTTCG | 1586 | TGG | 3 |
| | | | | | | | | GTGTTCCGA | | TTGTGTTCCGAG | | | |
| | | | | | | | | G | | TGGCTA | | | |
| STAT5B | IL2RA | IL2RA low | 6777 | NM_012448.3 | NC_000017.11 | 42216055 | sense | CAGCCAGGA | 1476. | ATGGCAGCCAG | 1587 | CGG | 12 |
| | | | | | | | | CAACAATGC | | GACAACAATGC | | | |
| | | | | | | | | GA | | GACGGCCA | | | |
| STAT5B | IL2RA | IL2RA low | 6777 | NM_012448.3 | NC_000017.11 | 42227658 | antisense | GTGGCCTTA | 1477. | CTGGGTGGCCT | 1588 | TGG | 3 |
| | | | | | | | | ATGTTCTCCT | | TAATGTTCTCCT | | | |
| | | | | | | | | G | | GTGGATT | | | |
| STAT5B | IL2RA | IL2RA low | 6777 | NM_012448.3 | NC_000017.11 | 42224822 | antisense | GTTCATTGT | 1478. | CTCTGTTCATTG | 1589 | CGG | 4 |
| | | | | | | | | ACAATATAT | | TACAATATATG | | | |
| | | | | | | | | GG | | GCGGATG | | | |
| STAT5B | IL2RA | IL2RA low | 6777 | NM_012448.3 | NC_000017.11 | 42217252 | sense | TAAGAGGTC | 1479. | GAATTAAGAGG | 1590 | GGG | 11 |
| | | | | | | | | AGACCGTCG | | TCAGACCGTCG | | | |
| | | | | | | | | TG | | TGGGGCAG | | | |
| FOXP1 | IL2RA | IL2RA low | 27086 | NM_032682.5 | NC_000003.12 | 71041428 | antisense | AGAGGAGG | 1480. | GTGCAGAGGAG | 1591 | TGG | 11 |
| | | | | | | | | AGACACATG | | GAGACACATGT | | | |
| | | | | | | | | TCG | | CGTGGTCA | | | |
| FOXP1 | IL2RA | IL2RA low | 27086 | NM_032682.5 | NC_000003.12 | 71015617 | antisense | CATACACCA | 1481. | CTTGCATACACC | 1592 | AGG | 12 |
| | | | | | | | | TGTCCATAG | | ATGTCCATAGA | | | |
| | | | | | | | | AG | | GAGGATG | | | |
| FOXP1 | IL2RA | IL2RA low | 27086 | NM_032682.5 | NC_000003.12 | 71046982 | sense | GCCTTCTGA | 1482. | CAAGGCCTTCT | 1593 | GGG | 10 |
| | | | | | | | | CAATTCAGC | | GACAATTCAGC | | | |
| | | | | | | | | CC | | CCGGGCAG | | | |
| FOXP1 | IL2RA | IL2RA low | 27086 | NM_032682.5 | NC_000003.12 | 70988031 | antisense | GTTCTGTAG | 1483. | TTGGGTTCTGTA | 1594 | AGG | 14 |
| | | | | | | | | ACTTCACAT | | GACTTCACATGC | | | |
| | | | | | | | | GC | | AGGTGG | | | |
| STAT5A | IL2RA | IL2RA low | 6776 | NM_003152.3 | NC_000017.11 | 42292035 | sense | ACATTCTGT | 1484. | CGGCACATTCT | 1595 | AGG | 5 |
| | | | | | | | | ACAATGAAC | | GTACAATGAAC | | | |
| | | | | | | | | AG | | AGAGGCTG | | | |
| STAT5A | IL2RA | IL2RA low | 6776 | NM_003152.3 | NC_000017.11 | 42304559 | sense | ATCAAGCGT | 1485. | GAGGATCAAGC | 1596 | GGG | 12 |
| | | | | | | | | GCTGACCGG | | GTGCTGACCGG | | | |
| | | | | | | | | CG | | CGGGGTGC | | | |
| STAT5A | IL2RA | IL2RA low | 6776 | NM_003152.3 | NC_000017.11 | 42305662 | sense | CAGCCAGGA | 1486. | ACGGCAGCCAG | 1597 | CGG | 13 |
| | | | | | | | | CCACAATGC | | GACCACAATGC | | | |
| | | | | | | | | CA | | CACGGCTA | | | |
| STAT5A | IL2RA | IL2RA low | 6776 | NM_003152.3 | NC_000017.11 | 42301385 | sense | CGTGCACAT | 1487. | TGAACGTGCAC | 1598 | AGG | 10 |
| | | | | | | | | GAATCCCCC | | ATGAATCCCCCC | | | |
| | | | | | | | | CC | | CAGGTGA | | | |
| GATA3 | IL2RA | IL2RA low | 2625 | NM_002051.2 | NC_000010.11 | 8055892 | sense | AGGTACCCT | 1488. | GCAGAGGTACC | 1599 | CGG | 2 |
| | | | | | | | | CCGACCCAC | | CTCCGACCCACC | | | |
| | | | | | | | | CA | | ACGGTGA | | | |
| GATA3 | IL2RA | IL2RA low | 2625 | NM_002051.2 | NC_000010.11 | 8064014 | sense | CAGGGAGT | 1489. | AAGGCAGGGA | 1600 | GGG | 4 |
| | | | | | | | | GTGTGAACT | | GTGTGTGAACT | | | |
| | | | | | | | | GTG | | GTGGGGCAA | | | |
| GATA3 | IL2RA | IL2RA low | 2625 | NM_002051.2 | NC_000010.11 | 8058740 | antisense | GGAGCTGTA | 1490. | GTCCGGAGCTG | 1601 | AGG | 3 |
| | | | | | | | | CTCGGGCAC | | TACTCGGGCAC | | | |
| | | | | | | | | GT | | GTAGGGCG | | | |
| GATA3 | IL2RA | IL2RA low | 2625 | NM_002051.2 | NC_000010.11 | 8058432 | sense | TCCAAGACG | 1491. | CTTCTCCAAGAC | 1602 | CGG | 3 |
| | | | | | | | | TCCATCCAC | | GTCCATCCACCA | | | |
| | | | | | | | | CA | | CGGCTC | | | |
| KMT2A | IL2RA | IL2RA low | 4297 | NM_005933.3 | NC_000011.10 | 1.19E+08 | antisense | AAGATCAGT | 1492. | ATTCAAGATCA | 1603 | TGG | 27 |
| | | | | | | | | AGCGGTCCC | | GTAGCGGTCCC | | | |
| | | | | | | | | GG | | GGTGGTGG | | | |
| KMT2A | IL2RA | IL2RA low | 4297 | NM_005933.3 | NC_000011.10 | 1.18E+08 | sense | AGAAAGGA | 1493. | GTAAAGAAAGG | 1604 | CGG | 5 |
| | | | | | | | | CGTCGATCG | | ACGTCGATCGA | | | |
| | | | | | | | | AGG | | GGCGGTGT | | | |
| KMT2A | IL2RA | IL2RA low | 4297 | NM_005933.3 | NC_000011.10 | 1.18E+08 | antisense | AGGGGTCTT | 1494. | GCCGAGGGGTC | 1605 | AGG | 3 |
| | | | | | | | | AATGATCCG | | TTAATGATCCGC | | | |
| | | | | | | | | CG | | GAGGAGA | | | |
| KMT2A | IL2RA | IL2RA low | 4297 | NM_005933.3 | NC_000011.10 | 1.18E+08 | sense | TTGACCATA | 1495. | TCACTTGACCAT | 1606 | TGG | 19 |
| | | | | | | | | ATTATGCTC | | AATTATGCTCAG | | | |
| | | | | | | | | AG | | TGGCAG | | | |
| PTEN | IL2RA | IL2RA low | 5728 | NM_001304718.1 | NC_000010.11 | 87961027 | sense | AGAGCGTGC | 1496. | GTATAGAGCGT | 1607 | AGG | 8 |
| | | | | | | | | AGATAATGA | | GCAGATAATGA | | | |
| | | | | | | | | CA | | CAAGGAAT | | | |
| PTEN | IL2RA | IL2RA low | 5728 | NM_001304718.1 | NC_000010.11 | 87957861 | antisense | AGCTGGCAG | 1497. | CTTTAGCTGGCA | 1608 | AGG | 7 |
| | | | | | | | | ACCACAAAC | | GACCACAAACT | | | |
| | | | | | | | | TG | | GAGGATC | | | |
| PTEN | IL2RA | IL2RA low | 5728 | NM_001304718.1 | NC_000010.11 | 87960940 | sense | ATTCTTCATA | 1498. | ATACATTCTTCA | 1609 | AGG | 8 |
| | | | | | | | | CCAGGACCA | | TACCAGGACCA | | | |
| | | | | | | | | G | | GAGGAAA | | | |
| PTEN | IL2RA | IL2RA low | 5728 | NM_001304718.1 | NC_000010.11 | 87957915 | sense | CCAATTCAG | 1499. | TCCTCCAATTCA | 1610 | CGG | 7 |
| | | | | | | | | GACCCACAC | | GGACCCACACG | | | |
| | | | | | | | | GA | | ACGGGAA | | | |
| RELA | IL2RA | IL2RA low | 5970 | NM_001243984.1 | NC_000011.10 | 65659757 | sense | ACTACGACC | 1500. | GGGGACTACGA | 1611 | CGG | 6 |
| | | | | | | | | TGAATGCTG | | CCTGAATGCTGT | | | |
| | | | | | | | | TG | | GCGGCTC | | | |
| RELA | IL2RA | IL2RA low | 5970 | NM_001243984.1 | NC_000011.10 | 65662009 | sense | GCTTCCGCT | 1501. | ATGCGCTTCCGC | 1612 | GGG | 3 |
| | | | | | | | | ACAAGTGCG | | TACAAGTGCGA | | | |
| | | | | | | | | AG | | GGGGCGC | | | |
| RELA | IL2RA | IL2RA low | 5970 | NM_001243984.1 | NC_000011.10 | 65658759 | antisense | GGAAGATCT | 1502. | AGTAGGAAGAT | 1613 | AGG | 7 |
| | | | | | | | | CATCCCCAC | | CTCATCCCCACC | | | |
| | | | | | | | | CG | | GAGGCAG | | | |
| RELA | IL2RA | IL2RA low | 5970 | NM_001243984.1 | NC_000011.10 | 65661818 | sense | TCAATGGCT | 1503. | CAGATCAATGG | 1614 | GGG | 4 |
| | | | | | | | | ACACAGGAC | | CTACACAGGAC | | | |
| | | | | | | | | CA | | CAGGGACA | | | |
| ETS1 | IL2RA | IL2RA low | 2113 | NM_005238.3 | NC_000011.10 | 1.28E+08 | antisense | CTTACTAAT | 1504. | TGAACTTACTAA | 1615 | AGG | 4 |
| | | | | | | | | GAAGTAATC | | TGAAGTAATCC | | | |
| | | | | | | | | CG | | GAGGTAT | | | |
| ETS1 | IL2RA | IL2RA low | 2113 | NM_005238.3 | NC_000011.10 | 1.28E+08 | antisense | GAGAAAGC | 1505. | GCTCGAGAAAG | 1616 | GGG | 3 |
| | | | | | | | | AGTCTTTAC | | CAGTCTTTACCC | | | |
| | | | | | | | | CCA | | AGGGCGC | | | |
| ETS1 | IL2RA | IL2RA low | 2113 | NM_005238.3 | NC_000011.10 | 1.28E+08 | antisense | GGTCTCGGA | 1506. | AGAGGGTCTCG | 1617 | GGG | 5 |
| | | | | | | | | GAATGACCG | | GAGAATGACCG | | | |
| | | | | | | | | AG | | AGGGGTAG | | | |
| ETS1 | IL2RA | IL2RA low | 2113 | NM_005238.3 | NC_000011.10 | 1.28E+08 | sense | TGCATGGGG | 1507. | CATGTGCATGG | 1618 | TGG | 5 |
| | | | | | | | | AGGACCAGT | | GGAGGACCAGT | | | |
| | | | | | | | | CG | | CGTGGTAG | | | |
| RBPJ | IL2RA | IL2RA low | 3516 | NM_005349.3 | NC_000004.12 | 26415547 | sense | AAAGAACAA | 1508. | AAAAAAAGAAC | 1619 | TGG | 5 |
| | | | | | | | | ATGGAACGC | | AAATGGAACGC | | | |
| | | | | | | | | GA | | GATGGTTG | | | |
| RBPJ | IL2RA | IL2RA low | 3516 | NM_005349.3 | NC_000004.12 | 26386378 | antisense | CACCTAGTA | 1509. | TACTCACCTAGT | 1620 | TGG | 3 |
| | | | | | | | | AGTCGTTTA | | AAGTCGTTTAG | | | |
| | | | | | | | | GG | | GTGGAGG | | | |
| RBPJ | IL2RA | IL2RA low | 3516 | NM_005349.3 | NC_000004.12 | 26424454 | sense | CATGCCAGT | 1510. | TTTTCATGCCAG | 1621 | GGG | 7 |
| | | | | | | | | TCACAGCAG | | TTCACAGCAGT | | | |
| | | | | | | | | TG | | GGGGAGC | | | |
| RBPJ | IL2RA | IL2RA low | 3516 | NM_005349.3 | NC_000004.12 | 26424363 | sense | CATTGCCTC | 1511. | TATGCATTGCCT | 1622 | TGG | 7 |
| | | | | | | | | AGGAACAAA | | CAGGAACAAAG | | | |
| | | | | | | | | GG | | GTGGCTC | | | |
| RXRB | IL2RA | IL2RA low | 6257 | NM_021976.4 | NC_000006.12 | 33198326 | sense | ACGGCTATG | 1512. | GCAAACGGCTA | 1623 | GGG | 3 |
| | | | | | | | | TGCAATCTG | | TGTGCAATCTGC | | | |
| | | | | | | | | CG | | GGGGACA | | | |
| RXRB | IL2RA | IL2RA low | 6257 | NM_021976.4 | NC_000006.12 | 33200341 | sense | GCCCTGGCT | 1513. | GACGGCCCTGG | 1624 | CGG | 1 |
| | | | | | | | | GGATCCCGC | | CTGGATCCCGC | | | |
| | | | | | | | | AG | | AGCGGCGG | | | |
| RXRB | IL2RA | IL2RA low | 6257 | NM_021976.4 | NC_000006.12 | 33197840 | sense | GGACAACAA | 1514. | GCCGGGACAAC | 1625 | TGG | 4 |
| | | | | | | | | AGACTGCAC | | AAAGACTGCAC | | | |
| | | | | | | | | AG | | AGTGGACA | | | |
| RXRB | IL2RA | IL2RA low | 6257 | NM_021976.4 | NC_000006.12 | 33198421 | antisense | GTGGCTTCA | 1515. | ACTGGTGGCTT | 1626 | GGG | 3 |
| | | | | | | | | CATCTTCAG | | CACATCTTCAGG | | | |
| | | | | | | | | GG | | GGGGCCA | | | |
| ZNF148 | IL2RA | IL2RA low | 7707 | NM_021964.2 | NC_000003.12 | 1.25E+08 | sense | AGATCGAAG | 1516. | TTCAAGATCGA | 1627 | AGG | 4 |
| | | | | | | | | TATGCCTCA | | AGTATGCCTCAC | | | |
| | | | | | | | | CC | | CAGGAGA | | | |
| ZNF148 | IL2RA | IL2RA low | 7707 | NM_021964.2 | NC_000003.12 | 1.25E+08 | antisense | AGTGCATAC | 1517. | ATTAAGTGCAT | 1628 | AGG | 4 |
| | | | | | | | | TGTAGTCCT | | ACTGTAGTCCTT | | | |
| | | | | | | | | TG | | GAGGAAG | | | |
| ZNF148 | IL2RA | IL2RA low | 7707 | NM_021964.2 | NC_000003.12 | 1.25E+08 | antisense | GAGCCCCCA | 1518. | ATGCGAGCCCC | 1629 | TGG | 9 |
| | | | | | | | | ACTGACGAA | | CAACTGACGAA | | | |
| | | | | | | | | TG | | TGTGGCAT | | | |
| ZNF148 | IL2RA | IL2RA low | 7707 | NM_021964.2 | NC_000003.12 | 1.25E+08 | antisense | TAATTAGTA | 1519. | ATCATAATTAGT | 1630 | AGG | 9 |
| | | | | | | | | CTACTATGC | | ACTACTATGCAC | | | |
| | | | | | | | | AC | | AGGTTT | | | |
| VPS52 | IL2RA | IL2RA low | 6293 | NM_001289174.1 | NC_000006.12 | 33267957 | sense | CAATGAACG | 1520. | TGGGCAATGAA | 1631 | AGG | 8 |
| | | | | | | | | AGCAACAGC | | CGAGCAACAGC | | | |
| | | | | | | | | AA | | AAAGGAGA | | | |
| VPS52 | IL2RA | IL2RA low | 6293 | NM_001289174.1 | NC_000006.12 | 33266562 | sense | CCGTACACT | 1521. | TGGGCCGTACA | 1632 | TGG | 11 |
| | | | | | | | | CAGCATGAC | | CTCAGCATGAC | | | |
| | | | | | | | | CC | | CCTGGTAA | | | |
| VPS52 | IL2RA | IL2RA low | 6293 | NM_001289174.1 | NC_000006.12 | 33269070 | sense | GAAATCGCC | 1522. | CTTCGAAATOGC | 1633 | GGG | 5 |
| | | | | | | | | AGGCAGTTC | | CAGGCAGTTCG | | | |
| | | | | | | | | GG | | GGGGAAA | | | |
| VPS52 | IL2RA | IL2RA low | 6293 | NM_001289174.1 | NC_000006.12 | 33264461 | antisense | TCCAGGATC | 1523. | CATCTCCAGGAT | 1634 | TGG | 13 |
| | | | | | | | | AGTTCAAAC | | CAGTTCAAACC | | | |
| | | | | | | | | CG | | GTGGCCA | | | |
| TFAP4 | IL2RA | IL2RA low | 7023 | NM_003223.2 | NC_000016.10 | 4262329 | sense | ACAGCTCAA | 1524. | ACACACAGCTC | 1635 | AGG | 3 |
| | | | | | | | | GCGCTTCAT | | AAGCGCTTCATC | | | |
| | | | | | | | | CC | | CAGGTGC | | | |
| TFAP4 | IL2RA | IL2RA low | 7023 | NM_003223.2 | NC_000016.10 | 4261877 | sense | AGGCTCCCC | 1525. | GCATAGGCTCC | 1636 | AGG | 4 |
| | | | | | | | | GGACATCTG | | CCGGACATCTG | | | |
| | | | | | | | | GG | | GGAGGACG | | | |
| TFAP4 | IL2RA | IL2RA low | 7023 | NM_003223.2 | NC_000016.10 | 4262671 | sense | CACTAACCC | 1526. | ATTCCACTAACC | 1637 | CGG | 2 |
| | | | | | | | | CCGAGACTC | | CCCGAGACTCA | | | |
| | | | | | | | | AG | | GCGGGAC | | | |
| TFAP4 | IL2RA | IL2RA low | 7023 | NM_003223.2 | NC_000016.10 | 4262597 | sense | CGCATGCAG | 1527. | GAGACGCATGC | 1638 | GGG | 2 |
| | | | | | | | | AGCATCAAC | | AGAGCATCAAC | | | |
| | | | | | | | | GC | | GCGGGATT | | | |
| IKZF3 | IL2RA | IL2RA low | 22806 | NM_012481.4 | NC_000017.11 | 39792732 | sense | AAGATGAAC | 1528. | TGGAAAGATGA | 1639 | TGG | 4 |
| | | | | | | | | TGCGATGTG | | ACTGCGATGTG | | | |
| | | | | | | | | TG | | TGTGGATT | | | |
| IKZF3 | IL2RA | IL2RA low | 22806 | NM_012481.4 | NC_000017.11 | 39788318 | sense | CAAGCAGAG | 1529. | GTTACAAGCAG | 1640 | AGG | 6 |
| | | | | | | | | AAGTTCCCT | | AGAAGTTCCCTT | | | |
| | | | | | | | | TG | | GAGGAGC | | | |
| IKZF3 | IL2RA | IL2RA low | 22806 | NM_012481.4 | NC_000017.11 | 39766413 | sense | GCTCATACA | 1530. | GTGAGCTCATA | 1641 | TGG | 8 |
| | | | | | | | | GACCCGCAT | | CAGACCCGCAT | | | |
| | | | | | | | | GA | | GATGGACC | | | |
| IKZF3 | IL2RA | IL2RA low | 22806 | NM_012481.4 | NC_000017.11 | 39777693 | sense | GGACAGATT | 1531 | TACTGGACAGA | 1642 | TGG | 7 |
| | | | | | | | | AGCAAGCAA | | TTAGCAAGCAA | | | |
| | | | | | | | | TG | | TGTGGCAA | | | |
| SRF | IL2RA | IL2RA low | 6722 | NM_003131.3 | NC_000006.12 | 43175724 | antisense | AGGTTGGTG | 1532. | CGGCAGGTTGG | 1643 | CGG | 3 |
| | | | | | | | | ACTGTGAAC | | TGACTGTGAAC | | | |
| | | | | | | | | GC | | GCCGGCTT | | | |
| SRF | IL2RA | IL2RA low | 6722 | NM_003131.3 | NC_000006.12 | 43172119 | sense | AGTTCATCG | 1533. | ATGGAGTTCAT | 1644 | CGG | 1 |
| | | | | | | | | ACAACAAGC | | CGACAACAAGC | | | |
| | | | | | | | | TG | | TGCGGCGC | | | |
| SRF | IL2RA | IL2RA low | 6722 | NM_003131.3 | NC_000006.12 | 43175844 | antisense | GGGCTGACA | 1534. | ACTGGGGCTGA | 1645 | TGG | 3 |
| | | | | | | | | CTAGCAGAC | | CACTAGCAGAC | | | |
| | | | | | | | | AC | | ACTGGTGC | | | |
| SRF | IL2RA | IL2RA low | 6722 | NM_003131.3 | NC_000006.12 | 43174015 | antisense | TCTGTTGTG | 1535. | CTGGTCTGTTGT | 1646 | GGG | 2 |
| | | | | | | | | GGGTCTGAA | | GGGGTCTGAAC | | | |
| | | | | | | | | CG | | GGGGTGG | | | |
| CEBPZ | IL2RA | L2RA low | 10153 | NM_005760.2 | NC_000002.12 | 37223251 | antisense | AAAGCTCCA | 1536. | ATATAAAGCTCC | 1647 | TGG | 3 |
| | | | | | | | | CATATAAAT | | ACATATAAATG | | | |
| | | | | | | | | GG | | GTGGCAT | | | |
| CEBPZ | IL2RA | IL2RA low | 10153 | NM_005760.2 | NC_000002.12 | 37228088 | antisense | ACAAAGCAG | 1537. | TGTTACAAAGC | 1648 | CGG | 2 |
| | | | | | | | | CTCATGAGC | | AGCTCATGAGC | | | |
| | | | | | | | | CA | | CACGGTAA | | | |
| CEBPZ | IL2RA | IL2RA low | 10153 | NM_005760.2 | NC_000002.12 | 37227623 | antisense | CAACATTAA | 1538. | AAAGCAACATT | 1649 | TGG | 2 |
| | | | | | | | | AGCCTGGAC | | AAAGCCTGGAC | | | |
| | | | | | | | | AC | | ACTGGTAT | | | |
| CEBPZ | IL2RA | IL2RA low | 10153 | NM_005760.2 | NC_000002.12 | 37228473 | sense | TGAAGGCAA | 1539. | TGGATGAAGGC | 1650 | GGG | 2 |
| | | | | | | | | TTGTGTCAT | | AATTGTGTCATC | | | |
| | | | | | | | | CG | | GGGGACA | | | |
| BATF | IL2RA | IL2RA low | 10538 | NM_006399.3 | NC_000014.9 | 75546499 | sense | ACAGAACGC | 1540. | AGAAACAGAAC | 1651 | AGG | 3 |
| | | | | | | | | GGCTCTACG | | GCGGCTCTACG | | | |
| | | | | | | | | CA | | CAAGGAGA | | | |
| BATF | IL2RA | IL2RA low | 10538 | NM_006399.3 | NC_000014.9 | 75522739 | antisense | AGGACTCTA | 1541. | AAGGAGGACTC | 1652 | GGG | 1 |
| | | | | | | | | CCTGTTTGC | | TACCTGTTTGCC | | | |
| | | | | | | | | CA | | AGGGGGA | | | |
| BATF | IL2RA | IL2RA low | 10538 | NM_006399.3 | NC_000014.9 | 75525114 | sense | ATGTGAGAA | 1542. | GATGATGTGAG | 1653 | AGG | 2 |
| | | | | | | | | GAGTTCAGA | | AAGAGTTCAGA | | | |
| | | | | | | | | GG | | GGAGGGAG | | | |
| BATF | IL2RA | IL2RA low | 10538 | NM_006399.3 | NC_000014.9 | 75546520 | sense | GGAGATCAA | 1543. | GCAAGGAGATC | 1654 | AGG | 3 |
| | | | | | | | | GCAGCTCAC | | AAGCAGCTCAC | | | |
| | | | | | | | | AG | | AGAGGAAC | | | |
| CIC | IL2RA | IL2RA low | 23152 | NM_015125.4 | NC_000019.10 | 42291060 | sense | ACTGTCACT | 1544. | TGCCACTGTCAC | 1655 | GGG | 10 |
| | | | | | | | | AACCTACTG | | TAACCTACTGGT | | | |
| | | | | | | | | GT | | GGGCAC | | | |
| CIC | IL2RA | IL2RA low | 23152 | NM_015125.4 | NC_000019.10 | 42292311 | antisense | CCCGCCCGC | 1545. | CGTGCCCGCCC | 1656 | AGG | 13 |
| | | | | | | | | TGACTGCAC | | GCTGACTGCAC | | | |
| | | | | | | | | AT | | ATAGGTGA | | | |
| CIC | IL2RA | IL2RA low | 23152 | NM_015125.4 | NC_000019.10 | 42287372 | sense | CTCTACCGC | 1546. | TTGCCTCTACCG | 1657 | CGG | 4 |
| | | | | | | | | CCGGAAAAC | | CCCGGAAAACG | | | |
| | | | | | | | | GT | | TCGGACC | | | |
| CIC | IL2RA | IL2RA low | 23152 | NM_015125.4 | NC_000019.10 | 42289198 | antisense | TTGGGCCAG | 1547. | GGCTTTGGGCC | 1658 | GGG | 8 |
| | | | | | | | | AGTACGATG | | AGAGTACGATG | | | |
| | | | | | | | | CA | | CAGGGCCA | | | |
| IKZF1 | IL2RA | IL2RA low | 10320 | NM_006060.5 | NC_000007.14 | 50376659 | sense | GAAAATGAA | 1548. | GAGAGAAAATG | 1659 | GGG | 4 |
| | | | | | | | | TGGCTCCCA | | AATGGCTCCCA | | | |
| | | | | | | | | CA | | CAGGGACC | | | |
| IKZF1 | IL2RA | IL2RA low | 10320 | NM_006060.5 | NC_000007.14 | 50399996 | antisense | GATGGCTTG | 1549. | TGTTGATGGCTT | 1660 | GGG | 8 |
| | | | | | | | | GTCCATCAC | | GGTCCATCACG | | | |
| | | | | | | | | GT | | TGGGACT | | | |
| IKZF1 | IL2RA | IL2RA low | 10320 | NM_006060.5 | NC_000007.14 | 50382586 | sense | GGGGCCTCA | 1550. | GTGCGGGGCCT | 1661 | GGG | 5 |
| | | | | | | | | TTCACCCAG | | CATTCACCCAGA | | | |
| | | | | | | | | AA | | AGGGCAA | | | |
| IKZF1 | IL2RA | IL2RA low | 10320 | NM_006060.5 | NC_000007.14 | 50327753 | sense | TCCAAGAGT | 1551. | AAGCTCCAAGA | 1662 | GGG | 3 |
| | | | | | | | | GACAGAGTC | | GTGACAGAGTC | | | |
| | | | | | | | | GT | | GTGGGTAA | | | |
| IRF4 | IL2RA | IL2RA low | 3662 | NM_001195286.1 | NC_000006.12 | 394977 | sense | CAGACCCGT | 1552. | ATCTCAGACCC | 1663 | AGG | 3 |
| | | | | | | | | ACAAAGTGT | | GTACAAAGTGT | | | |
| | | | | | | | | AC | | ACAGGATT | | | |
| IRF4 | IL2RA | IL2RA low | 3662 | NM_001195286.1 | NC_000006.12 | 397215 | sense | CCCATGACG | 1553. | ATGTCCCATGAC | 1664 | CGG | 5 |
| | | | | | | | | TTTGGACCC | | GTTTGGACCCC | | | |
| | | | | | | | | CG | | GCGGCCA | | | |
| IRF4 | IL2RA | IL2RA low | 3662 | NM_001195286.1 | NC_000006.12 | 401466 | sense | CTACCGGGA | 1554. | TGTACTACCGG | 1665 | AGG | 7 |
| | | | | | | | | AATCCTCGT | | GAAATCCTCGT | | | |
| | | | | | | | | GA | | GAAGGAGC | | | |
| IRF4 | IL2RA | IL2RA low | 3662 | NM_001195286.1 | NC_000006.12 | 393251 | sense | CTGATCGAC | 1555. | GTGGCTGATCG | 1666 | CGG | 2 |
| | | | | | | | | CAGATCGAC | | ACCAGATCGAC | | | |
| | | | | | | | | AG | | AGCGGCAA | | | |
| MBD2 | IL2RA | IL2RA low | 8932 | NM_003927.4 | NC_000018.10 | 54224170 | sense | AGCCGGTCC | 1556. | CGGGAGCCGGT | 1667 | GGG | 1 |
| | | | | | | | | CTTTCCCGTC | | CCCTTTCCCGTC | | | |
| | | | | | | | | G | | GGGGAGC | | | |
| MBD2 | IL2RA | IL2RA low | 8932 | NM_003927.4 | NC_000018.10 | 54205113 | sense | CCTCAGTTG | 1557. | CAAGCCTCAGTT | 1668 | GGG | 2 |
| | | | | | | | | GCAAGGTAC | | GGCAAGGTACC | | | |
| | | | | | | | | CT | | TGGGAAA | | | |
| MBD2 | IL2RA | IL2RA low | 8932 | NM_003927.4 | NC_000018.10 | 54204999 | sense | CCTCTCAATC | 1558. | CGATCCTCTCAA | 1669 | TGG | 2 |
| | | | | | | | | AAAATAAGG | | TCAAAATAAGG | | | |
| | | | | | | | | T | | TTGGTTA | | | |
| MBD2 | IL2RA | IL2RA low | 8932 | NM_003927.4 | NC_000018.10 | 54224048 | sense | CGAAAATCT | 1559. | GATCCGAAAAT | 1670 | TGG | 1 |
| | | | | | | | | GGGCTAAGT | | CTGGGCTAAGT | | | |
| | | | | | | | | GC | | GCTGGCAA | | | |
| JAK3 | IL2RA | IL2RA low | 3718 | NM_000215.3 | NC_000019.10 | 17835098 | antisense | ACTCTCCAG | 1560. | ACTTACTCTCCA | 1671 | CGG | 15 |
| | | | | | | | | GCTTAACAC | | GGCTTAACACA | | | |
| | | | | | | | | AG | | GCGGGGC | | | |
| JAK3 | IL2RA | IL2RA low | 3718 | NM_000215.3 | NC_000019.10 | 17839577 | antisense | AGCTCTCGA | 1561. | CAGGAGCTCTC | 1672 | GGG | 10 |
| | | | | | | | | AGACTGCTG | | GAAGACTGCTG | | | |
| | | | | | | | | TG | | TGGGGTCG | | | |
| JAK3 | IL2RA | IL2RA low | 3718 | NM_000215.3 | NC_000019.10 | 17836043 | antisense | GTGTACAAA | 1562. | CCAGGTGTACA | 1673 | TGG | 14 |
| | | | | | | | | TTCCTGCAC | | AATTCCTGCACC | | | |
| | | | | | | | | CA | | ATGGTGC | | | |
| JAK3 | IL2RA | IL2RA low | 3718 | NM_000215.3 | NC_000019.10 | 17842538 | sense | TGACGCGGA | 1563. | TTCGTGACGCG | 1674 | AGG | 6 |
| | | | | | | | | GGCGTATTC | | GAGGCGTATTC | | | |
| | | | | | | | | GG | | GGAGGACG | | | |
| BCL6 | IL2RA | IL2RA low | 604 | NM_001706.4 | NC_000003.12 | 1.88E+08 | sense | AAGTGATAT | 1564. | CACGAAGTGAT | 1675 | TGG | 5 |
| | | | | | | | | GCACTACAG | | ATGCACTACAG | | | |
| | | | | | | | | TG | | TGTGGCTG | | | |
| BCL6 | IL2RA | IL2RA low | 604 | NM_001706.4 | NC_000003.12 | 1.88E+08 | sense | CAAGACATC | 1565. | GCCCCAAGACA | 1676 | GGG | 5 |
| | | | | | | | | ATGGCCTAT | | TCATGGCCTATC | | | |
| | | | | | | | | CG | | GGGGTCG | | | |
| BCL6 | IL2RA | IL2RA low | 604 | NM_001706.4 | NC_000003.12 | 1.88E+08 | antisense | CATCTGCAG | 1566. | GCTCCATCTGCA | 1677 | TGG | 4 |
| | | | | | | | | GTACATAGC | | GGTACATAGCC | | | |
| | | | | | | | | CG | | GTGGCCA | | | |
| BCL6 | IL2RA | IL2RA low | 604 | NM_001706.4 | NC_000003.12 | 1.88E+08 | antisense | TCCCTCACCT | 1567. | CAGATCCCTCAC | 1678 | AGG | 3 |
| | | | | | | | | GCAGGCCAT | | CTGCAGGCCAT | | | |
| | | | | | | | | G | | GAGGACC | | | |
| ZNF236 | IL2RA | IL2RA low | 7776 | NM_007345.3 | NC_000018.10 | 76919836 | sense | ATCACGGAA | 1568. | ATGCATCACGG | 1679 | AGG | 20 |
| | | | | | | | | GTCTCGTCC | | AAGTCTCGTCCT | | | |
| | | | | | | | | TG | | GAGGTCA | | | |
| ZNF236 | IL2RA | IL2RA low | 7776 | NM_007345.3 | NC_000018.10 | 76913812 | sense | GCATGTGCG | 1569. | AGCAGCATGTG | 1680 | GGG | 18 |
| | | | | | | | | GTCGCACAC | | CGGTCGCACAC | | | |
| | | | | | | | | CG | | CGGGGAAA | | | |
| ZNF236 | IL2RA | IL2RA low | 7776 | NM_007345.3 | NC_000018.10 | 76905317 | sense | GGCAGCTTA | 1570. | TGGTGGCAGCT | 1681 | GGG | 13 |
| | | | | | | | | CGGCGACAC | | TACGGCGACAC | | | |
| | | | | | | | | AT | | ATGGGTAT | | | |
| ZNF236 | IL2RA | IL2RA low | 7776 | NM_007345.3 | NC_000018.10 | 76910097 | antisense | TGGTCTACG | 1571. | AAACTGGTCTA | 1682 | AGG | 15 |
| | | | | | | | | TGCCCTCGC | | CGTGCCCTCGC | | | |
| | | | | | | | | AG | | AGAGGGTC | | | |
| TCF3 | IL2RA | IL2RA low | 6929 | NM_003200.3 | NC_000019.10 | 1622055 | sense | CACCAGCAC | 1572. | CCTGCACCAGC | 1683 | AGG | 10 |
| | | | | | | | | GAGCGTATG | | ACGAGCGTATG | | | |
| | | | | | | | | GT | | GTAGGACC | | | |
| TCF3 | IL2RA | IL2RA low | 6929 | NM_003200.3 | NC_000019.10 | 1622322 | sense | CTATCCCGC | 1573. | GCACCTATCCCG | 1684 | TGG | 9 |
| | | | | | | | | CCCCTTCTAC | | CCCCCTTCTACG | | | |
| | | | | | | | | G | | TGGCAG | | | |
| TCF3 | IL2RA | IL2RA low | 6929 | NM_003200.3 | NC_000019.10 | 1619821 | antisense | GCTGGGCGA | 1574. | CGTAGCTGGGC | 1685 | GGG | 14 |
| | | | | | | | | TAAGGCACC | | GATAAGGCACC | | | |
| | | | | | | | | GG | | GGGGGCTC | | | |
| TCF3 | IL2RA | IL2RA low | 6929 | NM_003200.3 | NC_000019.10 | 1621034 | antisense | GTTATTGCTT | 1575. | AGAAGTTATTG | 1686 | GGG | 13 |
| | | | | | | | | GAGTGATCC | | CTTGAGTGATCC | | | |
| | | | | | | | | G | | GGGGAGT | | | |
| YY1 | IL2RA | IL2RA low | 7528 | NM_003403.4 | NC_000014.9 | 1E+08 | sense | AGATATTGA | 1576. | AAAAAGATATT | 1687 | TGG | 2 |
| | | | | | | | | CCATGAGAC | | GACCATGAGAC | | | |
| | | | | | | | | AG | | AGTGGTTG | | | |
| YY1 | IL2RA | IL2RA low | 7528 | NM_003403.4 | NC_000014.9 | 1E+08 | sense | GGAGACCAT | 1577. | CGGTGGAGACC | 1688 | TGG | 1 |
| | | | | | | | | CGAGACCAC | | ATCGAGACCAC | | | |
| | | | | | | | | AG | | AGTGGTGG | | | |
| YY1 | IL2RA | IL2RA low | 7528 | NM_003403.4 | NC_000014.9 | 1E+08 | sense | GGTCACCGA | 1578. | CGCTGGTCACC | 1689 | AGG | 1 |
| | | | | | | | | CGACCCGAC | | GACGACCCGAC | | | |
| | | | | | | | | CC | | CCAGGTGC | | | |
| YY1 | IL2RA | IL2RA low | 7528 | NM_003403.4 | NC_000014.9 | 1E+08 | sense | TGAACAAAC | 1579. | ACATTGAACAA | 1690 | TGG | 1 |
| | | | | | | | | GCTGGTCAC | | ACGCTGGTCAC | | | |
| | | | | | | | | CG | | CGTGGCGG | | | |
| PURA | IL2RA | IL2RA low | 5813 | NM_005859.4 | NC_000005.10 | 1.4E+08 | sense | CCTTACTCTC | 1580. | GCCGCCTTACTC | 1691 | TGG | 1 |
| | | | | | | | | TCCATGTCA | | TCTCCATGTCAG | | | |
| | | | | | | | | G | | TGGCCG | | | |
| PURA | IL2RA | IL2RA low | 5813 | NM_005859.4 | NC_000005.10 | 1.4E+08 | antisense | GAACTCGAT | 1581. | CACGGAACTCG | 1692 | CGG | 1 |
| | | | | | | | | GAGCCCCTG | | ATGAGCCCCTG | | | |
| | | | | | | | | CG | | CGCGGGCA | | | |
| PURA | IL2RA | IL2RA low | 5813 | NM_005859.4 | NC_000005.10 | 1.4E+08 | sense | GCTCATCGA | 1582. | CCAAGCTCATC | 1693 | TGG | 1 |
| | | | | | | | | CGACTACGG | | GACGACTACGG | | | |
| | | | | | | | | AG | | AGTGGAGG | | | |
| PURA | IL2RA | IL2RA low | 5813 | NM_005859.4 | NC_000005.10 | 1.4E+08 | sense | TCCGCCAGA | 1583. | CGCATCCGCCA | 1694 | GGG | 1 |
| | | | | | | | | CGGTCAACC | | GACGGTCAACC | | | |
| | | | | | | | | GG | | GGGGGCCT | | | |

TABLE 9
Nuclear factors that can be inhibited to increase IL2RA expression or overexpressed to decrease IL2RA expression
specifically in effector T cells

| | | | | | | Position | | | | | | | | |
|---|---|---|---|---|---|---|---|---|---|---|---|---|---|---|
| | | | Tar- | | | of Base | | | | | | | | |
| | | | get | | | After | | sgRNA | SEQ | Target | SEQ | | | Rule |
| gene_ | screen | screen | Gene | Target | Genomic | Cut (1- | | Target | ID | Context | ID | PAM | Exon | Set 2 |
| id | target | direction | ID | Transcript | Sequence | based) | Strand | Sequence | NO: | Sequence | NO: | Sequence | Number | score |
| CTCF | IL2RA Teffs | IL2RA high | 10664 | NM_006565.3 | NC_000016.10 | 67612001 | antisense | CGATCCAA | 1695. | GTGACGAT | 1743. | TGG | 4 | 0.6364 |
| | | | | | | | | ATTTGAAC | | CCAAATTT | | | | |
| | | | | | | | | GCCG | | GAACGCC | | | | |
| | | | | | | | | | | GTGGACA | | | | |
| CTCF | IL2RA Teffs | IL2RA high | 10664 | NM_006565.3 | NC_000016.10 | 67611476 | sense | GAGCAAA | 1696. | AAAAGAG | 1744. | AGG | 3 | 0.6793 |
| | | | | | | | | CTGCGTTA | | CAAACTGC | | | | |
| | | | | | | | | TACAG | | GTTATACA | | | | |
| | | | | | | | | | | GAGGAGG | | | | |
| CTCF | IL2RA Teffs | IL2RA high | 10664 | NM_006565.3 | NC_000016.10 | 67610967 | sense | TTACCCCA | 1697. | CCACTTAC | 1745. | TGG | 3 | 0.6378 |
| | | | | | | | | GAACCAG | | CCCAGAAC | | | | |
| | | | | | | | | ACGGA | | CAGACGG | | | | |
| | | | | | | | | | | ATGGGGG | | | | |
| CTCP | IL2RA Teffs | IL2RA high | 10664 | NM_006565.3 | NC_000016.10 | 67620773 | sense | TTTGTGCA | 1698. | GCAGTTTG | 1746. | GGG | 6 | 0.6444 |
| | | | | | | | | GTTATGCC | | TGCAGTTA | | | | |
| | | | | | | | | AGCA | | TGCCAGCA | | | | |
| | | | | | | | | | | GGGACA | | | | |
| PTPRC | IL2RA Teffs | IL2RA high | 5788 | NM_002838.4 | NC_000001.11 | 1.99E+08 | antisense | AGCATTAT | 1699. | CACTAGCA | 1747. | GGG | 4 | 0.6692 |
| | | | | | | | | CCAAAGA | | TTATCCAA | | | | |
| | | | | | | | | GTCCG | | AGAGTCC | | | | |
| | | | | | | | | | | GGGGATA | | | | |
| PTPRC | IL2RA Teffs | IL2RA high | 5788 | NM_002838.4 | NC_000001.11 | 1.99E+08 | antisense | GGAAACTT | 1700. | TATAGGAA | 1748. | CGG | 19 | 0.6482 |
| | | | | | | | | GCTGAACA | | ACTTGCTG | | | | |
| | | | | | | | | CCCG | | AACACCCG | | | | |
| | | | | | | | | | | CGGGAT | | | | |
| PTPRC | IL2RA Teffs | IL2RA high | 5788 | NM_002838.4 | NC_000001.11 | 1.99E+08 | antisense | TCCAAATG | 1701. | AACTTCCA | 1749. | GGG | 14 | 0.6167 |
| | | | | | | | | GTAACGTT | | AATGGTAA | | | | |
| | | | | | | | | CATG | | CGTTCATG | | | | |
| | | | | | | | | | | GGGGCC | | | | |
| PTPRC | IL2RA Teffs | IL2RA high | 5788 | NM_002838.4 | NC_000001.11 | 1.99E+08 | sense | TGTGGATT | 1702. | TCACTGTG | 1750. | AGG | 9 | 0.6937 |
| | | | | | | | | ACTTATAT | | GATTACTT | | | | |
| | | | | | | | | AACA | | ATATAACA | | | | |
| | | | | | | | | | | AGGAAA | | | | |
| NR2C2 | IL2RA Teffs | IL2RA high | 7182 | NM_003298.4 | NC_000003.12 | 15030406 | sense | CCAGTCGA | 1703. | GAGACCA | 1751. | AGG | 10 | 0.6976 |
| | | | | | | | | CACCCATC | | GTCGACAC | | | | |
| | | | | | | | | ATTG | | CCATCATT | | | | |
| | | | | | | | | | | GAGGTTG | | | | |
| NR2C2 | IL2RA Teffs | IL2RA high | 7182 | NM_003298.4 | NC_000003.12 | 15016174 | antisense | CCCCAGTA | 1704. | TCTTCCCC | 1752. | AGG | 5 | 0.7308 |
| | | | | | | | | AACGCTCC | | AGTAAAC | | | | |
| | | | | | | | | ACAG | | GCTCCACA | | | | |
| | | | | | | | | | | GAGGCAG | | | | |
| NR2C2 | IL2RA Teffs | IL2RA high | 7182 | NM_003298.4 | NC_000003.12 | 15024201 | antisense | GAACGTCA | 1705. | TAGAGAA | 1753. | TGG | 8 | 0.6168 |
| | | | | | | | | CCTTAGAA | | CGTCACCT | | | | |
| | | | | | | | | TCCG | | TAGAATCC | | | | |
| | | | | | | | | | | GTGGCCA | | | | |
| NR2C2 | IL2RA Teffs | IL2RA high | 7182 | NM_003298.4 | NC_000003.12 | 15023322 | antisense | TCTTTGTC | 1706. | TCCATCTT | 1754. | GGG | 7 | 0.6618 |
| | | | | | | | | TGCCACAA | | TGTCTGCC | | | | |
| | | | | | | | | ACGT | | ACAAACGT | | | | |
| | | | | | | | | | | GGGAGT | | | | |
| TP53 | IL2RA Teffs | IL2RA high | 7157 | NM_000546.5 | NC_000017.11 | 7676227 | antisense | CCATTGTT | 1707. | TGAACCAT | 1755. | GGG | 4 | 0.6899 |
| | | | | | | | | CAATATCG | | TGTTCAAT | | | | |
| | | | | | | | | TCCG | | ATCGTCCG | | | | |
| | | | | | | | | | | GGGACA | | | | |
| TP53 | IL2RA Teffs | IL2RA high | 7157 | NM_000546.5 | NC_000017.11 | 7675058 | sense | GAGCGCT | 1708. | CCATGAGC | 1756. | TGG | 5 | 0.6542 |
| | | | | | | | | GCTCAGAT | | GCTGCTCA | | | | |
| | | | | | | | | AGCGA | | GATAGCG | | | | |
| | | | | | | | | | | ATGGTGA | | | | |
| TP53 | IL2RA Teffs | IL2RA high | 7157 | NM_000546.5 | NC_000017.11 | 7676527 | antisense | GATCCACT | 1709. | AATGGATC | 1757. | AGG | 2 | 0.6108 |
| | | | | | | | | CACAGTTT | | CACTCACA | | | | |
| | | | | | | | | CCAT | | GTTTCCAT | | | | |
| | | | | | | | | | | AGGTCT | | | | |
| TP53 | IL2RA Teffs | IL2RA high | 7157 | NM_000546.5 | NC_000017.11 | 7674864 | sense | GGTGCCCT | 1710. | TGGTGGT | 1758. | AGG | 6 | 0.6259 |
| | | | | | | | | ATGAGCC | | GCCCTATG | | | | |
| | | | | | | | | GCCTG | | AGCCGCCT | | | | |
| | | | | | | | | | | GAGGTCT | | | | |
| PRDM1 | IL2RA Teffs | IL2RA high | 639 | NM_001198.3 | NC_000006.12 | 1.06E+08 | sense | AGGATGC | 1711. | ATGGAGG | 1759. | TGG | 2 | 0.7731 |
| | | | | | | | | GGATATG | | ATGCGGAT | | | | |
| | | | | | | | | ACTCTG | | ATGACTCT | | | | |
| | | | | | | | | | | GTGGACA | | | | |
| PRDM1 | IL2RA Teffs | IL2RA high | 639 | NM_001198.3 | NC_000006.12 | 1.06E+08 | antisense | GGACGCG | 1712. | CGTAGGA | 1760. | AGG | 5 | 0.6665 |
| | | | | | | | | TTCAAGTA | | CGCGTTCA | | | | |
| | | | | | | | | AGCGT | | AGTAAGC | | | | |
| | | | | | | | | | | GTAGGAG | | | | |
| | | | | | | | | | | T | | | | |
| PRDM1 | IL2RA Teffs | IL2RA high | 639 | NM_001198.3 | NC_000006.12 | 1.06E+08 | antisense | GGGGAGC | 1713. | AATGGGG | 1761. | GGG | 5 | 0.6871 |
| | | | | | | | | GAGTGAT | | GAGCGAG | | | | |
| | | | | | | | | GTACGT | | TGATGTAC | | | | |
| | | | | | | | | | | GTGGGTCT | | | | |
| PRDM1 | IL2RA Teffs | IL.2RA high | 639 | NM_001198.3 | NC_000006.12 | 1.06E+08 | sense | TTTGGACA | 1714. | CTCTTTTG | 1762. | AGG | 4 | 0.6708 |
| | | | | | | | | GATCTATT | | GACAGATO | | | | |
| | | | | | | | | CCAG | | TATTCCAG | | | | |
| | | | | | | | | | | AGGGGA | | | | |
| MYC | IL2RA Teffs | IL2RA high | 4609 | NM_002467.4 | NC_000008.11 | 1.28E+08 | sense | AGAGTGC | 1715. | CCTCAGAG | 1763. | TGG | 2 | 0.6543 |
| | | | | | | | | ATCGACCC | | TGCATCGA | | | | |
| | | | | | | | | CTCGG | | CCCCTCGG | | | | |
| | | | | | | | | | | TGGTCT | | | | |
| MYC | IL2RA Teffs | IL2RA high | 4609 | NM_002467.4 | NC_000008.11 | 1.28E+08 | antisense | CTGCGGG | 1716. | TGCCCTGC | 1764, | AGG | 2 | 0.6832 |
| | | | | | | | | GAGGACT | | GGGGAGG | | | | |
| | | | | | | | | CCGTCG | | ACTCCGTC | | | | |
| | | | | | | | | | | GAGGAGA | | | | |
| MYC | IL2RA Teffs | IL2RA high | 4609 | NM_002467.4 | NC_000008.11 | 1.28E+08 | sense | CTTCGGGG | 1717. | CTCCCTTC | 1765. | CGG | 2 | 0.6919 |
| | | | | | | | | AGACAAC | | GGGGAGA | | | | |
| | | | | | | | | GACGG | | CAACGAC | | | | |
| | | | | | | | | | | GGCGGTG | | | | |
| | | | | | | | | | | G | | | | |
| MYC | IL2RA Teffs | IL2RA high | 4609 | NM_002467.4 | NC_000008.11 | 1.28E+08 | antisense | GCTGCACC | 1718. | TACGGCTG | 1766. | AGG | 2 | 0.6842 |
| | | | | | | | | GAGTOGTA | | CACCGAGT | | | | |
| | | | | | | | | GTCG | | CGTAGTCG | | | | |
| | | | | | | | | | | AGGTCA | | | | |
| SMARCB1 | IL2RA Teffs | IL2RA high | 6598 | NM_003073.3 | NC_000022.11 | 23791773 | antisense | GAGAACCT | 1719. | TACAGAG | 1767. | AGG | 2 | 0.731 |
| | | | | | | | | CGGAACAT | | AACCTCGG | | | | |
| | | | | | | | | ACGG | | AACATACG | | | | |
| | | | | | | | | | | GAGGTAG | | | | |
| SMARCB1 | IL2RA Teffs | IL2RA high | 6598 | NM_003073.3 | NC_000022.11 | 23816887 | sense | GCAGATC | 1720. | GACAGCA | 1768. | CGG | 6 | 0.6894 |
| | | | | | | | | GAGTCCTA | | GATCGAGT | | | | |
| | | | | | | | | CCCCA | | CCTACCCC | | | | |
| | | | | | | | | | | ACGGACA | | | | |
| SMARCB1 | IL2RA Teffs | IL2RA high | 6598 | NM_003073.3 | NC_000022.11 | 23801049 | antisense | TCTTCTTG | 1721. | GTTCTCTT | 1769. | CGG | 4 | 0.6806 |
| | | | | | | | | TCTCGGCC | | CTTGTCTC | | | | |
| | | | | | | | | CATG | | GGCCCATG | | | | |
| | | | | | | | | | | CGGTTC | | | | |
| SMARCB1 | IL2RA Teffs | IL2RA high | 6598 | NM_003073.3 | NC_000022.11 | 23803342 | sense | TGAGAAC | 1722. | TCCATGAG | 1770. | AGG | 5 | 0.6804 |
| | | | | | | | | GCATCTCA | | AACGCATC | | | | |
| | | | | | | | | GCCCG | | TCAGCCCG | | | | |
| | | | | | | | | | | AGGTGC | | | | |
| IRF1 | IL2RA Teffs | IL2RA high | 3659 | NM_002198.2 | NC_000005.10 | 1.32E+08 | sense | GAACTCCC | 1723. | CCATGAAC | 1771. | AGG | 4 | 0.6297 |
| | | | | | | | | TGCCAGAT | | TCCCTGCC | | | | |
| | | | | | | | | ATCG | | AGATATCG | | | | |
| | | | | | | | | | | AGGAGG | | | | |
| IRF1 | IL2RA Teffs | IL2RA high | 3659 | NM_002198.2 | NC_000005.10 | 1.32E+08 | sense | TCTAGGCC | 1724. | GCCCTCTA | 1772. | GGG | 4 | 0.718 |
| | | | | | | | | GATACAAA | | GGCCGAT | | | | |
| | | | | | | | | GCAG | | ACAAAGC | | | | |
| | | | | | | | | | | AGGGGAA | | | | |
| | | | | | | | | | | A | | | | |
| IRF1 | IL2RA Teffs | IL2RA high | 3659 | NM_002198.2 | NC_000005.10 | 1.32E+08 | sense | TCTCCCTC | 1725. | GCTTTCTC | 1773. | GGG | 6 | 0.6137 |
| | | | | | | | | GACAGTCA | | CCTCGACA | | | | |
| | | | | | | | | TGTG | | GTCATGTG | | | | |
| | | | | | | | | | | GGGATT | | | | |
| IRF1 | IL2RA Teffs | IL2RA high | 3659 | NM_002198.2 | NC_000005.10 | 1.32E+08 | sense | TTAATTCC | 1726. | CAGATTAA | 1774. | GGG | 2 | 0.7113 |
| | | | | | | | | AACCAAAT | | TTCCAACC | | | | |
| | | | | | | | | CCCG | | AAATCCCG | | | | |
| | | | | | | | | | | GGGCTC | | | | |
| IRF2 | IL2RA Teffs | IL2RA high | 3660 | NM_002199.3 | NC_000004.12 | 1.84E+08 | sense | ACCTGATC | 1727. | ATAAACCT | 1775. | AGG | 4 | 0.6049 |
| | | | | | | | | CCAAAACA | | GATCCCAA | | | | |
| | | | | | | | | TGGA | | AACATGG | | | | |
| | | | | | | | | | | AAGGCGA | | | | |
| IRF2 | IL2RA Teffs | IL2RA high | 3660 | NM_002199.3 | NC_000004.12 | 1.84E+08 | antisense | CAGCATTC | 1728. | GGGGCAG | 1776. | AGG | 4 | 0.6028 |
| | | | | | | | | GGTAGAC | | CATTCGGT | | | | |
| | | | | | | | | CCTGA | | AGACCCTG | | | | |
| | | | | | | | | | | AAGGCAT | | | | |
| IRF2 | IL2RA Teffs | IL2RA high | 3660 | NM_002199.3 | NC_000004.12 | 1.84E+08 | sense | GGATGCAT | 1729. | CCCTGGAT | 1777. | GGG | 3 | 0.6231 |
| | | | | | | | | GCGGCTA | | GCATGCG | | | | |
| | | | | | | | | GACAT | | GCTAGACA | | | | |
| | | | | | | | | | | TGGGTGG | | | | |
| IRF2 | IL2RA Teffs | IL2RA high | 3660 | NM_002199.3 | NC_000004.12 | 1.84E+08 | antisense | TACCTGCA | 1730. | CGCTTACC | 1778. | GGG | 7 | 0.7207 |
| | | | | | | | | TAGGAAG | | TGCATAGG | | | | |
| | | | | | | | | ACACG | | AAGACAC | | | | |
| | | | | | | | | | | GGGGGAG | | | | |
| TNFAIP3 | IL2RA Teffs | IL2RA high | 7128 | NM_001270507.1 | NC_000006.12 | 1.38E+08 | sense | CCACTTGT | 1731. | TGTTCCAC | 1779. | GGG | 6 | 0.6901 |
| | | | | | | | | TAACAGA | | TTGTTAAC | | | | |
| | | | | | | | | GACCG | | AGAGACC | | | | |
| | | | | | | | | | | GGGGAAG | | | | |
| TNFAIP3 | IL2RA Teffs | IL2RA high | 7128 | NM_001270507.1 | NC_000006.12 | 1.38E+08 | sense | CTTGTGGC | 1732. | GAAGCTTG | 1780. | CGG | 2 | 0.6709 |
| | | | | | | | | GCTGAAA | | TGGCGCTG | | | | |
| | | | | | | | | ACGAA | | AAAACGA | | | | |
| | | | | | | | | | | ACGGTAA | | | | |
| TNFAIP3 | IL2RA Teffs | IL2RA high | 7128 | NM_001270507.1 | NC_000006.12 | 1.38E+08 | sense | TATGCCAT | 1733. | CCTTTATG | 1781. | AGG | 7 | 0.7533 |
| | | | | | | | | GAGTGCTC | | CCATGAGT | | | | |
| | | | | | | | | AGAG | | GCTCAGA | | | | |
| | | | | | | | | | | GAGGCGG | | | | |
| TNFAIP3 | IL2RA Teffs | IL2RA high | 7128 | NM_001270507.1 | NC_000006.12 | 1.38E+08 | antisense | TGAGAGA | 1734. | GATTTGAG | 1782. | CGG | 3 | 0.6393 |
| | | | | | | | | CTCCAGTT | | AGACTCCA | | | | |
| | | | | | | | | GCCAG | | GTTGCCAG | | | | |
| | | | | | | | | | | CGGAAT | | | | |
| HIVEP2 | IL2RA Teffs | IL2RA high | 3097 | NM_006734.3 | NC_000006.12 | 1.43E+08 | antisense | CCCTGGAT | 1735. | CGGTCCCT | 1783. | CGG | 5 | 0.6747 |
| | | | | | | | | AGAATACA | | GGATAGA | | | | |
| | | | | | | | | TCGT | | ATACATCG | | | | |
| | | | | | | | | | | TCGGAAC | | | | |
| HIVEP2 | IL2RA Teffs | IL2RA high | 3097 | NM_006734.3 | NC_000006.12 | 1.43E+08 | sense | GACAAGA | 1736. | TTCAGACA | 1784. | GGG | 5 | 0.7382 |
| | | | | | | | | TGTCAGAC | | AGATGTCA | | | | |
| | | | | | | | | CTAGG | | GACCTAG | | | | |
| | | | | | | | | | | GGGGCAG | | | | |
| HIVEP2 | IL2RA Teffs | IL2RA high | 3097 | NM_006734.3 | NC_000006.12 | 1.43E+08 | antisense | GAGGTGG | 1737. | GTCAGAG | 1785. | GGG | 5 | 0.6922 |
| | | | | | | | | AAGGTAA | | GTGGAAG | | | | |
| | | | | | | | | ACACAA | | GTAAACAC | | | | |
| | | | | | | | | | | AAGGGGA | | | | |
| | | | | | | | | | | T | | | | |
| HIVEP2 | IL2RA Teffs | IL2RA high | 3097 | NM_006734.3 | NC_000006.12 | 1.43E+08 | antisense | TTCTAGGA | 1738. | TTTTTTCTA | 1786. | TGG | 5 | 0.71 |
| | | | | | | | | TAACCACC | | GGATAACC | | | | |
| | | | | | | | | ACTG | | ACCACTGT | | | | |
| | | | | | | | | | | GGCCA | | | | |
| MED12 | IL2RA Teffs | IL2RA high | 9968 | NM_005120.2 | NC_000023.11 | 71130165 | sense | ACATCGAC | 1739. | ATCCACAT | 1787. | AGG | 28 | 0.6457 |
| | | | | | | | | TGCTGGAC | | CGACTGCT | | | | |
| | | | | | | | | AATG | | GGACAAT | | | | |
| | | | | | | | | | | GAGGATG | | | | |
| MED12 | IL2RA Teffs | IL2RA high | 9968 | NM_005120.2 | NC_000023.11 | 71122231 | antisense | CAGTGAGT | 1740. | CAGTCAGT | 1788. | AGG | 8 | 0.688 |
| | | | | | | | | AGTGCCAA | | GAGTAGT | | | | |
| | | | | | | | | ACCA | | GCCAAACC | | | | |
| | | | | | | | | | | AAGGCAC | | | | |
| MED12 | IL2RA Teffs | IL2RÅ high | 9968 | NM_005120.2 | NC_000023.11 | 71125111 | antisense | GTGGCGT | 1741. | ATGGGTG | 1789. | TGG | 15 | 0.6661 |
| | | | | | | | | ACTGCACG | | GCGTACTG | | | | |
| | | | | | | | | TGTCG | | CACGTGTC | | | | |
| | | | | | | | | | | GTGGCTG | | | | |
| MED12 | IL2RA Teffs | IL2RA high | 9968 | NM_005120.2 | NC_000023.11 | 71126138 | sense | TTCACATT | 1742. | ACCTTTCA | 1790. | AGG | 18 | 0.6594 |
| | | | | | | | | ATGACCAA | | CATTATGA | | | | |
| | | | | | | | | CACC | | CCAACACC | | | | |
| | | | | | | | | | | AGGTCA | | | | |

TABLE 10
Nuclear factors that can be inhibited to decrease IL2RA expression or overexpressed to increase IL2RA expression
specifically in effector T cells

| | | | | | | Position | | | | | | | | |
|---|---|---|---|---|---|---|---|---|---|---|---|---|---|---|
| | | | | | | of Base | | | | | | | | |
| | | | | | | After | | sgRNA | | Target | | | | Rule |
| | screen_ | screen_ | Target | Target | Genomic | Cut (1- | | Target | SEQ ID | Context | SEQ | PAM | Exon | Set 2 |
| gene_id | target | direction | Gene ID | Transcript | Sequence | based | Strand | Sequence | NO:: | Sequence | ID NO: | Sequence | Number | score |
| GATA3 | IL2RA Teffs | IL2RA low | 2625 | NM_002051.2 | NC_000010.11 | 8055892 | sense | AGGTACCC | 1791. | GCAGAGG | 1879. | CGG | 2 | 0.6857 |
| | | | | | | | | TCCGACCC | | TACCCTCC | | | | |
| | | | | | | | | ACCA | | GACCCACC | | | | |
| | | | | | | | | | | ACGGTGA | | | | |
| GATA3 | IL2RA Teffs | IL2RA low | 2625 | NM_002051.2 | NC_000010.11 | 8064014 | sense | CAGGGAG | 1792. | AAGGCAG | 1880. | GGG | 4 | 0.737 |
| | | | | | | | | TGTGTGAA | | GGAGTGT | | | | |
| | | | | | | | | CTGTG | | GTGAACTG | | | | |
| | | | | | | | | | | TGGGGCA | | | | |
| | | | | | | | | | | A | | | | |
| GATA3 | IL2RA Teffs | IL2RA low | 2625 | NM_002051.2 | NC_000010.11 | 8058740 | antisense | GGAGCTG | 1793. | GTCCGGA | 1881. | AGG | 3 | 0.6273 |
| | | | | | | | | TACTCGGG | | GCTGTACT | | | | |
| | | | | | | | | CACGT | | CGGGCAC | | | | |
| | | | | | | | | | | GTAGGGC | | | | |
| | | | | | | | | | | G | | | | |
| GATA3 | IL2RA Teffs | IL2RA low | 2625 | NM_002051.2 | NC_000010.11 | 8058432 | sense | TCCAAGAC | 1794. | CTTCTCCA | 1882. | CGG | 3 | 0.7243 |
| | | | | | | | | GTCCATCC | | AGACGTCC | | | | |
| | | | | | | | | ACCA | | ATCCACCA | | | | |
| | | | | | | | | | | CGGCTC | | | | |
| KMT2A | IL2RA Teffs | IL2RA low | 4297 | NM_005933.3 | NC_000011.10 | 1.19E+08 | antisense | AAGATCA | 1795. | ATTCAAGA | 1883. | TGG | 27 | 0.7172 |
| | | | | | | | | GTAGCGG | | TCAGTAGC | | | | |
| | | | | | | | | TCCCGG | | GGTCCCG | | | | |
| | | | | | | | | | | GTGGTGG | | | | |
| KMT2A | IL2RA Teffs | IL2RA low | 4297 | NM_005933.3 | NC_000011.10 | 1.18E+08 | sense | AGAAAGG | 1796. | GTAAAGA | 1884. | CGG | 5 | 0.6636 |
| | | | | | | | | ACGTCGAT | | AAGGACG | | | | |
| | | | | | | | | CGAGG | | TCGATCGA | | | | |
| | | | | | | | | | | GGCGGTG | | | | |
| | | | | | | | | | | T | | | | |
| KMT2A | IL2RA Teffs | IL2RA low | 4297 | NM_005933.3 | NC_000011.10 | 1.18E+08 | antisense | AGGGGTC | 1797. | GCCGAGG | 1885. | AGG | 3 | 0.757 |
| | | | | | | | | TTAATGAT | | GGTCTTAA | | | | |
| | | | | | | | | CCGCG | | TGATCCGC | | | | |
| | | | | | | | | | | GAGGAGA | | | | |
| KMT2A | IL2RA Teffs | IL2RA low | 4297 | NM_005933.3 | NC_000011.10 | 1.18E+08 | sense | TTGACCAT | 1798. | TCACTTGA | 1886. | TGG | 19 | 0.7229 |
| | | | | | | | | AATTATGC | | CCATAATT | | | | |
| | | | | | | | | TCAG | | ATGCTCAG | | | | |
| | | | | | | | | | | TGGCAG | | | | |
| ETS1 | IL2RA Teffs | IL2RA low | 2113 | NM_005238.3 | NC_000011.10 | 1.28E+08 | antisense | CTTACTAA | 1799. | TGAACTTA | 1887. | AGG | 4 | 0.6808 |
| | | | | | | | | TGAAGTAA | | CTAATGAA | | | | |
| | | | | | | | | TCCG | | GTAATCCG | | | | |
| | | | | | | | | | | AGGTAT | | | | |
| ETS1 | IL2RA Teffs | IL2RA low | 2113 | NM_005238.3 | NC_000011.10 | 1.28E+08 | antisense | GAGAAAG | 1800. | GCTCGAG | 1888. | GGG | 3 | 0.6487 |
| | | | | | | | | CAGTCTTT | | AAAGCAG | | | | |
| | | | | | | | | ACCCA | | TCTTTACC | | | | |
| | | | | | | | | | | CAGGGCG | | | | |
| | | | | | | | | | | C | | | | |
| ETS1 | IL2RA Teffs | IL2RA low | 2113 | NM_005238.3 | NC_000011.10 | 1.28E+08 | antisense | GGTCTCGG | 1801. | AGAGGGT | 1889. | GGG | 5 | 0.7649 |
| | | | | | | | | AGAATGA | | CTCGGAG | | | | |
| | | | | | | | | CCGAG | | AATGACCG | | | | |
| | | | | | | | | | | AGGGGTA | | | | |
| | | | | | | | | | | G | | | | |
| ETS1 | IL2RA Teffs | IL2RA low | 2113 | NM_005238.3 | NC_000011.10 | 1.28E+08 | sense | TGCATGG | 1802. | CATGTGCA | 1890. | TGG | 5 | 0.6503 |
| | | | | | | | | GGAGGAC | | TGGGGAG | | | | |
| | | | | | | | | CAGTCG | | GACCAGTC | | | | |
| | | | | | | | | | | GTGGTAG | | | | |
| RBPJ | IL2RA Teffs | IL2RA low | 3516 | NM_005349.3 | NC_000004.12 | 26415547 | sense | AAAGAAC | 1803. | AAAAAAA | 1891. | TGG | 5 | 0.6336 |
| | | | | | | | | AAATGGA | | GAACAAAT | | | | |
| | | | | | | | | ACGCGA | | GGAACGC | | | | |
| | | | | | | | | | | GATGGTTG | | | | |
| RBPJ | IL2RA Teffs | IL2RA low | 3516 | NM_005349.3 | NC_000004.12 | 26386378 | antisense | CACCTAGT | 1804. | TACTCACC | 1892. | TGG | 3 | 0.6435 |
| | | | | | | | | AAGTOGTT | | TAGTAAGT | | | | |
| | | | | | | | | TAGG | | CGTTTAGG | | | | |
| | | | | | | | | | | TGGAGG | | | | |
| RBPJ | IL2RA Teffs | IL2RA low | 3516 | NM_005349.3 | NC_000004.12 | 26424454 | sense | CATGCCAG | 1805. | TTTTCATG | 1893. | GGG | 7 | 0.6844 |
| | | | | | | | | TTCACAGC | | CCAGTTCA | | | | |
| | | | | | | | | AGTG | | CAGCAGT | | | | |
| | | | | | | | | | | GGGGAGC | | | | |
| RBPJ | IL2RA Teffs | IL2RA low | 3516 | NM_005349.3 | NC_000004.12 | 26424363 | sense | CATTGCCT | 1806. | TATGCATT | 1894. | TGG | 7 | 0.65 |
| | | | | | | | | CAGGAAC | | GCCTCAGG | | | | |
| | | | | | | | | AAAGG | | AACAAAG | | | | |
| | | | | | | | | | | GTGGCTC | | | | |
| RXRB | IL2RA Teffs | IL2RA low | 6257 | NM_021976.4 | NC_000006.12 | 33198326 | sense | ACGGCTAT | 1807. | GCAAACG | 1895. | GGG | 3 | 0.7124 |
| | | | | | | | | GTGCAATC | | GCTATGTG | | | | |
| | | | | | | | | TGCG | | CAATCTGC | | | | |
| | | | | | | | | | | GGGGACA | | | | |
| RXRB | IL2RA Teffs | IL2RA low | 6257 | NM_021976.4 | NC_000006.12 | 33200341 | sense | GCCCTGGC | 1808. | GACGGCC | 1896. | CGG | 1 | 0.6547 |
| | | | | | | | | TGGATCCC | | CTGGCTGG | | | | |
| | | | | | | | | GCAG | | ATCCCGCA | | | | |
| | | | | | | | | | | GCGGCGG | | | | |
| RXRB | IL2RA Teffs | IL2RA low | 6257 | NM_021976.4 | NC_000006.12 | 33197840 | sense | GGACAAC | 1809. | GCCGGGA | 1897. | TGG | 4 | 0.7879 |
| | | | | | | | | AAAGACT | | CAACAAA | | | | |
| | | | | | | | | GCACAG | | GACTGCAC | | | | |
| | | | | | | | | | | AGTGGAC | | | | |
| | | | | | | | | | | A | | | | |
| RXRB | IL2RA Teffs | IL2RA low | 6257 | NM_021976.4 | NC_000006.12 | 33198421 | antisense | GTGGCTTC | 1810. | ACTGGTG | 1898. | GGG | 3 | 0.6579 |
| | | | | | | | | ACATCTTC | | GCTTCACA | | | | |
| | | | | | | | | AGGG | | TCTTCAGG | | | | |
| | | | | | | | | | | GGGGCCA | | | | |
| ZNF148 | IL2RA Teffs | IL2RA low | 7707 | NM_021964.2 | NC_000003.12 | 1.25E+08 | sense | AGATCGA | 1811. | TTCAAGAT | 1899. | AGG | 4 | 0.5525 |
| | | | | | | | | AGTATGCC | | CGAAGTAT | | | | |
| | | | | | | | | TCACC | | GCCTCACC | | | | |
| | | | | | | | | | | AGGAGA | | | | |
| ZNF148 | IL2RA Teffs | IL2RA low | 7707 | NM_021964.2 | NC_000003.12 | 1.25E+08 | antisense | AGTGCATA | 1812. | ATTAAGTG | 1900. | AGG | 4 | 0.6259 |
| | | | | | | | | CTGTAGTC | | CATACTGT | | | | |
| | | | | | | | | CTTG | | AGTCCTTG | | | | |
| | | | | | | | | | | AGGAAG | | | | |
| ZNF148 | IL2RA Teffs | IL2RA low | 7707 | NM_021964.2 | NC_000003.12 | 1.25E+08 | antisense | GAGCCCCC | 1813. | ATGCGAG | 1901. | TGG | 9 | 0.6379 |
| | | | | | | | | AACTGACG | | CCCCCAAC | | | | |
| | | | | | | | | AATG | | TGACGAAT | | | | |
| | | | | | | | | | | GTGGCAT | | | | |
| ZNF148 | IL2RA Teffs | IL2RA low | 7707 | NM_021964.2 | NC_000003.12 | 1.25E+08 | antisense | TAATTAGT | 1814 | ATCATAAT | 1902. | AGG | 9 | 0.6151 |
| | | | | | | | | ACTACTAT | | TAGTACTA | | | | |
| | | | | | | | | GCAC | | CTATGCAC | | | | |
| | | | | | | | | | | AGGTTT | | | | |
| VPS52 | IL2RA Teffs | IL2RA low | 6293 | NM_001289174.1 | NC_000006.12 | 33267957 | sense | CAATGAAC | 1815. | TGGGCAAT | 1903. | AGG | 8 | 0.6103 |
| | | | | | | | | GAGCAAC | | GAACGAG | | | | |
| | | | | | | | | AGCAA | | CAACAGCA | | | | |
| | | | | | | | | | | AAGGAGA | | | | |
| VPS52 | IL2RA Teffs | IL2RA low | 6293 | NM_001289174.1 | NC_000006.12 | 33266562 | sense | CCGTACAC | 1816. | TGGGCCGT | 1904. | TGG | 11 | 0.6309 |
| | | | | | | | | TCAGCATG | | ACACTCAG | | | | |
| | | | | | | | | ACCC | | CATGACCC | | | | |
| | | | | | | | | | | TGGTAA | | | | |
| VPS52 | IL2RA Teffs | IL2RA low | 6293 | NM_001289174.1 | NC_000006.12 | 33269070 | sense | GAAATCGC | 1817. | CTTCGAAA | 1905. | GGG | 5 | 0.7039 |
| | | | | | | | | CAGGCAG | | TCGCCAGG | | | | |
| | | | | | | | | TTCGG | | CAGTTCGG | | | | |
| | | | | | | | | | | GGGAAA | | | | |
| VPS52 | IL2RA Teffs | IL2RA low | 6293 | NM_001289174.1 | NC_000006.12 | 33264461 | antisense | TCCAGGAT | 1818. | CATCTCCA | 1906. | TGG | 13 | 0.6852 |
| | | | | | | | | CAGTTCAA | | GGATCAGT | | | | |
| | | | | | | | | ACCG | | TCAAACCG | | | | |
| | | | | | | | | | | TGGCCA | | | | |
| TFAP4 | IL2RA Teffs | IL2RA low | 7023 | NM_003223.2 | NC_000016.10 | 4262329 | sense | ACAGCTCA | 1819. | ACACACAG | 1907. | AGG | 3 | 0.5694 |
| | | | | | | | | AGCGCTTC | | CTCAAGCG | | | | |
| | | | | | | | | ATCC | | CTTCATCC | | | | |
| | | | | | | | | | | AGGTGC | | | | |
| TFAP4 | IL2RA Teffs | IL2RA low | 7023 | NM_003223.2 | NC_000016.10 | 4261877 | sense | AGGCTCCC | 1820. | GCATAGG | 1908. | AGG | 4 | 0.6975 |
| | | | | | | | | CGGACATC | | CTCCCCGG | | | | |
| | | | | | | | | TGGG | | ACATCTGG | | | | |
| | | | | | | | | | | GAGGACG | | | | |
| TFAP4 | IL2RA Teffs | IL2RA low | 7023 | NM_003223.2 | NC_000016.10 | 4262671 | sense | CACTAACC | 1821. | ATTCCACT | 1909. | CGG | 2 | 0.6468 |
| | | | | | | | | CCCGAGAC | | AACCCCCG | | | | |
| | | | | | | | | TCAG | | AGACTCAG | | | | |
| | | | | | | | | | | CGGGAC | | | | |
| TFAP4 | IL2RA Teffs | IL2RA low | 7023 | NM_003223.2 | NC_000016.10 | 4262597 | sense | CGCATGCA | 1822. | GAGACGC | 1910 | GGG | 2 | 0.634 |
| | | | | | | | | GAGCATCA | | ATGCAGA | | | | |
| | | | | | | | | ACGC | | GCATCAAC | | | | |
| | | | | | | | | | | GCGGGAT | | | | |
| | | | | | | | | | | T | | | | |
| IKZF3 | IL2RA Teffs | IL2RA low | 22806 | NM_012481.4 | NC_000017.11 | 39792732 | sense | AAGATGA | 1823. | TGGAAAG | 1911. | TGG | 4 | 0.7182 |
| | | | | | | | | ACTGCGAT | | ATGAACTG | | | | |
| | | | | | | | | GTGTG | | CGATGTGT | | | | |
| | | | | | | | | | | GTGGATT | | | | |
| IKZF3 | IL2RA Teffs | IL2RA low | 22806 | NM_012481.4 | NC_000017.11 | 39788318 | sense | CAAGCAG | 1824 | GTTACAAG | 1912. | AGG | 6 | 0.6635 |
| | | | | | | | | AGAAGTTC | | CAGAGAA | | | | |
| | | | | | | | | CCTTG | | GTTCCCTT | | | | |
| | | | | | | | | | | GAGGAGC | | | | |
| IKZF3 | IL2RA Teffs | IL2RA low | 22806 | NM_012481.4 | NC_000017.11 | 39766413 | sense | GCTCATAC | 1825. | GTGAGCTC | 1913. | TGG | 8 | 0.6101 |
| | | | | | | | | AGACCCGC | | ATACAGAC | | | | |
| | | | | | | | | ATGA | | CCGCATGA | | | | |
| | | | | | | | | | | TGGACC | | | | |
| IKZF3 | IL2RA Teffs | IL2RA low | 22806 | NM_012481.4 | NC_000017.11 | 39777693 | sense | GGACAGA | 1826. | TACTGGAC | 1914. | TGG | 7 | 0.7336 |
| | | | | | | | | TTAGCAAG | | AGATTAGC | | | | |
| | | | | | | | | CAATG | | AAGCAAT | | | | |
| | | | | | | | | | | GTGGCAA | | | | |
| SRF | IL2RA Teffs | IL2RA low | 6722 | NM_003131.3 | NC_000006.12 | 43175724 | antisense | AGGTTGGT | 1827. | CGGCAGG | 1915. | CGG | 3 | 0.6646 |
| | | | | | | | | GACTGTGA | | TTGGTGAC | | | | |
| | | | | | | | | ACGC | | TGTGAACG | | | | |
| | | | | | | | | | | CCGGCTT | | | | |
| SRF | IL2RA Teffs | IL2RA low | 6722 | NM_003131.3 | NC_000006.12 | 43172119 | sense | AGTTCATC | 1828. | ATGGAGTT | 1916. | CGG | 1 | 0.7054 |
| | | | | | | | | GACAACA | | CATCGACA | | | | |
| | | | | | | | | AGCTG | | ACAAGCTG | | | | |
| | | | | | | | | | | CGGCGC | | | | |
| SRF | IL2RA Teffs | IL2RA low | 6722 | NM_003131.3 | NC_000006.12 | 43175844 | antisense | GGGCTGA | 1829. | ACTGGGG | 1917. | TGG | 3 | 0.65 |
| | | | | | | | | CACTAGCA | | CTGACACT | | | | |
| | | | | | | | | GACAC | | AGCAGAC | | | | |
| | | | | | | | | | | ACTGGTGC | | | | |
| SRF | IL2RA Teffs | IL2RA low | 6722 | NM_003131.3 | NC_000006.12 | 43174015 | antisense | TCTGTTGT | 1830. | CTGGTCTG | 1918. | GGG | 2 | 0.605 |
| | | | | | | | | GGGGTCT | | TTGTGGG | | | | |
| | | | | | | | | GAACG | | GTCTGAAC | | | | |
| | | | | | | | | | | GGGGTGG | | | | |
| CEBPZ | IL2RA Teffs | IL2RA low | 10153 | NM_005760.2 | NC_000002.12 | 37223251 | antisense | AAAGCTCC | 1831. | ATATAAAG | 1919. | TGG | 3 | 0.6404 |
| | | | | | | | | ACATATAA | | CTCCACAT | | | | |
| | | | | | | | | ATGG | | ATAAATGG | | | | |
| | | | | | | | | | | TGGCAT | | | | |
| CEBPZ | IL2RA Teffs | IL2RA low | 10153 | NM_005760.2 | NC_000002.12 | 37228088 | antisense | ACAAAGC | 1832. | TGTTACAA | 1920. | CGG | 2 | 0.63 |
| | | | | | | | | AGCTCATG | | AGCAGCTC | | | | |
| | | | | | | | | AGCCA | | ATGAGCCA | | | | |
| | | | | | | | | | | CGGTAA | | | | |
| CEBPZ | IL2RA Teffs | IL2RA low | 10153 | NM_005760.2 | NC_000002.12 | 37227623 | antisense | CAACATTA | 1833. | AAAGCAA | 1921. | TGG | 2 | 0.6374 |
| | | | | | | | | AAGCCTG | | CATTAAAG | | | | |
| | | | | | | | | GACAC | | CCTGGACA | | | | |
| | | | | | | | | | | CTGGTAT | | | | |
| CEBPZ | IL2RA Teffs | IL2RA low | 10153 | NM_005760.2 | NC_000002.12 | 37228473 | sense | TGAAGGC | 1834. | TGGATGA | 1922. | GGG | 2 | 0.7347 |
| | | | | | | | | AATTGTGT | | AGGCAATT | | | | |
| | | | | | | | | CATCG | | GTGTCATC | | | | |
| | | | | | | | | | | GGGGACA | | | | |
| BATF | IL2RA Teffs | IL2RA low | 10538 | NM_006399.3 | NC_000014.9 | 75546499 | sense | ACAGAAC | 1835. | AGAAACA | 1923. | AGG | 3 | 0.599 |
| | | | | | | | | GCGGCTCT | | GAACGCG | | | | |
| | | | | | | | | ACGCA | | GCTCTACG | | | | |
| | | | | | | | | | | CAAGGAG | | | | |
| | | | | | | | | | | A | | | | |
| BATF | IL2RA Teffs | IL2RA low | 10538 | NM_006399.3 | NC_000014.9 | 75522739 | antisense | AGGACTCT | 1836. | AAGGAGG | 1924. | GGG | 1 | 0.6137 |
| | | | | | | | | ACCTGTTT | | ACTCTACC | | | | |
| | | | | | | | | GCCA | | TGTTTGCC | | | | |
| | | | | | | | | | | AGGGGGA | | | | |
| BATF | IL2RA Teffs | IL2RA low | 10538 | NM_006399.3 | NC_000014.9 | 75525114 | sense | ATGTGAG | 1837. | GATGATGT | 1925. | AGG | 2 | 0.5377 |
| | | | | | | | | AAGAGTTC | | GAGAAGA | | | | |
| | | | | | | | | AGAGG | | GTTCAGAG | | | | |
| | | | | | | | | | | GAGGGAG | | | | |
| BATF | IL2RA Teffs | IL2RA low | 10538 | NM_006399.3 | NC_000014.9 | 75546520 | sense | GGAGATC | 1838. | GCAAGGA | 1926. | AGG | 3 | 0.7037 |
| | | | | | | | | AAGCAGCT | | GATCAAGC | | | | |
| | | | | | | | | CACAG | | AGCTCACA | | | | |
| | | | | | | | | | | GAGGAAC | | | | |
| CIC | IL2RA Teffs | IL2RA low | 23152 | NM_015125.4 | NC_000019.10 | 42291060 | sense | ACTGTCAC | 1839. | TGCCACTG | 1927. | GGG | 10 | 0.6591 |
| | | | | | | | | TAACCTAC | | TCACTAAC | | | | |
| | | | | | | | | TGGT | | CTACTGGT | | | | |
| | | | | | | | | | | GGGCAC | | | | |
| CIC | IL2RA Teffs | IL2RA low | 23152 | NM_015125.4 | NC_000019.10 | 42292311 | antisense | CCCGCCCG | 1840. | CGTGCCCG | 1928. | AGG | 13 | 0.6689 |
| | | | | | | | | CTGACTGC | | CCCGCTGA | | | | |
| | | | | | | | | ACAT | | CTGCACAT | | | | |
| | | | | | | | | | | AGGTGA | | | | |
| CIC | IL2RA Teffs | IL2RA low | 23152 | NM_015125.4 | NC_000019.10 | 42287372 | sense | CTCTACCG | 1841. | TTGCCTCT | 1929. | CGG | 4 | 0.6842 |
| | | | | | | | | CCCGGAA | | ACCGCCCG | | | | |
| | | | | | | | | AACGT | | GAAAACG | | | | |
| | | | | | | | | | | TCGGACC | | | | |
| CIC | IL2RA Teffs | IL2RA low | 23152 | NM_015125.4 | NC_000019.10 | 42289198 | antisense | TTGGGCCA | 1842. | GGCTTTGG | 1930. | GGG | 8 | 0.6248 |
| | | | | | | | | GAGTACG | | GCCAGAG | | | | |
| | | | | | | | | ATGCA | | TACGATGC | | | | |
| | | | | | | | | | | AGGGCCA | | | | |
| IKZF1 | IL2RA Teffs | IL2RA low | 10320 | NM_006060.5 | NC_000007.14 | 50376659 | sense | GAAAATG | 1843. | GAGAGAA | 1931. | GGG | 4 | 0.66 |
| | | | | | | | | AATGGCTC | | AATGAATG | | | | |
| | | | | | | | | CCACA | | GCTCCCAC | | | | |
| | | | | | | | | | | AGGGACC | | | | |
| IKZF1 | IL2RA Teffs | IL2RA low | 10320 | NM_006060.5 | NC_000007.14 | 50399996 | antisense | GATGGCTT | 1844 | TGTTGATG | 1932. | GGG | 8 | 0.7379 |
| | | | | | | | | GGTCCATC | | GCTTGGTC | | | | |
| | | | | | | | | ACGT | | CATCACGT | | | | |
| | | | | | | | | | | GGGACT | | | | |
| IKZF1 | IL2RA Teffs | IL2RA low | 10320 | NM_006060.5 | NC_000007.14 | 50382586 | sense | GGGGCCT | 1845. | GTGCGGG | 1933. | GGG | 5 | 0.6196 |
| | | | | | | | | CATTCACC | | GCCTCATT | | | | |
| | | | | | | | | CAGAA | | CACCCAGA | | | | |
| | | | | | | | | | | AGGGCAA | | | | |
| IKZF1 | IL2RA Teffs | IL2RA low | 10320 | NM_006060.5 | NC_000007.14 | 50327753 | sense | TCCAAGAG | 1846. | AAGCTCCA | 1934. | GGG | 3 | 0.617 |
| | | | | | | | | TGACAGA | | AGAGTGA | | | | |
| | | | | | | | | GTCGT | | CAGAGTC | | | | |
| | | | | | | | | | | GTGGGTA | | | | |
| | | | | | | | | | | A | | | | |
| IRF4 | IL2RA Teffs | IL2RA low | 3662 | NM_001195286.1 | NC_000006.12 | 394977 | sense | CAGACCCG | 1847. | ATCTCAGA | 1935. | AGG | 3 | 0.5822 |
| | | | | | | | | TACAAAGT | | CCCGTACA | | | | |
| | | | | | | | | GTAC | | AAGTGTAC | | | | |
| | | | | | | | | | | AGGATT | | | | |
| IRF4 | IL2RA Teffs | IL2RA low | 3662 | NM_001195286.1 | NC_000006.12 | 397215 | sense | CCCATGAC | 1848. | ATGTCCCA | 1936. | CGG | 5 | 0.7031 |
| | | | | | | | | GTTTGGAC | | TGACGTTT | | | | |
| | | | | | | | | CCCG | | GGACCCC | | | | |
| | | | | | | | | | | GCGGCCA | | | | |
| IRF4 | IL2RA Teffs | IL2RA low | 3662 | NM_001195286.1 | NC_000006.12 | 401466 | sense | CTACCGGG | 1849. | TGTACTAC | 1937. | AGG | 7 | 0.6428 |
| | | | | | | | | AAATCCTC | | CGGGAAA | | | | |
| | | | | | | | | GTGA | | TCCTCGTG | | | | |
| | | | | | | | | | | AAGGAGC | | | | |
| IRF4 | IL2RA Teffs | IL2RA low | 3662 | NM_001195286.1 | NC_000006.12 | 393251 | sense | CTGATCGA | 1850. | GTGGCTG | 1938. | CGG | 2 | 0.6887 |
| | | | | | | | | CCAGATCG | | ATCGACCA | | | | |
| | | | | | | | | ACAG | | GATCGACA | | | | |
| | | | | | | | | | | GCGGCAA | | | | |
| MBD2 | IL2RA Teffs | IL2RA low | 8932 | NM_003927.4 | NC_000018.10 | 54224170 | sense | AGCCGGTC | 1851. | CGGGAGC | 1939. | GGG | 1 | 0.5949 |
| | | | | | | | | CCTTTCCC | | CGGTCCCT | | | | |
| | | | | | | | | GTCG | | TTCCCGTC | | | | |
| | | | | | | | | | | GGGGAGC | | | | |
| MBD2 | IL2RA Teffs | IL2RA low | 8932 | NM_003927.4 | NC_000018.10 | 54205113 | sense | CCTCAGTT | 1852. | CAAGCCTC | 1940. | GGG | 2 | 0.5818 |
| | | | | | | | | GGCAAGG | | AGTTGGCA | | | | |
| | | | | | | | | TACCT | | AGGTACCT | | | | |
| | | | | | | | | | | GGGAAA | | | | |
| MBD2 | IL2RA Teffs | IL2RA low | 8932 | NM_003927.4 | NC_000018.10 | 54204999 | sense | CCTCTCAA | 1853. | CGATCCTC | 1941. | TGG | 2 | 0.5401 |
| | | | | | | | | TCAAAATA | | TCAATCAA | | | | |
| | | | | | | | | AGGT | | AATAAGGT | | | | |
| | | | | | | | | | | TGGTTA | | | | |
| MBD2 | IL2RA Teffs | IL2RA low | 8932 | NM_003927.4 | NC_000018.10 | 54224048 | sense | CGAAAATC | 1854. | GATCCGAA | 1942. | TGG | 1 | 0.494 |
| | | | | | | | | TGGGCTAA | | AATCTGGG | | | | |
| | | | | | | | | GTGC | | CTAAGTGC | | | | |
| | | | | | | | | | | TGGCAA | | | | |
| JAK3 | IL2RA Teffs | IL2RA low | 3718 | NM_000215.3 | NC_000019.10 | 17835098 | antisense | ACTCTCCA | 1855. | ACTTACTC | 1943. | CGG | 15 | 0.7301 |
| | | | | | | | | GGCTTAAC | | TCCAGGCT | | | | |
| | | | | | | | | ACAG | | TAACACAG | | | | |
| | | | | | | | | | | CGGGGC | | | | |
| JAK3 | IL2RA Teffs | IL2RA low | 3718 | NM_000215.3 | NC_000019.10 | 17839577 | antisense | AGCTCTCG | 1856. | CAGGAGC | 1944. | GGG | 10 | 0.7109 |
| | | | | | | | | AAGACTGC | | TCTCGAAG | | | | |
| | | | | | | | | TGTG | | ACTGCTGT | | | | |
| | | | | | | | | | | GGGGTCG | | | | |
| JAK3 | IL2RA Teffs | IL2RA low | 3718 | NM_000215.3 | NC_000019.10 | 17836043 | antisense | GTGTACAA | 1857. | CCAGGTGT | 1945. | TGG | 14 | 0.6764 |
| | | | | | | | | ATTCCTGC | | ACAAATTC | | | | |
| | | | | | | | | ACCA | | CTGCACCA | | | | |
| | | | | | | | | | | TGGTGC | | | | |
| JAK3 | IL2RA Teffs | IL2RA low | 3718 | NM_000215.3 | NC_000019.10 | 17842538 | sense | TGACGCG | 1858. | TTCGTGAC | 1946. | AGG | 6 | 0.6476 |
| | | | | | | | | GAGGCGT | | GCGGAGG | | | | |
| | | | | | | | | ATTCGG | | CGTATTCG | | | | |
| | | | | | | | | | | GAGGACG | | | | |
| BCL6 | IL2RA Teffs | IL2RA low | 604 | NM_001706.4 | NC_000003.12 | 1.88E+08 | sense | AAGTGATA | 1859. | CACGAAGT | 1947. | TGG | 5 | 0.8035 |
| | | | | | | | | TGCACTAC | | GATATGCA | | | | |
| | | | | | | | | AGTG | | CTACAGTG | | | | |
| | | | | | | | | | | TGGCTG | | | | |
| BCL6 | IL2RA Teffs | IL2RA low | 604 | NM_001706.4 | NC_000003.12 | 1.88E+08 | sense | CAAGACAT | 1860. | GCCCCAAG | 1948. | GGG | 5 | 0.6458 |
| | | | | | | | | CATGGCCT | | ACATCATG | | | | |
| | | | | | | | | ATCG | | GCCTATCG | | | | |
| | | | | | | | | | | GGGTCG | | | | |
| BCL6 | IL2RA Teffs | IL2RA low | 604 | NM_001706.4 | NC_000003.12 | 1.88E+08 | antisense | CATCTGCA | 1861. | GCTCCATC | 1949. | TGG | 4 | 0.6878 |
| | | | | | | | | GGTACATA | | TGCAGGTA | | | | |
| | | | | | | | | GCCG | | CATAGCCG | | | | |
| | | | | | | | | | | TGGCCA | | | | |
| BCL6 | IL2RA Teffs | IL2RA low | 604 | NM_001706.4 | NC_000003.12 | 1.88E+08 | antisense | TCCCTCAC | 1862. | CAGATCCC | 1950. | AGG | 3 | 0.6413 |
| | | | | | | | | CTGCAGGC | | TCACCTGC | | | | |
| | | | | | | | | CATG | | AGGCCAT | | | | |
| | | | | | | | | | | GAGGACC | | | | |
| ZNF236 | IL2RA Teffs | IL2RA low | 7776 | NM_007345.3 | NC_000018.10 | 76919836 | sense | ATCACGGA | 1863. | ATGCATCA | 1951. | AGG | 20 | 0.6507 |
| | | | | | | | | AGTCTCGT | | CGGAAGT | | | | |
| | | | | | | | | CCTG | | CTCGTCCT | | | | |
| | | | | | | | | | | GAGGTCA | | | | |
| ZNF236 | IL2RA Teffs | IL2RA low | 7776 | NM_007345.3 | NC_000018.10 | 76913812 | sense | GCATGTGC | 1864. | AGCAGCAT | 1952. | GGG | 18 | 0.7309 |
| | | | | | | | | GGTCGCAC | | GTGCGGTC | | | | |
| | | | | | | | | ACCG | | GCACACCG | | | | |
| | | | | | | | | | | GGGAAA | | | | |
| ZNF236 | IL2RA Teffs | IL2RA low | 7776 | NM_007345.3 | NC_000018.10 | 76905317 | sense | GGCAGCTT | 1865. | TGGTGGC | 1953. | GGG | 13 | 0.6112 |
| | | | | | | | | ACGGCGA | | AGCTTACG | | | | |
| | | | | | | | | CACAT | | GCGACAC | | | | |
| | | | | | | | | | | ATGGGTAT | | | | |
| ZNF236 | IL2RA Teffs | IL2RA low | 7776 | NM_007345.3 | NC_000018.10 | 76910097 | antisense | TGGTCTAC | 1866. | AAACTGGT | 1954. | AGG | 15 | 0.6536 |
| | | | | | | | | GTGCCCTC | | CTACGTGC | | | | |
| | | | | | | | | GCAG | | CCTCGCAG | | | | |
| | | | | | | | | | | AGGGTC | | | | |
| TCF3 | IL2RA Teffs | IL2RA low | 6929 | NM_003200.3 | NC_000019.10 | 1622055 | sense | CACCAGCA | 1867. | CCTGCACC | 1955. | AGG | 10 | 0.6193 |
| | | | | | | | | CGAGCGT | | AGCACGA | | | | |
| | | | | | | | | ATGGT | | GCGTATG | | | | |
| | | | | | | | | | | GTAGGAC | | | | |
| | | | | | | | | | | C | | | | |
| TCF3 | IL2RA Teffs | IL2RA low | 6929 | NM_003200.3 | NC_000019.10 | 1622322 | sense | CTATCCCG | 1868. | GCACCTAT | 1956. | TGG | 9 | 0.6605 |
| | | | | | | | | CCCCCTTC | | CCCGCCCC | | | | |
| | | | | | | | | TACG | | CTTCTACG | | | | |
| | | | | | | | | | | TGGCAG | | | | |
| TCF3 | IL2RA Teffs | IL2RA low | 6929 | NM_003200.3 | NC_000019.10 | 1619821 | antisense | GCTGGGC | 1869. | CGTAGCTG | 1957. | GGG | 14 | 0.6527 |
| | | | | | | | | GATAAGG | | GGCGATA | | | | |
| | | | | | | | | CACCGG | | AGGCACC | | | | |
| | | | | | | | | | | GGGGGCT | | | | |
| | | | | | | | | | | C | | | | |
| TCF3 | IL2RA Teffs | IL2RA low | 6929 | NM_003200.3 | NC_000019.10 | 1621034 | antisense | GTTATTGC | 1870. | AGAAGTTA | 1958. | GGG | 13 | 0.6808 |
| | | | | | | | | TTGAGTGA | | TTGCTTGA | | | | |
| | | | | | | | | TCCG | | GTGATCCG | | | | |
| | | | | | | | | | | GGGAGT | | | | |
| YY1 | IL2RA Teffs | IL2RA low | 7528 | NM_003403.4 | NC_000014.9 | 1E+08 | sense | AGATATTG | 1871. | AAAAAGA | 1959. | TGG | 2 | 0.667 |
| | | | | | | | | ACCATGAG | | TATTGACC | | | | |
| | | | | | | | | ACAG | | ATGAGAC | | | | |
| | | | | | | | | | | AGTGGTTG | | | | |
| YY1 | IL2RA Teffs | IL2RA low | 7528 | NM_003403.4 | NC_000014.9 | 1E+08 | sense | GGAGACC | 1872. | CGGTGGA | 1960. | TGG | 1 | 0.7975 |
| | | | | | | | | ATCGAGAC | | GACCATCG | | | | |
| | | | | | | | | CACAG | | AGACCACA | | | | |
| | | | | | | | | | | GTGGTGG | | | | |
| YY1 | IL2RA Teffs | IL2RA low | 7528 | NM_003403.4 | NC_000014.9 | 1E+08 | sense | GGTCACCG | 1873. | CGCTGGTC | 1961. | AGG | 1 | 0.6314 |
| | | | | | | | | ACGACCCG | | ACCGACG | | | | |
| | | | | | | | | ACCC | | ACCCGACC | | | | |
| | | | | | | | | | | CAGGTGC | | | | |
| YY1 | IL2RA Teffs | IL2RA low | 7528 | NM_003403.4 | NC_000014.9 | 1E+08 | sense | TGAACAAA | 1874. | ACATTGAA | 1962. | TGG | 1 | 0.7093 |
| | | | | | | | | CGCTGGTC | | CAAACGCT | | | | |
| | | | | | | | | ACCG | | GGTCACCG | | | | |
| | | | | | | | | | | TGGCGG | | | | |
| PURA | IL2RA Teffs | IL2RA low | 5813 | NM_005859.4 | NC_000005.10 | 1.4E+08 | sense | CCTTACTC | 1875. | GCCGCCTT | 1963. | TGG | 1 | 0.6294 |
| | | | | | | | | TCTCCATG | | ACTCTCTC | | | | |
| | | | | | | | | TCAG | | CATGTCAG | | | | |
| | | | | | | | | | | TGGCCG | | | | |
| PURA | IL2RA Teffs | IL2RA low | 5813 | NM_005859.4 | NC_000005.10 | 1.4E+08 | antisense | GAACTCGA | 1876. | CACGGAA | 1964. | CGG | 1 | 0.6814 |
| | | | | | | | | TGAGCCCC | | CTCGATGA | | | | |
| | | | | | | | | TGCG | | GCCCCTGC | | | | |
| | | | | | | | | | | GCGGGCA | | | | |
| PURA | IL2RA Teffs | IL2RA low | 5813 | NM_005859.4 | NC_000005.10 | 1.4E+08 | sense | GCTCATCG | 1877. | CCAAGCTC | 1965 | TGG | 1 | 0.6758 |
| | | | | | | | | ACGACTAC | | ATCGACGA | | | | |
| | | | | | | | | GGAG | | CTACGGA | | | | |
| | | | | | | | | | | GTGGAGG | | | | |
| PURA | IL2RA Teffs | IL2RA low | 5813 | NM_005859.4 | NC_000005.10 | 1.4E+08 | sense | TCCGCCAG | 1878. | CGCATCCG | 1966. | GGG | 1 | 0.6124 |
| | | | | | | | | ACGGTCAA | | CCAGACG | | | | |
| | | | | | | | | CCGG | | GTCAACCG | | | | |
| | | | | | | | | | | GGGGCCT | | | | |

TABLE 11
Nuclear factors that can be inhibited to increase IL2RA expression or overexpressed to decrease IL2RA expression
in regulatory T cells

| | | | | | | Position of | | sgRNA | | Target | | | |
|---|---|---|---|---|---|---|---|---|---|---|---|---|---|
| | | | Target | Target | Genomic | Base After | | Target | SEQ ID | Context | SEQ ID | PAM | Exon |
| gene_id | screen_target | screen_direction | Gene ID | Transcript | Sequence | Cut (1-based) | Strand | Sequence | NO: | Sequence | NO: | Sequence | Number |
| RAD21 | IL2RA Tregs | IL2RA | 5885 | NM_006265.2 | NC_000008.11 | 1.17E+08 | antisense | AAGTGTTGT | 1967. | GAACAAG | 1979. | TGG | 8 |
| | | high | | | | | | TTGATCAGT | | TGTTGTTT | | | |
| | | | | | | | | CA | | GATCAGT | | | |
| | | | | | | | | | | CATGGTT | | | |
| | | | | | | | | | | G | | | |
| RAD21 | IL2RA Tregs | IL2RA | 5885 | NM_006265.2 | NC_000008.11 | 1.17E+08 | antisense | ACATACTCT | 1968. | AGACACA | 1980. | TGG | 4 |
| | | high | | | | | | AAGTCAGGC | | TACTCTAA | | | |
| | | | | | | | | AG | | GTCAGGC | | | |
| | | | | | | | | | | AGTGGCT | | | |
| | | | | | | | | | | G | | | |
| RAD21 | IL2RA Tregs | IL2RA | 5885 | NM_006265.2 | NC_000008.11 | 1.17E+08 | sense | GTGTAATTT | 1969. | TCGAGTG | 1981. | TGG | 2 |
| | | high | | | | | | AGAGAGCA | | TAATTTA | | | |
| | | | | | | | | GCG | | GAGAGCA | | | |
| | | | | | | | | | | GCGTGGA | | | |
| | | | | | | | | | | GA | | | |
| RAD21 | IL2RA Tregs | IL2RA | 5885 | NM_006265.2 | NC_000008.11 | 1.17E+08 | antisense | TCTGTTCAG | 1970. | GTGCTCT | 1982. | AGG | 6 |
| | | high | | | | | | ACTCTAATA | | GTTCAGA | | | |
| | | | | | | | | GG | | CTCTAATA | | | |
| | | | | | | | | | | GGAGGTT | | | |
| | | | | | | | | | | A | | | |
| RNF20 | IL2RA Tregs | IL2RA | 56254 | NM_019592.6 | NC_000009.12 | 1.02E+08 | sense | ACTTCGGCA | 1971. | AGAAACT | 1983. | AGG | 9 |
| | | high | | | | | | AGACTTTGA | | TCGGCAA | | | |
| | | | | | | | | GG | | GACTTTG | | | |
| | | | | | | | | | | AGGAGGT | | | |
| | | | | | | | | | | CA | | | |
| RNF20 | IL2RA Tregs | IL2RA | 56254 | NM_019592.6 | NC_000009.12 | 1.02E+08 | sense | GCATCGCAC | 1972. | AAAAGCA | 1984. | AGG | 6 |
| | | high | | | | | | CATGTCTCA | | TCGCACC | | | |
| | | | | | | | | GG | | ATGTCTC | | | |
| | | | | | | | | | | AGGAGGT | | | |
| | | | | | | | | | | AC | | | |
| RNF20 | IL2RA Tregs | IL2RA | 56254 | NM_019592.6 | NC_000009.12 | 1.02E+08 | antisense | GGAGGGCA | 1973. | TGCAGGA | 1985. | AGG | 13 |
| | | high | | | | | | CTACCACTA | | GGGCACT | | | |
| | | | | | | | | CGC | | ACCACTA | | | |
| | | | | | | | | | | CGCAGGC | | | |
| | | | | | | | | | | GT | | | |
| RNF20 | IL2RA Tregs | IL2RA | 56254 | NM_019592.6 | NC_000009.12 | 1.02E+08 | antisense | TCGGTTGAC | 1974. | AGTATCG | 1986. | AGG | 3 |
| | | high | | | | | | AATCAATAG | | GTTGACA | | | |
| | | | | | | | | TG | | ATCAATA | | | |
| | | | | | | | | | | GTGAGGC | | | |
| | | | | | | | | | | AT | | | |
| ZNF626 | IL2RA Tregs | IL2RA | 199777 | NM_145297.3 | NC_000019.10 | 20646387 | sense | ACCATTGCA | 1975. | AGGGACC | 1987. | TGG | 2 |
| | | high | | | | | | ATTTAGAGA | | ATTGCAA | | | |
| | | | | | | | | TG | | TTTAGAG | | | |
| | | | | | | | | | | ATGTGGC | | | |
| | | | | | | | | | | CA | | | |
| ZNF626 | IL2RA Tregs | IL2RA | 199777 | NM_145297.3 | NC_000019.10 | 20645740 | sense | CTGATCACC | 1976. | AGACCTG | 1988. | AGG | 3 |
| | | high | | | | | | TGTCTGGAG | | ATCACCT | | | |
| | | | | | | | | CA | | GTCTGGA | | | |
| | | | | | | | | | | GCAAGGA | | | |
| | | | | | | | | | | AG | | | |
| ZNF626 | IL2RA Tregs | IL2RA | 199777 | NM_145297.3 | NC_000019.10 | 20646366 | sense | GGCCATAGA | 1977. | ATGTGGC | 1989. | AGG | 2 |
| | | high | | | | | | ATTCTCTCTG | | CATAGAA | | | |
| | | | | | | | | G | | TTCTCTCT | | | |
| | | | | | | | | | | GGAGGA | | | |
| | | | | | | | | | | GT | | | |
| ZNF626 | IL2RA Tregs | IL2RA | 199777 | NM_145297.3 | NC_000019.10 | 20646279 | sense | TAACCTGGT | 1978. | ACAGTAA | 1990. | AGG | 2 |
| | | high | | | | | | CTTCCTTGGT | | CCTGGTC | | | |
| | | | | | | | | G | | TTCCTTGG | | | |
| | | | | | | | | | | TGAGGAT | | | |
| | | | | | | | | | | A | | | |

TABLE 12
Nuclear factors that can be inhibited to decrease IL2RA expression or overexpressed to increase IL2RA expression
specifically in regulatory T cells

| | | | | | | Position of | | | | | | | |
|---|---|---|---|---|---|---|---|---|---|---|---|---|---|
| | | | | | | Base After | | sgRNA | SEQ | Target | SEQ | | |
| | | screen_ | Target | Target | Genomic | Cut (1- | | Target | ID | Context | ID | PAM | Exon |
| gene_id | screen_target | direction | Gene ID | Transcript | Sequence | based) | Strand | Sequence | NO: | Sequence | NO: | Sequence | Number |
| FOXP3 | IL2RA Tregs | IL2RA low | 50943 | NM_014009. | NC_000023. | 49257007 | sense | CCCACCCACA | 1991. | TGTCCCCACC | 2010. | TGG | 5 |
| | | | | 3 | 11 | | | GGGATCAAC | | CACAGGGATC | | | |
| | | | | | | | | G | | AACGTGGCCA | | | |
| FOXP3 | IL2RA Tregs | IL2RA low | 50943 | NM_014009. | NC_000023. | 49255795 | sense | CCTACTTAGG | 1992. | TCTCCCTACTT | 2011. | CGG | 7 |
| | | | | 3 | 11 | | | CACTGCCAGG | | AGGCACTGCC | | | |
| | | | | | | | | | | AGGCGGACC | | | |
| FOXP3 | IL2RA Tregs | IL2RA low | 50943 | NM_014009. | NC_000023. | 49257751 | antisense | GAGGGTGCC | 1993. | CCCGGAGGGT | 2012. | GGG | 3 |
| | | | | 3 | 11 | | | ACCATGACTA | | GCCACCATGA | | | |
| | | | | | | | | G | | CTAGGGGCA | | | |
| | | | | | | | | | | G | | | |
| TAF5L | IL2RA Tregs | IL2RA low | 27097 | NM_014409. | NC_000001. | 2.3E+08 | antisense | CGGGACACG | 1994. | GATGCGGGA | 2013. | GGG | 4 |
| | | | | 3 | 11 | | | TCTACTTGGT | | CACGTCTACT | | | |
| | | | | | | | | G | | TGGTGGGGCT | | | |
| | | | | | | | | | | C | | | |
| TAF5L | IL2RA Tregs | IL2RA low | 27097 | NM_014409. | NC_000001. | 2.3E+08 | sense | GCAGAACGA | 1995. | TTCTGCAGAA | 2014. | AGG | 4 |
| | | | | 3 | 11 | | | GGCTGCCCTA | | CGAGGCTGCC | | | |
| | | | | | | | | G | | CTAGAGGTCT | | | |
| TAF5L | IL2RA Tregs | IL2RA low | 27097 | NM_014409. | NC_000001. | 2.3E+08 | sense | GCGGACCAG | 1996. | CACTGCGGAC | 2015. | AGG | 5 |
| | | | | 3 | 11 | | | TGTACAGCAC | | CAGTGTACAG | | | |
| | | | | | | | | G | | CACGAGGTTC | | | |
| TAF5L | IL2RA Tregs | IL2RA low | 27097 | NM_014409. | NC_000001. | 2.3E+08 | antisense | TAAGGTGAG | 1997. | TATGTAAGGT | 2016. | GGG | 4 |
| | | | | 3 | 11 | | | GACTTTGCAC | | GAGGACTTTG | | | |
| | | | | | | | | A | | CACAGGGCA | | | |
| | | | | | | | | | | G | | | |
| USF2 | IL2RA Tregs | IL2RA low | 7392 | NM_003367. | NC_000019. | 35270541 | antisense | AGCCGTAGTA | 1998. | ACACAGCCGT | 2017. | TGG | 5 |
| | | | | 2 | 10 | | | TCTCCCACAC | | AGTATCTCCC | | | |
| | | | | | | | | | | ACACTGGACG | | | |
| USF2 | IL2RA Tregs | IL2RA low | 7392 | NM_003367. | NC_000019. | 35269463 | sense | CCACGACAAG | 1999 | GCAGCCACGA | 2018. | CGG | 2 |
| | | | | 2 | 10 | | | GGACCCGAG | | CAAGGGACCC | | | |
| | | | | | | | | G | | GAGGCGGAG | | | |
| | | | | | | | | | | G | | | |
| USF2 | IL2RA Tregs | IL2RA low | 7392 | NM_003367. | NC_000019. | 35270800 | antisense | CCTGCACATA | 2000. | GTCCCCTGCA | 2019. | GGG | 6 |
| | | | | 2 | 10 | | | CGGAGAGTA | | CATACGGAGA | | | |
| | | | | | | | | A | | GTAAGGGTGT | | | |
| USF2 | IL2RA Tregs | IL2RA low | 7392 | NM_003367. | NC_000019. | 35269690 | sense | TTCCGCACAG | 2001. | CCAGTTCCGC | 2020. | AGG | 3 |
| | | | | 2 | 10 | | | AGACAAATG | | ACAGAGACAA | | | |
| | | | | | | | | G | | ATGGAGGAC | | | |
| | | | | | | | | | | A | | | |
| GABPA | IL2RA Tregs | IL2RA low | 2551 | NM_001197297. | NC_000021 | 25741671 | sense | AAGCAGAGT | 2002. | GAGAAAGCA | 2021. | AGG | 2 |
| | | | | 1 | | | | GCACAGAAG | | GAGTGCACAG | | | |
| | | | | | | | | AA | | AAGAAAGGTT | | | |
| | | | | | | | | | | G | | | |
| GABPA | IL2RA Tregs | IL2RA low | 2551 | NM_001197297. | NC_000021. | 25758095 | antisense | CCGAAATGTT | 2003. | CTCCCCGAAA | 2022. | AGG | 6 |
| | | | | 1 | 9 | | | GAGTGTGGT | | TGTTGAGTGT | | | |
| | | | | | | | | G | | GGTGAGGTCT | | | |
| GABPA | IL2RA Tregs | IL2RA low | 2551 | NM_001197297. | NC_000021. | 25758165 | antisense | CTCCAGAGAA | 2004. | ATGACTCCAG | 2023. | AGG | 6 |
| | | | | 1 | 9 | | | TTTCTCCCCG | | AGAATTTCTC | | | |
| | | | | | | | | | | CCCGAGGAAC | | | |
| GABPA | IL2RA Tregs | IL2RA low | 2551 | NM_001197297. | NC_000021. | 25758018 | sense | TCTTTCTAGA | 2005. | TTTTTCTTTCT | 2024. | TGG | 6 |
| | | | | 1 | 9 | | | TCCCATACAG | | AGATOCCATA | | | |
| | | | | | | | | | | CAGTGGTCC | | | |
| MED30 | IL2RA Tregs | IL2RA low | 90390 | NM_080651. | NC_000008. | 1.18E+08 | sense | ACACTGGAAC | 2006. | TACCACACTG | 2025. | CGG | 2 |
| | | | | 3 | 11 | | | ATATCAAGAC | | GAACATATCA | | | |
| | | | | | | | | | | AGACCGGTTA | | | |
| MED30 | IL2RA Tregs | IL2RA low | 90390 | NM_080651. | NC_000008. | 1.18E+08 | sense | GACAAATGCA | 2007. | ATATGACAAA | 2026. | TGG | 2 |
| | | | | 3 | 11 | | | ATGAAAACTG | | TGCAATGAAA | | | |
| | | | | | | | | | | ACTGTGGTGG | | | |
| MED30 | IL2RA Tregs | IL2RA low | 90390 | NM_080651. | NC_000008. | 1.18E+08 | sense | GGACATCGT | 2008. | TGCAGGACAT | 2027. | TGG | 1 |
| | | | | 3 | 11 | | | GTACCGCACC | | CGTGTACCGC | | | |
| | | | | | | | | A | | ACCATGGAGA | | | |
| MED30 | IL2RA Tregs | IL2RA low | 90390 | NM_080651. | NC_000008. | 1.18E+08 | sense | GGCCGCCCG | 2009. | AGCAGGCCGC | 2028. | CGG | 1 |
| | | | | 3 | 11 | | | GGAAGTCAA | | CCGGGAAGTC | | | |
| | | | | | | | | CA | | AACACGGCGT | | | |

TABLE 13
Nuclear factors that can be inhibited to increase IL2RA expression or overexpressed to decrease IL2RA expression in
effector T cells and regulatory T cells

| | | | | | | Position | | | | | | | | |
|---|---|---|---|---|---|---|---|---|---|---|---|---|---|---|
| | | | | | | of Base | | sgRNA | SEQ | Target | SEQ | | | |
| | screen_ | screen_ | Target | Target | Genomic | After Cut | | Target | ID | Context | ID | PAM | Exon | Rule Set 2 |
| gene_id | target | direction | Gene ID | Transcript | Sequence | (1-based) | Strand | Sequence | NO: | Sequence | NO: | Sequence | Number | score |
| HNRNPK | IL2RA | IL2RA | 3190 | NM_ | NC_ | 83972098 | sense | ATGATGT | 2029. | TACAATG | 2157. | CGG | 11 | 0.7754 |
| | Tregs | high | | 002140.3 | 000009.12 | | | TTGATGA | | ATGTTTG | | | | |
| | | | | | | | | CCGTCG | | ATGACCG | | | | |
| | | | | | | | | | | TCGCGGA | | | | |
| | | | | | | | | | | CG | | | | |
| HNRNPK | IL2RA | IL2RA | 3190 | NM_ | NC_ | 83975465 | antisense | CTGTTGG | 2030. | TAAACTG | 2158. | GGG | 6 | 0.6273 |
| | Tregs | high | | 002140.3 | 000009.12 | | | GACATAC | | TTGGGAC | | | | |
| | | | | | | | | CGCTCG | | ATACCGC | | | | |
| | | | | | | | | | | TCGGGG | | | | |
| | | | | | | | | | | CCA | | | | |
| HNRNPK | IL2RA | IL2RA | 3190 | NM_ | NC_ | 83971978 | sense | GATGATA | 2031. | TTATGAT | 2159. | AGG | 11 | 0.7149 |
| | Tregs | high | | 002140.3 | 000009.12 | | | TGAGCCC | | GATATGA | | | | |
| | | | | | | | | TCGTCG | | GCCCTCG | | | | |
| | | | | | | | | | | TCGAGG | | | | |
| | | | | | | | | | | ACC | | | | |
| HNRNPK | IL2RA | IL2RA | 3190 | NM_ | NC_ | 83973291 | sense | TAAAATC | 2032. | GTGCTAA | 2160. | AGG | 9 | 0.6608 |
| | Tregs | high | | 002140.3 | 000009.12 | | | AAAGAA | | AATCAAA | | | | |
| | | | | | | | | CTTCGAG | | GAACTTC | | | | |
| | | | | | | | | | | GAGAGG | | | | |
| | | | | | | | | | | TAA | | | | |
| KLF13 | IL2RA | IL2RA | 51621 | NM_ | NC_ | 31327702 | sense | CCGACCT | 2033. | CGCGCCG | 2161. | AGG | 1 | 0.7221 |
| | Tregs | high | | 015995.3 | 000015.10 | | | CGAGTCC | | ACCTCGA | | | | |
| | | | | | | | | CCGCAG | | GTCCCCG | | | | |
| | | | | | | | | | | CAGAGG | | | | |
| | | | | | | | | | | AAG | | | | |
| KLF13 | IL2RA | IL2RA | 51621 | NM_ | NC_ | 31327415 | sense | CGTGGTG | 2034. | TCTTOGT | 2162. | CGG | 1 | 0.6798 |
| | Tregs | high | | 015995.3 | 000015.10 | | | GCGCGG | | GGTGGC | | | | |
| | | | | | | | | ATCCTAG | | GCGGATC | | | | |
| | | | | | | | | | | CTAGCGG | | | | |
| | | | | | | | | | | ACC | | | | |
| KLF13 | IL2RA | IL2RA | 51621 | NM_ | NC_ | 31327274 | sense | CGTGTCC | 2035. | GCCTCGT | 2163. | CGG | 1 | 0.7 |
| | Tregs | high | | 015995.3 | 000015.10 | | | ATGTCGA | | GTCCATG | | | | |
| | | | | | | | | GCCGCG | | TCGAGCC | | | | |
| | | | | | | | | | | GCGCGG | | | | |
| | | | | | | | | | | TCG | | | | |
| KLF13 | IL2RA | IL2RA | 51621 | NM_ | NC_ | 31327767 | antisense | GAGTTCT | 2036. | GTGTGA | 2164. | AGG | 1 | 0.592 |
| | Tregs | high | | 015995.3 | 000015.10 | | | CAGGTGC | | GTTCTCA | | | | |
| | | | | | | | | GCCTTG | | GGTGCG | | | | |
| | | | | | | | | | | CCTTGAG | | | | |
| | | | | | | | | | | GTGC | | | | |
| TFDP1 | IL2RA | IL2RA | 7027 | NM_ | NC_ | 1.14E+08 | sense | ACCGGCA | 2037. | AGAGAC | 2165. | TGG | 5 | 0.6315 |
| | Tregs | high | | 007111.4 | 000013.11 | | | GCGTCAA | | CGGCAG | | | | |
| | | | | | | | | ACACCC | | CGTCAAA | | | | |
| | | | | | | | | | | CACCCTG | | | | |
| | | | | | | | | | | GTGG | | | | |
| TFDP1 | IL2RA | IL2RA | 7027 | NM_ | NC_ | 1.14E+08 | sense | ATGACCA | 2038. | GCTTATG | 2166. | CGG | 7 | 0.6204 |
| | Tregs | high | | 007111.4 | 000013.11 | | | GAAAAA | | ACCAGAA | | | | |
| | | | | | | | | CATAAGA | | AAACATA | | | | |
| | | | | | | | | | | AGACGG | | | | |
| | | | | | | | | | | CGC | | | | |
| TFDP1 | IL2RA | IL2RA | 7027 | NM_ | NC_ | 1.14E+08 | antisense | CCTTCAT | 2039. | CAGACCT | 2167. | AGG | 6 | 0.6851 |
| | Tregs | high | | 007111.4 | 000013.11 | | | GGAGAA | | TCATGGA | | | | |
| | | | | | | | | ATGCCGT | | GAAATGC | | | | |
| | | | | | | | | | | CGTAGGC | | | | |
| | | | | | | | | | | CC | | | | |
| TFDP1 | IL2RA | IL2RA | 7027 | NM_ | NC_ | 1.14E+08 | sense | GGTGCA | 2040. | ACCTGGT | 2168. | CGG | 9 | 0.6402 |
| | Tregs | high | | 007111.4 | 000013.11 | | | GAGAAA | | GCAGAG | | | | |
| | | | | | | | | CCGGCAT | | AAACCG | | | | |
| | | | | | | | | G | | GCATGCG | | | | |
| | | | | | | | | | | GAGC | | | | |
| NFKB2 | IL2RA | IL2RA | 4791 | NM_ | NC_ | 1.02E+08 | sense | ACTCGAC | 2041. | CCCTACT | 2169. | CGG | 14 | 0.7089 |
| | Tregs | high | | 001077494.3 | 000010.11 | | | TACGGCG | | CGACTAC | | | | |
| | | | | | | | | TCACCG | | GGCGTCA | | | | |
| | | | | | | | | | | CCGCGG | | | | |
| | | | | | | | | | | ACG | | | | |
| NFKB2 | IL2RA | IL2RA | 4791 | NM_ | NC_ | 1.02E+08 | sense | CCCACTC | 2042. | ATATCCC | 2170. | GGG | 9 | 0.6913 |
| | Tregs | high | | 001077494.3 | 000010.11 | | | CATAGAA | | ACTCCAT | | | | |
| | | | | | | | | TCTCCG | | AGAATCT | | | | |
| | | | | | | | | | | CCGGGG | | | | |
| | | | | | | | | | | GCA | | | | |
| NFKB2 | IL2RA | IL2RA | 4791 | NM_ | NC_ | 1.02E+08 | sense | CTGCAAC | 2043. | GTTTCTG | 2171. | AGG | 11 | 0.6401 |
| | Tregs | high | | 001077494.3 | 000010.11 | | | TGAAACG | | CAACTGA | | | | |
| | | | | | | | | CAAGCG | | AACGCAA | | | | |
| | | | | | | | | | | GCGAGG | | | | |
| | | | | | | | | | | AGG | | | | |
| NFKB2 | IL2RA | IL2RA | 4791 | NM_ | NC_ | 1.02E+08 | sense | GGGACC | 2044. | ACGAGG | 2172. | TGG | 6 | 0.7003 |
| | Tregs | high | | 001077494.3 | 000010.11 | | | AGCCAA | | GACCAGC | | | | |
| | | | | | | | | GATCGA | | CAAGATC | | | | |
| | | | | | | | | GG | | GAGGTG | | | | |
| | | | | | | | | | | GACC | | | | |
| FOXK1 | IL2RA | IL2RA | 221937 | NM_ | NC_ | 4757132 | sense | AACAGG | 2045. | GTGGAA | 2173. | AGG | 5 | 0.6681 |
| | Tregs | high | | 001037165.1 | 000007.14 | | | CATTCCG | | CAGGCAT | | | | |
| | | | | | | | | GAAACG | | TCCGGAA | | | | |
| | | | | | | | | G | | ACGGAG | | | | |
| | | | | | | | | | | GCAG | | | | |
| FOXK1 | IL2RA | IL2RA | 221937 | NM_ | NC_ | 4754516 | antisense | AGGTCAC | 2046. | TCCGAGG | 2174. | CGG | 3 | 0.734 |
| | Tregs | high | | 001037165.1 | 000007.14 | | | GTTCTGC | | TCACGTT | | | | |
| | | | | | | | | ACAAAG | | CTGCACA | | | | |
| | | | | | | | | | | AAGCGG | | | | |
| | | | | | | | | | | TAA | | | | |
| FOXK1 | IL2RA | IL2RA | 221937 | NM_ | NC_ | 4759162 | antisense | CTAACTT | 2047. | GAAGCTA | 2175. | GGG | 6 | 0.6672 |
| | Tregs | high | | 001037165.1 | 000007.14 | | | GGACCCA | | ACTTGGA | | | | |
| | | | | | | | | AACTCA | | CCCAAAC | | | | |
| | | | | | | | | | | TCAGGGT | | | | |
| | | | | | | | | | | CG | | | | |
| FOXK1 | IL2RA | IL2RA | 221937 | NM_ | NC_ | 4755361 | sense | GCATTAC | 2048. | CCAAGCA | 2176. | CGG | 4 | 0.7131 |
| | Tregs | high | | 001037165.1 | 000007.14 | | | CCCTACT | | TTACCCC | | | | |
| | | | | | | | | ACCGGA | | TACTACC | | | | |
| | | | | | | | | | | GGACGG | | | | |
| | | | | | | | | | | CCG | | | | |
| MYB | IL2RA | IL2RA | 4602 | NM_ | NC_ | 1.35E+08 | sense | ACCAGGC | 2049. | ATTTACC | 2177. | GGG | 5 | 0.6859 |
| | Tregs | high | | 005375.2 | 000006.12 | | | ACACAAG | | AGGCAC | | | | |
| | | | | | | | | AGACTG | | ACAAGA | | | | |
| | | | | | | | | | | GACTGG | | | | |
| | | | | | | | | | | GGAAC | | | | |
| MYB | IL2RA | IL2RA | 4602 | NM_ | NC_ | 1.35E+08 | sense | AGAAATA | 2050. | GTACAGA | 2178. | TGG | 5 | 0.6009 |
| | Tregs | high | | 005375.2 | 000006.12 | | | CGGTCCG | | AATACGG | | | | |
| | | | | | | | | AAACGT | | TCCGAAA | | | | |
| | | | | | | | | | | CGTTGGT | | | | |
| | | | | | | | | | | CT | | | | |
| MYB | IL2RA | IL2RA | 4602 | NM_ | NC_ | 1.35E+08 | sense | AGTCTGG | 2051. | CCCAAGT | 2179. | GGG | 2 | 0.7063 |
| | Tregs | high | | 005375.2 | 000006.12 | | | AAAGCGT | | CTGGAAA | | | | |
| | | | | | | | | CACTTG | | GCGTCAC | | | | |
| | | | | | | | | | | TTGGGG | | | | |
| | | | | | | | | | | AAA | | | | |
| MYB | IL2RA | IL2RA | 4602 | NM_ | NC_ | 1.35E+08 | antisense | TATTTAC | 2052. | ACTATAT | 2180. | GGG | 7 | 0.6157 |
| | Tregs | high | | 005375.2 | 000006.12 | | | ATGTAAC | | TTACATG | | | | |
| | | | | | | | | GCTACA | | TAACGCT | | | | |
| | | | | | | | | | | ACAGGG | | | | |
| | | | | | | | | | | TAT | | | | |
| ZNF217 | IL2RA | IL2RA | 7764 | NM_ | NC_ | 53581993 | sense | CAAAATC | 2053. | AGACCAA | 2181. | GGG | 1 | 0.6608 |
| | Tregs | high | | 006526.2 | 000020.11 | | | TCACCCT | | AATCTCA | | | | |
| | | | | | | | | GAAACG | | CCCTGAA | | | | |
| | | | | | | | | | | ACGGGG | | | | |
| | | | | | | | | | | AAG | | | | |
| ZNF217 | IL2RA | IL2RA | 7764 | NM_ | NC_ | 53581749 | sense | CCACGGC | 2054. | ACTCCCA | 2182. | TGG | 1 | 0.6177 |
| | Tregs | high | | 006526.2 | 000020.11 | | | GAAGCG | | CGGCGA | | | | |
| | | | | | | | | CCCTCCG | | AGCGCCC | | | | |
| | | | | | | | | | | TCCGTGG | | | | |
| | | | | | | | | | | ACG | | | | |
| ZNF217 | IL2RA | IL2RA | 7764 | NM_ | NC_ | 53582284 | sense | GGACAC | 2055. | ATGCGG | 2183. | GGG | 1 | 0.7154 |
| | Tregs | high | | 006526.2 | 000020.11 | | | ATAATGG | | ACACATA | | | | |
| | | | | | | | | CAAATCG | | ATGGCAA | | | | |
| | | | | | | | | | | ATCGGG | | | | |
| | | | | | | | | | | GGCC | | | | |
| ZNF217 | IL2RA | IL2RA | 7764 | NM_ | NC_ | 53576811 | antisense | TGGGTG | 2056. | GTTATGG | 2184. | AGG | 3 | 0.6707 |
| | Tregs | high | | 006526.2 | 000020.11 | | | GTACTGC | | GTGGTAC | | | | |
| | | | | | | | | CATCCGG | | TGCCATC | | | | |
| | | | | | | | | | | CGGAGG | | | | |
| | | | | | | | | | | AGG | | | | |
| CBFB | IL2RA | IL2RA | 865 | NM_ | NC_ | 67036720 | antisense | AAGTCGA | 2057. | TTCTAAG | 2185. | AGG | 3 | 0.5993 |
| | Tregs | high | | 001755.2 | 000016.10 | | | CATACTC | | TCGACAT | | | | |
| | | | | | | | | TCGGCT | | ACTCTCG | | | | |
| | | | | | | | | | | GCTAGGT | | | | |
| | | | | | | | | | | GT | | | | |
| CBFB | IL2RA | IL2RA | 865 | NM_ | NC_ | 67029479 | antisense | CCTGCCT | 2058. | CCCGCCT | 2186 | CGG | 1 | 0.6743 |
| | Tregs | high | | 001755.2 | 000016.10 | | | CACCTCA | | GCCTCAC | | | | |
| | | | | | | | | CACTCG | | CTCACAC | | | | |
| | | | | | | | | | | TCGCGGC | | | | |
| | | | | | | | | | | TC | | | | |
| CBFB | IL2RA | IL2RA | 865 | NM_ | NC_ | 67029807 | antisense | GCCGACT | 2059. | GCCAGCC | 2187. | CGG | 2 | 0.7383 |
| | Tregs | high | | 001755.2 | 000016.10 | | | TACGATT | | GACTTAC | | | | |
| | | | | | | | | TCCGAG | | GATTTCC | | | | |
| | | | | | | | | | | GAGCGG | | | | |
| | | | | | | | | | | CCG | | | | |
| CBFB | IL2RA | IL2RA | 865 | NM_ | NC_ | 67066729 | sense | GGAGTCT | 2060. | GAATGG | 2188. | AGG | 4 | 0.5938 |
| | Tregs | high | | 001755.2 | 000016.10 | | | GTGTTAT | | AGTCTGT | | | | |
| | | | | | | | | CTGGAA | | GTTATCT | | | | |
| | | | | | | | | | | GGAAAG | | | | |
| | | | | | | | | | | GCTG | | | | |
| KLF2 | IL2RA | IL2RA | 10365 | NM_ | NC_ | 16325729 | antisense | AAACCAG | 2061. | GCCGAA | 2189. | CGG | 2 | 0.5997 |
| | Tregs | high | | 016270.2 | 000019.10 | | | GGCCACC | | ACCAGG | | | | |
| | | | | | | | | GAAAGG | | GCCACCG | | | | |
| | | | | | | | | | | AAAGGC | | | | |
| | | | | | | | | | | GGCGG | | | | |
| KLF2 | IL2RA | IL2RA | 10365 | NM_ | NC_ | 16325576 | antisense | CCCTCGC | 2062. | GGCGCCC | 2190. | CGG | 2 | 0.5868 |
| | Tregs | high | | 016270.2 | 000019.10 | | | GCTTGAG | | TCGCGCT | | | | |
| | | | | | | | | GCCGCG | | TGAGGCC | | | | |
| | | | | | | | | | | GCGCGG | | | | |
| | | | | | | | | | | TCC | | | | |
| KLF2 | IL2RA | IL2RA | 10365 | NM_ | NC_ | 16325811 | sense | CTTCGGT | 2063. | CAGCCTT | 2191. | CGG | 2 | 0.7252 |
| | Tregs | high | | 016270.2 | 000019.10 | | | CTCTTCG | | CGGTCTC | | | | |
| | | | | | | | | ACGACG | | TTCGACG | | | | |
| | | | | | | | | | | ACGCGG | | | | |
| | | | | | | | | | | CCG | | | | |
| KLF2 | IL2RA | IL2RA | 10365 | NM_ | NC_ | 16325354 | antisense | TCGGGGT | 2064. | GGGTTCG | 2192. | CGG | 2 | 0.6967 |
| | Tregs | high | | 016270.2 | 000019.10 | | | AATAGAA | | GGGTAAT | | | | |
| | | | | | | | | CGCAGG | | AGAACG | | | | |
| | | | | | | | | | | CAGGCG | | | | |
| | | | | | | | | | | GCGG | | | | |
| CTCF | IL2RA | IL2RA | 10664 | NM_ | NC_ | 67612001 | antisense | CGATCCA | 2065. | GTGACG | 2193. | TGG | 4 | 0.6364 |
| | Teffs | high | | 006565.3 | 000016.10 | | | AATTTGA | | ATCCAAA | | | | |
| | | | | | | | | ACGCCG | | TTTGAAC | | | | |
| | | | | | | | | | | GCCGTG | | | | |
| | | | | | | | | | | GACA | | | | |
| CTCF | IL2RA | IL2RA | 10664 | NM_ | NC_ | 67611476 | sense | GAGCAA | 2066. | AAAAGA | 2194. | AGG | 3 | 0.6793 |
| | Teffs | high | | 006565.3 | 000016.10 | | | ACTGCGT | | GCAAACT | | | | |
| | | | | | | | | TATACAG | | GOGTTAT | | | | |
| | | | | | | | | | | ACAGAG | | | | |
| | | | | | | | | | | GAGG | | | | |
| CTCF | IL2RA | IL2RA | 10664 | NM_ | NC_ | 67610967 | sense | TTACCCC | 2067. | CCACTTA | 2195. | TGG | 3 | 0.6378 |
| | Teffs | high | | 006565.3 | 000016.10 | | | AGAACCA | | CCCCAGA | | | | |
| | | | | | | | | GACGGA | | ACCAGAC | | | | |
| | | | | | | | | | | GGATGG | | | | |
| | | | | | | | | | | GGG | | | | |
| CTCF | IL2RA | IL2RA | 10664 | NM_ | NC_ | 67620773 | sense | TTTGTGC | 2068. | GCAGTTT | 2196. | GGG | 6 | 0.6444 |
| | Teffs | high | | 006565.3 | 000016.10 | | | AGTTATG | | GTGCAGT | | | | |
| | | | | | | | | CCAGCA | | TATGCCA | | | | |
| | | | | | | | | | | GCAGGG | | | | |
| | | | | | | | | | | ACA | | | | |
| PTPRC | IL2RA | IL2RA | 5788 | NM_ | NC_ | 1.99E+08 | antisense | AGCATTA | 2069. | CACTAGC | 2197. | GGG | 4 | 0.6692 |
| | Teffs | high | | 002838.4 | 000001.11 | | | TCCAAAG | | ATTATCC | | | | |
| | | | | | | | | AGTCCG | | AAAGAG | | | | |
| | | | | | | | | | | TCCGGG | | | | |
| | | | | | | | | | | GATA | | | | |
| PTPRC | IL2RA | IL2RA | 5788 | NM_ | NC_ | 1.99E+08 | antisense | GGAAACT | 2070. | TATAGGA | 2198. | CGG | 19 | 0.6482 |
| | Teffs | high | | 002838.4 | 000001.11 | | | TGCTGAA | | AACTTGC | | | | |
| | | | | | | | | CACCCG | | TGAACAC | | | | |
| | | | | | | | | | | CCGCGG | | | | |
| | | | | | | | | | | GAT | | | | |
| PTPRC | IL2RA | IL2RA | 5788 | NM_ | NC_ | 1.99E+08 | antisense | TCCAAAT | 2071. | AACTTCC | 2199. | GGG | 14 | 0.6167 |
| | Teffs | high | | 002838.4 | 000001.11 | | | GGTAAC | | AAATGGT | | | | |
| | | | | | | | | GTTCATG | | AACGTTC | | | | |
| | | | | | | | | | | ATGGGG | | | | |
| | | | | | | | | | | GCC | | | | |
| PTPRC | IL2RA | IL2RA | 5788 | NM_ | NC_ | 1.99E+08 | sense | TGTGGAT | 2072 | TCACTGT | 2200. | AGG | 9 | 0.6937 |
| | Teffs | high | | 002838.4 | 000001.11 | | | TACTTAT | | GGATTAC | | | | |
| | | | | | | | | ATAACA | | TTATATA | | | | |
| | | | | | | | | | | ACAAGG | | | | |
| | | | | | | | | | | AAA | | | | |
| NR2C2 | IL2RA | IL2RA | 7182 | NM_ | NC_ | 15030406 | sense | CCAGTCG | 2073. | GAGACC | 2201. | AGG | 10 | 0.6976 |
| | Teffs | high | | 003298.4 | 000003.12 | | | ACACCCA | | AGTCGAC | | | | |
| | | | | | | | | TCATTG | | ACCCATC | | | | |
| | | | | | | | | | | ATTGAGG | | | | |
| | | | | | | | | | | TTG | | | | |
| NR2C2 | IL2RA | IL2RA | 7182 | NM_ | NC_ | 15016174 | antisense | CCCCAGT | 2074. | TCTTCCC | 2202. | AGG | 5 | 0.7308 |
| | Teffs | high | | 003298.4 | 000003.12 | | | AAACGCT | | CAGTAAA | | | | |
| | | | | | | | | CCACAG | | CGCTCCA | | | | |
| | | | | | | | | | | CAGAGG | | | | |
| | | | | | | | | | | CAG | | | | |
| NR2C2 | IL2RA | IL2RA | 7182 | NM_ | NC_ | 15024201 | antisense | GAACGTC | 2075. | TAGAGA | 2203. | TGG | 8 | 0.6168 |
| | Teffs | high | | 003298.4 | 000003.12 | | | ACCTTAG | | ACGTCAC | | | | |
| | | | | | | | | AATCCG | | CTTAGAA | | | | |
| | | | | | | | | | | TCCGTGG | | | | |
| | | | | | | | | | | CCA | | | | |
| NR2C2 | IL2RA | IL2RA | 7182 | NM_ | NC_ | 15023322 | antisense | TCTTTGT | 2076. | TCCATCT | 2204. | GGG | 7 | 0.6618 |
| | Teffs | high | | 003298.4 | 000003.12 | | | CTGCCAC | | TTGTCTG | | | | |
| | | | | | | | | AAACGT | | CCACAAA | | | | |
| | | | | | | | | | | CGTGGG | | | | |
| | | | | | | | | | | AGT | | | | |
| TP53 | IL2RA | IL2RA | 7157 | NM_ | NC_ | 7676227 | antisense | CCATTGT | 2077. | TGAACCA | 2205. | GGG | 4 | 0.6899 |
| | Teffs | high | | 000546.5 | 000017.11 | | | TCAATAT | | TTGTTCA | | | | |
| | | | | | | | | CGTCCG | | ATATCGT | | | | |
| | | | | | | | | | | CCGGGG | | | | |
| | | | | | | | | | | ACA | | | | |
| TP53 | IL2RA | IL2RA | 7157 | NM_ | NC_ | 7675058 | sense | GAGCGCT | 2078. | CCATGAG | 2206. | TGG | 5 | 0.6542 |
| | Teffs | high | | 000546.5 | 000017.11 | | | GCTCAGA | | CGCTGCT | | | | |
| | | | | | | | | TAGCGA | | CAGATAG | | | | |
| | | | | | | | | | | CGATGGT | | | | |
| | | | | | | | | | | GA | | | | |
| TP53 | IL2RA | IL2RA | 7157 | NM_ | NC_ | 7676527 | antisense | GATCCAC | 2079. | AATGGAT | 2207. | AGG | 2 | 0.6108 |
| | Teffs | high | | 000546.5 | 000017.11 | | | TCACAGT | | CCACTCA | | | | |
| | | | | | | | | TTCCAT | | CAGTTTC | | | | |
| | | | | | | | | | | CATAGGT | | | | |
| | | | | | | | | | | CT | | | | |
| TP53 | IL2RA | IL2RA | 7157 | NM_ | NC_ | 7674864 | sense | GGTGCCC | 2080. | TGGTGGT | 2208. | AGG | 6 | 0.6259 |
| | Teff | high | | 000546.5 | 000017.11 | | | TATGAGC | | GCCCTAT | | | | |
| | | | | | | | | CGCCTG | | GAGCCG | | | | |
| | | | | | | | | | | CCTGAGG | | | | |
| | | | | | | | | | | TCT | | | | |
| ATXN7L3 | IL2RA | IL2RA | 56970 | NM_ | NC_ | 44197610 | sense | CACGGAC | 2081. | ACGACAC | 2209. | AGG | 2 | 0.6328 |
| | Teffs | high | | 001098833.1 | 000017.11 | | | CCTGATA | | GGACCCT | | | | |
| | | | | | | | | GCATGA | | GATAGCA | | | | |
| | | | | | | | | | | TGAAGG | | | | |
| | | | | | | | | | | ATT | | | | |
| ATXN7L3 | IL2RA | IL2RA | 56970 | NM_ | NC_ | 44197712 | sense | CATCGCT | 2082. | AGGCCAT | 2210. | CGG | 2 | 0.7491 |
| | Teffs | high | | 001098833.1 | 000017.11 | | | CAGGAG | | CGCTCAG | | | | |
| | | | | | | | | ATATACG | | GAGATAT | | | | |
| | | | | | | | | | | ACGCGG | | | | |
| | | | | | | | | | | ACC | | | | |
| ATXN7L3 | IL2RA | IL2RA | 56970 | NM_ | NC_ | 44197233 | sense | GCAGCC | 2083. | AACAGCA | 2211. | CGG | 3 | 0.6135 |
| | Teffs | high | | 001098833.1 | 000017.11 | | | GAATCGC | | GCCGAAT | | | | |
| | | | | | | | | CAACCGC | | CGCCAAC | | | | |
| | | | | | | | | | | CGCCGGT | | | | |
| | | | | | | | | | | GA | | | | |
| ATXN7L3 | IL2RA | IL2RA | 56970 | NM_ | NC_ | 44195424 | sense | GCTTCGC | 2084. | AGGAGC | 2212. | CGG | 8 | 0.6566 |
| | Teffs | high | | 001098833.1 | 000017.11 | | | AGCCTGC | | TTCGCAG | | | | |
| | | | | | | | | TAACCA | | CCTGCTA | | | | |
| | | | | | | | | | | ACCACGG | | | | |
| | | | | | | | | | | TGA | | | | |
| TFDP1 | IL2RA | IL2RA | 7027 | NM_ | NC_ | 1.14E+08 | sense | ACCGGCA | 2085. | AGAGAC | 2213. | TGG | 5 | 0.6315 |
| | Teffs | high | | 007111.4 | 000013.11 | | | GCGTCAA | | CGGCAG | | | | |
| | | | | | | | | ACACCC | | CGTCAAA | | | | |
| | | | | | | | | | | CACCCTG | | | | |
| | | | | | | | | | | GTGG | | | | |
| TFDP1 | IL2RA | IL2RA | 7027 | NM_ | NC_ | 1.14E+08 | sense | ATGACCA | 2086. | GCTTATG | 2214. | CGG | 7 | 0.6204 |
| | Teffs | high | | 007111.4 | 000013.11 | | | GAAAAA | | ACCAGAA | | | | |
| | | | | | | | | CATAAGA | | AAACATA | | | | |
| | | | | | | | | | | AGACGG | | | | |
| | | | | | | | | | | CGC | | | | |
| TFDP1 | IL2RA | IL2RA | 7027 | NM_ | NC_ | 1.14E+08 | antisense | CCTTCAT | 2087. | CAGACCT | 2215. | AGG | 6 | 0.6851 |
| | Teffs | high | | 007111.4 | 000013.11 | | | GGAGAA | | TCATGGA | | | | |
| | | | | | | | | ATGCCGT | | GAAATGC | | | | |
| | | | | | | | | | | CGTAGGC | | | | |
| | | | | | | | | | | CC | | | | |
| TFDP1 | IL2RA | IL2RA | 7027 | NM_ | NC_ | 1.14E+08 | sense | GGTGCA | 2088. | ACCTGGT | 2216. | CGG | 9 | 0.6402 |
| | Teffs | high | | 100711.4 | 000013.11 | | | GAGAAA | | GCAGAG | | | | |
| | | | | | | | | CCGGCAT | | AAACCG | | | | |
| | | | | | | | | G | | GCATGCG | | | | |
| | | | | | | | | | | GAGC | | | | |
| HNRNPK | IL2RA | IL2RA | 3190 | NM_ | NC_ | 83972098 | sense | ATGATGT | 2089. | TACAATG | 2217. | CGG | 11 | 0.7754 |
| | Teffs | high | | 002140.3 | 000009.12 | | | TTGATGA | | ATGTTTG | | | | |
| | | | | | | | | CCGTCG | | ATGACCG | | | | |
| | | | | | | | | | | TCGCGGA | | | | |
| | | | | | | | | | | CG | | | | |
| HNRNPK | IL2RA | IL2RA | 3190 | NM_ | NC_ | 83975465 | antisense | CTGTTGG | 2090. | TAAACTG | 2218. | GGG | 6 | 0.6273 |
| | Teffs | high | | 002140.3 | 000009.12 | | | GACATAC | | TTGGGAC | | | | |
| | | | | | | | | CGCTOG | | ATACCGC | | | | |
| | | | | | | | | | | TCGGGG | | | | |
| | | | | | | | | | | CCA | | | | |
| HNRNPK | IL2RA | IL2RA | 3190 | NM_ | NC_ | 83971978 | sense | GATGATA | 2091. | TTATGAT | 2219. | AGG | 11 | 0.7149 |
| | Teffs | high | | 002140.3 | 000009.12 | | | TGAGCCC | | GATATGA | | | | |
| | | | | | | | | TCGTCG | | GCCCTCG | | | | |
| | | | | | | | | | | TCGAGG | | | | |
| | | | | | | | | | | ACC | | | | |
| HNRNPK | IL2RA | IL2RA | 3190 | NM_ | NC_ | 83973291 | sense | TAAAATC | 2092. | GTGCTAA | 2220. | AGG | 9 | 0.6608 |
| | Teffs | high | | 002140.3 | 000009.12 | | | AAAGAA | | AATCAAA | | | | |
| | | | | | | | | CTTCGAG | | GAACTTC | | | | |
| | | | | | | | | | | GAGAGG | | | | |
| | | | | | | | | | | TAA | | | | |
| NFKB2 | IL2RA | IL2RA | 4791 | NM_ | NC_ | 1.02E+08 | sense | ACTCGAC | 2093. | CCCTACT | 2221. | CGG | 14 | 0.7089 |
| | Teffs | high | | 001077494.3 | 000010.11 | | | TACGGOG | | CGACTAC | | | | |
| | | | | | | | | TCACCG | | GGCGTCA | | | | |
| | | | | | | | | | | CCGCGG | | | | |
| | | | | | | | | | | ACG | | | | |
| NFKB2 | IL2RA | IL2RA | 4791 | NM_ | NC_ | 1.02E+08 | sense | CCCACTC | 2094. | ATATCCC | 2222. | GGG | 9 | 0.6913 |
| | Teffs | high | | 001077494.3 | 000010.11 | | | CATAGAA | | ACTCCAT | | | | |
| | | | | | | | | TCTCCG | | AGAATCT | | | | |
| | | | | | | | | | | CCGGGG | | | | |
| | | | | | | | | | | GCA | | | | |
| NFKB2 | IL2RA | IL2RA | 4791 | NM_ | NC_ | 1.02E+08 | sense | CTGCAAC | 2095. | GTTTCTG | 2223. | AGG | 11 | 0.6401 |
| | Teffs | high | | 001077494.3 | 000010.11 | | | TGAAACG | | CAACTGA | | | | |
| | | | | | | | | CAAGCG | | AACGCAA | | | | |
| | | | | | | | | | | GCGAGG | | | | |
| | | | | | | | | | | AGG | | | | |
| NFKB2 | IL2RA | IL2RA | 4791 | NM_ | NC_ | 1.02E+08 | sense | GGGACC | 2096. | ACGAGG | 2224. | TGG | 6 | 0.7003 |
| | Teffs | high | | 700107494.3 | 000010.11 | | | AGCCAA | | GACCAGC | | | | |
| | | | | | | | | GATCGA | | CAAGATC | | | | |
| | | | | | | | | GG | | GAGGTG | | | | |
| | | | | | | | | | | GACC | | | | |
| PRDM1 | IL2RA | IL2RA | 639 | NM_ | NC_ | 1.06E+08 | sense | AGGATG | 2097. | ATGGAG | 2225. | TGG | 2 | 0.7731 |
| | Teffs | high | | 001198.3 | 000006.12 | | | CGGATAT | | GATGCG | | | | |
| | | | | | | | | GACTCTG | | GATATGA | | | | |
| | | | | | | | | | | CTCTGTG | | | | |
| | | | | | | | | | | GACA | | | | |
| PRDM1 | IL2RA | IL2RA | 639 | NM_ | NC_ | 1.06E+08 | antisense | GGACGC | 2098. | CGTAGG | 2226. | AGG | 5 | 0.6665 |
| | Teffs | high | | 001198.3 | 000006.12 | | | GTTCAAG | | ACGCGTT | | | | |
| | | | | | | | | TAAGCGT | | CAAGTAA | | | | |
| | | | | | | | | | | GCGTAG | | | | |
| | | | | | | | | | | GAGT | | | | |
| PRDM1 | IL2RA | IL2RA | 639 | NM_ | NC_ | 1.06E+08 | antisense | GGGGAG | 2099. | AATGGG | 2227. | GGG | 5 | 0.6871 |
| | Teffs | high | | 001198.3 | 000006.12 | | | CGAGTG | | GGAGCG | | | | |
| | | | | | | | | ATGTACG | | AGTGATG | | | | |
| | | | | | | | | T | | TACGTGG | | | | |
| | | | | | | | | | | GTCT | | | | |
| PRDM1 | IL2RA | IL2RA | 639 | NM_ | NC_ | 1.06E+08 | sense | TTTGGAC | 2100. | CTCTTTT | 2228. | AGG | 4 | 0.6708 |
| | Teffs | high | | 001198.3 | 000006.12 | | | AGATCTA | | GGACAG | | | | |
| | | | | | | | | TTCCAG | | ATCTATT | | | | |
| | | | | | | | | | | CCAGAG | | | | |
| | | | | | | | | | | GGGA | | | | |
| MYC | IL2RA | IL2RA | 4609 | NM_ | NC_ | 1.28E+08 | sense | AGAGTG | 2101. | CCTCAGA | 2229. | TGG | 2 | 0.6543 |
| | Teffs | high | | 002467.4 | 000008.11 | | | CATCGAC | | GTGCATC | | | | |
| | | | | | | | | CCCTCGG | | GACCCCT | | | | |
| | | | | | | | | | | CGGTGGT | | | | |
| | | | | | | | | | | CT | | | | |
| MYC | IL2RA | IL2RA | 4609 | NM_ | NC_ | 1.28E+08 | antisense | CTGCGG | 2102. | TGCCCTG | 2230. | AGG | 2 | 0.6832 |
| | Teffs | high | | 002467.4 | 000008.11 | | | GGAGGA | | CGGGGA | | | | |
| | | | | | | | | CTCCGTC | | GGACTCC | | | | |
| | | | | | | | | G | | GTCGAG | | | | |
| | | | | | | | | | | GAGA | | | | |
| MYC | IL2RA | IL2RA | 4609 | NM_ | NC_ | 1.28E+08 | sense | CTTCGGG | 2103. | CTCCCTT | 2231 | CGG | 2 | 0.6919 |
| | Teffs | high | | 002467.4 | 000008.11 | | | GAGACA | | CGGGGA | | | | |
| | | | | | | | | ACGACG | | GACAAC | | | | |
| | | | | | | | | G | | GACGGC | | | | |
| | | | | | | | | | | GGTGG | | | | |
| MYC | IL2RA | IL2RA | 4609 | NM_ | NC_ | 1.28E+08 | antisense | GCTGCAC | 2104. | TACGGCT | 2232. | AGG | 2 | 0.6842 |
| | Teffs | high | | 002467.4 | 000008.11 | | | CGAGTCG | | GCACCGA | | | | |
| | | | | | | | | TAGTCG | | GTCGTAG | | | | |
| | | | | | | | | | | TCGAGGT | | | | |
| | | | | | | | | | | CA | | | | |
| SMARCB1 | IL2RA | IL2RA | 6598 | NM_ | NC_ | 23791773 | antisense | GAGAAC | 2105. | TACAGAG | 2233. | AGG | 2 | 0.731 |
| | Teffs | high | | 003073.3 | 000022.11 | | | CTCGGAA | | AACCTCG | | | | |
| | | | | | | | | CATACGG | | GAACATA | | | | |
| | | | | | | | | | | CGGAGG | | | | |
| | | | | | | | | | | TAG | | | | |
| SMARCB1 | IL2RA | IL2RA | 6598 | NM_ | NC_ | 23816887 | sense | GCAGATC | 2106. | GACAGC | 2234. | CGG | 6 | 0.6894 |
| | Teffs | high | | 003073.3 | 000022.11 | | | GAGTCCT | | AGATCGA | | | | |
| | | | | | | | | ACCCCA | | GTCCTAC | | | | |
| | | | | | | | | | | CCCACGG | | | | |
| | | | | | | | | | | ACA | | | | |
| SMARCB1 | IL2RA | IL2RA | 6598 | NM_ | NC_ | 23801049 | antisense | TCTTCTT | 2107. | GTTCTCT | 2235. | CGG | 4 | 0.6806 |
| | Teffs | high | | 003073.3 | 000022.11 | | | GTCTCGG | | TCTTGTC | | | | |
| | | | | | | | | CCCATG | | TCGGCCC | | | | |
| | | | | | | | | | | ATGCGGT | | | | |
| | | | | | | | | | | TC | | | | |
| SMARCB1 | IL2RA | IL2RA | 6598 | NM_ | NC_ | 23803342 | sense | TGAGAAC | 2108. | TCCATGA | 2236. | AGG | 5 | 0.6804 |
| | Teffs | high | | 003073.3 | 000022.11 | | | GCATCTC | | GAACGC | | | | |
| | | | | | | | | AGCCCG | | ATCTCAG | | | | |
| | | | | | | | | | | CCCGAG | | | | |
| | | | | | | | | | | GTGC | | | | |
| KLF13 | IL2RA | IL2RA | 51621 | NM_ | NC_ | 31327702 | sense | CCGACCT | 2109. | CGCGCCG | 2237. | AGG | 1 | 0.7221 |
| | Teffs | high | | 015995.3 | 000015.10 | | | CGAGTCC | | ACCTCGA | | | | |
| | | | | | | | | CCGCAG | | GTCCCCG | | | | |
| | | | | | | | | | | CAGAGG | | | | |
| | | | | | | | | | | AAG | | | | |
| KLF13 | IL2RA | IL2RA | 51621 | NM_ | NC_ | 31327415 | sense | CGTGGTG | 2110. | TCTTCGT | 2238. | CGG | 1 | 0.6798 |
| | Teffs | high | | 015995.3 | 000015.10 | | | GCGCGG | | GGTGGC | | | | |
| | | | | | | | | ATCCTAG | | GCGGATC | | | | |
| | | | | | | | | | | CTAGCGG | | | | |
| | | | | | | | | | | ACC | | | | |
| KLF13 | IL2RA | IL2RA | 51621 | NM_ | NC_ | 31327274 | sense | CGTGTCC | 2111. | GCCTCGT | 2239. | CGG | 1 | 0.7 |
| | Teffs | high | | 015995.3 | 000015.10 | | | ATGTCGA | | GTCCATG | | | | |
| | | | | | | | | GCCGCG | | TCGAGCC | | | | |
| | | | | | | | | | | GCGCGG | | | | |
| | | | | | | | | | | TCG | | | | |
| KLF13 | IL2RA | IL2RA | 51621 | NM_ | NC_ | 31327767 | antisense | GAGTTCT | 2112. | GTGTGA | 2240. | AGG | 1 | 0.592 |
| | Teffs | high | | 015995.3 | 000015.10 | | | CAGGTGC | | GTTCTCA | | | | |
| | | | | | | | | GCCTTG | | GGTGCG | | | | |
| | | | | | | | | | | CCTTGAG | | | | |
| | | | | | | | | | | GTGC | | | | |
| IRF1 | IL2RA | IL2RA | 3659 | NM_ | NC_ | 1.32E+08 | sense | GAACTCC | 2113. | CCATGAA | 2241. | AGG | 4 | 0.6297 |
| | Teffs | high | | 002198.2 | 000005.10 | | | CTGCCAG | | CTCCCTG | | | | |
| | | | | | | | | ATATCG | | CCAGATA | | | | |
| | | | | | | | | | | TCGAGG | | | | |
| | | | | | | | | | | AGG | | | | |
| IRF1 | IL2RA | IL2RA | 3659 | NM_ | NC_ | 1.32E+08 | sense | TCTAGGC | 2114. | GCCCTCT | 2242. | GGG | 4 | 0.718 |
| | Teffs | high | | 002198.2 | 000005.10 | | | CGATACA | | AGGCCG | | | | |
| | | | | | | | | AAGCAG | | ATACAAA | | | | |
| | | | | | | | | | | GCAGGG | | | | |
| | | | | | | | | | | GAAA | | | | |
| IRF1 | IL2RA | IL2RA | 3659 | NM_ | NC_ | 1.32E+08 | sense | TCTCCCT | 2115. | GCTTTCT | 2243. | GGG | 6 | 0.6137 |
| | Teffs | high | | 002198.2 | 000005.10 | | | CGACAGT | | CCCTCGA | | | | |
| | | | | | | | | CATGTG | | CAGTCAT | | | | |
| | | | | | | | | | | GTGGGG | | | | |
| | | | | | | | | | | ATT | | | | |
| IRF1 | IL2RA | IL2RA | 3659 | NM_ | NC_ | 1.32E+08 | sense | TTAATTC | 2116. | CAGATTA | 2244. | GGG | 2 | 0.7113 |
| | Teffs | high | | 002198.2 | 000005.10 | | | CAACCAA | | ATTCCAA | | | | |
| | | | | | | | | ATCCCG | | CCAAATC | | | | |
| | | | | | | | | | | CCGGGG | | | | |
| | | | | | | | | | | CTC | | | | |
| FOXO1 | IL2RA | IL2RA | 2308 | NM_ | NC_ | 40665740 | antisense | ACAGGTT | 2117. | TAGGACA | 2245. | CGG | 1 | 0.547 |
| | Teffs | high | | 002015.3 | 000013.11 | | | GCCCCAC | | GGTTGCC | | | | |
| | | | | | | | | GCGTTG | | CCACGCG | | | | |
| | | | | | | | | | | TTGCGGC | | | | |
| | | | | | | | | | | GG | | | | |
| FOXO1 | IL2RA | IL2RA | 2308 | NM_ | NC_ | 40666107 | sense | GGAGTTT | 2118. | GGCCGG | 2246. | CGG | 1 | 0.629 |
| | Teffs | high | | 002015.3 | 000013.11 | | | AGCCAGT | | AGTTTAG | | | | |
| | | | | | | | | CCAACT | | CCAGTCC | | | | |
| | | | | | | | | | | AACTCGG | | | | |
| | | | | | | | | | | CCA | | | | |
| FOXO1 | IL2RA | IL2RA | 2308 | NM_ | NC_ | 40560279 | antisense | GGTGGC | 2119. | GTTTGGT | 2247. | CGG | 2 | 0.6741 |
| | Teffs | high | | 002015.3 | 000013.11 | | | GCAAAC | | GGCGCA | | | | |
| | | | | | | | | GAGTAG | | AACGAGT | | | | |
| | | | | | | | | CA | | AGCACG | | | | |
| | | | | | | | | | | GCGT | | | | |
| FOXO1 | IL2RA | IL2RA | 2308 | NM_ | NC_ | 40560544 | antisense | TAGCATT | 2120. | GTACTAG | 2248. | GGG | 2 | 0.6665 |
| | Teffs | high | | 002015.3 | 000013.11 | | | TGAGCTA | | CATTTGA | | | | |
| | | | | | | | | GTTCGA | | GCTAGTT | | | | |
| | | | | | | | | | | CGAGGG | | | | |
| | | | | | | | | | | CGA | | | | |
| IRF2 | IL2RA | IL2RA | 3660 | NM_ | NC_ | 1.84E+08 | sense | ACCTGAT | 2121. | ATAAACC | 2249. | AGG | 4 | 0.6049 |
| | Teffs | high | | 002199.3 | 000004.12 | | | CCCAAAA | | TGATCCC | | | | |
| | | | | | | | | CATGGA | | AAAACAT | | | | |
| | | | | | | | | | | GGAAGG | | | | |
| | | | | | | | | | | CGA | | | | |
| IRF2 | IL2RA | IL2RA | 3660 | NM_ | NC_ | 1.84E+08 | antisense | CAGCATT | 2122. | GGGGCA | 2250. | AGG | 4 | 0.6028 |
| | Teffs | high | | 002199.3 | 000004.12 | | | CGGTAG | | GCATTCG | | | | |
| | | | | | | | | ACCCTGA | | GTAGACC | | | | |
| | | | | | | | | | | CTGAAG | | | | |
| | | | | | | | | | | GCAT | | | | |
| IRF2 | IL2RA | IL2RA | 3660 | NM_ | NC_ | 1.84E+08 | sense | GGATGC | 2123. | CCCTGGA | 2251. | GGG | 3 | 0.6231 |
| | Teffs | high | | 002199.3 | 000004.12 | | | ATGCGGC | | TGCATGC | | | | |
| | | | | | | | | TAGACAT | | GGCTAG | | | | |
| | | | | | | | | | | ACATGG | | | | |
| | | | | | | | | | | GTGG | | | | |
| IRF2 | IL2RA | IL2RA | 3660 | NM_ | NC_ | 1.84E+08 | antisense | TACCTGC | 2124. | CGCTTAC | 2252. | GGG | 7 | 0.7207 |
| | Teffs | high | | 002199.3 | 000004.12 | | | ATAGGA | | CTGCATA | | | | |
| | | | | | | | | AGACAC | | GGAAGA | | | | |
| | | | | | | | | G | | CACGGG | | | | |
| | | | | | | | | | | GGAG | | | | |
| KLF2 | IL2RA | IL2RA | 10365 | NM_ | NC_ | 16325729 | antisense | AAACCAG | 2125. | GCCGAA | 2253. | CGG | 2 | 0.5997 |
| | Teffs | high | | 016270.2 | 000019.10 | | | GGCCACC | | ACCAGG | | | | |
| | | | | | | | | GAAAGG | | GCCACCG | | | | |
| | | | | | | | | | | AAAGGC | | | | |
| | | | | | | | | | | GGCGG | | | | |
| KLF2 | IL2RA | IL2RA | 10365 | NM_ | NC_ | 16325576 | antisense | CCCTCGC | 2126. | GGCGCCC | 2254. | CGG | 2 | 0.5868 |
| | Teffs | high | | 016270.2 | 000019.10 | | | GCTTGAG | | TCGCGCT | | | | |
| | | | | | | | | GCCGCG | | TGAGGCC | | | | |
| | | | | | | | | | | GCGCGG | | | | |
| | | | | | | | | | | TCC | | | | |
| KLF2 | IL2RA | IL2RA | 10365 | NM_ | NC_ | 16325811 | sense | CTTCGGT | 2127. | CAGCCTT | 2255. | CGG | 2 | 0.7252 |
| | Teffs | high | | 016270.2 | 000019.10 | | | CTCTTCG | | CGGTCTC | | | | |
| | | | | | | | | ACGACG | | TTCGACG | | | | |
| | | | | | | | | | | ACGCGG | | | | |
| | | | | | | | | | | CCG | | | | |
| KLF2 | IL2RA | IL2RA | 10365 | NM_ | NC_ | 16325354 | antisense | TCGGGGT | 2128. | GGGTTCG | 2256. | CGG | 2 | 0.6967 |
| | Teffs | high | | 016270.2 | 000019.10 | | | AATAGAA | | GGGTAAT | | | | |
| | | | | | | | | CGCAGG | | AGAACG | | | | |
| | | | | | | | | | | CAGGCG | | | | |
| | | | | | | | | | | GCGG | | | | |
| ZNF217 | IL2RA | IL2RA | 7764 | NM_ | NC_ | 53581993 | sense | CAAAATC | 2129. | AGACCAA | 2257. | GGG | 1 | 0.6608 |
| | Teffs | high | | 006526.2 | 000020.11 | | | TCACCCT | | AATCTCA | | | | |
| | | | | | | | | GAAACG | | CCCTGAA | | | | |
| | | | | | | | | | | ACGGGG | | | | |
| | | | | | | | | | | ÅAG | | | | |
| ZNF217 | IL2RA | IL2RA | 7764 | NM_ | NC_ | 53581749 | sense | CCACGGC | 2130. | ACTCCCA | 2258. | TGG | 1 | 0.6177 |
| | Teffs | high | | 006526.2 | 000020.11 | | | GAAGCG | | CGGCGA | | | | |
| | | | | | | | | CCCTCCG | | AGCGCCC | | | | |
| | | | | | | | | | | TCCGTGG | | | | |
| | | | | | | | | | | ACG | | | | |
| ZNF217 | IL2RA | IL2RA | 7764 | NM_ | NC_ | 53582284 | sense | GGACAC | 2131. | ATGGGG | 2259. | GGG | 1 | 0.7154 |
| | Teffs | high | | 006526.2 | 000020.11 | | | ATAATGG | | ACACATA | | | | |
| | | | | | | | | CAAATCG | | ATGGCAA | | | | |
| | | | | | | | | | | ATCGGG | | | | |
| | | | | | | | | | | GGCC | | | | |
| ZNF217 | IL2RA | IL2RA | 7764 | NM_ | NC_ | 53576811 | antisense | TGGGTG | 2132. | GTTATGG | 2260. | AGG | 3 | 0.6707 |
| | Teffs | high | | 006526.2 | 000020.11 | | | GTACTGC | | GTGGTAC | | | | |
| | | | | | | | | CATCCGG | | TGCCATC | | | | |
| | | | | | | | | | | CGGAGG | | | | |
| | | | | | | | | | | AGG | | | | |
| TNFAIP3 | IL2RA | IL2RA | 7128 | NM_ | NC_ | 1.38E+08 | sense | CCACTTG | 2133. | TGTTCCA | 2261. | GGG | 6 | 0.6901 |
| | Teffs | high | | 001270507.1 | 000006.12 | | | TTAACAG | | CTTGTTA | | | | |
| | | | | | | | | AGACCG | | ACAGAG | | | | |
| | | | | | | | | | | ACCGGG | | | | |
| | | | | | | | | | | GAAG | | | | |
| TNFAIP3 | IL2RA | IL2RA | 7128 | NM_ | NC_ | 1.38E+08 | sense | CTTGTGG | 2134. | GAAGCTT | 2262. | CGG | 2 | 0.6709 |
| | Teffs | high | | 001270507.1 | 000006.12 | | | CGCTGAA | | GTGGCG | | | | |
| | | | | | | | | AACGAA | | CTGAAAA | | | | |
| | | | | | | | | | | CGAACG | | | | |
| | | | | | | | | | | GTAA | | | | |
| TNFAIP3 | IL2RA | IL2RA | 7128 | NM_ | NC_ | 1.38E+08 | sense | TATGCCA | 2135. | CCTTTAT | 2263. | AGG | 7 | 0.7533 |
| | Teffs | high | | 001270507.1 | 000006.12 | | | TGAGTGC | | GCCATGA | | | | |
| | | | | | | | | TCAGAG | | GTGCTCA | | | | |
| | | | | | | | | | | GAGAGG | | | | |
| | | | | | | | | | | CGG | | | | |
| TNFAIP3 | IL2RA | IL2RA | 7128 | NM_ | NC_ | 1.38E+08 | antisense | TGAGAG | 2136. | GATTTGA | 2264. | CGG | 3 | 0.6393 |
| | Teffs | high | | 001270507.1 | 000006.12 | | | ACTCCAG | | GAGACTC | | | | |
| | | | | | | | | TTGCCAG | | CAGTTGC | | | | |
| | | | | | | | | | | CAGCGG | | | | |
| | | | | | | | | | | AAT | | | | |
| FOXK1 | IL2RA | IL2RA | 221937 | NM_ | NC_ | 4757132 | sense | AACAGG | 2137. | GTGGAA | 2265. | AGG | 5 | 0.6681 |
| | Teffs | high | | 001037165.1 | 000007.14 | | | CATTCCG | | CAGGCAT | | | | |
| | | | | | | | | GAAACG | | TCCGGAA | | | | |
| | | | | | | | | G | | ACGGAG | | | | |
| | | | | | | | | | | GCAG | | | | |
| FOXK1 | IL2RA | IL2RA | 221937 | NM_ | NC_ | 4754516 | antisense | AGGTCAC | 2138. | TCCGAGG | 2266. | CGG | 3 | 0.734 |
| | Teffs | high | | 001037165.1 | 000007.14 | | | GTTCTGC | | TCACGTT | | | | |
| | | | | | | | | ACAAAG | | CTGCACA | | | | |
| | | | | | | | | | | AAGCGG | | | | |
| | | | | | | | | | | TAA | | | | |
| FOXK1 | IL2RA | IL2RA | 221937 | NM_ | NC_ | 4759162 | antisense | CTAACTT | 2139. | GAAGCTA | 2267 | GGG | 6 | 0.6672 |
| | Teffs | high | | 001037165.1 | 000007.14 | | | GGACCCA | | ACTTGGA | | | | |
| | | | | | | | | AACTCA | | CCCAAAC | | | | |
| | | | | | | | | | | TCAGGGT | | | | |
| | | | | | | | | | | CG | | | | |
| FOXK1 | IL2RA | IL2RA | 221937 | NM_ | NC_ | 4755361 | sense | GCATTAC | 2140. | CCAAGCA | 2268. | CGG | 4 | 0.7131 |
| | Teffs | high | | 001037165.1 | 000007.14 | | | CCCTACT | | TTACCCC | | | | |
| | | | | | | | | ACCGGA | | TACTACC | | | | |
| | | | | | | | | | | GGACGG | | | | |
| | | | | | | | | | | CCG | | | | |
| MYB | IL2RA | IL2RA | 4602 | NM_ | NC_ | 1.35E+08 | sense | ACCAGGC | 2141. | ATTTACC | 2269. | GGG | 5 | 0.6859 |
| | Teffs | high | | 005375.2 | 000006.12 | | | ACACAAG | | AGGCAC | | | | |
| | | | | | | | | AGACTG | | ACAAGA | | | | |
| | | | | | | | | | | GACTGG | | | | |
| | | | | | | | | | | GGAAC | | | | |
| MYB | IL2RA | IL2RA | 4602 | NM_ | NC_ | 1.35E+08 | sense | AGAAATA | 2142. | GTACAGA | 2270. | TGG | 5 | 0.6009 |
| | Teffs | high | | 005375.2 | 000006.12 | | | CGGTCCG | | AATACGG | | | | |
| | | | | | | | | AAACGT | | TCCGAAA | | | | |
| | | | | | | | | | | CGTTGGT | | | | |
| | | | | | | | | | | CT | | | | |
| MYB | IL2RA | IL2RA | 4602 | NM_ | NC_ | 1.35E+08 | sense | AGTCTGG | 2143. | CCCAAGT | 2271. | GGG | 2 | 0.7063 |
| | Teffs | high | | 005375.2 | 000006.12 | | | AAAGCGT | | CTGGAAA | | | | |
| | | | | | | | | CACTTG | | GCGTCAC | | | | |
| | | | | | | | | | | TTGGGG | | | | |
| | | | | | | | | | | AAA | | | | |
| MYB | IL2RA | IL2RA | 4602 | NM_ | NC_ | 1.35E+08 | antisense | TATTTAC | 2144. | ACTATAT | 2272. | GGG | 7 | 0.6157 |
| | Teffs | high | | 005375.2 | 000006.12 | | | ATGTAAC | | TTACATG | | | | |
| | | | | | | | | GCTACA | | TAACGCT | | | | |
| | | | | | | | | | | ACAGGG | | | | |
| | | | | | | | | | | TAT | | | | |
| CBFB | IL2RA | IL2RA | 865 | NM_ | NC_ | 67036720 | antisense | AAGTCGA | 2145. | TTCTAAG | 2273. | AGG | 3 | 0.5993 |
| | Teffs | high | | 001755.2 | 100006.10 | | | CATACTC | | TCGACAT | | | | |
| | | | | | | | | TCGGCT | | ACTCTCG | | | | |
| | | | | | | | | | | GCTAGGT | | | | |
| | | | | | | | | | | GT | | | | |
| CBFB | IL2RA | IL2RA | 865 | NM_ | NC_ | 67029479 | antisense | CCTGCCT | 2146. | CCCGCCT | 2274. | CGG | 1 | 0.6743 |
| | Teffs | high | | 001755.2 | 000016.10 | | | CACCTCA | | GCCTCAC | | | | |
| | | | | | | | | CACTCG | | CTCACAC | | | | |
| | | | | | | | | | | TCGCGGC | | | | |
| | | | | | | | | | | TC | | | | |
| CBFB | IL2RA | IL2RA | 865 | NM_ | NC_ | 67029807 | antisense | GCCGACT | 2147. | GCCAGCC | 2275. | CGG | 2 | 0.7383 |
| | Teffs | high | | 001755.2 | 000016.10 | | | TACGATT | | GACTTAC | | | | |
| | | | | | | | | TCCGAG | | GATTTCC | | | | |
| | | | | | | | | | | GAGCGG | | | | |
| | | | | | | | | | | CCG | | | | |
| CBFB | IL2RA | IL2RA | 865 | NM_ | NC_ | 67066729 | sense | GGAGTCT | 2148. | GAATGG | 2276. | AGG | 4 | 0.5938 |
| | Teffs | high | | 001755.2 | 000016.10 | | | GTGTTAT | | AGTCTGT | | | | |
| | | | | | | | | CTGGAA | | GTTATCT | | | | |
| | | | | | | | | | | GGAAAG | | | | |
| | | | | | | | | | | GCTG | | | | |
| HIVEP2 | IL2RA | IL2RA | 3097 | NM_ | NC_ | 1.43E+08 | antisense | CCCTGGA | 2149. | CGGTCCC | 2277. | CGG | 5 | 0.6747 |
| | Teffs | high | | 006734.3 | 000006.12 | | | TAGAATA | | TGGATAG | | | | |
| | | | | | | | | CATCGT | | AATACAT | | | | |
| | | | | | | | | | | CGTCGGA | | | | |
| | | | | | | | | | | AC | | | | |
| HIVEP2 | IL2RA | IL2RA | 3097 | NM_ | NC_ | 1.43E+08 | sense | GACAAG | 2150. | TTCAGAC | 2278. | GGG | 5 | 0.7382 |
| | Teffs | high | | 006734.3 | 000006.12 | | | ATGTCAG | | AAGATGT | | | | |
| | | | | | | | | ACCTAGG | | CAGACCT | | | | |
| | | | | | | | | | | AGGGGG | | | | |
| | | | | | | | | | | CAG | | | | |
| HIVEP2 | IL2RA | IL2RA | 3097 | NM_ | NC_ | 1.43E+08 | antisense | GAGGTG | 2151. | GTCAGA | 2279. | GGG | 5 | 0.6922 |
| | Teffs | high | | 006734.3 | 000006.12 | | | GAAGGT | | GGTGGA | | | | |
| | | | | | | | | AAACACA | | AGGTAA | | | | |
| | | | | | | | | A | | ACACAAG | | | | |
| | | | | | | | | | | GGGAT | | | | |
| HIVEP2 | IL2RA | IL2RA | 3097 | NM_ | NC_ | 1.43E+08 | antisense | TTCTAGG | 2152. | TTTTTTCT | 2280. | TGG | 5 | 0.71 |
| | Teffs | high | | 006734.3 | 000006.12 | | | ATAACCA | | AGGATA | | | | |
| | | | | | | | | CCACTG | | ACCACCA | | | | |
| | | | | | | | | | | CTGTGGC | | | | |
| | | | | | | | | | | CA | | | | |
| MED12 | IL2RA | IL2RA | 9968 | NM_ | NC_ | 71130165 | sense | ACATCGA | 2153. | ATCCACA | 2281. | AGG | 28 | 0.6457 |
| | Teffs | high | | 005120.2 | 000023.11 | | | CTGCTGG | | TCGACTG | | | | |
| | | | | | | | | ACAATG | | CTGGACA | | | | |
| | | | | | | | | | | ATGAGG | | | | |
| | | | | | | | | | | ATG | | | | |
| MED12 | IL2RA | IL2RA | 9968 | NM_0051 | NC_ | 7112223 | antisense | CAGTGA | 2154. | CAGTCAG | 2282. | AGG | 8 | 0.688 |
| | | | | 005120.2 | 000023.11 | | | CAAACCA | | TGAGTAG | | | | |
| | | | | | | | | | | TGCCAAA | | | | |
| | | | | | | | | | | CCAAGGC | | | | |
| | | | | | | | | | | AC | | | | |
| MED12 | IL2RA | IL2RA | 9968 | NM_ | NC_ | 71125111 | antisense | GTGGCGT | 2155. | ATGGGT | 2283. | TGG | 15 | 0.6661 |
| | Teffs | high | | 005120.2 | 000023.11 | | | ACTGCAC | | GGCGTAC | | | | |
| | | | | | | | | GTGTCG | | TGCACGT | | | | |
| | | | | | | | | | | GTCGTGG | | | | |
| | | | | | | | | | | CTG | | | | |
| MED12 | IL2RA | IL2RA | 9968 | NM_ | NC_ | 71126138 | sense | TTCACAT | 2156. | ACCTTTC | 2284. | AGG | 18 | 0.6594 |
| | Teffs | high | | 005120.2 | 000023.11 | | | TATGACC | | ACATTAT | | | | |
| | | | | | | | | AACACC | | GACCAAC | | | | |
| | | | | | | | | | | ACCAGGT | | | | |
| | | | | | | | | | | CA | | | | |

TABLE 14
Nuclear factors that can be inhibited to decrease IL2RA expression or overexpressed to increase IL2RA expression in
effector T cells and regulatory T cells

| | | | | | | Position of | | | | | | | | |
|---|---|---|---|---|---|---|---|---|---|---|---|---|---|---|
| | | | | | | Base After | | sgRNA | SEQ | Target | SEQ | | | |
| | screen_ | screen_ | Target | Target | Genomic | Cut (1- | | Target | ID | Context | ID | PAM | Exon | Rule Set |
| gene_id | target | direction | Gene ID | Transcript | Sequence | based) | Strand | Sequence | NO: | Sequence | NO: | Sequence | Number | 2 score |
| FOXO1 | IL2RA | IL2RA low | 2308 | NM_ | NC_ | 40665740 | antisense | ACAGGTT | 2285. | TAGGACA | 2429. | CGG | 1 | 0.547 |
| | Tregs | | | 002015.3 | 000013.11 | | | GCCCCACG | | GGTTGCCC | | | | |
| | | | | | | | | CGTTG | | CACGCGTT | | | | |
| | | | | | | | | | | GCGGCGG | | | | |
| FOXO1 | IL2RA | IL2RA low | 2308 | NM_ | NC_ | 40666107 | sense | GGAGTTTA | 2286. | GGCCGGA | 2430. | CGG | 1 | 0.629 |
| | Tregs | | | 002015.3 | 000013.11 | | | GCCAGTCC | | GTTTAGCC | | | | |
| | | | | | | | | AACT | | AGTCCAAC | | | | |
| | | | | | | | | | | TCGGCCA | | | | |
| FOXO1 | IL2RA | IL2RA low | 2308 | NM_ | NC_ | 40560279 | antisense | GGTGGCG | 2287. | GTTTGGTG | 2431. | CGG | 2 | 0.6741 |
| | Tregs | | | 002015.3 | 000013.11 | | | CAAACGA | | GCGCAAA | | | | |
| | | | | | | | | GTAGCA | | CGAGTAG | | | | |
| | | | | | | | | | | CACGGCG | | | | |
| | | | | | | | | | | T | | | | |
| FOXO1 | IL2RA | IL2RA low | 2308 | NM_ | NC_ | 40560544 | antisense | TAGCATTT | 2288. | GTACTAGC | 2432. | GGG | 2 | 0.6665 |
| | Tregs | | | 002015.3 | 000013.11 | | | GAGCTAG | | ATTTGAGC | | | | |
| | | | | | | | | TTCGA | | TAGTTCGA | | | | |
| | | | | | | | | | | GGGCGA | | | | |
| PTEN | IL2RA | IL2RA low | 5728 | NM_ | NC_ | 87961027 | sense | AGAGCGT | 2289. | GTATAGA | 2433. | AGG | 8 | 0.7179 |
| | Tregs | | | 001304718.1 | 000010.11 | | | GCAGATA | | GCGTGCA | | | | |
| | | | | | | | | ATGACA | | GATAATGA | | | | |
| | | | | | | | | | | CAAGGAA | | | | |
| | | | | | | | | | | T | | | | |
| PTEN | IL2RA | IL2RA low | 5728 | NM_ | NC_ | 87957861 | antisense | AGCTGGC | 2290. | CTTTAGCT | 2434. | AGG | 7 | 0.6493 |
| | Tregs | | | 001304718.1 | 000010.11 | | | AGACCACA | | GGCAGAC | | | | |
| | | | | | | | | AACTG | | CACAAACT | | | | |
| | | | | | | | | | | GAGGATC | | | | |
| PTEN | IL2RA | IL2RA low | 5728 | NM_ | NC_ | 87960940 | sense | ATTCTTCA | 2291. | ATACATTC | 2435. | AGG | 8 | 0.6697 |
| | Tregs | | | 001304718.1 | 000010.11 | | | TACCAGGA | | TTCATACC | | | | |
| | | | | | | | | CCAG | | AGGACCA | | | | |
| | | | | | | | | | | GAGGAAA | | | | |
| PTEN | IL2RA | IL2RA low | 5728 | NM_ | NC_ | 87957915 | sense | CCAATTCA | 2292. | TCCTCCAA | 2436. | CGG | 7 | 0.6752 |
| | Tregs | | | 001304718.1 | 000010.11 | | | GGACCCAC | | TTCAGGAC | | | | |
| | | | | | | | | ACGA | | CCACACGA | | | | |
| | | | | | | | | | | CGGGAA | | | | |
| STAT5B | IL2RA | IL2RA low | 6777 | NM_ | NC_ | 42216055 | sense | CAGCCAG | 2293. | ATGGCAG | 2437. | CGG | 12 | 0.6375 |
| | Tregs | | | 012448.3 | 000017.11 | | | GACAACA | | CCAGGAC | | | | |
| | | | | | | | | ATGCGA | | AACAATGC | | | | |
| | | | | | | | | | | GACGGCC | | | | |
| | | | | | | | | | | A | | | | |
| STAT5B | IL2RA | IL2RA low | 6777 | NM_ | NC_ | 42227658 | antisense | GTGGCCTT | 2294. | CTGGGTG | 2438. | TGG | 3 | 0.6157 |
| | Tregs | | | 012448.3 | 000017.11 | | | AATGTTCT | | GCCTTAAT | | | | |
| | | | | | | | | CCTG | | GTTCTCCT | | | | |
| | | | | | | | | | | GTGGATT | | | | |
| STAT5B | IL2RA | IL2RA low | 6777 | NM_ | NC_ | 42224822 | antisense | GTTCATTG | 2295. | CTCTGTTC | 2439. | CGG | 4 | 0.6583 |
| | Tregs | | | 012448.3 | 000017.11 | | | TACAATAT | | ATTGTACA | | | | |
| | | | | | | | | ATGG | | ATATATGG | | | | |
| | | | | | | | | | | CGGATG | | | | |
| STAT5B | IL2RA | IL2RA low | 6777 | NM_ | NC_ | 42217252 | sense | TAAGAGG | 2296. | GAATTAAG | 2440. | GGG | 11 | 0.7097 |
| | Tregs | | | 012448.3 | 000017.11 | | | TCAGACCG | | AGGTCAG | | | | |
| | | | | | | | | TCGTG | | ACCGTCGT | | | | |
| | | | | | | | | | | GGGGCAG | | | | |
| IL2RA | IL2RA | IL2RA low | 3559 | NM_ | NC_ | 6021673 | sense | CTGCAGG | 2297. | GTCACTGC | 2441. | GGG | 4 | 0.5815 |
| | Tregs | | | 000417.2 | 000010.11 | | | GAACCTCC | | AGGGAAC | | | | |
| | | | | | | | | ACCAT | | CTCCACCA | | | | |
| | | | | | | | | | | TGGGAAA | | | | |
| IL2RA | IL2RA | IL2RA low | 3559 | NM_ | NC_ | 6021582 | sense | GGATACA | 2298. | CCAGGGA | 2442. | AGG | 4 | 0.7695 |
| | Tregs | | | 000417.2 | 000010.11 | | | GGGCTCTA | | TACAGGG | | | | |
| | | | | | | | | CACAG | | CTCTACAC | | | | |
| | | | | | | | | | | AGAGGTC | | | | |
| | | | | | | | | | | C | | | | |
| IL2RA | IL2RA | IL2RA low | 3559 | NM_ | NC_ | 6025992 | antisense | TGGCTTTG | 2299. | GCCATGG | 2443. | GGG | 2 | 0.6265 |
| | Tregs | | | 000417.2 | 000010.11 | | | AATGTGG | | CTTTGAAT | | | | |
| | | | | | | | | CGTGT | | GTGGCGT | | | | |
| | | | | | | | | | | GTGGGAT | | | | |
| | | | | | | | | | | C | | | | |
| IL2RA | IL2RA | IL2RA low | 3559 | NM_ | NC_ | 6024349 | antisense | TTGTTTCG | 2300 | TCACTTGT | 2444. | TGG | 3 | 0.6476 |
| | Tregs | | | 000417.2 | 000010.11 | | | TTGTGTTC | | TTCGTTGT | | | | |
| | | | | | | | | CGAG | | GTTCCGAG | | | | |
| | | | | | | | | | | TGGCTA | | | | |
| ATXN7L3 | IL2RA | IL2RA low | S6970 | NM_ | NC_ | 44197610 | sense | CACGGACC | 2301. | ACGACAC | 2445. | AGG | 2 | 0.6328 |
| | Tregs | | | 800109833.1 | 000017.11 | | | CTGATAGC | | GGACCCT | | | | |
| | | | | | | | | ATGA | | GATAGCAT | | | | |
| | | | | | | | | | | GAAGGAT | | | | |
| | | | | | | | | | | T | | | | |
| ATXN7L3 | IL2RA | IL2RA low | 56970 | NM_ | NC_ | 44197712 | sense | CATCGCTC | 2302. | AGGCCATC | 2446. | CGG | 2 | 0.7491 |
| | Tregs | | | 001098833.1 | 000017.11 | | | AGGAGAT | | GCTCAGG | | | | |
| | | | | | | | | ATACG | | AGATATAC | | | | |
| | | | | | | | | | | GCGGACC | | | | |
| ATXN7L3 | IL2RA | IL2RA low | 56970 | NM_ | NC_ | 44197233 | sense | GCAGCCG | 2303. | AACAGCA | 2447. | CGG | 3 | 0.6135 |
| | Tregs | | | 001098833.1 | 000017.11 | | | AATCGCCA | | GCCGAATC | | | | |
| | | | | | | | | ACCGC | | GCCAACCG | | | | |
| | | | | | | | | | | CCGGTGA | | | | |
| ATXN7L3 | IL2RA | IL2RA low | S6970 | NM_ | NC_ | 44195424 | sense | GCTTCGCA | 2304. | AGGAGCT | 2448. | CGG | 8 | 0.6566 |
| | Tregs | | | 001098833.1 | 000017.11 | | | GCCTGCTA | | TCGCAGCC | | | | |
| | | | | | | | | ACCA | | TGCTAACC | | | | |
| | | | | | | | | | | ACGGTGA | | | | |
| RELA | IL2RA | IL2RA low | 5970 | NM_ | NC_ | 65659757 | sense | ACTACGAC | 2305. | GGGGACT | 2449. | CGG | 6 | 0.7259 |
| | Tregs | | | 001243984.1 | 000011.10 | | | CTGAATGC | | ACGACCTG | | | | |
| | | | | | | | | TGTG | | AATGCTGT | | | | |
| | | | | | | | | | | GCGGCTC | | | | |
| RELA | IL2RA | IL2RA low | 5970 | NM_ | NC_ | 65662009 | sense | GCTTCCGC | 2306. | ATGCGCTT | 2450. | GGG | 3 | 0.7137 |
| | Tregs | | | 001243984.1 | 000011.10 | | | TACAAGTG | | CCGCTACA | | | | |
| | | | | | | | | CGAG | | AGTGCGA | | | | |
| | | | | | | | | | | GGGGCGC | | | | |
| RELA | IL2RA | IL2RA low | 5970 | NM_ | NC_ | 65658759 | antisense | GGAAGAT | 2307. | AGTAGGA | 2451. | AGG | 7 | 0.7554 |
| | Tregs | | | 001243984.1 | 000011.10 | | | CTCATCCC | | AGATCTCA | | | | |
| | | | | | | | | CACCG | | TCCCCACC | | | | |
| | | | | | | | | | | GAGGCAG | | | | |
| RELA | IL2RA | IL2RA low | 5970 | NM_ | NC_ | 65661818 | sense | TCAATGGC | 2308. | CAGATCAA | 2452. | GGG | 4 | 0.7164 |
| | Tregs | | | 001243984.1 | 000011.10 | | | TACACAGG | | TGGCTACA | | | | |
| | | | | | | | | ACCA | | CAGGACC | | | | |
| | | | | | | | | | | AGGGACA | | | | |
| FOXP1 | IL2RA | ILZRA low | 27086 | NM_ | NC_ | 71041428 | antisense | AGAGGAG | 2309. | GTGCAGA | 2453. | TGG | 11 | 0.6926 |
| | Tregs | | | 032682.5 | 000003.12 | | | GAGACAC | | GGAGGAG | | | | |
| | | | | | | | | ATGTCG | | ACACATGT | | | | |
| | | | | | | | | | | CGTGGTCA | | | | |
| FOXP1 | IL2RA | IL2RA low | 27086 | NM_ | NC_ | 71015617 | antisense | CATACACC | 2310. | CTTGCATA | 2454. | AGG | 12 | 0.6547 |
| | Tregs | | | 032682.5 | 000003.12 | | | ATGTCCAT | | CACCATGT | | | | |
| | | | | | | | | AGAG | | CCATAGAG | | | | |
| | | | | | | | | | | AGGATG | | | | |
| FOXP1 | IL2RA | IL2RA low | 27086 | NM_ | NC_ | 71046982 | sense | GCCTTCTG | 2311. | CAAGGCCT | 2455. | GGG | 10 | 0.5683 |
| | Tregs | | | 032682.5 | 000003.12 | | | ACAATTCA | | TCTGACAA | | | | |
| | | | | | | | | GCCC | | TTCAGCCC | | | | |
| | | | | | | | | | | GGGCAG | | | | |
| FOXP1 | IL2RA | IL2RA low | 27086 | NM_ | NC_ | 70988031 | antisense | GTTCTGTA | 2312. | TTGGGTTC | 2456. | AGG | 14 | 0.5925 |
| | Tregs | | | 032682.5 | 000003.12 | | | GACTTCAC | | TGTAGACT | | | | |
| | | | | | | | | ATGC | | TCACATGC | | | | |
| | | | | | | | | | | AGGTGG | | | | |
| STAT5A | IL2RA | IL2RA low | 6776 | NM_ | NC_ | 42292035 | sense | ACATTCTG | 2313. | CGGCACAT | 2457. | AGG | 5 | 0.7341 |
| | Tregs | | | 003152.3 | 000017.11 | | | TACAATGA | | TCTGTACA | | | | |
| | | | | | | | | ACAG | | ATGAACA | | | | |
| | | | | | | | | | | GAGGCTG | | | | |
| STAT5A | IL2RA | ILZRA low | 6776 | NM_ | NC_ | 42304559 | sense | ATCAAGCG | 2314. | GAGGATC | 2458. | GGG | 12 | 0.6531 |
| | Tregs | | | 003152.3 | 000017.11 | | | TGCTGACC | | AAGCGTG | | | | |
| | | | | | | | | GGCG | | CTGACCG | | | | |
| | | | | | | | | | | GCGGGGT | | | | |
| | | | | | | | | | | GC | | | | |
| STAT5A | IL2RA | IL2RA low | 6776 | NM_ | NC_ | 42305662 | sense | CAGCCAG | 2315. | ACGGCAG | 2459. | CGG | 13 | 0.6255 |
| | Tregs | | | 003152.3 | 000017.11 | | | GACCACAA | | CCAGGACC | | | | |
| | | | | | | | | TGCCA | | ACAATGCC | | | | |
| | | | | | | | | | | ACGGCTA | | | | |
| STAT5A | IL2RA | IL2RA low | 6776 | NM_ | NC_ | 42301385 | sense | CGTGCACA | 2316. | TGAACGT | 2460. | AGG | 10 | 0.5812 |
| | Tregs | | | 003152.3 | 000017.11 | | | TGAATCCC | | GCACATGA | | | | |
| | | | | | | | | CCCC | | ATCCCCCC | | | | |
| | | | | | | | | | | CAGGTGA | | | | |
| IL2RA | IL2RA Teffs | IL2RA low | 3559 | NM_ | NC_ | 6021673 | sense | CTGCAGG | 2317. | GTCACTGC | 2461. | GGG | 4 | 0.5815 |
| | | | | 000417.2 | 000010.11 | | | GAACCTCC | | AGGGAAC | | | | |
| | | | | | | | | ACCAT | | CTCCACCA | | | | |
| | | | | | | | | | | TGGGAAA | | | | |
| IL2RA | IL2RA Teffs | IL2RA low | 3559 | NM_ | NC_ | 6021582 | sense | GGATACA | 2318. | CCAGGGA | 2462. | AGG | 4 | 0.7695 |
| | | | | 000417.2 | 000010.11 | | | GGGCTCTA | | TACAGGG | | | | |
| | | | | | | | | CACAG | | CTCTACAC | | | | |
| | | | | | | | | | | AGAGGTC | | | | |
| | | | | | | | | | | C | | | | |
| IL2RA | IL2RA Teffs | IL2RA low | 3559 | NM_ | NC_ | 6025992 | antisense | TGGCTTTG | 2319. | GCCATGG | 2463. | GGG | 2 | 0.6265 |
| | | | | 000417.2 | 000010.11 | | | AATGTGG | | CTTTGAAT | | | | |
| | | | | | | | | CGTGT | | GTGGCGT | | | | |
| | | | | | | | | | | GTGGGAT | | | | |
| | | | | | | | | | | C | | | | |
| IL2RA | IL2RA Teffs | IL2RA low | 3559 | NM_ | NC_ | 6024349 | antisense | TTGTTTCG | 2320. | TCACTTGT | 2464. | TGG | 3 | 0.6476 |
| | | | | 000417.2 | 000010.11 | | | TTGTGTTC | | TTCGTTGT | | | | |
| | | | | | | | | CGAG | | GTTCCGAG | | | | |
| | | | | | | | | | | TGGCTA | | | | |
| STAT5B | IL2RA Teffs | IL2RA low | 6777 | NM_ | NC_ | 42216055 | sense | CAGCCAG | 2321. | ATGGCAG | 2465. | CGG | 12 | 0.6375 |
| | | | | 012448.3 | 000017.11 | | | GACAACA | | CCAGGAC | | | | |
| | | | | | | | | ATGCGA | | AACAATGC | | | | |
| | | | | | | | | | | GACGGCC | | | | |
| | | | | | | | | | | A | | | | |
| STAT5B | IL2RA Teffs | IL2RA low | 6777 | NM_ | NC_ | 42227658 | antisense | GTGGCCTT | 2322. | CTGGGTG | 2466. | TGG | 3 | 0.6157 |
| | | | | 012448.3 | 000017.11 | | | AATGTTCT | | GCCTTAAT | | | | |
| | | | | | | | | CCTG | | GTTCTCCT | | | | |
| | | | | | | | | | | GTGGATT | | | | |
| STAT5B | IL2RA Teffs | IL2RA low | 6777 | NM_ | NC_ | 42224822 | antisense | GTTCATTG | 2323. | CTCTGTTC | 2467. | CGG | 4 | 0.6583 |
| | | | | 012448.3 | 000017.11 | | | TACAATAT | | ATTGTACA | | | | |
| | | | | | | | | ATGG | | ATATATGG | | | | |
| | | | | | | | | | | CGGATG | | | | |
| STAT5B | IL2RA Teffs | IL2RA low | 6777 | NM_ | NC_ | 42217252 | sense | TAAGAGG | 2324. | GAATTAAG | 2468. | GGG | 11 | 0.7097 |
| | | | | 012448.3 | 000017.11 | | | TCAGACCG | | AGGTCAG | | | | |
| | | | | | | | | TCGTG | | ACCGTCGT | | | | |
| | | | | | | | | | | GGGGCAG | | | | |
| FOXP1 | IL2RA Teffs | IL2RA low | 27086 | NM_ | NC_ | 71041428 | antisense | AGAGGAG | 2325. | GTGCAGA | 2469 | TGG | 11 | 0.6926 |
| | | | | 032682.5 | 000003.12 | | | GAGACAC | | GGAGGAG | | | | |
| | | | | | | | | ATGTCG | | ACACATGT | | | | |
| | | | | | | | | | | CGTGGTCA | | | | |
| FOXP1 | IL2RA Teffs | IL2RA low | 27086 | NM_ | NC_ | 71015617 | antisense | CATACACC | 2326. | CTTGCATA | 2470. | AGG | 12 | 0.6547 |
| | | | | 032682.5 | 000003.12 | | | ATGTCCAT | | CACCATGT | | | | |
| | | | | | | | | AGAG | | CCATAGAG | | | | |
| | | | | | | | | | | AGGATG | | | | |
| FOXP1 | IL2RA Teffs | IL2RA low | 27086 | NM_ | NC_ | 71046982 | sense | GCCTTCTG | 2327. | CAAGGCCT | 2471 | GGG | 10 | 0.5683 |
| | | | | 032682.5 | 000003.12 | | | ACAATTCA | | TCTGACAA | | | | |
| | | | | | | | | GCCC | | TTCAGCCC | | | | |
| | | | | | | | | | | GGGCAG | | | | |
| FOXP1 | IL2RA Teffs | IL2RA low | 27086 | NM_ | NC_ | 70988031 | antisense | GTTCTGTA | 2328. | TTGGGTTC | 2472. | AGG | 14 | 0.5925 |
| | | | | 032682.5 | 000003.12 | | | GACTTCAC | | TGTAGACT | | | | |
| | | | | | | | | ATGC | | TCACATGC | | | | |
| | | | | | | | | | | AGGTGG | | | | |
| STAT5A | IL2RA Teffs | IL2RA low | 6776 | NM_ | NC_ | 42292035 | sense | ACATTCTG | 2329. | CGGCACAT | 2473. | AGG | 5 | 0.7341 |
| | | | | 003152.3 | 000017.11 | | | TACAATGA | | TCTGTACA | | | | |
| | | | | | | | | ACAG | | ATGAACA | | | | |
| | | | | | | | | | | GAGGCTG | | | | |
| STAT5A | IL2RA Teffs | IL2RA low | 6776 | NM_ | NC_ | 42304559 | sense | ATCAAGCG | 2330. | GAGGATC | 2474. | GGG | 12 | 0.6531 |
| | | | | 003152.3 | 000017.11 | | | TGCTGACC | | AAGCGTG | | | | |
| | | | | | | | | GGCG | | CTGACCG | | | | |
| | | | | | | | | | | GCGGGGT | | | | |
| | | | | | | | | | | GC | | | | |
| STAT5A | IL2RA Teffs | IL2RA low | 6776 | NM_ | NC_ | 42305662 | sense | CAGCCAG | 2331. | ACGGCAG | 2475. | CGG | 13 | 0.6255 |
| | | | | 003152.3 | 000017.11 | | | GACCACAA | | CCAGGACC | | | | |
| | | | | | | | | TGCCA | | ACAATGCC | | | | |
| | | | | | | | | | | ACGGCTA | | | | |
| STAT5A | IL2RA Teffs | IL2RA low | 6776 | NM_ | NC_ | 42301385 | sense | CGTGCACA | 2332. | TGAACGT | 2476. | AGG | 10 | 0.5812 |
| | | | | 003152.3 | 000017.11 | | | TGAATCCC | | GCACATGA | | | | |
| | | | | | | | | CCCC | | ATCCCCCC | | | | |
| | | | | | | | | | | CAGGTGA | | | | |
| GATA3 | IL2RA Teffs | IL2RA low | 2625 | NM_ | NC_ | 8055892 | sense | AGGTACCC | 2333. | GCAGAGG | 2477. | CGG | 2 | 0.6857 |
| | | | | 002051.2 | 000010.11 | | | TCCGACCC | | TACCCTCC | | | | |
| | | | | | | | | ACCA | | GACCCACC | | | | |
| | | | | | | | | | | ACGGTGA | | | | |
| GATA3 | IL2RA Teffs | IL2RA low | 2625 | NM_ | NC_ | 8064014 | sense | CAGGGAG | 2334. | AAGGCAG | 2478 | GGG | 4 | 0.737 |
| | | | | 002051.2 | 000010.11 | | | TGTGTGAA | | GGAGTGT | | | | |
| | | | | | | | | CTGTG | | GTGAACT | | | | |
| | | | | | | | | | | GTGGGGC | | | | |
| | | | | | | | | | | AA | | | | |
| GATA3 | IL2RA Teffs | IL2RA low | 2625 | NM_ | NC_ | 8058740 | antisense | GGAGCTG | 2335. | GTCCGGA | 2479. | AGG | 3 | 0.6273 |
| | | | | 002051.2 | 000010.11 | | | TACTCGGG | | GCTGTACT | | | | |
| | | | | | | | | CACGT | | CGGGCAC | | | | |
| | | | | | | | | | | GTAGGGC | | | | |
| | | | | | | | | | | G | | | | |
| GATA3 | IL2RA Teffs | IL2RA low | 2625 | NM_ | NC_ | 8058432 | sense | TCCAAGAC | 2336. | CTTCTCCA | 2480. | CGG | 3 | 0.7243 |
| | | | | 002051.2 | 000010.11 | | | GTCCATCC | | AGACGTCC | | | | |
| | | | | | | | | ACCA | | ATCCACCA | | | | |
| | | | | | | | | | | CGGCTC | | | | |
| KMT2A | IL2RA Teffs | IL2RA low | 4297 | NM_ | NC_ | 1.19E+08 | antisense | AAGATCA | 2337. | ATTCAAGA | 2481. | TGG | 27 | 0.7172 |
| | | | | 005933.3 | 000011.10 | | | GTAGCGG | | TCAGTAGC | | | | |
| | | | | | | | | TCCCGG | | GGTCCCG | | | | |
| | | | | | | | | | | GTGGTGG | | | | |
| KMT2A | IL2RA Teffs | ILZRA low | 4297 | NM_ | NC_ | 1.18E+08 | sense | AGAAAGG | 2338. | GTAAAGA | 2482. | CGG | 5 | 0.6636 |
| | | | | 005933.3 | 000011.10 | | | ACGTCGAT | | AAGGACG | | | | |
| | | | | | | | | CGAGG | | TCGATCGA | | | | |
| | | | | | | | | | | GGCGGTG | | | | |
| | | | | | | | | | | T | | | | |
| KMT2A | IL2RA Teffs | IL2RA low | 4297 | NM_ | NC_ | 1.18E+08 | antisense | AGGGGTC | 2339. | GCCGAGG | 2483. | AGG | 3 | 0.757 |
| | | | | 005933.3 | 000011.10 | | | TTAATGAT | | GGTCTTAA | | | | |
| | | | | | | | | CCGCG | | TGATCCGC | | | | |
| | | | | | | | | | | GAGGAGA | | | | |
| KMT2A | IL2RA Teffs | IL2RA low | 4297 | NM_ | NC_ | 1.18E+08 | sense | TTGACCAT | 2340 | TCACTTGA | 2484. | TGG | 19 | 0.7229 |
| | | | | 005933.3 | 000011.10 | | | AATTATGC | | CCATAATT | | | | |
| | | | | | | | | TCAG | | ATGCTCAG | | | | |
| | | | | | | | | | | TGGCAG | | | | |
| PTEN | IL2RA Teffs | ILZRA low | 5728 | NM_ | NC_ | 87961027 | sense | AGAGCGT | 2341. | GTATAGA | 2485. | AGG | 8 | 0.7179 |
| | | | | 001304718.1 | 000010.11 | | | GCAGATA | | GCGTGCA | | | | |
| | | | | | | | | ATGACA | | GATAATGA | | | | |
| | | | | | | | | | | CAAGGAA | | | | |
| | | | | | | | | | | T | | | | |
| PTEN | IL2RA Teffs | IL2RA low | 5728 | NM_ | NC_ | 87957861 | antisense | AGCTGGC | 2342. | CTTTAGCT | 2486. | AGG | 7 | 0.6493 |
| | | | | 001304718.1 | 000010.11 | | | AGACCACA | | GGCAGAC | | | | |
| | | | | | | | | AACTG | | CACAAACT | | | | |
| | | | | | | | | | | GAGGATC | | | | |
| PTEN | IL2RA Teffs | IL2RA low | 5728 | NM_ | NC_ | 87960940 | sense | ATTCTTCA | 2343. | ATACATTC | 2487 | AGG | 8 | 0.6697 |
| | | | | 001304718.1 | 000010.11 | | | TACCAGGA | | TTCATACC | | | | |
| | | | | | | | | CCAG | | AGGACCA | | | | |
| | | | | | | | | | | GAGGAAA | | | | |
| PTEN | IL2RA Teffs | IL2RA low | 5728 | NM_ | NC_ | 87957915 | sense | CCAATTCA | 2344. | TCCTCCAA | 2488. | CGG | 7 | 0.6752 |
| | | | | 001304718.1 | 000010.11 | | | GGACCCAC | | TTCAGGAC | | | | |
| | | | | | | | | ACGA | | CCACACGA | | | | |
| | | | | | | | | | | CGGGAA | | | | |
| RELA | IL2RA Teffs | IL2RA low | 5970 | NM_ | NC_ | 65659757 | sense | ACTACGAC | 2345. | GGGGACT | 2489. | CGG | 6 | 0.7259 |
| | | | | 001243984.1 | 000011.10 | | | CTGAATGC | | ACGACCTG | | | | |
| | | | | | | | | TGTG | | AATGCTGT | | | | |
| | | | | | | | | | | GCGGCTC | | | | |
| RELA | IL2RA Teffs | IL2RA low | 5970 | NM_ | NC_ | 65662009 | sense | GCTTCCGC | 2346. | ATGCGCTT | 2490. | GGG | 3 | 0.7137 |
| | | | | 001243984.1 | 000011.10 | | | TACAAGTG | | CCGCTACA | | | | |
| | | | | | | | | CGAG | | AGTGCGA | | | | |
| | | | | | | | | | | GGGGCGC | | | | |
| RELA | IL2RA Teffs | IL2RA low | 5970 | NM_ | NC_ | 65658759 | antisense | GGAAGAT | 2347. | AGTAGGA | 2491. | AGG | 7 | 0.7554 |
| | | | | 001243984.1 | 000011.10 | | | CTCATCCC | | AGATCTCA | | | | |
| | | | | | | | | CACCG | | TCCCCACC | | | | |
| | | | | | | | | | | GAGGCAG | | | | |
| RELA | IL2RA Teffs | IL2RA low | 5970 | NM_ | NC_ | 65661818 | sense | TCAATGGC | 2348. | CAGATCAA | 2492. | GGG | 4 | 0.7164 |
| | | | | 001243984.1 | 000011.10 | | | TACACAGG | | TGGCTACA | | | | |
| | | | | | | | | ACCA | | CAGGACC | | | | |
| | | | | | | | | | | AGGGACA | | | | |
| ETS1 | IL2RA Teffs | IL2RA low | 2113 | NM_ | NC_ | 1.28E+08 | antisense | CTTACTAA | 2349. | TGAACTTA | 2493. | AGG | 4 | 0.6808 |
| | | | | 005238.3 | 000011.10 | | | TGAAGTAA | | CTAATGAA | | | | |
| | | | | | | | | TCCG | | GTAATCCG | | | | |
| | | | | | | | | | | AGGTAT | | | | |
| ETS1 | IL2RA Teffs | IL2RA low | 2113 | NM_ | NC_ | 1.28E+08 | antisense | GAGAAAG | 2350. | GCTCGAG | 2494. | GGG | 3 | 0.6487 |
| | | | | 005238.3 | 000011.10 | | | CAGTCTTT | | AAAGCAG | | | | |
| | | | | | | | | ACCCA | | TCTTTACC | | | | |
| | | | | | | | | | | CAGGGCG | | | | |
| | | | | | | | | | | C | | | | |
| ETS1 | IL2RA Teffs | IL2RA low | 2113 | NM_ | NC_ | 1.28E+08 | antisense | GGTCTCG | 2351. | AGAGGGT | 2495. | GGG | 5 | 0.7649 |
| | | | | 005238.3 | 000011.10 | | | GAGAATG | | CTCGGAG | | | | |
| | | | | | | | | ACCGAG | | AATGACCG | | | | |
| | | | | | | | | | | AGGGGTA | | | | |
| | | | | | | | | | | G | | | | |
| ETS1 | IL2RA Teffs | IL2RA low | 2113 | NM_ | NC_ | 1.28E+08 | sense | TGCATGG | 2352. | CATGTGCA | 2496. | TGG | 5 | 0.6503 |
| | | | | 005238.3 | 000011.10 | | | GGAGGAC | | TGGGGAG | | | | |
| | | | | | | | | CAGTCG | | GACCAGTC | | | | |
| | | | | | | | | | | GTGGTAG | | | | |
| RBPJ | IL2RA Teffs | IL2RA low | 3516 | NM_ | NC_ | 26415547 | sense | AAAGAAC | 2353. | AAAAAAA | 2497. | TGG | 5 | 0.6336 |
| | | | | 005349.3 | 000004.12 | | | AAATGGA | | GAACAAAT | | | | |
| | | | | | | | | ACGCGA | | GGAACGC | | | | |
| | | | | | | | | | | GATGGTT | | | | |
| | | | | | | | | | | G | | | | |
| RBPJ | IL2RA Teffs | ILZRA low | 3516 | NM_ | NC_ | 26386378 | antisense | CACCTAGT | 2354. | TACTCACC | 2498. | TGG | 3 | 0.6435 |
| | | | | 005349.3 | 000004.12 | | | AAGTCGTT | | TAGTAAGT | | | | |
| | | | | | | | | TAGG | | CGTTTAGG | | | | |
| | | | | | | | | | | TGGAGG | | | | |
| RBPJ | IL2RA Teffs | IL2RA low | 3516 | NM_ | NC_ | 26424454 | sense | CATGCCAG | 2355. | TTTTCATG | 2499. | GGG | 7 | 0.6844 |
| | | | | 005349.3 | 000004.12 | | | TTCACAGC | | CCAGTTCA | | | | |
| | | | | | | | | AGTG | | CAGCAGT | | | | |
| | | | | | | | | | | GGGGAGC | | | | |
| RBPI | IL2RA Teffs | IL2RA low | 3516 | NM_ | NC_ | 26424363 | sense | CATTGCCT | 2356. | TATGCATT | 2500. | TGG | 7 | 0.65 |
| | | | | 005349.3 | 000004.12 | | | CAGGAAC | | GCCTCAG | | | | |
| | | | | | | | | AAAGG | | GAACAAA | | | | |
| | | | | | | | | | | GGTGGCT | | | | |
| | | | | | | | | | | C | | | | |
| RXRB | IL2RA Teffs | IL2RA low | 6257 | NM_ | NC_ | 33198326 | sense | ACGGCTAT | 2357. | GCAAACG | 2501. | GGG | 3 | 0.7124 |
| | | | | 021976.4 | 000006.12 | | | GTGCAATC | | GCTATGTG | | | | |
| | | | | | | | | TGCG | | CAATCTGC | | | | |
| | | | | | | | | | | GGGGACA | | | | |
| RXRB | IL2RA Teffs | IL2RA low | 6257 | NM_ | NC_ | 33200341 | sense | GCCCTGGC | 2358. | GACGGCC | 2502. | CGG | 1 | 0.6547 |
| | | | | 021976.4 | 000006.12 | | | TGGATCCC | | CTGGCTG | | | | |
| | | | | | | | | GCAG | | GATCCCGC | | | | |
| | | | | | | | | | | AGCGGCG | | | | |
| | | | | | | | | | | G | | | | |
| RXRB | IL2RA Teffs | IL2RA low | 6257 | NM_ | NC_ | 33197840 | sense | GGACAAC | 2359. | GCCGGGA | 2503. | TGG | 4 | 0.7879 |
| | | | | 021976.4 | 000006.12 | | | AAAGACT | | CAACAAA | | | | |
| | | | | | | | | GCACAG | | GACTGCAC | | | | |
| | | | | | | | | | | AGTGGAC | | | | |
| | | | | | | | | | | A | | | | |
| RXRB | IL2RA Teffs | IL2RA low | 6257 | NM_ | NC_ | 33198421 | antisense | GTGGCTTC | 2360 | ACTGGTG | 2504 | GGG | 3 | 0.6579 |
| | | | | 021976.4 | 000006.12 | | | ACATCTTC | | GCTTCACA | | | | |
| | | | | | | | | AGGG | | TCTTCAGG | | | | |
| | | | | | | | | | | GGGGCCA | | | | |
| ZNF148 | IL2RA Teffs | IL2RA low | 7707 | NM_ | NC_ | 1.25E+08 | sense | AGATCGA | 2361. | TTCAAGAT | 2505. | AGG | 4 | 0.5525 |
| | | | | 021964.2 | 000003.12 | | | AGTATGCC | | CGAAGTAT | | | | |
| | | | | | | | | TCACC | | GCCTCACC | | | | |
| | | | | | | | | | | AGGAGA | | | | |
| ZNF148 | IL2RA Teffs | IL2RA low | 7707 | NM_ | NC_ | 1.25E+08 | antisense | AGTGCATA | 2362. | ATTAAGTG | 2506. | AGG | 4 | 0.6259 |
| | | | | 021964.2 | 000003.12 | | | CTGTAGTC | | CATACTGT | | | | |
| | | | | | | | | CTTG | | AGTCCTTG | | | | |
| | | | | | | | | | | AGGAAG | | | | |
| ZNF148 | IL2RA Teffs | IL2RA low | 7707 | NM_ | NC_ | 1.25E+08 | antisense | GAGCCCCC | 2363. | ATGCGAG | 2507. | TGG | 9 | 0.6379 |
| | | | | 021964.2 | 000003.12 | | | AACTGACG | | CCCCCAAC | | | | |
| | | | | | | | | AATG | | TGACGAAT | | | | |
| | | | | | | | | | | GTGGCAT | | | | |
| ZNF148 | IL2RA Teffs | IL2RA low | 7707 | NM_ | NC_ | 1.25E+08 | antisense | TAATTAGT | 2364. | ATCATAAT | 2508. | AGG | 9 | 0.6151 |
| | | | | 021964.2 | 000003.12 | | | ACTACTAT | | TAGTACTA | | | | |
| | | | | | | | | GCAC | | CTATGCAC | | | | |
| | | | | | | | | | | AGGTTT | | | | |
| VPS52 | IL2RA Teffs | IL2RA low | 6293 | NM_ | NC_ | 33267957 | sense | CAATGAAC | 2365. | TGGGCAA | 2509 | AGG | 8 | 0.6103 |
| | | | | 001289174.1 | 000006.12 | | | GAGCAAC | | TGAACGA | | | | |
| | | | | | | | | AGCAA | | GCAACAG | | | | |
| | | | | | | | | | | CAAAGGA | | | | |
| | | | | | | | | | | GA | | | | |
| VPS52 | IL2RA Teffs | IL2RA low | 6293 | NM_ | NC_ | 33266562 | sense | CCGTACAC | 2366. | TGGGCCG | 2510. | TGG | 11 | 0.6309 |
| | | | | 001289174.1 | 000006.12 | | | TCAGCATG | | TACACTCA | | | | |
| | | | | | | | | ACCC | | GCATGACC | | | | |
| | | | | | | | | | | CTGGTAA | | | | |
| VPS52 | IL2RA Teffs | IL2RA low | 6293 | NM_ | NC_ | 33269070 | sense | GAAATCGC | 2367. | CTTCGAAA | 2511. | GGG | 5 | 0.7039 |
| | | | | 001289174.1 | 000006.12 | | | CAGGCAG | | TCGCCAG | | | | |
| | | | | | | | | TTCGG | | GCAGTTCG | | | | |
| | | | | | | | | | | GGGGAAA | | | | |
| VPS52 | IL2RA Teffs | ILZRA low | 6293 | NM_ | NC_ | 33264461 | antisense | TCCAGGAT | 2368. | CATCTCCA | 2512. | TGG | 13 | 0.6852 |
| | | | | 001289174.1 | 000006.12 | | | CAGTTCAA | | GGATCAG | | | | |
| | | | | | | | | ACCG | | TTCAAACC | | | | |
| | | | | | | | | | | GTGGCCA | | | | |
| TFAP4 | IL2RA Teffs | IL2RA low | 7023 | NM_ | NC_ | 4262329 | sense | ACAGCTCA | 2369. | ACACACAG | 2513. | AGG | 3 | 0.5694 |
| | | | | 003223.2 | 000016.10 | | | AGCGCTTC | | CTCAAGCG | | | | |
| | | | | | | | | ATCC | | CTTCATCC | | | | |
| | | | | | | | | | | AGGTGC | | | | |
| TFAP4 | IL2RA Teffs | IL2RA low | 7023 | NM_ | NC_ | 4261877 | sense | AGGCTCCC | 2370. | GCATAGG | 2514. | AGG | 4 | 0.6975 |
| | | | | 003223.2 | 000016.10 | | | CGGACATC | | CTCCCCGG | | | | |
| | | | | | | | | TGGG | | ACATCTGG | | | | |
| | | | | | | | | | | GAGGACG | | | | |
| TFAP4 | IL2RA Teffs | IL2RA low | 7023 | NM_ | NC_ | 4262671 | sense | CACTAACC | 2371. | ATTCCACT | 2515. | CGG | 2 | 0.6468 |
| | | | | 003223.2 | 000016.10 | | | CCCGAGAC | | AACCCCCG | | | | |
| | | | | | | | | TCAG | | AGACTCAG | | | | |
| | | | | | | | | | | CGGGAC | | | | |
| TFAP4 | IL2RA Teffs | IL2RA low | 7023 | NM_ | NC_ | 4262597 | sense | CGCATGCA | 2372. | GAGACGC | 2516. | GGG | 2 | 0.634 |
| | | | | 003223.2 | 000016.10 | | | GAGCATCA | | ATGCAGA | | | | |
| | | | | | | | | ACGC | | GCATCAAC | | | | |
| | | | | | | | | | | GCGGGAT | | | | |
| | | | | | | | | | | T | | | | |
| IKZF3 | IL2RA Teffs | IL2RA low | 22806 | NM_ | NC_ | 39792732 | sense | AAGATGA | 2373. | TGGAAAG | 2517. | TGG | 4 | 0.7182 |
| | | | | 012481.4 | 000017.11 | | | ACTGCGAT | | ATGAACTG | | | | |
| | | | | | | | | GTGTG | | CGATGTGT | | | | |
| | | | | | | | | | | GTGGATT | | | | |
| IKZF3 | IL2RA Teffs | IL2RA low | 22806 | NM_ | NC_ | 39788318 | sense | CAAGCAG | 2374. | GTTACAAG | 2518. | AGG | 6 | 0.6635 |
| | | | | 012481.4 | 000017.11 | | | AGAAGTTC | | CAGAGAA | | | | |
| | | | | | | | | CCTTG | | GTTCCCTT | | | | |
| | | | | | | | | | | GAGGAGC | | | | |
| IKZF3 | IL2RA Teffs | IL2RA low | 22806 | NM_ | NC_ | 39766413 | sense | GCTCATAC | 2375 | GTGAGCTC | 2519 | TGG | 8 | 0.6101 |
| | | | | 012481.4 | 000017.11 | | | AGACCCGC | | ATACAGAC | | | | |
| | | | | | | | | ATGA | | CCGCATGA | | | | |
| | | | | | | | | | | TGGACC | | | | |
| IKZF3 | IL2RA Teffs | IL2RA low | 22806 | NM_ | NC_ | 39777693 | sense | GGACAGA | 2376. | TACTGGAC | 2520. | TGG | 7 | 0.7336 |
| | | | | 012481.4 | 000017.11 | | | TTAGCAAG | | AGATTAGC | | | | |
| | | | | | | | | CAATG | | AAGCAAT | | | | |
| | | | | | | | | | | GTGGCAA | | | | |
| SRF | IL2RA Teffs | IL2RA low | 6722 | NM_ | NC_ | 43175724 | antisense | AGGTTGG | 2377. | CGGCAGG | 2521. | CGG | 3 | 0.6646 |
| | | | | 003131.3 | 000006.12 | | | TGACTGTG | | TTGGTGAC | | | | |
| | | | | | | | | AACGC | | TGTGAAC | | | | |
| | | | | | | | | | | GCCGGCTT | | | | |
| SRF | IL2RA Teffs | ILZRA low | 6722 | NM_ | NC_ | 43172119 | sense | AGTTCATC | 2378. | ATGGAGTT | 2522. | CGG | 1 | 0.7054 |
| | | | | 003131.3 | 000006.12 | | | GACAACA | | CATCGACA | | | | |
| | | | | | | | | AGCTG | | ACAAGCTG | | | | |
| | | | | | | | | | | CGGCGC | | | | |
| SRE | IL2RA Teffs | IL2RA low | 6722 | NM_ | NC_ | 43175844 | antisense | GGGCTGA | 2379. | ACTGGGG | 2523. | TGG | 3 | 0.65 |
| | | | | 003131.3 | 000006.12 | | | CACTAGCA | | CTGACACT | | | | |
| | | | | | | | | GACAC | | AGCAGAC | | | | |
| | | | | | | | | | | ACTGGTGC | | | | |
| SRE | IL2RA Teffs | IL2RA low | 6722 | NM_ | NC_ | 43174015 | antisense | TCTGTTGT | 2380. | CTGGTCTG | 2524. | GGG | 2 | 0.605 |
| | | | | 003131.3 | 000006.12 | | | GGGGTCT | | TTGTGGG | | | | |
| | | | | | | | | GAACG | | GTCTGAAC | | | | |
| | | | | | | | | | | GGGGTGG | | | | |
| CEBPZ | IL2RA Teffs | IL2RA low | 10153 | NM_ | NC_ | 37223251 | antisense | AAAGCTCC | 2381. | ATATAAAG | 2525. | TGG | 3 | 0.6404 |
| | | | | 005760.2 | 000002.12 | | | ACATATAA | | CTCCACAT | | | | |
| | | | | | | | | ATGG | | ATAAATGG | | | | |
| | | | | | | | | | | TGGCAT | | | | |
| CEBPZ | IL2RA Teffs | IL2RA low | 10153 | NM_ | NC_ | 37228088 | antisense | ACAAAGC | 2382. | TGTTACAA | 2526. | CGG | 2 | 0.63 |
| | | | | 000576.2 | 000002.12 | | | AGCTCATG | | AGCAGCTC | | | | |
| | | | | | | | | AGCCA | | ATGAGCCA | | | | |
| | | | | | | | | | | CGGTAA | | | | |
| CEBPZ | IL2RA Teffs | ILZRA low | 10153 | NM_ | NC_ | 37227623 | antisense | CAACATTA | 2383. | AAAGCAA | 2527. | TGG | 2 | 0.6374 |
| | | | | 005760.2 | 000002.12 | | | AAGCCTG | | CATTAAAG | | | | |
| | | | | | | | | GACAC | | CCTGGACA | | | | |
| | | | | | | | | | | CTGGTAT | | | | |
| CEBPZ | IL2RA Teffs | IL2RA low | 10153 | NM_ | NC_ | 37228473 | sense | TGAAGGC | 2384. | TGGATGA | 2528. | GGG | 2 | 0.7347 |
| | | | | 005760.2 | 000002.12 | | | AATTGTGT | | AGGCAATT | | | | |
| | | | | | | | | CATCG | | GTGTCATC | | | | |
| | | | | | | | | | | GGGGACA | | | | |
| BATF | IL2RA Teffs | IL2RA low | 10538 | NM_ | NC_ | 75546499 | sense | ACAGAAC | 2385. | AGAAACA | 2529. | AGG | 3 | 0.599 |
| | | | | 006399.3 | 000014.9 | | | GCGGCTCT | | GAACGCG | | | | |
| | | | | | | | | ACGCA | | GCTCTACG | | | | |
| | | | | | | | | | | CAAGGAG | | | | |
| | | | | | | | | | | A | | | | |
| BATF | IL2RA Teffs | IL2RA low | 10538 | NM_ | NC_ | 75522739 | antisense | AGGACTCT | 2386. | AAGGAGG | 2530. | GGG | 1 | 0.6137 |
| | | | | 006399.3 | 000014.9 | | | ACCTGTTT | | ACTCTACC | | | | |
| | | | | | | | | GCCA | | TGTTTGCC | | | | |
| | | | | | | | | | | AGGGGGA | | | | |
| BATF | IL2RA Teffs | IL2RA low | 10538 | NM_ | NC_ | 75525114 | sense | ATGTGAG | 2387. | GATGATGT | 2531. | AGG | 2 | 0.5377 |
| | | | | 006399.3 | 000014.9 | | | AAGAGTTC | | GAGAAGA | | | | |
| | | | | | | | | AGAGG | | GTTCAGA | | | | |
| | | | | | | | | | | GGAGGGA | | | | |
| | | | | | | | | | | G | | | | |
| BATF | IL2RA Teffs | IL2RA low | 10538 | NM_ | NC_ | 75546520 | sense | GGAGATC | 2388. | GCAAGGA | 2532. | AGG | 3 | 0.7037 |
| | | | | 006399.3 | 000014.9 | | | AAGCAGCT | | GATCAAGC | | | | |
| | | | | | | | | CACAG | | AGCTCACA | | | | |
| | | | | | | | | | | GAGGAAC | | | | |
| CIC | IL2RA Teffs | IL2RA low | 23152 | NM_ | NC_ | 42291060 | sense | ACTGTCAC | 2389. | TGCCACTG | 2533. | GGG | 10 | 0.6591 |
| | | | | 015125.4 | 000019.10 | | | TAACCTAC | | TCACTAAC | | | | |
| | | | | | | | | TGGT | | CTACTGGT | | | | |
| | | | | | | | | | | GGGCAC | | | | |
| CIC | IL2RA Teffs | IL2RA low | 23152 | NM_ | NC_ | 42292311 | antisense | CCCGCCCG | 2390. | CGTGCCCG | 2534 | AGG | 13 | 0.6689 |
| | | | | 015125.4 | 000019.10 | | | CTGACTGC | | CCCGCTGA | | | | |
| | | | | | | | | ACAT | | CTGCACAT | | | | |
| | | | | | | | | | | AGGTGA | | | | |
| CIC | IL2RA Teffs | IL2RA low | 23152 | NM_ | NC_ | 42287372 | sense | CTCTACCG | 2391. | TTGCCTCT | 2535. | CGG | 4 | 0.6842 |
| | | | | 015125.4 | 000019.10 | | | CCCGGAA | | ACCGCCCG | | | | |
| | | | | | | | | AACGT | | GAAAACG | | | | |
| | | | | | | | | | | TCGGACC | | | | |
| CIC | IL2RA Teffs | IL2RA low | 23152 | NM_ | NC_ | 42289198 | antisense | TTGGGCCA | 2392. | GGCTTTGG | 2536. | GGG | 8 | 0.6248 |
| | | | | 015125.4 | 000019.10 | | | GAGTACG | | GCCAGAG | | | | |
| | | | | | | | | ATGCA | | TACGATGC | | | | |
| | | | | | | | | | | AGGGCCA | | | | |
| IKZF1 | IL2RA Teffs | IL2RA low | 10320 | NM_ | NC_ | 50376659 | sense | GAAAATG | 2393. | GAGAGAA | 2537. | GGG | 4 | 0.66 |
| | | | | 006060.5 | 000007.14 | | | AATGGCTC | | AATGAATG | | | | |
| | | | | | | | | CCACA | | GCTCCCAC | | | | |
| | | | | | | | | | | AGGGACC | | | | |
| IKZF1 | IL2RA Teffs | IL2RA low | 10320 | NM_ | NC_ | 50399996 | antisense | GATGGCTT | 2394. | TGTTGATG | 2538. | GGG | 8 | 0.7379 |
| | | | | 006060.5 | 000007.14 | | | GGTCCATC | | GCTTGGTC | | | | |
| | | | | | | | | ACGT | | CATCACGT | | | | |
| | | | | | | | | | | GGGACT | | | | |
| IKZF1 | IL2RA Teffs | IL2RA low | 10320 | NM_ | NC_ | 50382586 | sense | GGGGCCT | 2395. | GTGCGGG | 2539 | GGG | 5 | 0.6196 |
| | | | | 006060.5 | 000007.14 | | | CATTCACC | | GCCTCATT | | | | |
| | | | | | | | | CAGAA | | CACCCAGA | | | | |
| | | | | | | | | | | AGGGCAA | | | | |
| IKZF1 | IL2RA Teffs | IL2RA low | 10320 | NM_ | NC_ | S0327753 | sense | TCCAAGAG | 2396. | AAGCTCCA | 2540. | GGG | 3 | 0.617 |
| | | | | 006060.5 | 000007.14 | | | TGACAGA | | AGAGTGA | | | | |
| | | | | | | | | GTCGT | | CAGAGTC | | | | |
| | | | | | | | | | | GTGGGTA | | | | |
| | | | | | | | | | | A | | | | |
| IRF4 | IL2RA Teffs | IL2RA low | 3662 | NM_ | NC_ | 394977 | sense | CAGACCCG | 2397. | ATCTCAGA | 2541. | AGG | 3 | 0.5822 |
| | | | | 001195286.1 | 000006.12 | | | TACAAAGT | | CCCGTACA | | | | |
| | | | | | | | | GTAC | | AAGTGTAC | | | | |
| | | | | | | | | | | AGGATT | | | | |
| IRF4 | IL2RA Teffs | IL2RA low | 3662 | NM_ | NC_ | 397215 | sense | CCCATGAC | 2398. | ATGTCCCA | 2542. | CGG | 5 | 0.7031 |
| | | | | 001195286.1 | 000006.12 | | | GTTTGGAC | | TGACGTTT | | | | |
| | | | | | | | | CCCG | | GGACCCC | | | | |
| | | | | | | | | | | GCGGCCA | | | | |
| IRF4 | IL2RA Teffs | IL2RA low | 3662 | NM_ | NC_ | 401466 | sense | CTACCGG | 2399. | TGTACTAC | 2543. | AGG | 7 | 0.6428 |
| | | | | 001195286.1 | 000006.12 | | | GAAATCCT | | CGGGAAA | | | | |
| | | | | | | | | CGTGA | | TCCTCGTG | | | | |
| | | | | | | | | | | AAGGAGC | | | | |
| IRF4 | IL2RA Teffs | IL2RA low | 3662 | NM_ | NC_ | 393251 | sense | CTGATCGA | 2400. | GTGGCTG | 2544. | CGG | 2 | 0.6887 |
| | | | | 001195286.1 | 000006.12 | | | CCAGATCG | | ATCGACCA | | | | |
| | | | | | | | | ACAG | | GATCGACA | | | | |
| | | | | | | | | | | GCGGCAA | | | | |
| MBD2 | IL2RA Teffs | ILZRA low | 8932 | NM_ | NC_ | 54224170 | sense | AGCCGGT | 2401. | CGGGAGC | 2545. | GGG | 1 | 0.5949 |
| | | | | 003927.4 | 000018.10 | | | CCCTTTCC | | CGGTCCCT | | | | |
| | | | | | | | | CGTCG | | TTCCCGTC | | | | |
| | | | | | | | | | | GGGGAGC | | | | |
| MBD2 | IL2RA Teffs | IL2RA low | 8932 | NM_ | NC_ | 54205113 | sense | CCTCAGTT | 2402. | CAAGCCTC | 2546. | GGG | 2 | 0.5818 |
| | | | | 003927.4 | 000018.10 | | | GGCAAGG | | AGTTGGC | | | | |
| | | | | | | | | TACCT | | AAGGTACC | | | | |
| | | | | | | | | | | TGGGAAA | | | | |
| MBD2 | IL2RA Teffs | IL2RA low | 8932 | NM_ | NC_ | 54204999 | sense | CCTCTCAA | 2403. | CGATCCTC | 2547. | TGG | 2 | 0.5401 |
| | | | | 003927.4 | 000018.10 | | | TCAAAATA | | TCAATCAA | | | | |
| | | | | | | | | AGGT | | AATAAGGT | | | | |
| | | | | | | | | | | TGGTTA | | | | |
| MBD2 | IL2RA Teffs | IL2RA low | 8932 | NM_ | NC_ | 54224048 | sense | CGAAAATC | 2404. | GATCCGAA | 2548. | TGG | 1 | 0.494 |
| | | | | 003927.4 | 000018.10 | | | TGGGCTA | | AATCTGG | | | | |
| | | | | | | | | AGTGC | | GCTAAGT | | | | |
| | | | | | | | | | | GCTGGCA | | | | |
| | | | | | | | | | | A | | | | |
| JAK3 | IL2RA Teffs | IL2RA low | 3718 | NM_ | NC_ | 17835098 | antisense | ACTCTCCA | 2405. | ACTTACTO | 2549 | CGG | 15 | 0.7301 |
| | | | | 000215.3 | 000019.10 | | | GGCTTAAC | | TCCAGGCT | | | | |
| | | | | | | | | ACAG | | TAACACAG | | | | |
| | | | | | | | | | | CGGGGC | | | | |
| JAK3 | IL2RA Teffs | ILZRA low | 3718 | NM_ | NC_ | 17839577 | antisense | AGCTCTCG | 2406. | CAGGAGC | 2550. | GGG | 10 | 0.7109 |
| | | | | 000215.3 | 000019.10 | | | AAGACTGC | | TCTCGAAG | | | | |
| | | | | | | | | TGTG | | ACTGCTGT | | | | |
| | | | | | | | | | | GGGGTCG | | | | |
| JAK3 | IL2RA Teffs | IL2RA low | 3718 | NM_ | NC_ | 17836043 | antisense | GTGTACAA | 2407. | CCAGGTGT | 2551. | TGG | 14 | 0.6764 |
| | | | | 000215.3 | 000019.10 | | | ATTCCTGC | | ACAAATTC | | | | |
| | | | | | | | | ACCA | | CTGCACCA | | | | |
| | | | | | | | | | | TGGTGC | | | | |
| JAK3 | IL2RA Teffs | IL2RA low | 3718 | NM_ | NC_ | 17842538 | sense | TGACGCG | 2408. | TTCGTGAC | 2552. | AGG | 6 | 0.6476 |
| | | | | 000215.3 | 000019.10 | | | GAGGCGT | | GCGGAGG | | | | |
| | | | | | | | | ATTCGG | | CGTATTCG | | | | |
| | | | | | | | | | | GAGGACG | | | | |
| BCL6 | IL2RA Teffs | IL2RA low | 604 | NM_ | NC_ | 1.88E+08 | sense | AAGTGATA | 2409. | CACGAAGT | 2553. | TGG | 5 | 0.8035 |
| | | | | 001706.4 | 000003.12 | | | TGCACTAC | | GATATGCA | | | | |
| | | | | | | | | AGTG | | CTACAGTG | | | | |
| | | | | | | | | | | TGGCTG | | | | |
| BCL6 | IL2RA Teffs | IL2RA low | 604 | NM_ | NC_ | 1.88E+08 | sense | CAAGACAT | 2410. | GCCCCAAG | 2554. | GGG | 5 | 0.6458 |
| | | | | 001706.4 | 000003.12 | | | CATGGCCT | | ACATCATG | | | | |
| | | | | | | | | ATCG | | GCCTATCG | | | | |
| | | | | | | | | | | GGGTCG | | | | |
| BCL6 | IL2RA Teffs | ILZRA low | 604 | NM_ | NC_ | 1.88E+08 | antisense | CATCTGCA | 2411. | GCTCCATC | 2555. | TGG | 4 | 0.6878 |
| | | | | 001706.4 | 000003.12 | | | GGTACATA | | TGCAGGT | | | | |
| | | | | | | | | GCCG | | ACATAGCC | | | | |
| | | | | | | | | | | GTGGCCA | | | | |
| BCL6 | IL2RA Teffs | IL2RA low | 604 | NM_ | NC_ | 1.88E+08 | antisense | TCCCTCAC | 2412. | CAGATCCC | 2556. | AGG | 3 | 0.6413 |
| | | | | 001706.4 | 000003.12 | | | CTGCAGG | | TCACCTGC | | | | |
| | | | | | | | | CCATG | | AGGCCAT | | | | |
| | | | | | | | | | | GAGGACC | | | | |
| ZNF236 | IL2RA Teffs | IL2RA low | 7776 | NM_ | NC_ | 76919836 | sense | ATCACGGA | 2413. | ATGCATCA | 2557. | AGG | 20 | 0.6507 |
| | | | | 007345.3 | 000018.10 | | | AGTCTCGT | | CGGAAGT | | | | |
| | | | | | | | | CCTG | | CTCGTCCT | | | | |
| | | | | | | | | | | GAGGTCA | | | | |
| ZNF236 | IL2RA Teffs | IL2RA low | 7776 | NM_ | NC_ | 76913812 | sense | GCATGTGC | 2414. | AGCAGCAT | 2558. | GGG | 18 | 0.7309 |
| | | | | 007345.3 | 000018.10 | | | GGTCGCA | | GTGCGGT | | | | |
| | | | | | | | | CACCG | | CGCACACC | | | | |
| | | | | | | | | | | GGGGAAA | | | | |
| ZNF236 | IL2RA Teffs | IL2RA low | 7776 | NM_ | NC_ | 76905317 | sense | GGCAGCTT | 2415. | TGGTGGC | 2559 | GGG | 13 | 0.6112 |
| | | | | 007345.3 | 000018.10 | | | ACGGCGA | | AGCTTACG | | | | |
| | | | | | | | | CACAT | | GCGACAC | | | | |
| | | | | | | | | | | ATGGGTAT | | | | |
| ZNF236 | IL2RA Teffs | IL2RA low | 7776 | NM_ | NC_ | 76910097 | antisense | TGGTCTAC | 2416. | AAACTGGT | 2560. | AGG | 15 | 0.6536 |
| | | | | 007345.3 | 000018.10 | | | GTGCCCTC | | CTACGTGC | | | | |
| | | | | | | | | GCAG | | CCTCGCAG | | | | |
| | | | | | | | | | | AGGGTC | | | | |
| TCF3 | IL2RA Teffs | IL2RA low | 6929 | NM_ | NC_ | 1622055 | sense | CACCAGCA | 2417. | CCTGCACC | 2561. | AGG | 10 | 0.6193 |
| | | | | 003200.3 | 000019.10 | | | CGAGCGT | | AGCACGA | | | | |
| | | | | | | | | ATGGT | | GCGTATG | | | | |
| | | | | | | | | | | GTAGGAC | | | | |
| | | | | | | | | | | C | | | | |
| TCF3 | IL2RA Teffs | IL2RA low | 6929 | NM_ | NC_ | 1622322 | sense | CTATCCCG | 2418. | GCACCTAT | 2562. | TGG | 9 | 0.6605 |
| | | | | 003200.3 | 000019.10 | | | CCCCCTTC | | CCCGCCCC | | | | |
| | | | | | | | | TACG | | CTTCTACG | | | | |
| | | | | | | | | | | TGGCAG | | | | |
| TCF3 | IL2RA Teffs | IL2RA low | 6929 | NM_ | NC_ | 1619821 | antisense | GCTGGGC | 2419. | CGTAGCTG | 2563. | GGG | 14 | 0.6527 |
| | | | | 003200.3 | 000019.10 | | | GATAAGG | | GGCGATA | | | | |
| | | | | | | | | CACCGG | | AGGCACC | | | | |
| | | | | | | | | | | GGGGGCT | | | | |
| | | | | | | | | | | C | | | | |
| TCF3 | IL2RA Teffs | IL2RA low | 6929 | NM_ | NC_ | 1621034 | antisense | GTTATTGC | 2420 | AGAAGTTA | 2564. | GGG | 13 | 0.6808 |
| | | | | 003200.3 | 000019.10 | | | TTGAGTGA | | TTGCTTGA | | | | |
| | | | | | | | | TCCG | | GTGATCCG | | | | |
| | | | | | | | | | | GGGAGT | | | | |
| YY1 | IL2RA Teffs | IL2RA low | 7528 | NM_ | NC_ | 1E+08 | sense | AGATATTG | 2421. | AAAAAGA | 2565. | TGG | 2 | 0.667 |
| | | | | 003403.4 | 000014.9 | | | ACCATGAG | | TATTGACC | | | | |
| | | | | | | | | ACAG | | ATGAGAC | | | | |
| | | | | | | | | | | AGTGGTT | | | | |
| | | | | | | | | | | G | | | | |
| YY1 | IL2RA Teffs | IL2RA low | 7528 | NM_ | NC_ | 1E+08 | sense | GGAGACC | 2422. | CGGTGGA | 2566. | TGG | 1 | 0.7975 |
| | | | | 003403.4 | 000014.9 | | | ATCGAGAC | | GACCATCG | | | | |
| | | | | | | | | CACAG | | AGACCACA | | | | |
| | | | | | | | | | | GTGGTGG | | | | |
| YY1 | IL2RA Teffs | IL2RA low | 7528 | NM_ | NC_ | 1E+08 | sense | GGTCACC | 2423. | CGCTGGTC | 2567. | AGG | 1 | 0.6314 |
| | | | | 003403.4 | 000014.9 | | | GACGACCC | | ACCGACG | | | | |
| | | | | | | | | GACCC | | ACCCGACC | | | | |
| | | | | | | | | | | CAGGTGC | | | | |
| YY1 | IL2RA Teffs | IL2RA low | 7528 | NM_ | NC_ | 1E+08 | sense | TGAACAAA | 2424. | ACATTGAA | 2568. | TGG | 1 | 0.7093 |
| | | | | 003403.4 | 000014.9 | | | CGCTGGTC | | CAAACGCT | | | | |
| | | | | | | | | ACCG | | GGTCACC | | | | |
| | | | | | | | | | | GTGGCGG | | | | |
| PURA | IL2RA Teffs | IL2RA low | 5813 | NM_ | NC_ | 1.4E+08 | sense | CCTTACTC | 2425. | GCCGCCTT | 2569. | TGG | 1 | 0.6294 |
| | | | | 005859.4 | 000005.10 | | | TCTCCATG | | ACTCTCTC | | | | |
| | | | | | | | | TCAG | | CATGTCAG | | | | |
| | | | | | | | | | | TGGCCG | | | | |
| PURA | IL2RA Teffs | IL2RA low | 5813 | NM_ | NC_ | 1.4E+08 | antisense | GAACTCGA | 2426. | CACGGAA | 2570. | CGG | 1 | 0.6814 |
| | | | | 005859.4 | 000005.10 | | | TGAGCCCC | | CTCGATGA | | | | |
| | | | | | | | | TGCG | | GCCCCTGC | | | | |
| | | | | | | | | | | GCGGGCA | | | | |
| PURA | IL2RA Teffs | IL2RA low | 5813 | NM_ | NC_ | 1.4E+08 | sense | GCTCATCG | 2427. | CCAAGCTC | 2571. | TGG | 1 | 0.6758 |
| | | | | 005859.4 | 000005.10 | | | ACGACTAC | | ATCGACGA | | | | |
| | | | | | | | | GGAG | | CTACGGA | | | | |
| | | | | | | | | | | GTGGAGG | | | | |
| PURA | IL2RA Teffs | IL2RA low | 5813 | NM_ | NC_ | 1.4E+08 | sense | TCCGCCAG | 2428. | CGCATCCG | 2572. | GGG | 1 | 0.6124 |
| | | | | 005859.4 | 000005.10 | | | ACGGTCAA | | CCAGACG | | | | |
| | | | | | | | | CCGG | | GTCAACCG | | | | |
| | | | | | | | | | | GGGGCCT | | | | |

In some embodiments, inhibition of one or more nuclear factors set forth in Table 1 and/or overexpression of one or more nuclear factors set forth in Table 2 a may increase CTLA4 expression in the T cell. In some embodiments, inhibition of one or more nuclear factors set forth in Table 2, and/or overexpression of one or more nuclear factor set forth in Table 1 may decrease CTLA4 expression in the T cell.

In some embodiments, the T cell comprises: (a) a genetic modification or a heterologous polynucleotide that inhibits expression of CBFB, MYB, ZNF217, FOXK1, FLI1, FOS, SATB1, IL2 or ATXN7L3, wherein expression of CTLA4 is increased in the T cell relative to expression of CTLA4 in a T cell not comprising the genetic modification or the heterologous polynucleotide that inhibits expression of CBFB, MYB, ZNF217, FOXK1, FLI1, FOS, SATB1, IL2 or ATXN7L3; and/or (b) a heterologous polypeptide that encodes MTF1, RELA, IRF1, BCL11B, STAT3, MED30, MED14, MED11, IKZF3, KMT2A, IKZF1, MED12, TAF5L, PTEN, IRF4, FOXO1, FOXP1 or CTLA4, wherein expression of CTLA4 is increased in the T cell relative to expression of CTLA4 in a T cell not comprising the heterologous polypeptide that encodes MTF1, RELA, IRF1, BCL11B, STAT3, MED30, MED14, MED11, IKZF3, KMT2A, IKZF1, MED12, TAF5L, PTEN, IRF4, FOXO1, FOXP1 or CTLA4.

In some embodiments, the T cell comprises: (a) a genetic modification or heterologous polynucleotide that inhibits expression of MTF1, RELA, IRF1, BCL11B, STAT3, MED30, MED14, MED11, IKZF3, KMT2A, IKZF1, MED12, TAF5L, PTEN, IRF4, FOXO1, FOXP1 or CTLA4, wherein expression of CTLA4 is decreased in the T cell relative to expression of CTLA4 in a T cell not comprising the genetic modification or the heterologous polynucleotide that inhibits expression of MTF1, RELA, IRF1, BCL11B, STAT3, MED30, MED14, MED11, IKZF3, KMT2A, IKZF1, MED12, TAF5L, PTEN, IRF4, FOXO1, FOXP1 or CTLA4; and/or (b) a heterologous polypeptide that encodes CBTB, MYB, ZNF217, FOXK1, FLI1, FOX, SATB1, IL2 or ATXN7L3, wherein expression of CTLA4 is decreased in the T cell relative to expression of CTLA4 in a T cell not comprising the heterologous polypeptide that encodes CBTB, MYB, ZNF217, FOXK1, FLI1, FOX, SATB1, IL2 or ATXN7L3.

In some embodiments, inhibition of one or more nuclear factors set forth in Table 3 and/or overexpression of one or more nuclear factors set forth in Table 4 may increase FOXP3 expression in the T cell. In some embodiments, inhibition of one or more nuclear factors set forth in Table 4, and/or overexpression of one or more nuclear factor set forth in Table 3 may decrease FOXP3 expression in the T cell.

In some embodiments, the T cell comprises: (a) a genetic modification or heterologous polynucleotide that inhibits expression of ETS1, MYBL2, MYB, TP53, FLI1, SATB1, MBD2, ZBTB7A, DNMT1, TFDP1, SMARCB1 or MAF, wherein expression of FOXP3 is increased in the T cell relative to expression of FOXP3 in a T cell not comprising the genetic modification or heterologous polynucleotide that inhibits expression of ETS1, MYBL2, MYB, TP53, FLI1, SATB1, MBD2, ZBTB7A, DNMT1, TFDP1, SMARCB1 or MAF; and/or (b) a heterologous polypeptide that encodes a TAF5L, FOXP3, GATA3, STAT5B, FOXP1, STAT5A, PTEN or FOXO1, wherein expression of FOXP3 is increased in the T cell relative to expression of FOXP3 in a T cell not comprising a heterologous polypeptide that encodes a TAF5L, FOXP3, GATA3, STAT5B, FOXP1, STAT5A, PTEN or FOXO1.

In some embodiments, the T cell comprises: (a) a genetic modification or heterologous polynucleotide that inhibits expression of TAF5L, FOXP3, GATA3, STAT5B, FOXP1, STAT5A, PTEN or FOXO1, wherein expression of FOXP3 is decreased in the T cell relative to expression of FOXP3 in a T cell not comprising the genetic modification or heterologous polynucleotide that inhibits expression of TAF5L, FOXP3, GATA3, STAT5B, FOXP1, STAT5A, PTEN or FOXO1; and/or (b) a heterologous polypeptide that encodes ETS1, MYBL2, MYB, TP53, FLI1, SATB1, MBD2, ZBTB7A, DNMT1, TFDP1, SMARCB1 or MAF, wherein expression of FOXP3 is decreased in the T cell relative to expression of FOXP3 in a T cell not comprising a heterologous polypeptide that encodes ETS1, MYBL2, MYB, TP53, FLI1, SATB1, MBD2, ZBTB7A, DNMT1, TFDP1, SMARCB1 or MAF.

In some embodiments, inhibition of one or more nuclear factors set forth in Table 5 and/or overexpression of one or more nuclear factors set forth in Table 6 may increase IL-2 expression in the T cell. In some embodiments, inhibition of one or more nuclear factors set forth in Table 6, and/or overexpression of one or more nuclear factor set forth in Table 5 may decrease IL-2 expression in the T cell.

In some embodiments, the T cell comprises: (a) a genetic modification or heterologous polynucleotide that inhibits expression of MED12, FOXP1, PTEN, IKZF1, TAF5L, PRDM1, TFDP1, CXXC1, IKZF3 or TP53, wherein expression of IL-2 is increased in the T cell relative to expression of IL-2 in a T cell not comprising the genetic modification or heterologous polynucleotide that inhibits expression of MED12, FOXP1, PTEN, IKZF1, TAF5L, PRDM1, TFDP1, CXXC1, IKZF3 or TP53; and/or (b) a heterologous polypeptide that encodes NFATC2, MAF, ZBTB7A, MBD2, GATA3, MED14, IRF2, MED30, ZBTB11, RELA, JAK3, MED11, BCL11B, MTF1, ATXN7L3, YY1, ETS1, IL2, DNMT1, GTF2B or SMARCB1, wherein expression of IL-2 is increased in the T cell relative to expression of IL-2 in a T cell not comprising heterologous polypeptide that encodes NFATC2, MAF, ZBTB7A, MBD2, GATA3, MED14, IRF2, MED30, ZBTB11, RELA, JAK3, MED11, BCL11B, MTF1, ATXN7L3, YY1, ETS1, IL2, DNMT1, GTF2B or SMARCB1.

In some embodiments, the T cell comprises: (a) genetic modification or heterologous polynucleotide that inhibits expression of NFATC2, MAF, ZBTB7A, MBD2, GATA3, MED14, IRF2, MED30, ZBTB11, RELA, JAK3, MED11, BCL11B, MTF1, ATXN7L3, YY1, ETS1, IL2, DNMT1, GTF2B or SMARCB1, wherein expression of IL-2 is decreased in the T cell relative to expression of IL-2 in a T cell not comprising the genetic modification or heterologous polynucleotide that inhibits expression of NFATC2, MAF, ZBTB7A, MBD2, GATA3, MED14, IRF2, MED30, ZBTB1L, RELA, JAK3, MED11, BCL11B, MTF1, ATXN7L3, YY1, ETS1, IL2, DNMT1, GTF2B or SMARCB1; and/or (b) a heterologous polypeptide that encodes MED12, FOXP1, PTEN, IKZF1, TAF5L, PRDM1, TFDP1, CXXC1, IKZF3 or TP53, wherein expression of IL-2 is decreased in the T cell relative to expression of IL-2 in a T cell not comprising heterologous polypeptide that encodes MED12, FOXP1, PTEN, IKZF1, TAF5L, PRDM1, TFDP1, CXXC1, IKZF3 or TP53.

In some embodiments, inhibition of one or more nuclear factors set forth in Table 7 and/or overexpression of one or more nuclear factors set forth in Table 8 may increase IL2RA expression in the T cell. In some embodiments, inhibition of one or more nuclear factors set forth in Table 8, and/or overexpression of one or more nuclear factor set forth in Table 7 may decrease IL2RA expression in the T cell.

In some embodiments, the T cell comprises: (a) a genetic modification or heterologous polynucleotide that inhibits expression of MED12, CBFB, HIVEP2, KLF2, MYB, FOXK1, ZNF217, IRF2, TNFAIP3, MYC, PRDM1, TFDP1, IRF1, FOXO1, ATXN7L3 or TP53, wherein expression of IL2RA is increased in the T cell relative to expression of IL2RA in a T cell not comprising the genetic modification or heterologous polynucleotide that inhibits expression of MED12, CBFB, HIVEP2, KLF2, MYB, FOXK1, ZNF217, IRF2, TFNAIP3, MYC, PRDM1, TFDP1, IRF1, FOXO1, ATXN7L3 or TP53; and/or (b) a heterologous polypeptide that encodes IKZF3, YY1, MBD2, IRF4, IKZF1, RXRB, RELA, ETS1, KMT2A, PTEN, JAK3, STAT5A, GATA3, FOXP1, STAT5B, or IL2RA, wherein expression of IL2RA is increased in the T cell relative to expression of IL2RA in a T cell not comprising the heterologous polypeptide that encodes IKZF3, YY1, MBD2, IRF4, IKZF1, RXRB, RELA, ETS1, KMT2A, PTEN, JAK3, STAT5A, GATA3, FOXP1, STAT5B, or IL2RA.

In some embodiments, the T cell comprises: (a) a genetic modification or heterologous polynucleotide that inhibits expression of IKZF3, YY1, MBD2, IRF4, IKZF1, RXRB, RELA, ETS1, KMT2A, PTEN, JAK3, STAT5A, GATA3, FOXP1, STAT5B, or IL2RA, wherein expression of IL2RA is decreased in the T cell relative to expression of IL2RA in a T cell not comprising the genetic modification or heterologous polynucleotide that inhibits expression of IKZF3, YY1, MBD2, IRF4, IKZF1, RXRB, RELA, ETS1, KMT2A, PTEN, JAK3, STAT5A, GATA3, FOXP1, STAT5B, or IL2RA; and/or (b) a heterologous polypeptide that encodes MED12, CBFB, HIVEP2, KLF2, MYB, FOXK1, ZNF217, IRF2, TFNAIP3, MYC, PRDM1, TFDP1, IRF1, FOXO1, ATXN7L3 or TP53, wherein expression of IL2RA is decreased in the T cell relative to expression of IL2RA in a T cell not comprising heterologous polypeptide that encodes MED12, CBFB, HIVEP2, KLF2, MYB, FOXK1, ZNF217, IRF2, TFNAIP3, MYC, PRDM1, TFDP1, IRF1, FOXO1, ATXN7L3 or TP53.

In some embodiments, inhibition of one or more nuclear factors set forth in Table 9 and/or overexpression of one or more nuclear factors set forth in Table 10 may increase IL2RA expression in an effector T cell. In some embodiments, IL2RA is specifically increased in an effector T cell as compared to a regulatory T cell. In some embodiments, inhibition of one or more nuclear factors set forth in Table 10, and/or overexpression of one or more nuclear factor set forth in Table 9 may decrease IL2RA expression in an effector T cell. In some embodiments, IL2RA is specifically decreased in an effector T cell as compared to a regulatory T cell.

In some embodiments, inhibition of one or more nuclear factors set forth in Table 11 and/or overexpression of one or more nuclear factors set forth in Table 12 may increase IL2RA expression in a regulatory T cell. In some embodiments, IL2RA is specifically increased in a regulatory T cell as compared to an effector T cell. In some embodiments, inhibition of one or more nuclear factors set forth in Table 12, and/or overexpression of one or more nuclear factor set forth in Table 11 may decrease IL2RA expression in a regulatory T cell. In some embodiments, IL2RA is specifically decreased in a regulatory T cell as compared to an effector T cell.

In some embodiments, inhibition of one or more nuclear factors set forth in Table 13 and/or overexpression of one or more nuclear factors set forth in Table 14 may increase IL2RA expression in an effector T cell and a regulatory T cell. In some embodiments, inhibition of one or more nuclear factors set forth in Table 14, and/or overexpression of one or more nuclear factor set forth in Table 13 may decrease IL2RA expression in an effector T cell and a regulatory T cell.

Table 1 provides nuclear factors that, when inhibited, increase CTLA4 expression (CTLA4 high). Overexpression of a nuclear factor set forth in Table 1 may decrease CTLA4 expression. Table 2 provides nuclear factors that, when inhibited, decrease CTLA4 expression (CTLA4 low). Overexpression of a nuclear factor set forth in Table 2 may increase CTLA4 expression.

Table 3 provides nuclear factors that, when inhibited, increase FOXP3 expression (FOXP3 high). Overexpression of a nuclear factor set forth in Table 3 may decrease FOXP3 expression. Table 4 provides nuclear factors that, when inhibited, decrease FOXP3 expression (FOXP3 low). Overexpression of a nuclear factor set forth in Table 4 may increase FOXP3 expression.

Table 5 provides nuclear factors that, when inhibited, increase IL-2 expression (IL-2 high). Overexpression of a nuclear factor set forth in Table 5 may decrease IL-2 expression (IL-2 low). Table 6 provides nuclear factors that, when inhibited, decrease IL-2 expression. Overexpression of a nuclear factor set forth in Table 6 may increase IL-2 expression.

Table 7 provides nuclear factors that, when inhibited, increase IL2RA expression (IL2RA high). Overexpression of a nuclear factor set forth in Table 7 may decrease IL-2RA expression. Table 8 provides nuclear factors that, when inhibited, decrease IL2RA expression (IL2RA low). Overexpression of a nuclear factor set forth in Table 8 may increase IL2RA expression.

Table 9 provides nuclear factors that, when inhibited, increase IL2RA expression in effector T cells as compared to regulatory T cells (IL2RA high). Overexpression of a nuclear factor set forth in Table 9 may decrease IL-2RA expression. Table 10 provides nuclear factors that, when inhibited, decrease IL2RA expression in effector T cells as compared to regulatory T cells (IL2RA low). Overexpression of a nuclear factor set forth in Table 10 may increase IL-2RA expression.

Table 11 provides nuclear factors that, when inhibited, increase IL2RA expression in regulatory T cells as compared to effector T cells (IL2RA high). Overexpression of a nuclear factor set forth in Table 11 may decrease IL-2RA expression. Table 12 provides nuclear factors that, when inhibited, decrease IL2RA expression in regulatory T cells as compared to effector T cells (IL2RA low). Overexpression of a nuclear factor set forth in Table 12 may increase IL-2RA expression.

Table 13 provides nuclear factors that, when inhibited, increase IL2RA expression in regulatory T cells and effector T cells (IL2RA high). Overexpression of a nuclear factor set forth in Table 13 may decrease IL-2RA expression. Table 14 provides nuclear factors that, when inhibited, decrease IL2RA expression in regulatory T cells and effector T cells (IL2RA low). Overexpression of a nuclear factor set forth in Table 14 may increase IL-2RA expression.

In some embodiments, expression of an amino acid sequence having at least about 80%, 85%, 90%, 95% or 99% identity to an amino acid sequence set forth in Table 1, Table 2, Table 3, Table 4, Table 5, Table 6, Table 7, Table 8, Table 9, Table 10, Table 11, Table 12, Table 13 or Table 14 is inhibited. In some embodiments, an amino acid sequence having at least about 80%, 85%, 90%, 95% or 99% identity to an amino acid sequence set forth in Table 1, Table 2, Table 3, Table 4, Table 5, Table 6, Table 7, Table 8, Table 9, Table 10, Table 11, Table 12, Table 13 or Table 14 is overexpressed. It is understood that, when referring to one or more nuclear factors set forth in Table 1, Table 2, Table 3, Table 4, Table 5, Table 6, Table 7, Table 8, Table 9, Table 10, Table 11, Table 12, Table 13 or Table 14, this can be the protein, i.e., the nuclear factor, or the polynucleotide encoding the nuclear factor.

In some embodiments of the methods described herein, inhibiting the expression of a nuclear factor set forth in Table 1, Table 2, Table 3, Table 4, Table 5, Table 6, Table 7, Table 8, Table 9, Table 10, Table 11, Table 12, Table 13 or Table 14 may comprise reducing expression of the nuclear factor or reducing expression of a polynucleotide, for example, an mRNA, encoding the nuclear factor in the T cell. In some embodiments expression of one or more nuclear factors set forth in Table 1, Table 2, Table 3, Table 4, Table 5, Table 6, Table 7, Table 8, Table 9, Table 10, Table 11, Table 12, Table 13 or Table 14 is inhibited in the T cell. As described in detail further herein, one or more available methods may be used to inhibit the expression of one or more nuclear factors set forth in Table 1, Table 2, Table 3, Table 4, Table 5, Table 6, Table 7, Table 8, Table 9, Table 10, Table 11, Table 12, Table 13 or Table 14.

In some embodiments of the methods described herein, overexpressing a nuclear factor set forth in Table 1, Table 2, Table 3, Table 4, Table 5, Table 6, Table 7, Table 8, Table 9, Table 10, Table 11, Table 12, Table 13 or Table 14 may comprise introducing a polynucleotide encoding the nuclear factor into the T cell. In other embodiments of the methods described herein, overexpressing a nuclear factor set forth in Table 1, Table 2, Table 3, Table 4, Table 5, Table 6, Table 7, Table 8, Table 9, Table 10, Table 11, Table 12, Table 13 or Table 14 may comprise introducing an agent that induces expression of the endogenous gene encoding the nuclear factor in the T cell. For example, RNA activation, where short double-stranded RNAs induce endogenous gene expression by targeting promoter sequences, can be used to induce endogenous gene expression (See, for example, Wang et al. “Inducing gene expression by targeting promoter sequences using small activating RNAs,” J. Biol. Methods 2(1): e14 (2015). In another example, artificial transcription factors containing zinc-finger binding domains can be used to activate or repress expression of endogenous genes. See, for example, Dent et al., “Regulation of endogenous gene expressing using small molecule-controlled engineered zinc-finger protein transcription factors,” Gene Ther. 14(18): 1362-9 (2007).

In some embodiments, inhibiting expression may comprise contacting a polynucleotide encoding the nuclear factor, with a target nuclease, a guide RNA (gRNA), an siRNA, an antisense RNA, microRNA (miRNA), or short hairpin RNA (shRNA). In particular embodiments, if a gRNA and a target nuclease (e.g., Cas9) are used to inhibit the expression of a polynucleotide encoding a human nuclear factor set forth in Table 1, Table 2, Table 3, Table 4, Table 5, Table 6, Table 7, Table 8, Table 9, Table 10, Table 11, Table 12, Table 13 or Table 14, the gRNA may comprise a sequence set forth in Tables 1-8, a sequence complementary to a sequence set forth in Tables 1-14, or a portion thereof. Table 1, Table 2, Table 3, Table 4, Table 5, Table 6, Table 7, Table 8, Table 9, Table 10, Table 11, Table 12, Table 13 or Table 14 provide the Gene ID number, Genbank Accession No. for mRNA, genomic sequence, position in the genome after nuclease cutting, sgRNA target sequence, target context sequence, PAM sequence, and the exon targeted by the sgRNA for each nuclear factor set forth in Table 1, Table 2, Table 3, Table 4, Table 5, Table 6, Table 7, Table 8, Table 9, Table 10, Table 11, Table 12, Table 13 or Table 14

As described herein. T cells may be modified by inhibiting the expression of the one or more nuclear factors set forth in Table 1, Table 2, Table 3, Table 4, Table 5, Table 6, Table 7, Table 8, Table 9, Table 10, Table 11, Table 12, Table 13 or Table 14. For example, one or more nuclear factors selected from the group consisting of CBFB, MYB, ZNF217, FOXK1, FLI1, FOS, SATB1, IL2, ATXN7L3, MTF1, RELA, IRF1, BCL11B, STAT3, MED30, MED14, MED11, IKZF3, KMT2A, IKZF1, MED12, TAF5L, PTEN, IRF4, FOXO1, FOXP1, CTLA4, ETS1, MYBL2, TP53, MBD2, ZBTB7A, DNMT1, HIVEP2, KLF2, TFDP1, SMARCB1, MAF, FOXP3, GATA3, STAT5B, STAT5A, PRDM1, TNFAIP3, RXRB, TFDP1, CXXC1, NFATC2, MAF, IRF2, ZBTB11, JAK3, YY1, IL2RA and GTF2B, can be inhibited in a T cell.

T cells may also be modified by overexpressing one or more nuclear factors set forth in Table 1, Table 2, Table 3, Table 4, Table 5, Table 6, Table 7, Table 8, Table 9, Table 10, Table 11, Table 12, Table 13 or Table 14. For example, one or more nuclear factors selected from the group consisting of CBFB, MYB, ZNF217, FOXK1, FLI1, FOS, SATB1, IL2, ATXN7L3, MTF1, RELA, IRF1, BCL11B, STAT3, MED30, MED14, MED11, IKZF3. KMT2A, IKZF1, MED12, TAF5L, PTEN, IRF4, FOXO1, FOXP1, CTLA4, ETS1, MYBL2, TP53, MBD2, ZBTB7A, DNMT1, HIVEP2, KLF2, TFDP1, SMARCB1, MAF, FOXP3, GATA3, STAT5B, STAT5A, PRDM1, TNFAIP3, RXRB, TFDP1, CXXC1, NFATC2, MAF, IRF2, ZBTB11, JAK3, YY1, IL2RA and GTF2B, can be overexpressed in a T cell.

Subsequently, once modified T cells, for example, human T cells, are created, the modified T cells may be administered to a human. Depending on the modification, the modified T cells may be used to treat different indications. For example, T cells may be isolated from a whole blood sample of a human and expanded ex vivo. The expanded T cells may then be treated to inhibit the expression of a nuclear factor set forth in Table 1, Table 2, Table 3, Table 4, Table 5, Table 6, Table 7, Table 8, Table 9, Table 10, Table 11, Table 12, Table 13 or Table 14 thus, creating modified T cells. For example, one or more nuclear factors set forth in Table 1, Table 2, Table 3, Table 4, Table 5, Table 6, Table 7, Table 8, Table 9, Table 10, Table 11, Table 12, Table 13 or Table 14. For example, one or more nuclear factors selected from the group consisting of CBFB, MYB, ZNF217, FOXK1, FLI1, FOS, SATB1, IL2, ATXN7L3, MTF1, RELA, IRF1, BCL11B, STAT3, MED30, MED14, MED11, IKZF3, KMT2A, IKZF1, MED12, TAF5L, PTEN, IRF4, FOXO1, FOXP1, CTLA4, ETS1, MYBL2, TP53, MBD2, ZBTB7A, DNMT1, HIVEP2, KLF2, TFDP1, SMARCB1, MAF, FOXP3, GATA3, STAT5B, STAT5A, PRDM1, TNFAIP3, RXRB, TFDP1, CXXC1, NFATC2, MAF, IRF2, ZBTB11, JAK3, YY1, IL2RA and GTF2B, can be inhibited in the T cell.

The modified T cells may be reintroduced to the human to treat certain indications. In some embodiments, T cells having less immunosuppressive effects or enhanced cytotoxic or cell-killing effects may be used to treat cancer. In some embodiments, T cells having improved immunosuppressive effects may be used to treat autoimmune diseases.

In other cases, T cells in a subject can be modified in vivo, for example, by using a targeted vector, such as, a lentiviral vector, a retroviral vector an adenoviral or adeno-associated viral vector. In vivo delivery of targeted nucleases that modify the genome of a T cell can also be used. See for example, U.S. Pat. No. 9,737,604 and Zhang et al. “Lipid nanoparticle-mediated efficient delivery of CRISPR-Cas9 for tumor therapy,” NPG Asia Materials Volume 9, page e441 (2017).

Also provided is a T cell wherein expression of one or more nuclear factors set forth in Table 1, Table 2, Table 3, Table 4, Table 5, Table 6, Table 7, Table 8, Table 9, Table 10, Table 11, Table 12, Table 13 or Table 14 is inhibited. In some embodiments, expression of one or more nuclear factors selected from the group consisting of CBFB, MYB, ZNF217, FOXK1, FLI1, FOS, SATB1, IL2, ATXN7L3, MTF1, RELA, IRF1, BCL11B, STAT3, MED30, MED14, MED11, IKZF3, KMT2A, IKZF1, MED12, TAF5L, PTEN, IRF4, FOXO1, FOXP1, CTLA4, ETS1, MYBL2, TP53, MBD2, ZBTB7A, DNMT1, HIVEP2, KLF2, TFDP1, SMARCB1, MAF, FOXP3, GATA3, STAT5B, STAT5A, PRDM1, TNFAIP3, RXRB, TFDP1, CXXC1, NFATC2, MAF, IRF2, ZBTB11, JAK3, YY1, IL2RA and GTF2B, is inhibited in a T cell.

Further provided is a T cell wherein one or more nuclear factors set forth in Table 1. Table 2, Table 3, Table 4, Table 5, Table 6, Table 7, Table 8, Table 9, Table 10, Table 11, Table 12, Table 13 or Table 14 is overexpressed. In some embodiments, one or more nuclear factors selected from the group consisting of CBFB, MYB, ZNF217, FOXK1, FLI1, FOS, SATB1, IL2, ATXN7L3, MTF1, RELA, IRF1, BCL11B, STAT3, MED30, MED14, MED11, IKZF3, KMT2A, IKZF1, MED12, TAF5L, PTEN, IRF4, FOXO1, FOXP1, CTLA4, ETS1, MYBL2, TP53, MBD2, ZBTB7A, DNMT1, HIVEP2, KLF2, TFDP1, SMARCB1, MAF, FOXP3, GATA3, STAT5B, STAT5A, PRDM1, TNFAIP3, RXRB, TFDP1, CXXC1, NFATC2, MAF, IRF2, ZBTB11, JAK3, YY1, IL2RA and GTF2B, is overexpressed in a T cell

The disclosure also features a T cell comprising a genetic modification or heterologous polynucleotide that inhibits expression of one or more nuclear factors set forth in Table 1, Table 2, Table 3, Table 4, Table 5, Table 6, Table 7, Table 8, Table 9, Table 10, Table 11, Table 12, Table 13 or Table 14 and/or a heterologous polynucleotide that encodes a nuclear factor set forth in Table 1, Table 2, Table 3, Table 4, Table 5, Table 6, Table 7, Table 8, Table 9, Table 10, Table 11, Table 12, Table 13 or Table 14.

In some embodiments, the T cell comprises (a) a genetic modification or heterologous polynucleotide that inhibits expression of CBFB, MYB, ZNF217, FOXK1. FLI1, FOS, SATB1, IL2, ATXN7L3, MTF1, RELA, IRF1, BCL11B, STAT3, MED30, MED14, MED11, IKZF3, KMT2A, IKZF1, MED12, TAF5L, PTEN, IRF4, FOXO1, FOXP1, CTLA4, ETS1, MYBL2, TP53, MBD2, ZBTB7A, DNMT1, HIVEP2, KLF2, TFDP1, SMARCB1, MAF, FOXP3, GATA3, STAT5B. STAT5A, PRDM1, TNFAIP3, RXRB, TFDP1, CXXC1, NFATC2, MAF, IRF2, ZBTB11, JAK3, YY1, IL2RA or GTF2B; and/or (b) a heterologous polynucleotide that encodes CBFB, MYB, ZNF217, FOXK1, FLI1, FOS, SATB1, IL2. ATXN7L3, MTF1, RELA, IRF1, BCL11B, STAT3, MED30, MED14. MED11, IKZF3, KMT2A, IKZF1, MED12, TAF5L, PTEN, IRF4, FOXO1, FOXP1, CTLA4, ETS1, MYBL2, TP53, MBD2, ZBTB7A, DNMT1, TFDP1, SMARCB1, MAF, FOXP3, GATA3, STAT5B, STAT5A, PRDM1, TNFAIP3, RXRB, TFDP1, CXXC1, NFATC2, MAF, IRF2, ZBTB11, JAK3, YY1, IL2RA or GTF2B.

It is understood that one or more nuclear factors selected from the group consisting of CBFB, MYB, ZNF217, FOXK1, FLI1, FOS, SATB1, IL2, ATXN7L3, MTF1, RELA, IRF1, BCL11B, STAT3, MED30, MED14, MED11, IKZF3, KMT2A, IKZF1, MED12, TAF5L. PTEN, IRF4, FOXO1, FOXP1, CTLA4, ETS1, MYBL2. TP53, MBD2, ZBTB7A, DNMT1, HIVEP2, KLF2, TFDP1, SMARCB1, MAF, FOXP3, GATA3, STAT5B, STAT5A, PRDM1, TNFAIP3, RXRB, TFDP1, CXXC1, NFATC2, MAF, IRF2, ZBTB11, JAK3, YY1, IL2RA and GTF2B, can be inhibited and/or overexpressed in the T cells provided herein.

In some embodiments, the T cell comprises a genetic modification or heterologous polynucleotide that inhibits expression of a nuclear factor set forth in Table 1 and/or a heterologous polynucleotide that encodes a nuclear factor set forth in Table 2, and wherein expression of CTLA4 is increased in the T cell relative to expression of CTLA4 in a T cell not comprising the genetic modification or heterologous polynucleotide.

In some embodiments, the T cell comprises: (a) a genetic modification or a heterologous polynucleotide that inhibits expression of CBFB, MYB, ZNF217, FOXK1, FLI1, FOS, SATB1, IL2 or ATXN7L3, wherein expression of CTLA4 is increased in the T cell relative to expression of CTLA4 in a T cell not comprising the genetic modification or the heterologous polynucleotide that inhibits expression of CBFB, MYB, ZNF217, FOXK1, FLI1, FOS, SATB1, IL2 or ATXN7L3; and/or (b) a heterologous polynucleotide that encodes MTF1, RELA, IRF1, BCL11B, STAT3, MED30, MED14, MED11, IKZF3, KMT2A, IKZF1, MED12, TAF5L, PTEN, IRF4, FOXO1, FOXP1 or CTLA4, wherein expression of CTLA4 is increased in the T cell relative to expression of CTLA4 in a T cell not comprising the heterologous polynucleotide that encodes MTF1, RELA, IRF1, BCL11B, STAT3, MED30, MED14, MED11, IKZF3, KMT2A, IKZF1, MED12, TAF5L, PTEN, IRF4, FOXO1, FOXP1 or CTLA4.

In some embodiments, the T cell comprises a genetic modification or heterologous polynucleotide that inhibits expression of a nuclear factor set forth in Table 2, and/or a heterologous polynucleotide that encodes a nuclear factor set forth in Table 1, and wherein expression of CTLA4 is decreased in the T cell relative to expression of CTLA4 in a T cell not comprising the genetic modification or heterologous polynucleotide.

In some embodiments, the T cell comprises: (a) a genetic modification or heterologous polynucleotide that inhibits expression of MTF1, RELA, IRF1, BCL11B, STAT3, MED30, MED14, MED11, IKZF3, KMT2A, IKZF1, MED12, TAF5L, PTEN, IRF4, FOXO1, FOXP1 or CTLA4, wherein expression of CTLA4 is decreased in the T cell relative to expression of CTLA4 in a T cell not comprising the genetic modification or the heterologous polynucleotide that inhibits expression of MTF1, RELA, IRF1, BCL11B, STAT3. MED30, MED14, MED11, IKZF3, KMT2A, IKZF1, MED12, TAF5L, PTEN, IRF4, FOXO1, FOXP1 or CTLA4; and/or (b) a heterologous polynucleotide that encodes CBTB, MYB, ZNF217, FOXK1, FLI1, FOX, SATB1, IL2 or ATXN7L3, wherein expression of CTLA4 is decreased in the T cell relative to expression of CTLA4 in a T cell not comprising the heterologous polynucleotide that encodes CBTB, MYB, ZNF217, FOXK1, FLI1, FOX, SATB1, IL2 or ATXN7L3.

In some embodiments, the T cell comprises a genetic modification or heterologous polynucleotide that inhibits expression of a nuclear factor set forth in Table 3 and/or a heterologous polypeptide that encodes a nuclear factor set forth in Table 4, and wherein expression of FOXP3 is increased in the T cell relative to expression of FOXP3 in a T cell not comprising the genetic modification or heterologous polynucleotide.

In some embodiments, the T cell comprises: (a) a genetic modification or heterologous polynucleotide that inhibits expression of ETS1, MYBL2, MYB, TP53, FLI1, SATB1, MBD2. ZBTB7A, DNMT1, TFDP1, SMARCB1 or MAF, wherein expression of FOXP3 is increased in the T cell relative to expression of FOXP3 in a T cell not comprising the genetic modification or heterologous polynucleotide that inhibits expression of ETS1, MYBL2, MYB, TP53, FLI1, SATB1, MBD2, ZBTB7A, DNMT1, TFDP1, SMARCB1 or MAF; and/or (b) a heterologous polynucleotide that encodes a TAF5L, FOXP3, GATA3. STAT5B, FOXP1, STAT5A, PTEN or FOXO1, wherein expression of FOXP3 is increased in the T cell relative to expression of FOXP3 in a T cell not comprising a heterologous polynucleotide that encodes a TAF5L, FOXP3, GATA3, STAT5B, FOXP1, STAT5A, PTEN or FOXO1.

In some embodiments, the T cell is a Treg cell and increasing FOXP3 expression in the cell stabilizes the Treg cells. In some examples, stabilized Treg cells are used to treat autoimmune disorders, assist in organ transplantation, to treat graft versus host disease, or inflammation.

In some embodiments, the T cell comprises a genetic modification or heterologous polynucleotide that inhibits expression of a nuclear factor set forth in Table 4, and/or a heterologous polynucleotide that encodes a nuclear factor set forth in Table 3, and wherein expression of FOXP3 is decreased in the T cell relative to expression of FOXP3 in a T cell not comprising the genetic modification or heterologous polynucleotide.

In some embodiments, the T cell comprises: (a) a genetic modification or heterologous polynucleotide that inhibits expression of TAF5L, FOXP3, GATA3, STAT5B, FOXP1, STAT5A, PTEN or FOXO1, wherein expression of FOXP3 is decreased in the T cell relative to expression of FOXP3 in a T cell not comprising the genetic modification or heterologous polynucleotide that inhibits expression of TAF5L, FOXP3, GATA3, STAT5B, FOXP1, STAT5A, PTEN or FOXO1; and/or (b) a heterologous polynucleotide that encodes ETS1, MYBL2, MYB, TP53, FLI1, SATB1, MBD2, ZBTB7A, DNMT1, TFDP1, SMARCB1 or MAF, wherein expression of FOXP3 is decreased in the T cell relative to expression of FOXP3 in a T cell not comprising a heterologous polynucleotide that encodes ETS1, MYBL2, MYB, TP53, FLI1, SATB1, MBD2, ZBTB7A, DNMT1, TFDP1, SMARCB1 or MAF.

In some embodiments, the T cell is a Treg cell and decreasing FOXP3 expression in the cell destabilizes the Treg cells. In some examples, destabilized Treg cells are used to treat cancer.

In some embodiments, the T cell comprises a genetic modification or heterologous polynucleotide that inhibits expression of a nuclear factor set forth in Table 5, and/or a heterologous polynucleotide that encodes a nuclear factor set forth in Table 6, and wherein expression of IL-2 is increased in the T cell relative to expression of IL-2 in a T cell not comprising the genetic modification or heterologous polynucleotide.

In some embodiments, the T cell comprises: (a) a genetic modification or heterologous polynucleotide that inhibits expression of MED12, FOXP1, PTEN, IKZF1, TAF5L, PRDM1, TFDP1, CXXC1. IKZF3 or TP53, wherein expression of IL-2 is increased in the T cell relative to expression of IL-2 in a T cell not comprising the genetic modification or heterologous polynucleotide that inhibits expression of MED12, FOXP1, PTEN, IKZF1, TAF5L, PRDM1, TFDP1, CXXC1, IKZF3 or TP53; and/or (b) a heterologous polynucleotide that encodes NFATC2, MAF, ZBTB7A, MBD2, GATA3, MED14, IRF2. MED30. ZBTB11. RELA, JAK3, MED11, BCL11B, MTF1, ATXN7L3, YY1, ETS1, IL2, DNMT1, GTF2B or SMARCB1, wherein expression of IL-2 is increased in the T cell relative to expression of IL-2 in a T cell not comprising heterologous polynucleotide that encodes NFATC2, MAF, ZBTB7A, MBD2, GATA3, MED14. IRF2, MED30, ZBTB11, RELA, JAK3, MED11, BCL11B, MTF1, ATXN7L3, YY1, ETS1, IL2, DNMT1, GTF2B or SMARCB1.

In some examples, a Treg cell having increased IL-2 expression can be used to treat autoimmune disease or cancer. In some embodiments, the T cell is a conventional T cell, for example, CD4+ or CD8+ T cell, with increased IL-2 expression. In some examples, a conventional T cell having increased IL-2 expression can be used to treat cancer.

In some embodiments, the T cell comprises a genetic modification or heterologous polynucleotide that inhibits expression of a nuclear factor set forth in Table 6, and/or a heterologous polypeptide that encodes a nuclear factor set forth in Table 5, and wherein expression of IL-2 is decreased in the T cell relative to expression of IL-2 in a T cell not comprising the genetic modification or heterologous polynucleotide.

In some embodiments, the T cell comprises: (a) genetic modification or heterologous polynucleotide that inhibits expression of NFATC2, MAF, ZBTB7A, MBD2. GATA3, MED14, IRF2, MED30, ZBTB11, RELA, JAK3, MED11, BCL11B, MTF1, ATXN7L3, YY1, ETS1, IL2, DNMT1, GTF2B or SMARCB1, wherein expression of IL-2 is decreased in the T cell relative to expression of IL-2 in a T cell not comprising the genetic modification or heterologous polynucleotide that inhibits expression of NFATC2, MAF, ZBTB7A, MBD2, GATA3, MED14. IRF2, MED30, ZBTB11, RELA, JAK3, MED11, BCL11B, MTF1, ATXN7L3, YY1, ETS1, IL2, DNMT1, GTF2B or SMARCB1; and/or (b) a heterologous polynucleotide that encodes MED12, FOXP1, PTEN, IKZF1, TAF5L, PRDM1, TFDP1, CXXC1, IKZF3 or TP53, wherein expression of IL-2 is decreased in the T cell relative to expression of IL-2 in a T cell not comprising heterologous polynucleotide that encodes MED12, FOXP1, PTEN, IKZF1, TAF5L, PRDM1, TFDP1, CXXC1, IKZF3 or TP53.

In some embodiments, the T cell is a conventional T cell, for example, CD4+ or CD8+ T cell, with decreased IL-2 expression. In some examples, a conventional T cell having decreased IL-2 expression can be used to treat autoimmune disease.

In some embodiments, the T cell comprises a genetic modification or heterologous polynucleotide that inhibits expression of a nuclear factor set forth in Table 7, and/or a heterologous polypeptide that encodes a nuclear factor set forth in Table 8, and wherein expression of IL2RA is increased in the T cell relative to expression of IL2RA in a T cell not comprising the genetic modification or heterologous polynucleotide.

In some embodiments, the T cell comprises: (a) a genetic modification or heterologous polynucleotide that inhibits expression of MED12, CBFB, HIVEP2, KLF2, MYB, FOXK1, ZNF217, IRF2, TNFAIP3, MYC, PRDM1, TFDP1, IRF1, FOXO1, ATXN7L3 or TP53, wherein expression of IL2RA is increased in the T cell relative to expression of IL2RA in a T cell not comprising the genetic modification or heterologous polynucleotide that inhibits expression of MED12, CBFB, HIVEP2, KLF2, MYB, FOXK1, ZNF217, IRF2, TFNAIP3, MYC, PRDM1, TFDP1, IRF1, FOXO1, ATXN7L3 or TP53; and/or (b) a heterologous polynucleotide that encodes IKZF3, YY1, MBD2, IRF4, IKZF1, RXRB, RELA, ETS1. KMT2A, PTEN, JAK3, STAT5A, GATA3, FOXP1, STAT5B, or IL2RA, wherein expression of IL2RA is increased in the T cell relative to expression of IL2RA in a T cell not comprising the heterologous polynucleotide that encodes IKZF3, YY1, MBD2, IRF4, IKZF1, RXRB, RELA, ETS1, KMT2A. PTEN, JAK3, STAT5A, GATA3, FOXP1, STAT5B, or IL2RA.

In some examples, a Treg cell having increased IL-2RA expression can be used to treat autoimmune disease. In some embodiments, the T cell is a conventional T cell, for example, CD4+ or CD8+ T cell, with increased IL-2RA expression. In some examples, a conventional T cell having increased IL-2RA expression can be used to treat cancer.

In some embodiments, the T cell comprises a genetic modification or heterologous polynucleotide that inhibits expression of a nuclear factor set forth in Table 8, and/or a heterologous polypeptide that encodes a nuclear factor set forth in Table 7, and wherein expression of IL2RA is decreased in the T cell relative to expression of IL2RA in a T cell not comprising the genetic modification or heterologous polynucleotide.

In some embodiments, the T cell comprises: (a) a genetic modification or heterologous polynucleotide that inhibits expression of IKZF3, YY1, MBD2, IRF4, IKZF1, RXRB, RELA, ETS1, KMT2A, PTEN, JAK3, STAT5A, GATA3, FOXP1, STAT5B, or IL2RA, wherein expression of IL2RA is decreased in the T cell relative to expression of IL2RA in a T cell not comprising the genetic modification or heterologous polynucleotide that inhibits expression of IKZF3, YY1, MBD2, IRF4, IKZF1, RXRB, RELA, ETS1, KMT2A, PTEN, JAK3, STAT5A, GATA3, FOXP1, STAT5B, or IL2RA; and/or (b) a heterologous polynucleotide that encodes MED12, CBFB, HIVEP2, KLF2, MYB, FOXK1, ZNF217, IRF2, TFNAIP3, MYC, PRDM1, TFDP1, IRF1, FOXO1, ATXN7L3 or TP53, wherein expression of IL2RA is decreased in the T cell relative to expression of IL2RA in a T cell not comprising heterologous polynucleotide that encodes MED12, CBFB, HIVEP2, KLF2, MYB, FOXK1, ZNF217, IRF2, TFNAIP3, MYC, PRDM1, TFDP1, IRF1, FOXO1, ATXN7L3 or TP53.

In some examples, a Treg cell having decreased IL-2RA expression can be used to treat cancer. In some embodiments, the T cell is a conventional T cell, for example, CD4+ or CD8+ T cell, with decreased IL-2RA expression. In some examples, a conventional T cell having decreased IL-2RA expression can be used to treat autoimmune disease.

In some embodiments, the T cell is a Treg cell. In some embodiments, the T cell is a CD8+, a CD4+ or a CD8+CD4+ T cell. Also provided, are populations of cells comprising any of the genetically modified T cells described herein.

A genetic modification may be a nucleotide mutation or any sequence alteration in the polynucleotide encoding the nuclear factor that results in the inhibition of the expression of the nuclear factor. A heterologous polynucleotide may refer to a polynucleotide originally encoding the nuclear factor but is altered, i.e., comprising one or more nucleotide mutations or sequence alterations. In some embodiments, the heterologous polynucleotide is inserted into the genome of the T cell by introducing a vector, for example, a viral vector, comprising the polynucleotide. Examples of viral vectors include, but are not limited to adeno-associated viral (AAV) vectors, retroviral vectors or lentiviral vectors. In some embodiments, the lentiviral vector is an integrase-deficient lentiviral vector.

Also disclosed herein are T cells comprising at least one guide RNA (gRNA) comprising a sequence selected from Table 1, Table 2, Table 3, Table 4, Table 5, Table 6, Table 7, Table 8, Table 9, Table 10, Table 11, Table 12. Table 13 or Table 14. The expression of one or more nuclear factors set forth in Table 1, Table 2, Table 3, Table 4, Table 5, Table 6, Table 7, Table 8, Table 9, Table 10, Table 11, Table 12, Table 13 or Table 14, in the T cells comprising the gRNAs, may be reduced in the T cells relative to the expression of the one or more nuclear factors in T cells not comprising the gRNAs. In other examples, an endogenous nuclear factor set forth in Table 1, Table 2, Table 3, Table 4, Table 5, Table 6, Table 7, Table 8, Table 9, Table 10, Table 11, Table 12, Table 13 or Table 14 can be inhibited by targeting a deactivated targeted nuclease, for example dCAs9, fused to a transcriptional repressor, to the promoter region of the endogenous nuclear factor gene. In other examples, an endogenous nuclear factor set forth in Table 1, Table 2, Table 3, Table 4, Table 5, Table 6, Table 7, Table 8, Table 9, Table 10, Table 11, Table 12, Table 13 or Table 14 can be upregulated or overexpressed by targeting a deactivated targeted nuclease, for example dCAs9, fused to a transcriptional activator, to the promoter region of the endogenous nuclear factor gene. See, for example. Qi et al. “The New State of the Art: Cas9 for Gene Activation and Repression,” Mol. and Cell. BioL, 35(22): 3800-3809 (2015).

### II. Methods of Inhibiting Expression

### CRISPR/Cas Genome Editing

The CRISPR (Clustered Regularly Interspaced Short Palindromic Repeats)/Cas (CRISPR-associated protein) nuclease system is an engineered nuclease system based on a bacterial system that can be used for genome engineering. It is based on part of the adaptive immune response of many bacteria and archaea. When a virus or plasmid invades a bacterium, segments of the invader's DNA are converted into CRISPR RNAs (crRNA) by the “immune” response. The crRNA then associates, through a region of partial complementarity, with another type of RNA called tracrRNA to guide the Cas (e.g., Cas9) nuclease to a region homologous to the crRNA in the target DNA called a “protospacer.” The Cas (e.g., Cas9) nuclease cleaves the DNA to generate blunt ends at the double-strand break at sites specified by a 20-nucleotide guide sequence contained within the crRNA transcript. The Cas (e.g., Cas9) nuclease can require both the crRNA and the tracrRNA for site-specific DNA recognition and cleavage. This system has now been engineered such that the crRNA and tracrRNA can be combined into one molecule (the “guide RNA” or “gRNA”), and the crRNA equivalent portion of the single guide RNA can be engineered to guide the Cas (e.g., Cas9) nuclease to target any desired sequence (see, e.g., Jinek et al. (2012) Science 337:816-821; Jinek et aL (2013) eLife 2:e00471; Segal (2013) eLife 2:e00563). Thus, the CRISPR/Cas system can be engineered to create a double-strand break at a desired target in a genome of a cell, and harness the cell's endogenous mechanisms to repair the induced break by homology-directed repair (HDR) or nonhomologous end-joining (NHEJ).

In some embodiments of the methods described herein, CRISPR/Cas genome editing may be used to inhibit the expression of one or more nuclear factors set forth in Table 1, Table 2, Table 3, Table 4, Table 5, Table 6, Table 7, Table 8, Table 9. Table 10, Table 11. Table 12, Table 13 or Table 14. For example, CRISPR/Cas genome editing may be used to inhibit expression of one or more nuclear factors selected from the group consisting of CBFB, MYB, ZNF217, FOXK1, FLI1, FOS, SATB1, 1L2, ATXN7L3, MTF1, RELA, IRF1, BCL11B. STAT3, MED30, MED14, MED11, IKZF3, KMT2A, IKZF1, MED12, TAF5L, PTEN, IRF4, FOXO1, FOXP1, CTLA4, ETS1, MYBL2, TP53, MBD2, ZBTB7A, DNMT1, HIVEP2, KLF2, TFDP1, SMARCB1, MAF, FOXP3, GATA3, STAT5B, STAT5A, PRDM1, TNFAIP3, RXRB, TFDP1, CXXC1, NFATC2, MAF, IRF2, ZBTB11, JAK3, YY1, IL2RA and GTF2B

In some embodiments, the Cas nuclease has DNA cleavage activity. The Cas nuclease can direct cleavage of one or both strands at a location in a target DNA sequence, i.e., a location in a polynucleotide encoding a nuclear factor set forth in Table 1, Table 2, Table 3, Table 4, Table 5, Table 6, Table 7, Table 8, Table 9, Table 10, Table 11, Table 12, Table 13 or Table 14. In some embodiments, the Cas nuclease can be a nickase having one or more inactivated catalytic domains that cleaves a single strand of a target DNA sequence.

Non-limiting examples of Cas nucleases include Cas1, Cas1B, Cas2, Cas3, Cas4, Cas5, Cas6, Cas7, Cas8, Cas9 (also known as Csn1 and Csx12), Cas10, Csy1, Csy2, Csy3. Cse1, Cse2, Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX. Csx3, Csx1, Csx15, Csf1, Csf2, Csf3, Csf4, homologs thereof, variants thereof, mutants thereof, and derivatives thereof. There are three main types of Cas nucleases (type I, type II, and type III), and 10 subtypes including 5 type I. 3 type II, and 2 type III proteins (see, e.g., Hochstrasser and Doudna, Trends Biochem Sci, 2015:40(1):58-66). Type II Cas nucleases include Cas1, Cas2, Csn2, and Cas9. These Cas nucleases are known to those skilled in the art. For example, the amino acid sequence of the Streptococcus pyogenes wild-type Cas9 polypeptide is set forth, e.g., in NBCI Ref. Seq. No. NP_269215, and the amino acid sequence of Streptococcus thermophilus wild-type Cas9 polypeptide is set forth, e.g., in NBCI Ref. Seq. No. WP_011681470. Some CRISPR-related endonucleases that may be used in methods described herein are disclosed, e.g., in U.S. Application Publication Nos. 2014/0068797, 2014/0302563, and 2014/0356959.

Cas nucleases, e.g., Cas9 polypeptides, can be derived from a variety of bacterial species including, but not limited to, Veillonella atypical, Fusobacterium nucleatum, Filifactor alocis. Solobacterium moorei, Coprococcus catus, Treponema denticola. Peptoniphilus duerdenii, Catenibacterium mitsuokai, Streptococcus mutans. Listeria innocua, Staphylococcus pseudintermedius, Acidaminococcus intestine. Olsenella uli, Oenococcus kitaharae, Bifidobacterium bifidum, Lactobacillus rhamnosus. Lactobacillus gasseri, Finegoldia magna. Micoplasma mobile. Mvcoplasma gallisepticum. Mycoplasma ovipneumoniae, Mycoplasma canis, Mycoplasma synoviae, Eubacterium rectale. Streptococcus thermophilus, Eubacterium dolichum, Lactobacillus corynmformis subsp. Torquens. Ilyobacter polytropus, Ruminococcus albus, Akkermansia muciniphila, Acidothermus cellulolyticus. Biftdobacterium longum. Bifidobacterium dentium, Corynebacterium diphtheria. Elusimicrobium minutum, Nitratifractor salsuginis, Sphaerochaeta globus. Fibrobacter succinogenes subsp. Succinogenes, Bacteroides fragilis, Capnocvtophaga ochracea. Rhodopseudomonas palustris. Prevotella micans. Prevotella ruminicola, Flavobacterium columnare, Aminomonas paucivorans, Rhodospirillum rubrum. Candidatus Puniceispirillum marinum, Verminephrobacter eiseniae, Ralstonia syzvgii, Dinoroseobacter shibae. Azospirillum. Nitrobacter hamburgensis, Bradyrhizobium, Wolinella succinogenes, Campylobacter jejuni subsp. Jejuni, Helicobacter mustelae, Bacillus cereus, Acidovorax ebreus, Clostridium perfringens. Parvibaculum lavamentivorans, Roseburia intestinalis, Neisseria meningitidis. Pasteurella multocida subsp. Multocida, Sutterella wadsworthensis, proteobacterium. Legionella pneumophila, Parasutterella excrementihominis, Wolinella succinogenes, and Francisella novicida.

Wild-type Cas9 nuclease has two functional domains, e.g., RuvC and HNH, that cut different DNA strands. Cas9 can induce double-strand breaks in genomic DNA (target DNA) when both functional domains are active. The Cas9 enzyme can comprise one or more catalytic domains of a Cas9 protein derived from bacteria belonging to the group consisting of Corynebacter, Sutterella, Legionella, Treponema, Filifactor, Eubacterium, Streptococcus, Lactobacillus, Mycoplasma, Bacteroides, Flaviivola. Flavobacterium, Sphaerochaeta, Azospirillum, Gluconacetobacter. Neisseria. Roseburia, Parvibaculum, Staphylococcus. Nitratifractor, and Campylobacter. In some embodiments, the Cas9 may be a fusion protein, e.g., the two catalytic domains are derived from different bacteria species.

Useful variants of the Cas9 nuclease can include a single inactive catalytic domain, such as a RuvC⁻ or HNH⁻ enzyme or a nickase. A Cas9 nickase has only one active functional domain and can cut only one strand of the target DNA, thereby creating a single strand break or nick. In some embodiments, the Cas9 nuclease may be a mutant Cas9 nuclease having one or more amino acid mutations. For example, the mutant Cas9 having at least a D10A mutation is a Cas9 nickase. In other embodiments, the mutant Cas9 nuclease having at least a H840A mutation is a Cas9 nickase. Other examples of mutations present in a Cas9 nickase include, without limitation, N854A and N863A. A double-strand break may be introduced using a Cas9 nickase if at least two DNA-targeting RNAs that target opposite DNA strands are used. A double-nicked induced double-strand break can be repaired by NHEJ or HDR (Ran et al., 2013, Cell, 154:1380-1389). This gene editing strategy favors HDR and decreases the frequency of INDEL mutations at off-target DNA sites. Non-limiting examples of Cas9 nucleases or nickases are described in, for example, U.S. Pat. Nos. 8,895,308; 8,889,418; and 8,865,406 and U.S. Application Publication Nos. 2014/0356959, 2014/0273226 and 2014/0186919. The Cas9 nuclease or nickase can be codon-optimized for the target cell or target organism.

In some embodiments, the Cas nuclease can be a Cas9 polypeptide that contains two silencing mutations of the RuvC1 and HNH nuclease domains (D10A and H840A), which is referred to as dCas9 (Jinek et al., Science, 2012, 337:816-821; Qi et al., Cell, 152(5):1173-1183). In one embodiment, the dCas9 polypeptide from Streptococcus pyogenes comprises at least one mutation at position D10, G12, G17, E762, H840, N854, N863, H982, H983, A984, D986, A987 or any combination thereof. Descriptions of such dCas9 polypeptides and variants thereof are provided in, for example, International Patent Publication No. WO 2013/176772. The dCas9 enzyme may contain a mutation at D10, E762, H983, or D986, as well as a mutation at H840 or N863. In some instances, the dCas9 enzyme may contain a D10A or D10N mutation. Also, the dCas9 enzyme may contain a H840A, H840Y, or H840N. In some embodiments, the dCas9 enzyme may contain D10A and H840A; D10A and H840Y; D10A and H840N; D10N and H840A; D10N and H840Y; or D10N and H840N substitutions. The substitutions can be conservative or non-conservative substitutions to render the Cas9 polypeptide catalytically inactive and able to bind to target DNA.

In some embodiments, the Cas nuclease can be a high-fidelity or enhanced specificity Cas9 polypeptide variant with reduced off-target effects and robust on-target cleavage. Non-limiting examples of Cas9 polypeptide variants with improved on-target specificity include the SpCas9 (K855A), SpCas9 (K810A/K1003A/R1060A) (also referred to as eSpCas9 (1.0)), and SpCas9 (K848A/K1003A/R1060A) (also referred to as eSpCas9 (1.1)) variants described in Slaymaker et al., Science, 351(6268):84-8 (2016), and the SpCas9 variants described in Kleinstiver et al., Nature, 529(7587):490-5 (2016) containing one, two, three, or four of the following mutations: N497A, R661A, Q695A, and Q926A (e.g., SpCas9-HF1 contains all four mutations).

As described above, a gRNA may comprise a crRNA and a tracrRNAs. The gRNA can be configured to form a stable and active complex with a gRNA-mediated nuclease (e.g., Cas9 or dCas9). The gRNA contains a binding region that provides specific binding to the target genetic element. Exemplary gRNAs that may be used to target a region in a polynucleotide encoding a nuclear factor described herein are set forth in Table 1, Table 2, Table 3, Table 4, Table 5, Table 6, Table 7, Table 8, Table 9, Table 10, Table 11, Table 12, Table 13 or Table 14. A gRNA used to target a region in a polynucleotide encoding a nuclear factor set forth in Table 1, Table 2, Table 3, Table 4, Table 5, Table 6, Table 7, Table 8, Table 9, Table 10, Table 11, Table 12, Table 13 or Table 14 may comprise a sequence selected from Table 1, Table 2, Table 3, Table 4, Table 5, Table 6, Table 7, Table 8, Table 9, Table 10, Table 11, Table 12, Table 13 or Table 14, or a portion thereof.

In some embodiments, the targeted nuclease, for example, a Cpf1 nuclease or a Cas9 nuclease and the gRNA are introduced into the T cell as a ribonucleoprotein (RNP) complex. In some embodiments, the RNP complex may be introduced into about 1×10⁵ to about 2×10⁶ cells (e.g., 1×10⁵ cells to about 5×10⁵ cells, about 1×10⁵ cells to about 1×10⁶ cells, 1×10⁵ cells to about 1.5×10⁶ cells, 1×10⁵ cells to about 2×10⁶ cells, about 1×10⁶ cells to about 1.5×10⁶ cells, or about 1×10⁶ cells to about 2×10⁶ cells). In some embodiments, the T cells are cultured under conditions effective for expanding the population of modified T cells. Also disclosed herein is a population of T cells, in which the genome of at least 20%, 30%, 40%, 50%, 60%, 70%, 80%, 90%, 95%, 99% or greater of the cells comprises a genetic modification or heterologous polynucleotide that inhibits expression of one or more nuclear factors set forth in Table 1, Table 2, Table 3, Table 4, Table 5, Table 6, Table 7, Table 8, Table 9, Table 10, Table 11, Table 12, Table 13 or Table 14.

In some embodiments, the RNP complex is introduced into the T cells by electroporation. Methods, compositions, and devices for electroporating cells to introduce a RNP complex are available in the art, see, e.g., WO 2016/123578, WO/2006/001614, and Kim, J. A. et al. Biosens. Bioelectron. 23, 1353-1360 (2008). Additional or alternative methods, compositions, and devices for electroporating cells to introduce a RNP complex can include those described in U.S. Patent Appl. Pub. Nos. 2006/0094095; 2005/0064596; or 2006/0087522; Li, L. H. et al. Cancer Res. Treat. 1, 341-350 (2002); U.S. Pat. Nos.: 6,773,669; 7,186,559; 7,771,984; 7,991,559; 6,485,961; 7,029,916; and U.S. Patent Appl. Pub. Nos: 2014/0017213; and 2012/0088842; Geng, T. et al., J. Control Release 144, 91-100 (2010); and Wang, J., et al. Lab. Chip 10, 2057-2061 (2010).

In some embodiments, the sequence of the gRNA or a portion thereof is designed to complement (e.g., perfectly complement) or substantially complement (e.g., 50%, 55%, 60%, 65%, 70%, 75%, 80%, 85%, 90%, 91%, 92%, 93%, 94% 95%, 96%, 97%, 98%, or 99% complement) the target region in the polynucleotide encoding the protein. In some embodiments, the portion of the gRNA that complements and binds the targeting region in the polynucleotide is, or is about, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39 or 40 or more nucleotides in length. In some cases, the portion of the gRNA that complements and binds the targeting region in the polynucleotide is between about 19 and about 21 nucleotides in length. In some cases, the gRNA may incorporate wobble or degenerate bases to bind target regions. In some cases, the gRNA can be altered to increase stability. For example, non-natural nucleotides, can be incorporated to increase RNA resistance to degradation. In some cases, the gRNA can be altered or designed to avoid or reduce secondary structure formation. In some cases, the gRNA can be designed to optimize G-C content. In some cases, G-C content is between about 40% and about 60% (e.g., 40%, 45%, 50%, 55%, 60%). In some cases, the binding region can contain modified nucleotides such as, without limitation, methylated or phosphorylated nucleotides

In some embodiments, the gRNA can be optimized for expression by substituting, deleting, or adding one or more nucleotides. In some cases, a nucleotide sequence that provides inefficient transcription from an encoding template nucleic acid can be deleted or substituted. For example, in some cases, the gRNA is transcribed from a nucleic acid operably linked to an RNA polymerase III promoter. In such cases, gRNA sequences that result in inefficient transcription by RNA polymerase III, such as those described in Nielsen et al., Science. 2013 Jun. 28:340(6140):1577-80, can be deleted or substituted. For example, one or more consecutive uracils can be deleted or substituted from the gRNA sequence. In some cases, if the uracil is hydrogen bonded to a corresponding adenine, the gRNA sequence can be altered to exchange the adenine and uracil. This “A-U flip” can retain the overall structure and function of the gRNA molecule while improving expression by reducing the number of consecutive uracil nucleotides.

In some embodiments, the gRNA can be optimized for stability. Stability can be enhanced by optimizing the stability of the gRNA:nuclease interaction, optimizing assembly of the gRNA:nuclease complex, removing or altering RNA destabilizing sequence elements, or adding RNA stabilizing sequence elements. In some embodiments, the gRNA contains a 5′ stem-loop structure proximal to, or adjacent to, the region that interacts with the gRNA-mediated nuclease. Optimization of the 5′ stem-loop structure can provide enhanced stability or assembly of the gRNA:nuclease complex. In some cases, the 5′ stem-loop structure is optimized by increasing the length of the stem portion of the stem-loop structure.

gRNAs can be modified by methods known in the art. In some cases, the modifications can include, but are not limited to, the addition of one or more of the following sequence elements: a 5′ cap (e.g., a 7-methylguanylate cap); a 3′ polyadenylated tail; a riboswitch sequence; a stability control sequence; a hairpin; a subcellular localization sequence; a detection sequence or label; or a binding site for one or more proteins. Modifications can also include the introduction of non-natural nucleotides including, but not limited to, one or more of the following: fluorescent nucleotides and methylated nucleotides.

Also described herein are expression cassettes and vectors for producing gRNAs in a host cell. The expression cassettes can contain a promoter (e.g., a heterologous promoter) operably linked to a polynucleotide encoding a gRNA. The promoter can be inducible or constitutive. The promoter can be tissue specific. In some cases, the promoter is a U6. H1, or spleen focus-forming virus (SFFV) long terminal repeat promoter. In some cases, the promoter is a weak mammalian promoter as compared to the human elongation factor 1 promoter (EF1A). In some cases, the weak mammalian promoter is a ubiquitin C promoter or a phosphoglycerate kinase 1 promoter (PGK). In some cases, the weak mammalian promoter is a TetOn promoter in the absence of an inducer. In some cases, when a TetOn promoter is utilized, the host cell is also contacted with a tetracycline transactivator. In some embodiments, the strength of the selected gRNA promoter is selected to express an amount of gRNA that is proportional to the amount of Cas9 or dCas9. The expression cassette can be in a vector, such as a plasmid, a viral vector, a lentiviral vector, etc. In some cases, the expression cassette is in a host cell. The gRNA expression cassette can be episomal or integrated in the host cell.

### Zinc-Finger Nucleases (ZFNs)

“Zinc finger nucleases” or “ZFNs” are a fusion between the cleavage domain of FokI and a DNA recognition domain containing 3 or more zinc finger motifs. The heterodimerization at a particular position in the DNA of two individual ZFNs in precise orientation and spacing leads to a double-strand break in the DNA. In some embodiments of the methods described herein, ZFNs may be used to inhibit the expression of one or more nuclear factors set forth in Table 1 or Table 2, i.e., by cleaving the polynucleotide encoding the protein.

In some cases, ZFNs fuse a cleavage domain to the C-terminus of each zinc finger domain. In order to allow the two cleavage domains to dimerize and cleave DNA, the two individual ZFNs bind opposite strands of DNA with their C-termini at a certain distance apart. In some cases, linker sequences between the zinc finger domain and the cleavage domain requires the 5′ edge of each binding site to be separated by about 5-7 bp. Exemplary ZFNs that may be used in methods described herein include, but are not limited to, those described in Umov et al., Nature Reviews Genetics, 2010, 11:636-646; Gaj et al., Nat Methods, 2012, 9(8):805-7; U.S. Pat. Nos. 6,534,261; 6,607,882; 6,746,838; 6,794,136; 6,824,978; 6,866,997; 6,933,113; 6,979,539; 7,013,219; 7,030,215; 7,220,719; 7,241,573; 7,241,574; 7,585,849; 7,595,376; 6,903,185; 6,479,626; and U.S. Application Publication Nos. 2003/0232410 and 2009/0203140.

ZFNs can generate a double-strand break in a target DNA, resulting in DNA break repair which allows for the introduction of gene modification. DNA break repair can occur via non-homologous end joining (NHEJ) or homology-directed repair (HDR). In HDR, a donor DNA repair template that contains homology arms flanking sites of the target DNA can be provided.

In some embodiments, a ZFN is a zinc finger nickase which can be an engineered ZFN that induces site-specific single-strand DNA breaks or nicks, thus resulting in HDR. Descriptions of zinc finger nickases are found, e.g., in Ramirez et al., Nucl Acids Res. 2012, 40(12):5560-8; Kim et al., Genome Res, 2012, 22(7):1327-33.

### TALENs

TALENS may also be used to inhibit the expression of one or more nuclear factors set forth in Table 1, Table 2, Table 3, Table 4, Table 5, Table 6, Table 7 or Table 8. “TALENs” or “TAL-effector nucleases” are engineered transcription activator-like effector nucleases that contain a central domain of DNA-binding tandem repeats, a nuclear localization signal, and a C-terminal transcriptional activation domain. In some instances, a DNA-binding tandem repeat comprises 33-35 amino acids in length and contains two hypervariable amino acid residues at positions 12 and 13 that can recognize one or more specific DNA base pairs. TALENs can be produced by fusing a TAL effector DNA binding domain to a DNA cleavage domain. For instance, a TALE protein may be fused to a nuclease such as a wild-type or mutated FokI endonuclease or the catalytic domain of FokI. Several mutations to FokI have been made for its use in TALENs, which, for example, improve cleavage specificity or activity. Such TALENs can be engineered to bind any desired DNA sequence.

TALENs can be used to generate gene modifications by creating a double-strand break in a target DNA sequence, which in turn, undergoes NHEJ or HDR. In some cases, a single-stranded donor DNA repair template is provided to promote HDR.

Detailed descriptions of TALENs and their uses for gene editing are found, e.g., in U.S. Pat. Nos. 8,440,431; 8,440,432; 8,450.471; 8,586,363; and 8,697,853; Scharenberg et al., Curr Gene Ther, 2013, 13(4):291-303; Gaj et al., Nat Methods, 2012, 9(8):805-7; Beurdeley et al., Nat Commun, 2013, 4:1762; and Joung and Sander, Nat Rev Mol Cell Biol, 2013, 14(1):49.

### Meganucleases

Meganucleases” are rare-cutting endonucleases or homing endonucleases that can be highly specific, recognizing DNA target sites ranging from at least 12 base pairs in length, e.g., from 12 to 40 base pairs or 12 to 60 base pairs in length. Meganucleases can be modular DNA-binding nucleases such as any fusion protein comprising at least one catalytic domain of an endonuclease and at least one DNA binding domain or protein specifying a nucleic acid target sequence. The DNA-binding domain can contain at least one motif that recognizes single- or double-stranded DNA. The meganuclease can be monomeric or dimeric.

In some embodiments of the methods described herein, meganucleases may be used to inhibit the expression of one or more nuclear factors set forth in Table 1, Table 2, Table 3, Table 4, Table 5, Table 6, Table 7 or Table 8 i.e., by cleaving in a target region within the polynucleotide encoding the nuclear factor. In some instances, the meganuclease is naturally-occurring (found in nature) or wild-type, and in other instances, the meganuclease is non-natural, artificial, engineered, synthetic, or rationally designed. In certain embodiments, the meganucleases that may be used in methods described herein include, but are not limited to, an I-CreI meganuclease, I-CeuI meganuclease, I-MsoI meganuclease, I-SceI meganuclease, variants thereof, mutants thereof, and derivatives thereof.

Detailed descriptions of useful meganucleases and their application in gene editing are found, e.g., in Silva et al., Curr Gene Ther, 2011, 11(1):11-27; Zaslavoskiy et al., BAC Bioinformatics, 2014, 15:191; Takeuchi et al., Proc Natl Acad Sci USA, 2014, 111(11):4061-4066, and U.S. Pat. Nos. 7,842,489; 7,897,372; 8,021,867; 8,163,514; 8,133,697; 8,021,867; 8,119,361; 8,119,381; 8,124,36; and 8,129,134.

### RNA-Based Technologies

Various RNA-based technologies may also be used in methods described herein to inhibit the expression of one or more nuclear factors set forth in Table 1, Table 2, Table 3, Table 4, Table 5, Table 6, Table 7 or Table 8. Examples of RNA-based technologies include, but are not limited to, small interfering RNA (siRNA), antisense RNA, microRNA (miRNA), and short hairpin RNA (shRNA)

RNA-based technologies may use an siRNA, an antisense RNA, a miRNA, or a shRNA to target a sequence, or a portion thereof, that encodes a transcription factor. In some embodiments, one or more genes regulated by a transcription factor may also be targeted by an siRNA, an antisense RNA, a miRNA, or a shRNA. An siRNA, an antisense RNA, a miRNA, or a shRNA may target a sequence comprising at least 10, at least 20, at least 30, at least 40, at least 50, at least 60, at least 70, at least 80, at least 90, or at least 100 contiguous nucleotides.

An siRNA may be produced from a short hairpin RNA (shRNA). A shRNA is an artificial RNA molecule with a hairpin turn that can be used to silence target gene expression via the siRNA it produces in cells. See, e.g., Fire et. al., Nature 391:806-811, 1998; Elbashir et al., Nature 411:494-498, 2001; Chakraborty et al., Mol Ther Nucleic Acids 8:132-143, 2017; and Bouard et al., Br. J. Pharmacol. 157:153-165, 2009. Expression of shRNA in cells is typically accomplished by delivery of plasmids or through viral or bacterial vectors. Suitable bacterial vectors include but not limited to adeno-associated viruses (AAVs), adenoviruses, and lentiviruses. After the vector has integrated into the host genome, the shRNA is then transcribed in the nucleus by polymerase II or polymerase III (depending on the promoter used). The resulting pre-shRNA is exported from the nucleus, then processed by a protein called Dicer and loaded into the RNA-induced silencing complex (RISC). The sense strand is degraded by RISC and the antisense strand directs RISC to an mRNA that has a complementary sequence. A protein called Ago2 in the RISC then cleaves the mRNA, or in some cases, represses translation of the mRNA, leading to its destruction and an eventual reduction in the protein encoded by the mRNA. Thus, the shRNA leads to targeted gene silencing.

The shRNA or siRNA may be encoded in a vector. In some embodiments, the vector further comprises appropriate expression control elements known in the art, including, e.g., promoters (e.g., inducible promoters or tissue specific promoters), enhancers, and transcription terminators.

### III. Methods of Treatment

Any of the methods described herein may be used to modify T cells in a human subject or obtained from a human subject. Any of the methods and compositions described herein may be used to modify T cells obtained from a human subject to treat or prevent a disease (e.g., cancer, an autoimmune disease, an infectious disease, transplantation rejection, graft vs. host disease or other inflammatory disorder in a subject).

Provided herein is a method of treating an autoimmune disorder in a subject, the method comprising administering a population of T cells comprising: (a) a genetic modification or heterologous polynucleotide that inhibits expression of a nuclear factor set forth in Table 1, Table 3, Table 6 or Table 8; and/or a (b) heterologous polynucleotide that encodes a nuclear factor set forth in Table 2, Table 4, Table 5 or Table 7, to a subject that has an autoimmune disorder.

In some embodiments, a T cell wherein expression of one or more nuclear factors selected from the group consisting of CBFB, MYB, ZNF217, FOXK1, FLI1, FOS, SATB1, IL2 and ATXN7L3 is inhibited, is administered to a subject having an autoimmune disorder.

In some embodiments, a T cell, for example, a regulatory T cell, wherein expression of one or more nuclear factors selected from the group consisting of ETS1. MYBL2, MYB, TP53, FLI1, SATB1, MBD2, ZBTB7A, DNMT1, TFDP1, SMARCB1 and MAF is inhibited, is administered to a subject having an autoimmune disorder.

In some embodiments, a T cell, for example, a conventional T cell, wherein expression of one or more nuclear factors selected from the group consisting of NFATC2, MAF, ZBTB7A, MBD2, GATA3, MED14, IRF2, MED30, ZBTB11, RELA, JAK3, MED111, BCL11B, MTF1, ATXN7L3, YY1, ETS1, IL2, DNMT1, GTF2B and SMARCB1 is inhibited, is administered to a subject having an autoimmune disorder.

In some embodiments, a T cell, for example, a conventional T cell, wherein expression of one or more nuclear factors selected from the group consisting of IKZF3, YY1, MBD2, IRF4, IKZF1, RXRB, RELA, ETS1, KMT2A, PTEN, JAK3, STAT5A, GATA3, FOXP1, STAT5B and IL2RA is inhibited, is administered to a subject having an autoimmune disorder.

In some embodiments, a T cell comprising a heterologous polynucleotide that encodes a nuclear factor selected from the group consisting of MTF1, RELA, IRF1, BCL11B, STAT3, MED30, MED14, MED11, IKZF3, KMT2A, IKZF1, MED12, TAF5L, PTEN. IRF4, FOXO1, FOXP1 and CTLA4 is administered to a subject having an autoimmune disorder.

In some embodiments, a T cell comprising a heterologous polynucleotide that encodes a nuclear factor selected from the group consisting of TAF5L, FOXP3, GATA3, STAT5B, FOXP1, STAT5A, PTEN and FOXO1 is administered to a subject having an autoimmune disorder.

In some embodiments, a T cell comprising a heterologous polynucleotide that encodes a nuclear factor selected from the group consisting of MED12, FOXP1, PTEN, IKZF1, TAF5L. PRDM1, TFDP1, CXXC1, IKZF3 and TP53 is administered to a subject having an autoimmune disorder.

In some embodiments, a T cell comprising a heterologous polynucleotide that encodes a nuclear factor selected from the group consisting of MED12, CBFB, HIVEP2, KLF2, MYB, FOXK1, ZNF217, IRF2, TNFAIP3, MYC, PRDM1, TFDP1, IRF1, FOXO1, ATXN7L3 or TP53 is administered to a subject having an autoimmune disorder.

Also provided is a method of treating cancer in a subject, the method comprising administering a population of T cells comprising a genetic modification or heterologous polynucleotide that inhibits expression of a nuclear factor set forth in Table 2, Table 4, Table 5 or Table 7 and/or a heterologous polynucleotide that encodes a nuclear factor set forth in Table 1, Table 3, Table 6 to a subject that has cancer.

In some embodiments, a T cell wherein expression of one or more nuclear factors selected from the group consisting of MTF1, RELA, IRF1, BCL11B, STAT3, MED30, MED14, MED11, IKZF3, KMT2A, IKZF1, MED12, TAF5L, PTEN, IRF4, FOXO1, FOXP1 and CTLA4 is inhibited, is administered to a subject having cancer.

In some embodiments, a T cell wherein expression of one or more nuclear factors selected from the group consisting of TAF5L, FOXP3, GATA3, STAT5B, FOXP1, STAT5A, PTEN and FOXO1 is inhibited, is administered to a subject having cancer.

In some embodiments, a T cell wherein expression of one or more nuclear factors selected from the group consisting of MED12, FOXP1, PTEN, IKZF1, TAF5L. PRDM1, TFDP1, CXXC1, IKZF3 and TP53 is inhibited, is administered to a subject having cancer.

In some embodiments, a T cell wherein expression of one or more nuclear factors selected from the group consisting of MED12, CBFB, HIVEP2, KLF2, MYB, FOXK1, ZNF217, IRF2, TNFAIP3, MYC, PRDM1. TFDP1, IRF1, FOXO1, ATXN7L3 and TP53, is inhibited, is administered to a subject having cancer or an autoimmune disorder. In some embodiments, inhibition of one or more nuclear factors that increase IL-2 in effector T cells, for example, one or more nuclear factors selected from the group consisting of MED12, CBFB, HIVEP2, KLF2, MYB, FOXK1, ZNF217, IRF2, TNFAIP3, MYC, PRDM1, TFDP1, IRF1, FOXO1, ATXN7L3 or TP53 can be used to treat cancer. In some embodiments, inhibition of one or more nuclear factors that increase IL-2 in regulatory T cells, for example, one or more nuclear factors selected from the group consisting of MED12, CBFB, HIVEP2. KLF2, MYB, FOXK1, ZNF217, IRF2, TNFAIP3, MYC, PRDM1, TFDP1, IRF1, FOXO1, ATXN7L3 or TP53 can be used to treat an autoimmune disorder.

In some embodiments, a T cell comprising a heterologous polynucleotide encoding a nuclear factor selected from the group consisting of CBFB, MYB, ZNF217, FOXK1, FLI1, FOS, SATB, IL2 and ATXN7L3 is administered to a subject having cancer or an autoimmune disorder. In some embodiments, inhibition of one or more nuclear factors that increase IL-2 in effector T cells, for example, inhibition of one or more factors selected from the group consisting of CBFB, MYB, ZNF217, FOXK1, FLI1, FOS, SATB1, IL2 and ATXN7L3, can be used to treat cancer in a subject. In some embodiments, inhibition of one or more nuclear factors that increase IL-2 in regulatory T cells, for example, inhibition of one or more factors selected from the group consisting of CBFB, MYB, ZNF217, FOXK1, FLI1, FOS, SATB1, IL2 and ATXN7L3, can be used to autoimmune disease in a subject.

In some embodiments, a T cell comprising a heterologous polynucleotide encoding a nuclear factor selected from the group consisting of ETS1, MYBL2, MYB, TP53, FLI1. SATB1, MBD2, ZBTB7A, DNMT1, TFDP1, SMARCB1 and MAF is administered to a subject having cancer.

In some embodiments, a T cell comprising a heterologous polynucleotide encoding a nuclear factor selected from the group consisting of NFATC2, MAF, ZBTB7A, MBD2, GATA3, MED14, IRF2, MED30, ZBTB11, RELA, JAK3, MED11, BCL11B, MTF1, ATXN7L3, YY1, ETS1, IL2, DNMT1, GTF2B and SMARCB1 is administered to a subject having cancer.

In some embodiments, a T cell comprising a heterologous polynucleotide encoding a nuclear factor selected from the group consisting of IKZF3, YY1, MBD2, IRF4, IKZF1. RXRB, RELA, ETS1, KMT2A, PTEN, JAK3, STAT5A, GATA3, FOXP1, STAT5B is administered to a subject having cancer.

Provided herein is a method of treating cancer in a human subject comprising: a) obtaining T cells from the subject; b) modifying the T cells using any of the methods provided herein; and c) administering the modified T cells to the subject, wherein the human subject has cancer.

In some embodiments, the method for treating cancer comprises method comprises: a) obtaining T cells from the subject; b) modifying the T cells by inhibiting expression of one or more nuclear factors selected from the group consisting of MTF1, RELA, IRF1, BCL11B, STAT3, MED30, MED14, MED11, IKZF3, KMT2A, IKZF1, TAF5L, IRF4, FOXP1, CTLA4, FOXP3, GATA3, STAT5B, STAT5A, PTEN, FOXO1, MED12. FOXP1, PTEN, IKZF1, TAF5L, PRDM1, TFDP1, CXXC1, IKZF3, TP53, CBFB, HIVEP2, KLF2, MYB, FOXK1, ZNF217, IRF2, TFNAIP3, MYC, PRDM1, TFDP1, IRF1, ATXN7L3 and TP53; and c) administering the T cells to the subject.

In some embodiments, the method for treating cancer comprises: a) obtaining T cells from the subject; b) modifying the T cells by overexpressing one or more nuclear factors selected from the group consisting of CBFB, MYB, ZNF217, FOXK1, FLI1, FOS, IL2, ATXN7L3, ETS1, MYBL2, MYB, TP53, FLI1, SATB1, ZBTB7A, DNMT1, TFDP1, SMARCB1, MAF, NFATC2, MAF, ZBTB7A, MED14, IRF2, MED30, ZBTB11, MED11, BCL11B, MTF1, ATXN7L3, YY1, ETS1, IL2. DNMT1, GTF2B, IKZF3, MBD2, IRF4, IKZF1, RXRB, RELA, ETS1, KMT2A, PTEN, JAK3, STAT5A, GATA3, FOXP1, STAT5B and IL2RA; and c) administering the T cells to the subject.

Also provided herein is a method of treating an autoimmune disease in a human subject comprising: a) obtaining T cells from the subject; b) modifying the T cells using any of the methods provided herein; and c) administering the modified T cells to the subject, wherein the human subject has an autoimmune disease.

In some embodiments, the method for treating autoimmune disease comprises a) obtaining T cells from the subject; b) modifying the T cells by inhibiting expression of one or more nuclear factors selected from the group consisting of CBFB, MYB, ZNF217, FOXK1, FLI1, FOS, IL2, ATXN7L3, ETS1, MYBL2, MYB, TP53, FLI1, SATB1, ZBTB7A, DNMT1, TFDP1, SMARCB1, MAF, NFATC2, MAF, ZBTB7A, MED14, IRF2, MED30, ZBTB11, MED11, BCL11B, MTF1, ATXN7L3, YY1, ETS1, IL2, DNMT1. GTF2B. IKZF3. MBD2, IRF4, IKZF1, RXRB, RELA, ETS1, KMT2A, PTEN, JAK3, STAT5A, GATA3, FOXP1, STAT5B and IL2RA; and c) administering the T cells to the subject.

In some embodiments, the method for treating an autoimmune disorder comprises: a) obtaining T cells from the subject; b) modifying the T cells by overexpressing one or more nuclear factors selected from the group consisting of MTF1, RELA, IRF1, BCL11B, STAT3, MED30, MED14, MED11, IKZF3, KMT2A, IKZF1, TAF5L, IRF4, FOXP1, CTLA4, FOXP3, GATA3, STAT5B, STAT5A, PTEN, FOXO1, MED12, FOXP1, PTEN, IKZF1, TAF5L, PRDM1, TFDP1, CXXC1, IKZF3, TP53, CBFB, HIVEP2, KLF2, MYB, FOXK1, ZNF217, IRF2, TFNAIP3, MYC, PRDM1, TFDP1, IRF1, ATXN7L3 and TP53; and c) administering the T cells to the subject.

In some embodiments, T cells obtained from a cancer subject may be expanded ex vivo. The characteristics of the subject's cancer may determine a set of tailored cellular modifications (i.e., which nuclear factors from Table 1, Table 2, Table 3, Table 4, Table 5, Table 6, Table 7, Table 8, Table 9, Table 10, Table 11, Table 12, Table 13 and/or Table 14 to target), and these modifications may be applied to the T cells using any of the methods described herein. Modified T cells may then be reintroduced to the subject. This strategy capitalizes on and enhances the function of the subject's natural repertoire of cancer specific T cells, providing a diverse arsenal to eliminate mutagenic cancer cells quickly. Similar strategies may be applicable for the treatment of autoimmune diseases.

In other cases, T cells in a subject can be targeted for in vivo modification. See, for example, See, for example, U.S. Pat. No. 9,737,604 and Zhang et al. “Lipid nanoparticle-mediated efficient delivery of CRISPR/Cas9 for tumor therapy.” NPG Asia Materials Volume 9, page e441 (2017).

Disclosed are materials, compositions, and components that can be used for, can be used in conjunction with, can be used in preparation for, or are products of the disclosed methods and compositions. These and other materials are disclosed herein, and it is understood that when combinations, subsets, interactions, groups, etc. of these materials are disclosed that while specific reference of each various individual and collective combinations and permutations of these compounds may not be explicitly disclosed, each is specifically contemplated and described herein. For example, if a method is disclosed and discussed and a number of modifications that can be made to one or more molecules including in the method are discussed, each and every combination and permutation of the method, and the modifications that are possible are specifically contemplated unless specifically indicated to the contrary. Likewise, any subset or combination of these is also specifically contemplated and disclosed. This concept applies to all aspects of this disclosure including, but not limited to, steps in methods using the disclosed compositions. Thus, if there are a variety of additional steps that can be performed, it is understood that each of these additional steps can be performed with any specific method steps or combination of method steps of the disclosed methods, and that each such combination or subset of combinations is specifically contemplated and should be considered disclosed.

Publications cited herein and the material for which they are cited are hereby specifically incorporated by reference in their entireties.

### EXAMPLES

The following examples are provided by way of illustration only and not by way of limitation. Those of skill in the art will readily recognize a variety of non-critical parameters that could be changed or modified to yield essentially the same or similar results.

### Example 1

### Materials and Methods

Buffers/media
cRPMI

| | Stock | | | Final | | | |
|---|---|---|---|---|---|---|---|
| Name | Concentration | | Purpose | Concentration | | Source | Cat No |
| Media | N/A | | Support cells in | N/A | | Sigma | R0883 |
| (1640 RPMI) | | | suspension culture | | | | |
| FCS | N/A | | Growth | 10% | | Sigma | F0926 |
| | | | promotion/survival | | | | |
| | | | enhancement | | | | |
| Penicillin- | 10,000 | U/mL | Prevention of bacterial | 100 | U/mL | Gibco | 15140-122 |
| Streptamycin | | | contamination | | | | |
| L-Glutamine | 200 | mM | Energy source | 1% | (2 mM) | Sigma | G7513 |
| HEPES | 1M, pH 7.0- | | organic zwitterionic | 1% | (10 mM) | Sigma | H0887 |
| | 7.6 | | buffering agent | | | | |
| MEM Non- | 100X | | Increase cell | 1% | (1X) | Gibco | 11140-050 |
| essential Amino | | | growth/viability | | | | |
| Acids | | | | | | | |
| Sodium | 100 | mM | Carbon source | 1% | (1 mM) | Gibco | 11360-070 |

Pyruvate

X-VIVO 15 Serum-Free Hematopoietic Cell Medium

| | Stock | | Final | | | |
|---|---|---|---|---|---|---|
| Name | Concentration | Purpose | Concentration | | Source | Cat No |
| Media | N/A | Support cells in | N/A | | Lonza | 04-418Q |
| (X-VIVO 15*) | | suspension culture | | | | |
| FCS | N/A | Growth | 5% | | Sigma | F0926 |
| | | promotion/survival | | | | |
| | | enhancement | | | | |
| N-acetyl L-cysteine | 1-10M | NAC is an | 10 | mM | Sigma | A9165 |
| | (diluted in | antioxidant | | | | |
| | ddH₂O) | molecule that inhibit | | | | |
| | | ROS activity by | | | | |
| | | scavenging activity. | | | | |
| 2-mercaptoethanol | 55 mM | A thiol compound, | 55 | uM | Gibco | 21985023 |

| | |
|---|---|
| (β-me) | commonly used as a |
| | reducing agent in |
| | organic reactions |

Complete DMEM:

| | Stock | | | Final | | | |
|---|---|---|---|---|---|---|---|
| Name | Concentration | | Purpose | Concentration | | Source | Cat No |
| DMEM high | N/A | | Cell media | N/A | | UCSF | CCFAA005 |
| glucose | | | | | | CCF | |
| FCS | N/A | | Growth | 10% | | Sigma | F0926 |
| | | | promotion/survival | | | | |
| | | | enhancement | | | | |
| Penicillin- | 10,000 | U/mL | Prevention of bacterial | 100 | U/mL | Gibco | 15140-122 |
| Streptamycin | | | contamination | | | | |
| L-Glutamine | 200 | mM | Energy source | 1% | (2 mM) | Sigma | G7513 |
| HEPES | 1M, pH 7.0- | | organic zwitterionic | 1% | (10 mM) | Sigma | H0887 |
| | 7.6 | | buffering agent | | | | |
| MEM Non- | 100X | | Increase cell | 1% | (1X) | Gibco | 11140-050 |
| essential Amino | | | growth/viability | | | | |
| Acids | | | | | | | |
| Sodium | 100 | mM | Carbon source | 1% | (1 mM) | Gibco | 11360-070 |

Pyruvate

Fluorescence- activated cell sorting (FACS) buffer

| | Stock | | Final | | |
|---|---|---|---|---|---|
| Name | Concentration | Purpose | Concentration | Source | Cat No |
| PBS | N/A | Support cells in suspension | N/A | Sigma | R0883 |
| | | culture | | | |
| FCS | N/A | Protein Carrier to reduce | 2% | Sigma | F0926 |
| | | nonspecific antibody binding | | | |
| EDTA | 0.5M, pH 8.0 | Chealator (prevents cell | 1 mM | Gibco | 15140-122 |
| | | clumping) | | | |

Pooled sgRNA Library Construction

We selected transcription factors (TFs) with known or inferred motifs from Lambert et al (Lambert et al., “The human transcription factors,” Cell 172(4): 650-665 (2018)), non-target controls from the Brunello sgRNA library (Doench et al., 2016) and several immune genes of interest from the lab. All sgRNA sequences were from the Brunello sgRNA library (Doench et al., “Optimized sgRNA design to maximize activity and minimize off-target effects of CRISPR-Cas9,” Nat Biotechnol 34(2): 184-192 (2016)). In total we included 1349 genes with an average of 4 guides per gene, 13 guides against GFP as a positive control for editing, and 593 non-targeting controls. Following the custom sgRNA library cloning protocol as described by Joung et al. (Joung et al., “Genome-scale CRISPR-Cas9 knockout and transcriptional activation screening,” Nat Protoc 12(4): 828-863 (2017)), we integrated our TF sgRNA library into the LRG2.1 backbone (Addgene, plasmid #108098) based off data from the study by Grevet et al. (Grevet et al., “Domain-focused CRISPR screen identifies HRI as a fetal hemoglobin regulator in human erythroid cells,” Science 361(6399): 285-290 (2018)). Our pooled oligo library was ordered from Twist Bioscience with flanking sequences allowing for integration into the LRG2.1 backbone using NEBuilder HiFi DNA Assembly master mix (NEB, Cat #E2621X) according to the manufacturer's protocol. We used Endura ElectroCompetent Cells to amplify the TF library per the manufacturer's protocol (Endura, Cat #60242-1).

### Lentiviral Production

HEK 293T cells were seeded at 14 million cells in a 15 cm tissue culture treated culture dish (Corning, Cat #430599) in Opti-MEM 24 hours prior to transfection. Using Lipofectamin 3000 (Lifetech, Cat #L3000075) according to the manufacturer's protocol, cells were transfected with the sgRNA transfer plasmid, and two lentiviral packaging plasmids, pMD2.G (Addgene, Cat #12259) and psPAX2 (Addgene, Cat #12260). Cells were incubated for 5 hours at 37° C., after which time the transfection media was removed and replaced with fresh Opti-MEM containing ViralBoost at 1× (Alstem, Cat #VB100). Cells were returned to the incubator for 24 hours after which time the viral supernatant was collected and spun down at 300 g for 5 minutes to remove cellular debris. The supernatant was then passed through a 0.45-μm filter, and subsequently mixed with one volume of cold Lentivirus Precipitation Solution (Alstem, Cat #VC125) at 4° C. to every 4 volumes of lentivirus-containing supernatant. Samples were mixed well and placed at 4° C. overnight. The virus was then concentrated by centrifugation at 1500 g for 30 minutes at 4° C., after which the supernatant was discarded, and the residual sample underwent additional centrifugation at 1500 g for 5 minutes to remove any residual supernatant. The viral pellet was then resuspended at a ratio of 1:100 of the original volume using PBS (Fisher Scientific, Cat #10010049) at 4° C. Virus was then stored until use at −80° C.

### Isolation, Culture and Expansion of Human CD4+T-Effector Cells and CD4+CD127lowCD25+ Regulatory T Cells for Screening Experiments

Primary human T cells were obtained from residuals from ***leukoreduction chambers after apheresis (Blood Centers of the Pacific) for experiments not involving RNA-Seq or high throughput amplicon sequencing. For sequencing experiments, primary human T cells were obtained from whole blood donors through a protocol approved by the UCSF Committee on Human Research (CHR #13-11950). Peripheral blood mononuclear cells (PBMCs) were isolated by size separation using Lymphoprep (STEMCELL, Cat #07861) in SepMate tubes (STEMCELL, Cat #85460), according to the manufacturer's protocol. Isolated PBMCs were then subjected to antibody mediated magnetic separation to isolate either CD4+CD127lowCD25+ effector T cells or CD4+CD127lowCD25+ regulatory T cells. In order to ensure that out CD4+ population did not also contain CD4+CD127lowCD25+ regulatory T cells, we utilized the CD4+ negative isolation protocol from the StemCell EasySep™ Human CD4+CD127lowCD25+ Regulatory T Cell Isolation Kit (Catalog #18063). This same kit was used when CD4+CD127lowCD25+ regulatory T cells were desired. Effector T cells were cultured in cRPMI, while Regulatory T cells were cultured in X-Vivo (formulations above). After isolation, cells were stimulated with Immunocult Human CD3/CD28/CD2 T Cell Activator (STEMCELL, Cat #10970) at 6.25 uL per 1E6 cells, with IL-2 (Amerisource Bergen, Cat #10101641) at 50 U/mL for effector T cells and 300 U/mL for regulatory T cells, at a concentration of 1E6 cells/mL.

### Regulatory T Cell Expansion

In order to achieve the necessary number of cells to maintain power in the results of the screen, regulatory T cells needed to undergo a period of expansion and restimulation prior to lentiviral transduction and Cas9 electroporation. Five days after the initial isolation and stimulation as described above, cells were passaged and subsequently cultured at 2.5E5 cells/mL in XVIVO containing 300 U/mL human IL-2. After an additional 4 days (9 days from initial stimulation), cells were restimulated with 6.25 uL of Immunocult per million cells as previously described. These cells underwent lentiviral transduction and Cas9 electroporation as described below according to the same schedule as effector T cells.

### Lentiviral Transduction and Cas9 Electroporation

Twenty-four hours post stimulation, lentivirus containing the TF library was added directly to cultured T cells in a drop-wise fashion and tilting the plates to distribute evenly, targeting a multiplicity of infection (MOI) of 0.4 (Ellis & Delbralck, “The growth of bacteriophage,” J Gen Physiol 22(3): 365-384 (1939)). After an additional 24 hours, excess lentivirus was removed from the supernatant and washed off the cells by collecting the cells as a single cell suspension in a 50 mL conical, centrifuging at 300 g, discarding the supernatant, and resuspending the cells in fresh media (cRPMI or X-vivo if effector T cells or regulatory T cells, respectively). Cells were then incubated at 37° C.

### Cas9-Ribonucleotide Protein (RNP) Preparation

Cas9 protein (MacroLab, Berkeley, 40 μM stock) was delivered into the cells using a modified Guide Swap technique (Ting P Y, et al., “Guide Swap enables genome-scale pooled CRISPR-Cas9 screening in human primary cells,” Nat Methods 15(11): 941-946 (2018). To do this, on the day of electroporation, lyophilized Dharmacon Edit-R crRNA Non-targeting Control #3 (Dharmacon, Cat #U-007503-01-05) and Dharmacon Edit-R CRISPR-Cas9 Synthetic tracrRNA (Dharmacon, Cat #U-002005-20) were resuspended at a stock concentration of 160 mM in 10 mM Tris-HCl (pH 7.4) with 150 mM KCl. They were mixed at a 1:1 ratio, creating an 80 mM solution, and incubated on a heat block at 37° C. for minutes. Single-stranded donor oligonucleotides (ssODN; sequence: TTAGCTCTGTTTACGTCCCAGCGGGCATGAGAGTAACAAGAGGGTGTGGTAATAT TACGGTACCGAGCACTATCGATACAATATGTGTCATACGGACACG) (SEQ ID NO: 2573) was then added at a 1:1 molar ratio of the final Cas9-Guide complex, and mixed well by pipetting. The solution was incubated for an additional 5 minutes at 37° C. on the heat block. Cas9 was then added slowly at a 1:1 volume to volume ratio, taking care to avoid precipitation, pipetting up and down several times to ensure complete resuspension of the RNP complex, and incubated at 37° C. for 15 minutes completing the process of creating the assembled RNP-ssODN complex.

### Electroporation

Following 24 hours after residual virus was washed from the culture, cells were centrifuged at 100 g for 10 minutes to pellet them, and resuspended in room temperature Lonza P3 electroporation buffer (Lonza, Cat #V4XP-3032) at 1-2E6 cells per 17.8 μL. 7.2 μL of the RNP-ssODN complex were added for every 17.8 μL of cells and mixed well. Using a multichannel pipette, 23 uL of the cells-RNP-ssODN mixture were added per well to a 96 well electroporation cuvette plate (Lonza, Cat #VVPA-1002), and nucleofected using the pulse code EH-115. Immediately after electroporation, 90 μL of prewarmed media were added to each well and incubated at 37° C. for 15 minutes. Cells were then pooled, transferred to incubation flasks, and diluted with pre-warmed media to a final concentration of 1E6 cells/mL and incubated at 37° C. Cells were passaged at 48 hours post electroporation, and subsequently maintained in culture at 1E6 cells/mL.

### Screen Phenotyping and Cell Sorting

Cells were screened 6 days following electroporation. 10-20E⁶ cells were portioned off and sorted based on GFP expression only. The remaining cells were sorted based on GFP positivity, as well as a target phenotype using an APC fluorescent antibody targeting either CD25 (Tonbo, Cat #20-0259-T100), IL-2 (Biolegend, Cat #500310), CTLA-4 (Biolegend, Cat #349908), or Foxp3 (eBiosciences, Cat #17-4777-42). Cells sorted for CD25 underwent surface staining according to the manufacturer's protocol. Cells sorted for IL-2 were treated with Cell Activation Cocktail with Brefeldin A (Biolegend, Cat #423304) for 4 hours prior to fixation, and were fixed using the CD Cytofix/Cytoperm kit (Becton Dickinson, Cat #554714) according to the manufacturer's protocol. Cells sorted for CTLA-4 were treated with Cell Activation Cocktail without Brefeldin A (Biolegend, Cat #423302) for 4 hours prior to fixation, and were fixed using the Foxp3 Fix/Perm buffer set (Biolegend, Cat #421403) according to the manufacturer's protocol. Cells sorted for Foxp3 were fixed using the True-Nuclear Transcription Factor buffer set (Biolegend, Cat #424401) according to the manufacturer's protocol. Cells were sorted using a BD FACS Aria II.

### Genomic DNA Extraction and Preparation for Next Generation Sequencing

After sorting, cells were washed with PBS, counted, pelleted, and resuspending at up to 5E6 cells per 400 μl of ChIP lysis buffer (1% SDS, 50 mM Tris, pH 8, 10 mM EDTA). The remaining protocol reflects additives/procedures performed on each 400 μl sample. 16 μl of NaCl (5M) was added, and the sample was incubated on a heat block overnight at 66° C. The next morning, 8 μl of RNAse A (10 mg/ml, resuspended in ddH-20) (Zymo, Cat #E1008) was added, and the sample was vortexed briefly, and incubated at 37° C. for 1 hour. Next, 8 μl of Proteinase K (20 mg/ml) (Zymo, Cat #D3001) was added, the sample was vortexed briefly, and incubated at 55° C. for 1 hour. A phase lock tube (Quantabio, Cat #2302820) was prepared for each sample by spinning down the gel to the bottom of the tube at 20,000 g for 1 minute, after which 400 μl of Phenol:Chloroform:Isoamyl Alcohol (25:24:1) was added to each tube. 400 μl of the sample was then added to the phase lock tube, which was then shaken vigorously. The sample was then centrifuged at maximum speed at room temperature for 5 minutes. The aqueous phase was transferred to a low-binding eppendorf tube (Eppendorf, Cat #022431021) to which was added 40 μl of Sodium Acetate (3M). 1 μl GlycoBlue and 600 μl of isopropanol at room temperature. The sample was then vortexed and stored at −80° C. for 30 minutes or until the sample had frozen solid. Next the sample was centrifuged at maximum speed at 4° C. for 30 minutes, the pellet was washed with fresh 70% room temperature Ethanol, and allowed to air dry for 15 minutes. Pellets were then resuspended in Zymo DNA elution buffer (Zymo, Cat No: D3004-4-10), and placed on the heat block at 65° C. for 1 hour to completely dissolve the genomic DNA.

sgRNA was amplified and barcoded from the genomic DNA according to the protocol by Joung et al. (Joung et al., 2017). Up to 2.5 μg of genomic DNA were added to each 50 μL reaction, which included 25 μL of NEBNext Ultra II Q5 master mix (NEB, Cat #M0544L), 1.25 μL of the 10 μM forward primer (AATGATACGGCGACCACCGAGATCTACAC GCTTTATATATCTTGTGGAAAGGACGAAACACC) (SEQ ID NO: 2574), and 1.25 μL of the 10 μM reverse primer (CAAGCAGAAGACGGCATACGAGAT) (SEQ ID NO: 2575) i7 index (GTGACTGGAGTTCAGACGTGctttgctgtttccagcaaagttgataacg) (SEQ ID NO: 2576), with the remaining volume as water. PCR cycling conditions were: 98° C. for 3 minutes, followed by 23 cycles at 98° C. for 10 seconds. 63° C. for 10 seconds, and 72° C. for 25 seconds, and ending with 2 minutes at 72° C. Samples were then cleaned and concentrated in Zymo Spin-V columns (Zymo, Cat #C1016-50) following Joung et al., and eluted in 150 μL of Zymo DNA elution buffer. Up to 2 μg of each library were loaded on a 2% agarose gel, and the band at ˜250 base pairs was extracted using the Zymoclean Gel DNA recovery kit (Zymo, Cat #D4008). The concentration of each sample was then measured using the Qubit dsDNA high sensitivity assay kit (Thermo Fisher Scientific. Cat #Q32854). Samples were then sequenced on an Illumina HiSeq 4000 using 10-30% PhiX (Illumina, Cat #15017872), and a custom primer (sequence: CCGAGATCTACACGCTTTATATATCTTGTGGAAAGGACGAAACACC) (SEQ ID NO: 2576).

### Arrayed Validation Isolation, Culture, and Electroporation

Based on the screen results, we chose to pursue the top two performing guides for 60 target genes (including 4 NTCs per plate). Guides were selected both for their overlap across screens, as well as some that were unique to only a single screen. The complete guide list can be found in supplemental table ***. Primary human T cells were obtained from whole blood donors through a protocol approved by the UCSF Committee on Human Research (CHR #13-11950), isolated and stimulated as described above. Custom crRNA plates were ordered from Dharmacon, and were assembled as RNP-ssODN complexes as described above. 48 hours after stimulation, cells were counted, pelleted, and resuspended in room temperature Lonza P3 buffer (Lonza. Cat #V4XP-3032) at 1E6 cells per 20 μL. These were then mixed with 100 pmol of RNP each mixed well, and transferred to a 96 well electroporation cuvette plate (Lonza, Cat #VVPA-1002), and nucleofected using the pulse code EH-115. After electroporation, 90 μL of pre-warmed media was immediately added to each well and plates were incubated at 37° C. for 15 minutes. Wells were then split to a target culture population of 1E6 cells/mL filling all edge wells in the 96-well plate with PBS in order to avoid edge-effects (*** reference), and incubated at 37° C.

### Arrayed Validation Phenotyping Using Flow Cytometry and Genotyping

Arrayed validation plates were phenotyped at 3, 5, and 7 days after electroporation using the sample protocol and materials as outlined in the screen in a 96-well plate format. Cells were checked for expression of CD25 (Tonbo, Cat #20-0259-T100), IL-2 (Biolegend, Cat #500310), CTLA-4 (Biolegend, Cat #349908), or Foxp3 (eBiosciences, Cat #17-4777-42) using an Attune NxT Flow Cytometer with a 96-well plate-reader.

On day 5 post-electroporation, genomic DNA was isolated from each sample using DNA QuickExtract (Lucigen, Cat #QE09050) according to the manufacturer's protocol. Custom forward and reverse primers were ordered from IDT (Supplementary table ***). Amplicons containing CRISPR edit sites were generated by adding 1.25 μL each of forward and reverse primer at 10 nM to 5 μL of sample in QuickExtract, 12.5 μL of NEBNext Ultra II Q5 master mix (NEB, Cat #M0544L), and water to a total 25 μL reaction volume. The PCR cycling conditions were 98° C. for 3 minutes, 15 cycles of 94° C. for 20 seconds followed by 65° C.-57.5° C. for 20 seconds (0.5° C. incremental decreases per cycle), and 72° C. for 1 minute, and a subsequent 20 cycles at 94° C. for 20 seconds, 58° C. for 20 seconds and 72° C. for 1 minute, and a final 10 minute extension at 72° C. Samples were then diluted 1:200 and subsequently indexed using primers listed in Supplemental Table ***. Indexing reactions included 1 μL of the diluted sample, 2.5 μL of each the forward and reverse indexing primers at 10 μM each, 12.5 μL of NEB Q5 master mix, and water to a total 25 μL reaction volume. The indexing PCR cycling conditions were 98° C. for 30 seconds, followed by 98° C. for 10 seconds, 60° C. for 30 seconds, and 72° C. for 30 seconds for 12 cycles, and a final extension period at 72° C. for 2 minutes. Samples were quantified in a 96-well plate reader using the Quant-IT DNA high sensitivity assay kit (Invitrogen, Cat #Q33232) according to the manufacturer's protocol. Post pooling, samples were then SPRI purified, and quantified using an Agilent 4200 TapeStation. Samples were then sequenced on an Illumina MiniSeq with PE 300 reads.

### Quantification and Statistical Analysis

### Analysis of Pooled Screens

Counts for sgRNA libraries were generated using the count command in MAGeCK version 0.5.8 (mageck count—norm-method none). High outlier counts were filtered out before calculating differentially enriched sgRNAs between the low and high bins using the mageck test command (mageck test-k countfile-t low_rep1,low_rep2-c high_rep1,high_rep2—sort-criteria pos). We used an FDR <0.05 as a cutoff to call significantly differentially enriched sgRNAs.

### Analysis of Arrayed Validation

Cells were gated on lymphocytes and singlets in FlowJo Version and fluorescence area for each stain was exported to csv files. Fluorescence data was imported into R version 3.6.0. The median fluorescence across 4 non-targeting controls was calculated per donor per plate and the fluorescence of each well on the plate was normalized to the median control fluorescence.

### Results

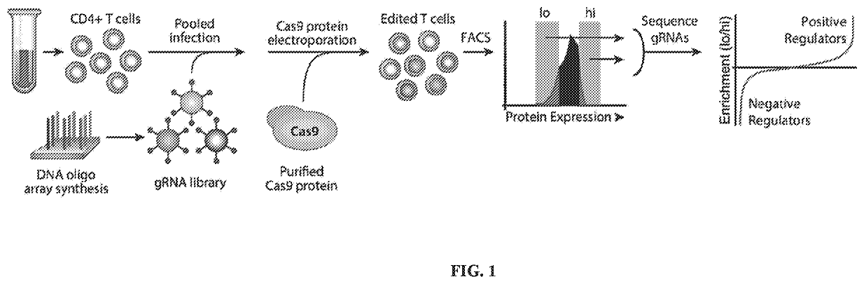

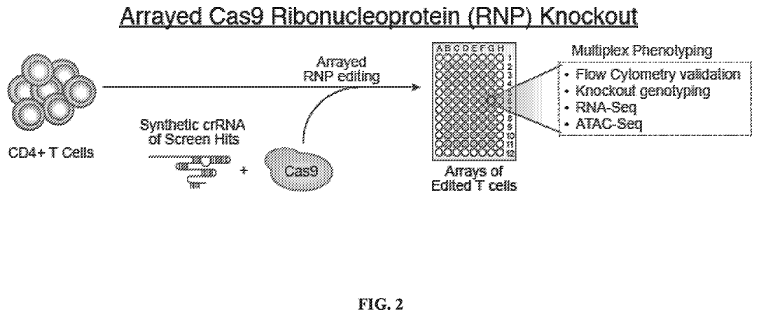

As shown in FIG. 1, SLICE was used to identify nuclear factors that modulate expression of CTLA4, IL-2, IL2RA and FOXP3 in T cells. As shown in FIG. 2 an arrayed Cas9 ribonucleoprotein (RNP) approach was used to individually knock out transcription factor hits from SLICE Flow-Seq screens.

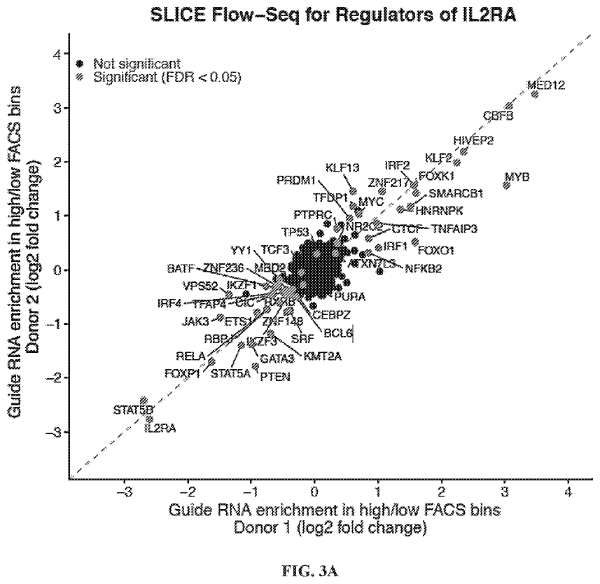

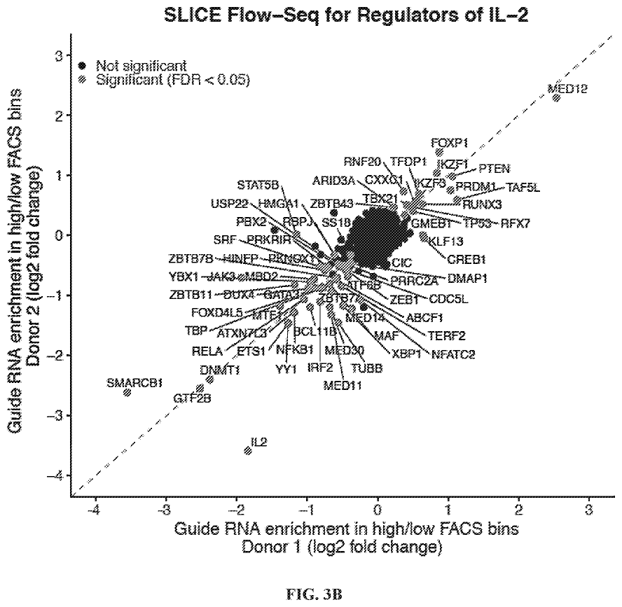

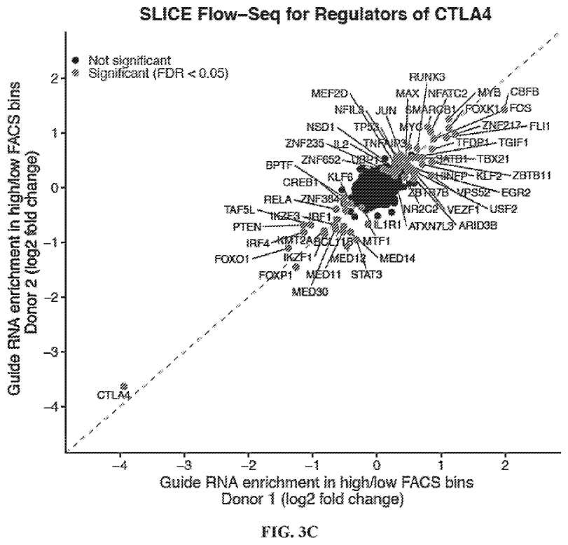

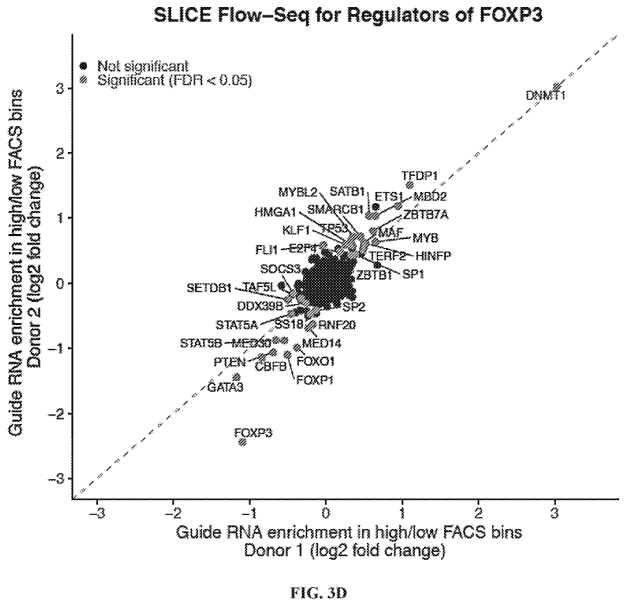

As shown in FIGS. 3A-3D transcription factors that regulate protein levels of four key immune genes IL2RA (FIG. 3A), IL-2 (FIG. 3B), CTLA4 (FIG. 3C) and FOXP3 (FIG. 3D) were discovered using SLICE Flow-Seq. These transcription factors are also listed in Tables 1-8. Cells were stained for the target of interest, sorted into high and low expression bins using fluorescent activated cell sorting, and the guide RNAs in each bin were sequenced. Red points highlight transcription factors that are significantly differently enriched between the high and low bins. Each dot represents the signal across four independent guide RNAs targeting that transcription factor.

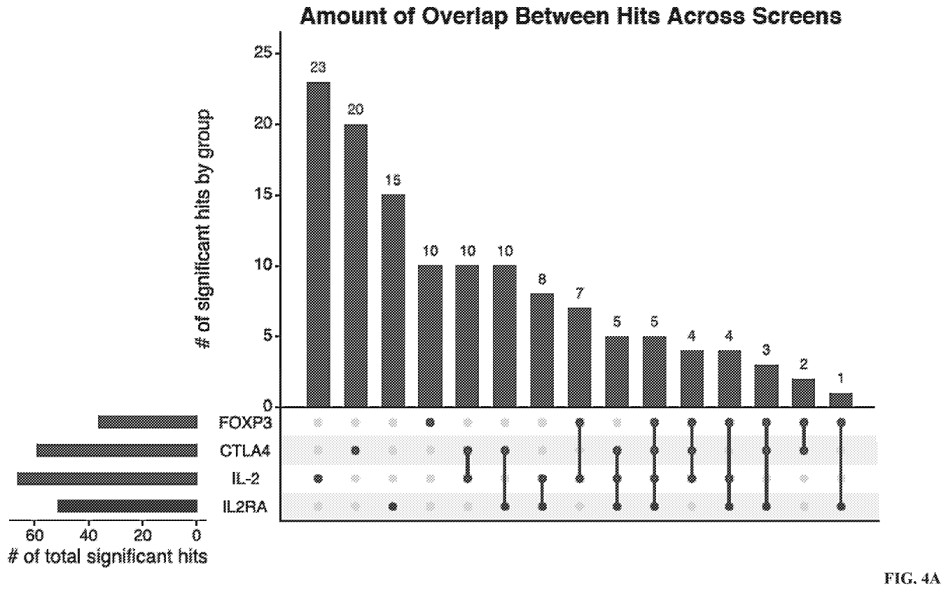

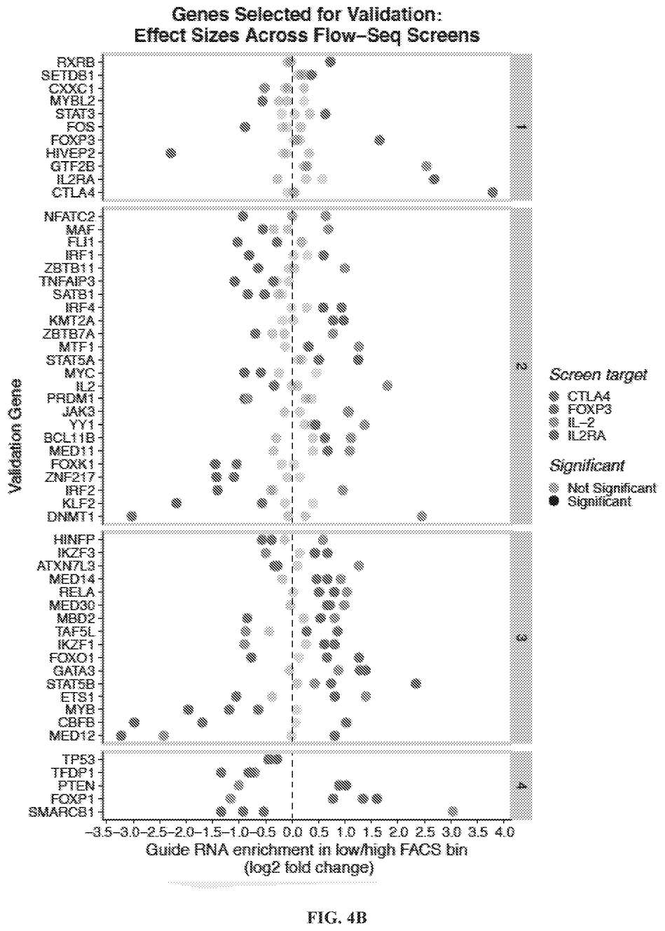

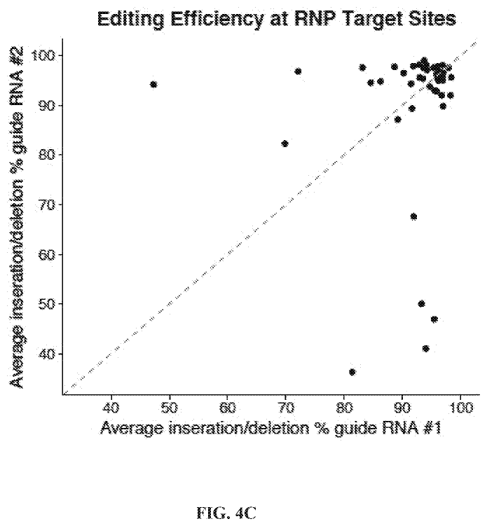

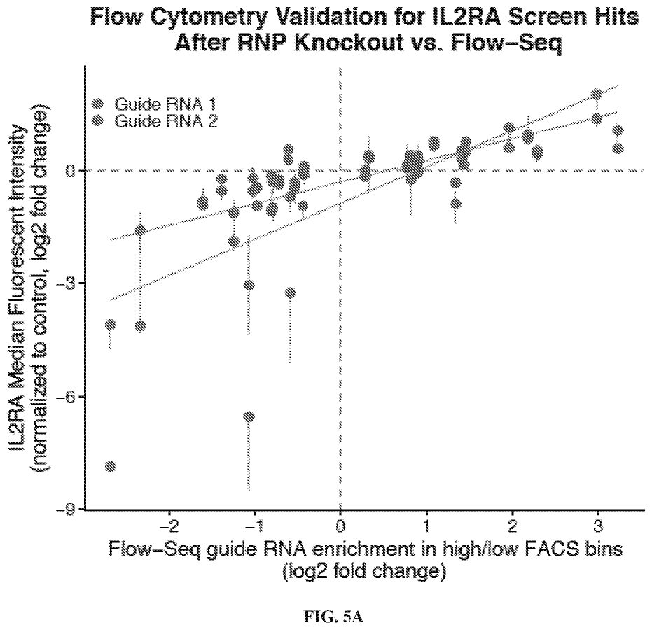

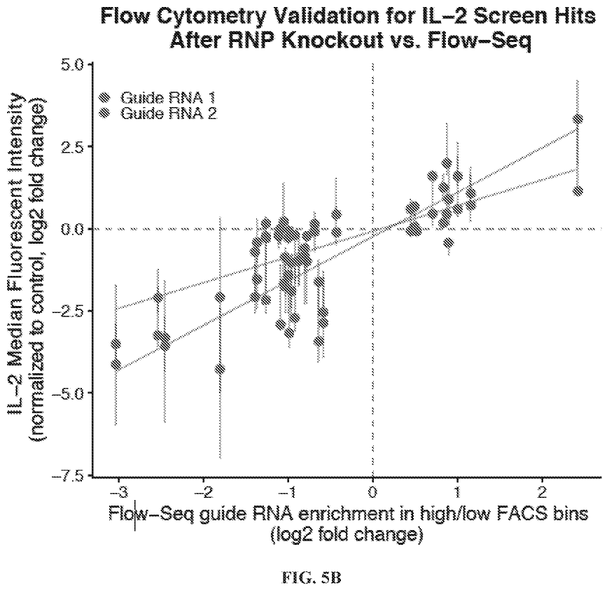

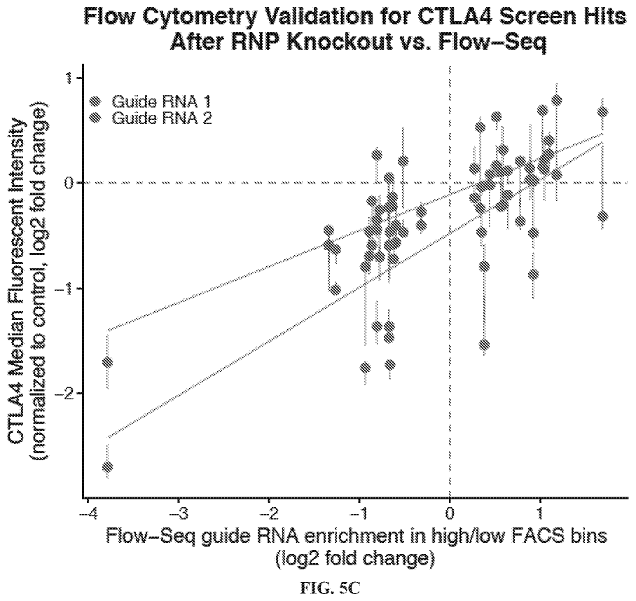

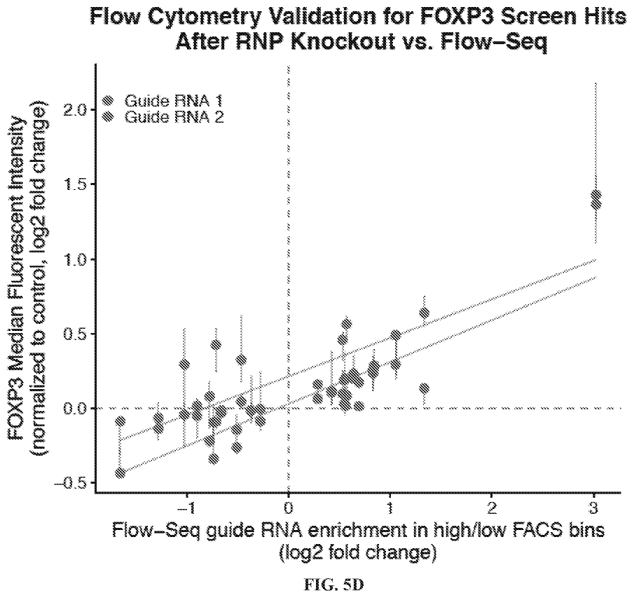

As shown in FIGS. 4A-4C show there was a high degree of overlap between hits from the four screens. The hits from each screen were validated via flow cytometery. FIGS. 5A-5D show flow cytometry validation of screen hits following RNP knockout. Cells were stained for the target of interest (IL2RA (FIG. 5A), IL-2 (FIG. 5B), CRLA4 (FIG. 5C) and FOXP3 (FIG. 5D)) and analyzed using flow cytometry. Median fluorescent intensity was normalized to four non-targeting controls per donor. Points are colored based on two independent guide RNAs. Points show the median of 3 biological donors and error bars show the range.

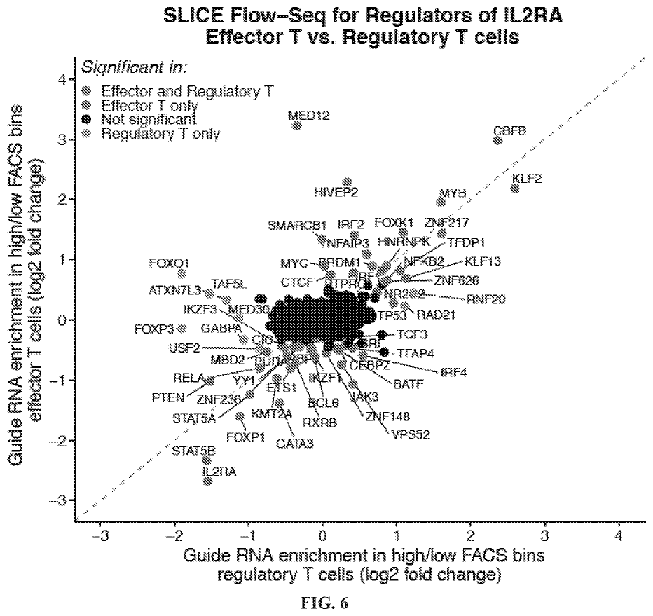

As shown in FIG. 6, numerous cell type-specific transcription factors that regulate the protein levels of IL2RA were discovered using SLICE Flow-Seq in effector T cells vs. regulatory T cells. Effector and regulatory T cells were stained for IL2RA, sorted into high and low expression bins using fluorescent activated cell sorting, and the guide RNAs in each bin were sequenced.

In summary, SLICE Flow-Seq identified 40-60 transcription factors per target that regulate protein levels of IL2RA, IL-2, CTLA4 and FOXP3.

### Example 2

### Validation Studies

### Arrayed Validation Isolation, Culture, and Electroporation

Based on the screen results, the top two performing guides for 57 target genes (including 4 non-targeting controls per plate) were chosen. Primary human T cells were obtained from whole blood donors through a protocol approved by the UCSF Committee on Human Research (CHR #13-11950), isolated and stimulated as described below. Custom crRNA plates were ordered from Dharmacon, and were assembled as RNP-ssODN complexes as described below. 48 hours after stimulation, cells were counted, pelleted, and resuspended in room temperature Lonza P3 buffer (Lonza, Cat #V4XP-3032) at 1E⁶ cells per 20 μL. Cells were then mixed with 100 pmol of RNP, transferred to a 96 well electroporation cuvette plate (Lonza, Cat #VVPA-1002), and nucleofected using the pulse code EH-115. After electroporation, 90 μL of pre-warmed media was immediately added to each well and plates were incubated at 37° C. for 15 minutes. Wells were then split to a target culture population of 1E¹ cells/mL filling all edge wells in the 96-well plate with PBS in order to avoid edge-effects and incubated at 37° C.

### Arrayed Validation Phenotyping Using Flow Cytometry and Genotyping

Arrayed validation plates were phenotyped at 5 days after electroporation using the sample protocol and materials as outlined in the screen in a 96-well plate format. Cells were checked for expression of IL2RA (CD25) (Tonbo, Cat #20-0259-T100), IL-2 (Biolegend, Cat #500310), or CTLA-4 (Biolegend, Cat #349908) using an Attune NxT Flow Cytometer with a 96-well plate-reader.

On day 5 (sgRNA #1 Donor 1-3) or day 7 (sgRNA #2 Donor 1, 3) post-electroporation, genomic DNA was isolated from each sample using DNA QuickExtract (Lucigen, Cat #QE09050) according to the manufacturer's protocol. Custom forward and reverse primers were ordered from IDT. Amplicons containing CRISPR edit sites were generated by adding 1.25 μL each of forward and reverse primer at 10 nM to 5 μL of sample in QuickExtract, 12.5 μL of NEBNext Ultra 11 Q5 master mix (NEB, Cat #M0544L), and water to a total 25 μL reaction volume. The PCR cycling conditions were 98° C. for 3 minutes. 15 cycles of 94° C. for 20 seconds followed by 65° C.-57.5° C. for 20 seconds (0.5° C. incremental decreases per cycle), and 72° C. for 1 minute, and a subsequent 20 cycles at 94° C. for 20 seconds, 58° C. for 20 seconds and 72° C. for 1 minute, and a final 10 minute extension at 72° C. Samples were then diluted 1:200 and subsequently indexed using primers. Indexing reactions included 1 μL of the diluted sample, 2.5 μL of each the forward and reverse indexing primers at 10 μM each, 12.5 μL of NEB Q5 master mix, and water to a total 25 μL reaction volume. The indexing PCR cycling conditions were 98° C. for 30 seconds, followed by 98° C. for 10 seconds, 60° C. for 30 seconds, and 72° C. for 30 seconds for 12 cycles, and a final extension period at 72° C. for 2 minutes. Samples were quantified in a 96-well plate reader using the Quant-IT DNA high sensitivity assay kit (Invitrogen, Cat #Q33232) according to the manufacturer's protocol. Post pooling, samples were then SPRI purified, and quantified using an Agilent 4200 TapeStation. Samples were then sequenced on an Illumina MiniSeq with PE 300 reads.

### Pooled CRISPR Screen

### Lentiviral Transduction

Twenty-four hours post stimulation, lentivirus containing the TF library was added directly to cultured T cells in a drop-wise fashion and tilting the plates to distribute evenly, targeting a multiplicity of infection (MOI) of 0.4. After an additional 24 hours, excess lentivirus was removed from the supernatant and washed off the cells. Cells were then incubated at 37° C.

### Cas9-Ribonucleotide Protein (RNP) Preparation

Cas9 protein (MacroLab, Berkeley, 40 μM stock) was delivered into the cells using a modified Guide Swap technique (Ting P Y, et al. 2018). To do this, on the day of electroporation, lyophilized Dharmacon Edit-R crRNA Non-targeting Control #3 (Dharmacon, Cat #U-007503-01-05) and Dharmacon Edit-R CRISPR-Cas9 Synthetic tracrRNA (Dharmacon, Cat #U-002005-20) were resuspended at a stock concentration of 160 mM in 10 mM Tris-HCl (pH 7.4) with 150 mM KCl. They were mixed at a 1:1 ratio, creating an 80 mM solution, and incubated on a heat block at 37° C. for 30 minutes. Single-stranded donor oligonucleotides (ssODN; sequence: TTAGCTCTGTTTACGTCCCAGCGGGCATGAGAGTAACAAGAGGGTGTGGTAATAT TACGGTACCGAGCACTATCGATACAATATGTGTCATACGGACACG) (SEQ ID NO: 2577) was then added at a 1:1 molar ratio of the final Cas9-Guide complex, and mixed well by pipetting. The solution was incubated for an additional 5 minutes at 37° C. on the heat block. Cas9 was then added slowly at a 1:1 volume to volume ratio, taking care to avoid precipitation, pipetting up and down several times to ensure complete resuspension of the RNP complex, and incubated at 37° C. for 15 minutes completing the process of creating the assembled RNP-ssODN complex.

### Electroporation

24 hours after virus was washed from the culture, cells were centrifuged at 100 g for 10 minutes to pellet them, and resuspended in room temperature Lonza P3 electroporation buffer (Lonza, Cat #V4XP-3032) at 1-2E6 cells per 17.8 μL. 7.2 μL of the RNP-ssODN complex were added for every 17.8 μL of cells and mixed well. Using a multichannel pipette, 23 μL of the cells-RNP-ssODN mixture were added per well to a 96 well electroporation cuvette plate (Lonza, Cat #VVPA-1002), and nucleofected using the pulse code EH-115. Immediately after electroporation, 90 μL of prewarmed media were added to each well and incubated at 37° C. for 15 minutes. Cells were then pooled, transferred to incubation flasks, and diluted with pre-warmed media to a final concentration of 1E⁶ cells/mL and incubated at 37° C. Cells were passaged at 48 hours post electroporation, and subsequently maintained in culture at 1E6 cells/mL.

### Results

FIG. 7A shows a xchematic of synthetic crRNA/Cas9 ribonucleoprotein arrayed knockout (KO) followed by in depth characterization of the KOs. FIG. 7B shows representative flow cytometry density plots for top hits in the IL2RA, IL-2, and CTLA4 screens. All plots were normalized to a maximum height of 1. KO of hits that decrease target levels are shown in orange and KO of hits that increase target levels are shown in blue.

FIGS. 7C-E show flow cytometry results for IL2RA, IL-2, and CTLA4 5 days after arrayed RNP KO. Screen hits analyzed are displayed on the Y axis ordered by their effect size in the pooled CRISPR screen. Changes in IL2RA, IL-2, and CTLA4 median fluorescence intensity relative to non-targeting controls is shown on the X-axis. Dots represent individual data points, bars depict average, and error bars depict standard deviation across 2 guide RNAs and 3 donors per guide RNA. Bars are colored by whether the flow cytometry effect matched the pooled CRISPR screen effect and whether the KO increased or decreased the level of IL2RA, IL-2, or CTLA4. The average insertion/deletion (indel) percentage across multiple donors for guide RNA 1 (n=3) and guide RNA 2 (n=2) at the genomic target site is shown to the right of each graph.

As shown in FIG. 7C, knockout of MED12, CBFB, HIVEP2, KLF2, MYB, FOXK1, ZNF217, IRF2, TNFAIP3, MYC, PRDM1, TFDP1, IRF1, FOXO1, ATXN7L3 or TP53, increased expression of IL2RA in cells. FIG. 7C also shows that knockout of IKZF3, YY1, MBD2, IRF4, IKZF1, RXRB, RELA, ETS1, KMT2A, PTEN, JAK3, STAT5A, GATA3, FOXP1, STAT5B, or IL2RA decreased expression of IL2RA in cells.

As shown in FIG. 7D, knockout of MED12, FOXP1, PTEN, IKZF1, TAF5L, PRDM1, TFDP1, CXXC1, IKZF3 or TP53 increased expression of IL2 in cells. FIG. 7D also shows that knockout of NFATC2, MAF, ZBTB7A, MBD2, GATA3, MED14, IRF2, MED30, ZBTB11, RELA, JAK3, MED11, BCL11B, MTF1, ATXN7L3, YY1, ETS1, IL2, DNMT1, GTF2B or SMARCB1, decreased expression of IL2 in cells.

As shown in FIG. 7E, knockout of CBFB, MYB, ZNF217, FOXK1, FLI1, FOS, SATB1, 1L2 or ATXN7L3, increased CTLA4 expression in cells. FIG. 7E also shows that knockout of MTF1, RELA, IRF1, BCL11B, STAT3, MED30, MED14, MED11, IKZF3, KMT2A, IKZF1, MED12, TAF5L, PTEN, IRF4, FOXO1, FOXP1 or CTLA4 decreased expression of CTLA4 in cells.

These studies also shows that knockout of ETS1, MYBL2, MYB, TP53, FLI1, SATB1, MBD2, ZBTB7A, DNMT1, TFDP1, SMARCB1 or MAF, increased expression of FOXP3 in cells (FIG. 7F). Further, knockout of TAF5L, FOXP3, GATA3, STAT5B, FOXP1, STAT5A, PTEN or FOXO1 decreased expression of FOXP3 in cells.

1. A T cell comprising:
(a) a genetic modification or heterologous polynucleotide that inhibits expression of CBFB, MYB, ZNF217, FOXK1, FLI1, FOS, SATB1, IL2, ATXN7L3, MTF1, RELA, IRF1, BCL11B, STAT3, MED30, MED14, MED11, IKZF3, KMT2A, IKZF1, MED12, TAF5L, PTEN, IRF4, FOXO1, FOXP1, CTLA4, ETS1, MYBL2, TP53, MBD2, ZBTB7A, DNMT1, HIVEP2, KLF2, TFDP1, SMARCB1, MAF, FOXP3, GATA3, STAT5B, STAT5A, PRDM1, TNFAIP3, RXRB, TFDP1, CXXC1, NFATC2, MAF, IRF2, ZBTB11, JAK3, YY1, IL2RA or GTF2B; and/or
(b) a heterologous polynucleotide that encodes CBFB, MYB, ZNF217, FOXK1, FLI1, FOS, SATB1, IL2, ATXN7L3, MTF1, RELA, IRF1, BCL11B, STAT3, MED30, MED14, MED11, IKZF3, KMT2A, IKZF1, MED12, TAF5L, PTEN, IRF4, FOXO1, FOXP1, CTLA4, ETS1, MYBL2, TP53, MBD2, ZBTB7A, DNMT1, TFDP1, SMARCB1, MAF, FOXP3, GATA3, STAT5B, STAT5A, PRDM1, TNFAIP3, RXRB, TFDP1, CXXC1, NFATC2, MAF, IRF2, ZBTB11, JAK3, YY1, IL2RA or GTF2B.

2. The T cell of claim 1, wherein the T cell comprises:
(a) a genetic modification or a heterologous polynucleotide that inhibits expression of CBFB, MYB, ZNF217, FOXK1, FLI1, FOS, SATB1, 1L2 or ATXN7L3, wherein expression of CTLA4 is increased in the T cell relative to expression of CTLA4 in a T cell not comprising the genetic modification or the heterologous polynucleotide that inhibits expression of CBFB, MYB, ZNF217, FOXK1, FLI1, FOS, SATB1, 1L2 or ATXN7L3; and/or
(b) a heterologous polynucleotide that encodes MTF1, RELA, IRF1, BCL11B, STAT3, MED30, MED14, MED11, IKZF3, KMT2A, IKZF1, MED12, TAF5L, PTEN, IRF4, FOXO1, FOXP1 or CTLA4, wherein expression of CTLA4 is increased in the T cell relative to expression of CTLA4 in a T cell not comprising the heterologous polynucleotide that encodes MTF1, RELA, IRF1, BCL11B, STAT3, MED30, MED14, MED11, IKZF3, KMT2A, IKZF1, MED12, TAF5L, PTEN, IRF4, FOXO1, FOXP1 or CTLA4.

3. The T cell of claim 1, wherein the T cell comprises:
(a) a genetic modification or heterologous polynucleotide that inhibits expression of MTF1, RELA, IRF1, BCL11B, STAT3, MED30, MED14, MED11, IKZF3, KMT2A, IKZF1, MED12, TAF5L, PTEN, IRF4, FOXO1, FOXP1 or CTLA4, wherein expression of CTLA4 is decreased in the T cell relative to expression of CTLA4 in a T cell not comprising the genetic modification or the heterologous polynucleotide that inhibits expression of MTF1, RELA, IRF1, BCL11B, STAT3, MED30, MED14, MED11, IKZF3, KMT2A, IKZF1, MED12, TAF5L, PTEN, IRF4, FOXO1, FOXP1 or CTLA4; and/or
(b) a heterologous polynucleotide that encodes CBTB, MYB, ZNF217, FOXK1, FLI1, FOX, SATB1, 1L2 or ATXN7L3, wherein expression of CTLA4 is decreased in the T cell relative to expression of CTLA4 in a T cell not comprising the heterologous polynucleotide that encodes CBTB, MYB, ZNF217, FOXK1, FLI1, FOX, SATB1, IL2 or ATXN7L3.

4. The T cell of claim 1, wherein the T cell comprises:
(a) a genetic modification or heterologous polynucleotide that inhibits expression of ETS1, MYBL2, MYB, TP53, FLI1, SATB1, MBD2, ZBTB7A, DNMT1, TFDP1, SMARCB1 or MAF, wherein expression of FOXP3 is increased in the T cell relative to expression of FOXP3 in a T cell not comprising the genetic modification or heterologous polynucleotide that inhibits expression of ETS1, MYBL2, MYB, TP53, FLI1, SATB1, MBD2, ZBTB7A, DNMT1, TFDP1, SMARCB1 or MAF; and/or
(b) a heterologous polynucleotide that encodes a TAF5L, FOXP3, GATA3, STAT5B, FOXP1, STAT5A, PTEN or FOXO1, wherein expression of FOXP3 is increased in the T cell relative to expression of FOXP3 in a T cell not comprising a heterologous polynucleotide that encodes a TAF5L, FOXP3, GATA3, STAT5B, FOXP1, STAT5A, PTEN or FOXO1.

5. The T cell of claim 1, wherein the T cell comprises:
(a) a genetic modification or heterologous polynucleotide that inhibits expression of TAF5L, FOXP3, GATA3, STAT5B, FOXP1, STAT5A, PTEN or FOXO1, wherein expression of FOXP3 is decreased in the T cell relative to expression of FOXP3 in a T cell not comprising the genetic modification or heterologous polynucleotide that inhibits expression of TAF5L, FOXP3, GATA3, STAT5B, FOXP1, STAT5A, PTEN or FOXO1; and/or
(b) a heterologous polynucleotide that encodes ETS1, MYBL2, MYB, TP53, FLI1, SATB1, MBD2, ZBTB7A, DNMT1, TFDP1, SMARCB1 or MAF, wherein expression of FOXP3 is decreased in the T cell relative to expression of FOXP3 in a T cell not comprising a heterologous polynucleotide that encodes ETS1, MYBL2, MYB, TP53, FLI1, SATB1, MBD2, ZBTB7A, DNMT1, TFDP1, SMARCB1 or MAF.

6. The T cell of claim 1, wherein the T cell comprises:
(a) a genetic modification or heterologous polynucleotide that inhibits expression of MED12, FOXP1, PTEN, IKZF1, TAF5L, PRDM1, TFDP1, CXXC1, IKZF3 or TP53, wherein expression of IL-2 is increased in the T cell relative to expression of IL-2 in a T cell not comprising the genetic modification or heterologous polynucleotide that inhibits expression of MED12, FOXP1, PTEN, IKZF1, TAF5L, PRDM1, TFDP1, CXXC1, IKZF3 or TP53; and/or
(b) a heterologous polynucleotide that encodes NFATC2, MAF, ZBTB7A, MBD2, GATA3, MED14, IRF2, MED30, ZBTB11, RELA, JAK3, MED11, BCL11B, MTF1, ATXN7L3, YY1, ETS1, IL2, DNMT1, GTF2B or SMARCB1, wherein expression of IL-2 is increased in the T cell relative to expression of IL-2 in a T cell not comprising heterologous polynucleotide that encodes NFATC2, MAF, ZBTB7A, MBD2, GATA3, MED14, IRF2, MED30, ZBTB11, RELA, JAK3, MED11, BCL11B, MTF1, ATXN7L3, YY1, ETS1, IL2, DNMT1, GTF2B or SMARCB1.

7. The T cell of claim 1, wherein the T cell comprises:
(a) genetic modification or heterologous polynucleotide that inhibits expression of NFATC2, MAF, ZBTB7A, MBD2, GATA3, MED14, IRF2, MED30, ZBTB11, RELA, JAK3, MED11, BCL11B, MTF1, ATXN7L3, YY1, ETS1, IL2, DNMT1, GTF2B or SMARCB1, wherein expression of IL-2 is decreased in the T cell relative to expression of IL-2 in a T cell not comprising the genetic modification or heterologous polynucleotide that inhibits expression of NFATC2, MAF, ZBTB7A, MBD2, GATA3, MED14, IRF2, MED30, ZBTB11, RELA, JAK3, MED11, BCL11B, MTF1, ATXN7L3, YY1, ETS1, IL2, DNMT1, GTF2B or SMARCB1; and/or
(b) a heterologous polynucleotide that encodes MED12, FOXP1, PTEN, IKZF1, TAF5L, PRDM1, TFDP1, CXXC1, IKZF3 or TP53, wherein expression of IL-2 is decreased in the T cell relative to expression of IL-2 in a T cell not comprising heterologous polynucleotide that encodes MED12, FOXP1, PTEN, IKZF1, TAF5L, PRDM1, TFDP1, CXXC1, IKZF3 or TP53.

8. The T cell of claim 1, wherein the T cell comprises:
(a) a genetic modification or heterologous polynucleotide that inhibits expression of MED12, CBFB, HIVEP2, KLF2, MYB, FOXK1, ZNF217, IRF2, TNFAIP3, MYC, PRDM1, TFDP1, IRF1, FOXO1, ATXN7L3 or TP53, wherein expression of IL2RA is increased in the T cell relative to expression of IL2RA in a T cell not comprising the genetic modification or heterologous polynucleotide that inhibits expression of MED12, CBFB, HIVEP2, KLF2, MYB, FOXK1, ZNF217, IRF2, TFNAIP3, MYC, PRDM1, TFDP1, IRF1, FOXO1, ATXN7L3 or TP53; and/or
(b) a heterologous polynucleotide that encodes IKZF3, YY1, MBD2, IRF4, IKZF1, RXRB, RELA, ETS1, KMT2A, PTEN, JAK3, STAT5A, GATA3, FOXP1, STAT5B, or IL2RA, wherein expression of IL2RA is increased in the T cell relative to expression of IL2RA in a T cell not comprising the heterologous polynucleotide that encodes IKZF3, YY1, MBD2, IRF4, IKZF1, RXRB, RELA, ETS1, KMT2A, PTEN, JAK3, STAT5A, GATA3, FOXP1, STAT5B, or IL2RA.

9. The T cell of claim 1, wherein the T cell comprises:
(a) a genetic modification or heterologous polynucleotide that inhibits expression of IKZF3, YY1, MBD2, IRF4, IKZF1, RXRB, RELA, ETS1, KMT2A, PTEN, JAK3, STAT5A, GATA3, FOXP1, STAT5B, or IL2RA, wherein expression of IL2RA is decreased in the T cell relative to expression of IL2RA in a T cell not comprising the genetic modification or heterologous polynucleotide that inhibits expression of IKZF3, YY1, MBD2, IRF4, IKZF1, RXRB, RELA, ETS1, KMT2A, PTEN, JAK3, STAT5A, GATA3, FOXP1, STAT5B, or IL2RA; and/or
(b) a heterologous polynucleotide that encodes MED12, CBFB, HIVEP2, KLF2, MYB, FOXK1, ZNF217, IRF2, TFNAIP3, MYC, PRDM1, TFDP1, IRF1, FOXO1, ATXN7L3 or TP53, wherein expression of IL2RA is decreased in the T cell relative to expression of IL2RA in a T cell not comprising heterologous polynucleotide that encodes MED12, CBFB, HIVEP2, KLF2, MYB, FOXK1, ZNF217, IRF2, TFNAIP3, MYC, PRDM1, TFDP1, IRF1, FOXO1, ATXN7L3 or TP53.

10. The T cell of claim 1, wherein the T cell is a Treg cell.

11. The T cell of claim 1, wherein the T cell is a CD8+ or a CD4+ T cell.

12. A population of cells comprising the genetically modified T cell of claim 1.

13. A method of making a modified T cell, the method comprising:
(a) inhibiting expression of one or more nuclear factors selected from the group consisting of CBFB, MYB, ZNF217, FOXK1, FLI1, FOS, SATB1, IL2, ATXN7L3, MTF1, RELA, IRF1, BCL11B, STAT3, MED30, MED14, MED11, IKZF3, KMT2A, IKZF1, MED12, TAF5L, PTEN, IRF4, FOXO1, FOXP1, CTLA4, ETS1, MYBL2, TP53, MBD2, ZBTB7A, DNMT1, HIVEP2, KLF2, TFDP1, SMARCB1, MAF, FOXP3, GATA3, STAT5B, STAT5A, PRDM1, TNFAIP3, RXRB, TFDP1, CXXC1, NFATC2, MAF, IRF2, ZBTB11, JAK3, YY1, IL2RA and GTF2B; and/or
(b) overexpressing one or more nuclear factors selected from the group consisting of CBFB, MYB, ZNF217, FOXK1, FLI1, FOS, SATB1, IL2, ATXN7L3, MTF1, RELA, IRF1, BCL11B, STAT3, MED30, MED14, MED11, IKZF3, KMT2A, IKZF1, MED12, TAF5L, PTEN, IRF4, FOXO1, FOXP1, CTLA4, ETS1, MYBL2, TP53, MBD2, ZBTB7A, DNMT1, HIVEP2, KLF2, TFDP1, SMARCB1, MAF, FOXP3, GATA3, STAT5B, STAT5A, PRDM1, TNFAIP3, RXRB, TFDP1, CXXC1, NFATC2, MAF, IRF2, ZBTB11, JAK3, YY1, IL2RA and GTF2B.

14. The method of claim 13, wherein the inhibiting comprises reducing expression of the nuclear factor, or reducing expression of a polynucleotide encoding the nuclear factor.

15.-24. (canceled)

25. The method of claim 13, wherein the T cell is obtained from a human prior to treating the T cell to inhibit expression of the nuclear factor, and the treated T cell is reintroduced into a human.

26. The method of claim 25, wherein the T cell is a Treg cell.

27. The method of claim 25, wherein the T cell is a is a CD8+ or a CD4+ T cell.

28. The method of claim 25, wherein expression of one or more nuclear factors selected from the group consisting of CBFB, MYB, ZNF217, FOXK1, FLI1, FOS, SATB1, 1L2, and ATXN7L3 is inhibited in the T cell.

29.-41. (canceled)

42. A method of modifying T cells in a subject in need thereof, comprising inhibiting expression of a one or more nuclear factors selected from the group consisting of CBFB, MYB, ZNF217, FOXK1, FLI1, FOS, SATB1, IL2, ATXN7L3, MTF1, RELA, IRF1, BCL11B, STAT3, MED30, MED14, MED11, IKZF3, KMT2A, IKZF1, MED12, TAF5L, PTEN, IRF4, FOXO1, FOXP1, CTLA4, ETS1, MYBL2, TP53, MBD2, ZBTB7A, DNMT1, HIVEP2, KLF2, TFDP1, SMARCB1, MAF, FOXP3, GATA3, STAT5B, STAT5A, PRDM1, TNFAIP3, RXRB, TFDP1, CXXC1, NFATC2, MAF, IRF2, ZBTB11, JAK3, YY1, IL2RA and GTF2B in the human T cells of the subject.

43. (canceled)

44. The method of claim 42, wherein the method comprises:
a) obtaining T cells from the subject;
b) modifying the T cells by inhibiting expression of one or more nuclear factors selected from the group consisting of MTF1, RELA, IRF1, BCL11B, STAT3, MED30, MED14, MED11, IKZF3, KMT2A, IKZF1, TAF5L, IRF4, FOXP1, CTLA4, FOXP3, GATA3, STAT5B, STAT5A, PTEN, FOXO1, MED12, FOXP1, PTEN, IKZF1, TAF5L, PRDM1, TFDP1, CXXC1, IKZF3, TP53, CBFB, HIVEP2, KLF2, MYB, FOXK1, ZNF217, IRF2, TFNAIP3, MYC, PRDM1, TFDP1, IRF1, ATXN7L3 and TP53; and
c) administering the T cells to the subject.

45. The method of claim 42, wherein the method comprises:
a) obtaining T cells from the subject;
b) modifying the T cells by overexpressing one or more nuclear factors selected from the group consisting of CBFB, MYB, ZNF217, FOXK1, FLI1, FOS, IL2, ATXN7L3, ETS1, MYBL2, MYB, TP53, FLI1, SATB1, ZBTB7A, DNMT1, TFDP1, SMARCB1, MAF, NFATC2, MAF, ZBTB7A, MED14, IRF2, MED30, ZBTB11, MED11, BCL11B, MTF1, ATXN7L3, YY1, ETS1, IL2, DNMT1, GTF2B, IKZF3, MBD2, IRF4, IKZF1, RXRB, RELA, ETS1, KMT2A, PTEN, JAK3, STAT5A, GATA3, FOXP1, STAT5B and IL2RA; and
c) administering the T cells to the subject.

46. The method of claim 44, wherein the subject has cancer.

47. The method of claim 42, wherein the method comprises:
a) obtaining T cells from the subject;
b) modifying the T cells by inhibiting expression of one or more nuclear factors selected from the group consisting of CBFB, MYB, ZNF217, FOXK1, FLI1, FOS, IL2, ATXN7L3, ETS1, MYBL2, MYB, TP53, FLI1, SATB1, ZBTB7A, DNMT1, TFDP1, SMARCB1, MAF, NFATC2, MAF, ZBTB7A, MED14, IRF2, MED30, ZBTB11, MED11, BCL11B, MTF1, ATXN7L3, YY1, ETS1, IL2, DNMT1, GTF2B, IKZF3, MBD2, IRF4, IKZF1, RXRB, RELA, ETS1, KMT2A, PTEN, JAK3, STAT5A, GATA3, FOXP1, STAT5B and IL2RA; and
c) administering the T cells to the subject.

48. The method of claim 42, wherein the method comprises:
a) obtaining T cells from the subject;
b) modifying the T cells by overexpressing one or more nuclear factors selected from the group consisting of MTF1, RELA, IRF1, BCL11B, STAT3, MED30, MED14, MED11, IKZF3, KMT2A, IKZF1, TAF5L, IRF4, FOXP1, CTLA4, FOXP3, GATA3, STAT5B, STAT5A, PTEN, FOXO1, MED12, FOXP1, PTEN, IKZF1, TAF5L, PRDM1, TFDP1, CXXC1, IKZF3, TP53, CBFB, HIVEP2, KLF2, MYB, FOXK1, ZNF217, IRF2, TFNAIP3, MYC, PRDM1, TFDP1, IRF1, ATXN7L3 and TP53; and
c) administering the T cells to the subject.

49. The method of claim 47, wherein the subject has an autoimmune disorder.

50.-97. (canceled)
